(19) United States
(12) Patent Application Publication
(10) Pub. No.: US 2022/0332701 A1
(43) Pub. Date: Oct. 20, 2022

(54) HETEROCYCLIC COMPOUNDS AS BCR-ABL INHIBITORS

(71) Applicants: ASCENTAGE PHARMA (SUZHOU) CO., LTD., SUZHOU (CN); ASCENTAGE PHARMA GROUP CORP LIMITED, Hong Kong (CN)

(72) Inventors: Yunlong Zhou, SUZHOU (CN); Guozhi TANG, SUZHOU (CN); Chao LI, SUZHOU (CN); Fang LIU, SUZHOU (CN); Yu JING, SUZHOU (CN); Renlin WANG, SUZHOU (CN); Lingling JIAO, SUZHOU (CN)

(21) Appl. No.: 17/262,533

(22) PCT Filed: Jul. 29, 2020
(86) PCT No.: PCT/CN2020/105519

§ 371 Date: Jan. 22, 2021

(30) Foreign Application Priority Data
Jul. 29, 2019 (CN) PCT/CN2019/098229

(51) Int. Cl. C07D 403/04; C07D 413/04; C07D 401/04; A61P 35/00

(52) U.S. Cl. CPC C07D 403/04; C07D 413/04; C07D 401/04; A61P 35/00

(57) ABSTRACT

The present disclosure provides compounds represented by Formula I:

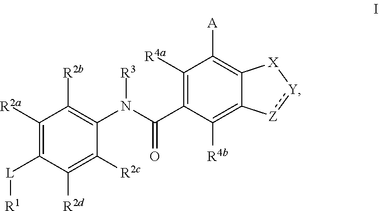

wherein R¹, R^{2a}, R^{2b}, R^{2c}, R^{2d}, R³, R^{4a}, R^{4b}, A, L, X, Y, Z, and [symbol] are as defined in the specification, and the pharmaceutically acceptable salts and solvates thereof. Compounds of Formula I are BCR-ABL inhibitors. BCR-ABL inhibitors are useful for the treatment of cancer and other diseases.

### BACKGROUND OF THE INVENTION

### Field of the Invention

The present disclosure provides heterocyclic compounds that inhibit the enzymatic activity of the Abelson protein (ABL1), Abelson-related protein (ABL2), and related chimeric proteins, in particular BCR-ABL1. The disclosure also provides processes for preparing these compounds, pharmaceutical compositions comprising these compounds, and methods of using these compounds to treat diseases, disorders, or conditions responsive to inhibition of BCR-ABL1.

### Background

Abelson murine leukemia viral oncogene homolog 1 also known as ABL1 is a protein that, in humans, is encoded by the ABL1 gene located on chromosome 9. The ABL1 proto-oncogene encodes a cytoplasmic and nuclear protein tyrosine kinase that is involved in processes of cell differentiation, cell division, cell adhesion and stress response. Activity of ABL1 protein is self-inhibited by its SH3 domain, and deletion of the SH3 domain turns ABL1 into an oncogene. The hallmark of chronic myelogenous leukemia (CML) is the Philadelphia chromosome (Ph), formed by the t(9;22) translocation which causes the expression of the BCR-ABL tyrosine kinase fusion gene. This fusion gene encodes the chimeric BCR-ABL protein, that loses the self-regulatory of the SH3 domain.

Although there are effective drugs in the treatment of CML by inhibiting the tyrosine kinase activity of BCR-ABL via an ATP-competitive mechanism, such as Imatinib, Nilotinib, Dasatinib, and Bosutinib, some patients relapse due to the emergence of drug-resistant clones, in which mutations in the SH1 comprise inhibitor binding. Thus compounds that inhibit the BCR-ABL protein activities via a different binding mode have the potential to overcome the resistance and expanding the treatment choice for AML patients.

Agents targeting the myristoyl binding site (referred as allosteric inhibitors) have potential for the treatment of BCR-ABL disorders (Zhang et al., Nature, 2010, 463:501-6). Potentially, an allosteric inhibitor that binds to the myristoyl binding site might be useful to prevent the emergence of drug resistance from ATP inhibitors. More importantly, a combination treatment using both types of inhibitor can be developed for the treatment of BCR-ABL related disorders (Wylie et al., Nature, 2017, 543: 733-7).

There exist a need in the art for BCR-ABL inhibitors.

### BRIEF SUMMARY OF THE INVENTION

In one aspect, the present disclosure provides compounds represented by any one of Formulae I, II-A, II-B, or III-XIII, below, and the pharmaceutically acceptable salts and solvates, e.g., hydrates, thereof, collectively referred to as “Compounds of the Disclosure.” Compounds of the Disclosure are BCR-ABL inhibitors and/or synthetic intermediates used to prepare BCR-ABL inhibitors. BCR-ABL inhibitors are useful in treating or preventing diseases or conditions such as cancer wherein the inhibition of BCR-ABL protein provides a benefit.

In another aspect, the present disclosure provides methods of treating or preventing a condition or disease by administering a therapeutically effective amount of a Compound of the Disclosure to a subject, e.g., a human patient, in need thereof. The disease or condition of interest that is treatable or preventable by inhibition or of BCR-ABL is, for example, cancer, neurodegenerative diseases, muscular dystrophies, autoimmune, diseases, inflammatory diseases, viral infections, and prion diseases. Also provided are methods of preventing the proliferation of unwanted proliferating cells, such as in cancer, in a subject comprising administering a therapeutically effective amount of a Compound of the Disclosure to a subject at risk of developing a condition characterized by unwanted proliferating cells. In some embodiments, the Compounds of the Disclosure may reduce the proliferation of unwanted cells by inhibiting their driving oncogene.

In another aspect, the present disclosure provides a method of inhibiting BCR-ABL in a subject, comprising administering to the subject a therapeutically effective amount of a Compound of the Disclosure.

In another aspect, the present disclosure provides a pharmaceutical composition comprising a Compound of the Disclosure and an excipient and/or pharmaceutically acceptable carrier.

In another aspect, the present disclosure provides a composition comprising a Compound of the Disclosure and an excipient and/or pharmaceutically acceptable carrier for use treating or preventing diseases or conditions wherein inhibition of BCR-ABL provides a benefit, e.g., cancer e.g., chronic myeloid leukemia.

In another aspect, the present disclosure provides a composition comprising: (a) a Compound of the Disclosure; (b) a second therapeutically active agent; and (c) optionally an excipient and/or pharmaceutically acceptable carrier.

In another aspect, the present disclosure provides a Compound of the Disclosure for use in the treatment or prevention of a disease or condition of interest, e.g., cancer e.g., chronic myeloid leukemia.

In another aspect, the present disclosure provides a use of a Compound of the Disclosure for the manufacture of a medicament for treating a disease or condition of interest, e.g., cancer, e.g., chronic myeloid leukemia.

In another aspect, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

In another aspect, the present disclosure provides methods of preparing Compounds of the Disclosure and Intermediates of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF THE INVENTION

### I. Compounds of the Disclosure

Compounds of the Disclosure are BCR-ABL inhibitors and/or synthetic intermediates used to prepare BCR-ABL inhibitors.

In one embodiment, Compounds of the Disclosure are compounds of Formula I:

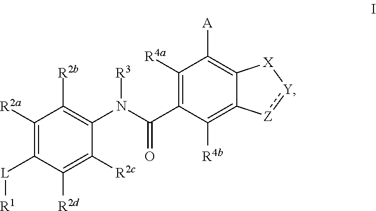

wherein:

R¹ is C₁-C₃ haloalkyl;

L is selected from the group consisting of —S— and —O—;

R^{2a}, R^{2b}, R^{2c}, and R^{2d} are independently selected from the group consisting of hydrogen, halo, C₁-C₃ alkyl, and C₁-C₃ alkoxy;

R³ is selected from the group consisting of hydrogen and C₁-C₃ alkyl;

R^{4a} and R^{4b} are independently selected from the group consisting of hydrogen, halo, C₁-C₃ alkyl, and C₁-C₃ alkoxy;

A is selected from the group consisting of optionally substituted 5-membered heteroaryl and optionally substituted 6-membered heteroaryl;

X is —C(R^{5a})(R^{5b})—; Y is —C(R^{5c})(R^{5d}); Z is —N(R^{5e})—; and [symbol] is a single bond;

R^{5a} and R^{5b} are independently selected from the group consisting of hydrogen and C₁-C₄ alkyl; or

R^{5a} and R^{5b} taken together with the carbon atom to which they are attached form an optionally substituted C₃-C₈ cycloalkyl or optionally substituted 4- to 8-membered heterocyclo;

R^{5c} and R^{5d} are independently selected from the group consisting of hydrogen and C₁-C₄ alkyl;

R^{5e} is selected from the group consisting of hydrogen, C₁-C₆ alkyl, hydroxyalkyl, (amino)alkyl, (heterocyclo)alkyl, optionally substituted C₃-C₆ cycloalkyl, optionally substituted 4- to 8-membered heterocyclo, —C(═O)R⁶, and —S(═O)₂R⁷;

R⁶ is selected from the group consisting of C₁-C₆ alkyl, optionally substituted C₃-C₆ cycloalkyl, and optionally substituted 4- to 8-membered heterocyclo;

R⁷ is selected from the group consisting of C₁-C₆ alkyl optionally substituted C₃-C₆ cycloalkyl, and optionally substituted 4- to 8-membered heterocyclo; or

X is —C(R^{8a})(R^{8b})—; Y is —N(R^{8c})—; Z is —C(═O)—; and [symbol] is a single bond;

R^{8a} and R^{8b} are independently selected from the group consisting of hydrogen and C₁-C₄ alkyl; or

R^{8a} and R^{8b} taken together with the carbon atom to which they are attached form an optionally substituted C₃-C₈ cycloalkyl or optionally substituted 4- to 8-membered heterocyclo;

R^{8c} is selected from the group consisting of hydrogen, optionally substituted C₁-C₆ alkyl, hydroxyalkyl, (amino)alkyl, optionally substituted C₃-C₆ cycloalkyl, optionally substituted 4- to 8-membered heterocyclo, and (heterocyclo)alkyl; or

X is —C(R^{9a})(R^{9b})—; Y is —C(═O)—; Z is —N(R^{9c})—; and [symbol] is a single bond;

R^{9a} and R^{9b} are independently selected from the group consisting of hydrogen and C₁-C₄ alkyl; or

R^{9a} and R^{9b} taken together with the carbon atom to which they are attached form an optionally substituted C₃-C₈ cycloalkyl or optionally substituted 4- to 8-membered heterocyclo;

R^{9c} is selected from the group consisting of hydrogen, optionally substituted C₁-C₆ alkyl, hydroxyalkyl, (amino)alkyl, optionally substituted C₃-C₆ cycloalkyl, optionally substituted 4- to 8-membered heterocyclo, and (heterocyclo)alkyl; or

X is —N(R^{10a})—; Y is —C(R^{10b})(R^{10c})—; Z is —C(R^{10d})(R^{10e})—; and [symbol] is a single bond;

R^{10a} is selected from the group consisting of hydrogen, C₁-C₆ alkyl, hydroxyalkyl, (amino)alkyl, (heterocyclo)alkyl, optionally substituted C₃-C₆ cycloalkyl, optionally substituted 4- to 8-membered heterocyclo, —C(═O)R^{11a}, and —S(═O)₂R^{12a};

R^{10b} is selected from the group consisting of hydrogen, —CO₂H, C₁-C₄ alkyl, hydroxyalkyl, (amino)alkyl, (heterocyclo)alkyl, optionally substituted C₃-C₆ cycloalkyl, optionally substituted 4- to 8-membered heterocyclo, —C(═O)R^{11b}, S(═O)₂R^{12b}, —(CH₂)ₘ—C(═O)N(R^{13a})(R^{13b}), and —(CH₂)ₙ—N(R¹⁴)C(═O)R¹⁵;

R^{10c} is selected from the group consisting of hydrogen and C₁-C₄ alkyl;

R^{10d} and R^{10e} are independently selected from the group consisting of hydrogen, C₁-C₄ alkyl, (amino)alkyl, and hydroxyalkyl;

R^{11a} is selected from the group consisting of C₁-C₆ alkyl, optionally substituted C₃-C₆ cycloalkyl, and optionally substituted 4- to 8-membered heterocyclo;

R^{11b} is selected from the group consisting of hydroxy, C₁-C₆ alkyl and optionally substituted C₃-C₆ cycloalkyl;

R^{12a} is selected from the group consisting of C₁-C₆ alkyl, optionally substituted C₃-C₆ cycloalkyl, and optionally substituted 4- to 8-membered heterocyclo;

R^{12b} is selected from the group consisting of C₁-C₆ alkyl, optionally substituted C₃-C₆ cycloalkyl, and optionally substituted 4- to 8-membered heterocyclo;

R^{13a} is selected from the group consisting of hydrogen, optionally substituted C₁-C₆ alkyl, alkoxyalkyl, (amino)alkyl, (heterocyclo)alkyl, substituted C₃-C₆ cycloalkyl, and optionally substituted 4- to 8-membered heterocyclo;

R^{13b} is selected from the group consisting of hydrogen, C₁-C₆ alkyl, and alkoxyalkyl; or

R^{13a} and R^{13b} taken together form an optionally substituted 4- to 8-membered heterocyclo;

R¹⁴ is selected from the group consisting of hydrogen and C₁-C₄ alkyl;

R¹⁵ is selected from the group consisting of C₁-C₆ alkyl, substituted C₃-C₆ cycloalkyl, optionally substituted 4- to 8-membered heterocyclo;

m is 0, 1, or 2; and

n is 0, 1, or 2; or

X is —N(R^{16a})—; Y is —C(═O)—; Z is —C(R^{16b})(R^{16c})—; and [symbol] is a single bond;

R^{16a} is selected from the group consisting of hydrogen, C₁-C₆ alkyl, and optionally substituted C₃-C₆ cycloalkyl;

R^{16b} is selected from the group consisting of hydrogen and C₁-C₄ alkyl;

R^{16c} is selected from the group consisting of hydrogen and C₁-C₄ alkyl; or

R^{16b} and R^{16c} taken together with the carbon atom to which they are attached form an optionally substituted C₃-C₈ cycloalkyl or optionally substituted 4- to 8-membered heterocyclo; or

X is —N(R^{17a})—; Y is —C(R^{17b})(R^{17c})ₒ—; Z is selected from the group consisting of —O—, —S—, —N(R^{17d})— and —C(R^{17e})(R^{17f})—; and [symbol] is a single bond;

R^{17a} is selected from the group consisting of hydrogen, C₁-C₆ alkyl, and optionally substituted C₃-C₆ cycloalkyl;

each R^{17b} is independently selected from the group consisting of hydrogen and C₁-C₄ alkyl;

each R^{17c} is independently selected from the group consisting of hydrogen and C₁-C₄ alkyl;

R^{17d} is selected from the group consisting of hydrogen, C₁-C₆ alkyl, and optionally substituted C₃-C₆ cycloalkyl;

R^{17e} is selected from the group consisting of hydrogen and C₁-C₄ alkyl;

R^{17f} is selected from the group consisting of hydrogen and C₁-C₄ alkyl; and

o is 1 or 2; or

X is —N(R^{18a})—; Y is —C(R^{18b})═; Z is —C(R^{18c})═; and [symbol] is a double bond;

R^{18a} is selected from the group consisting of hydrogen, C₁-C₆ alkyl, and optionally substituted C₃-C₆ cycloalkyl;

R^{18b} is selected from the group consisting of hydrogen and C₁-C₄ alkyl; and

R^{18c} is selected from the group consisting of hydrogen, halo, C₁-C₄ alkyl, and hydroxyalkyl; or

X is —N(R^{19a})—; Y is —N═; Z is —C(R^{19b})═; and [symbol] is a double bond;

R^{19a} is selected from the group consisting of hydrogen, C₁-C₆ alkyl, and optionally substituted C₃-C₆ cycloalkyl; and

R^{19b} is selected from the group consisting of hydrogen and C₁-C₄ alkyl, or a pharmaceutically acceptable salt or solvate thereof.

In another embodiment, Compounds of the Disclosure are compounds of Formula II-A:

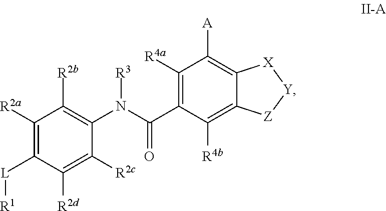

wherein:

X is —C(R^{5a})(R^{5b})—; Y is —C(R^{5c})(R^{5d}); and Z is —N(R^{5e})—; or

X is —C(R^{8a})(R^{8b})—; Y is —N(R^{8c})—; and Z is —C(═O)—; or

X is —C(R^{9a})(R^{9b})—; Y is —C(═O)—; and Z is —N(R^{9c})—; or

X is —N(R^{10a})—; Y is —C(R^{10b})(R^{10c})—; and Z is —C(R^{10d})(R^{10e})—; or

X is —N(R^{16a})—; Y is —C(═O)—; and Z is —C(R^{16b})(R^{16c})—; and

R¹, R^{2a}, R^{2b}, R^{2c}, R^{2d}, R³, R^{4a}, R^{4b}, R^{5a}, R^{5b}, R^{5c}, R^{5d}, R^{5e}, R^{8a}, R^{8b}, R^{5c}, R^{9a}, R^{9b}, R^{9c}, R^{10a}, R^{10b}, R^{10c}, R^{10d}, R^{10e}, R^{16a}, R^{16b}, R^{16c}, A, and L are as defined in connection with Formula I, or a pharmaceutically acceptable salt or solvate thereof.

In another embodiment, Compounds of the Disclosure are compounds of Formula II-B:

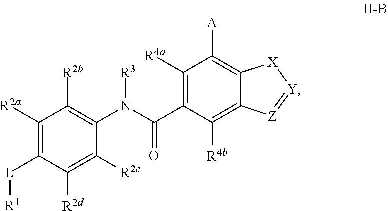

wherein:

X is —N(R^{18a})—; Y is —C(R^{18b})═; and Z is —C(R^{18c})═; or

X is —N(R^{19a})—; Y is —N═; and Z is —C(R^{19b})═; and

R¹, R^{2a}, R^{2b}, R^{2c}, R^{2a}, R³, R^{4a}, R^{4b}, R^{18a}, R^{18b}, R^{18c}, R^{19a}, R^{19b}, A, and L are as defined in connection with Formula I, or a pharmaceutically acceptable salt or solvate thereof.

In another embodiment, Compounds of the Disclosure are compounds of Formula III:

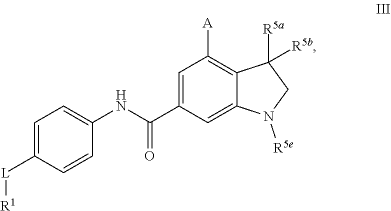

wherein R¹, R^{5a}, R^{5b}, R^{5e}, A, and L are as defined in connection with Formula I, or a pharmaceutically acceptable salt or solvate thereof.

In another embodiment, Compounds of the Disclosure are compounds of Formula III, wherein R^{5a} and R^{5b} are independently selected from the group consisting of hydrogen and C₁-C₄ alkyl, or a pharmaceutically acceptable salt or solvate thereof.

In another embodiment, Compounds of the Disclosure are compounds of Formula III, wherein R^{5e} is independently selected from the group consisting of hydrogen, C₁-C₆ alkyl, —C(═O)R⁶, and —S(═O)₂R⁷, or a pharmaceutically acceptable salt or solvate thereof.

In another embodiment, Compounds of the Disclosure are compounds of Formula IV:

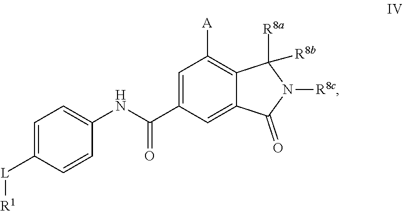

wherein R¹, R^{8a}, R^{8b}, R^{8c}, A, and L are as defined in connection with Formula I, or a pharmaceutically acceptable salt or solvate thereof.

In another embodiment, Compounds of the Disclosure are compounds of Formula IV, wherein R^{8a} and R^{8b} are independently selected from the group consisting of hydrogen and C₁-C₄ alkyl, or a pharmaceutically acceptable salt or solvate thereof.

In another embodiment, Compounds of the Disclosure are compounds of Formula IV, wherein R^{8c} is selected from the group consisting of optionally substituted C₁-C₆ alkyl, hydroxyalkyl, (amino)alkyl, C₃-C₆ cycloalkyl, optionally substituted 4- to 8-membered heterocyclo, and (heterocyclo)alkyl, or a pharmaceutically acceptable salt or solvate thereof.

In another embodiment, Compounds of the Disclosure are compounds of Formula IV, wherein R^{8c} is selected from the group consisting of:

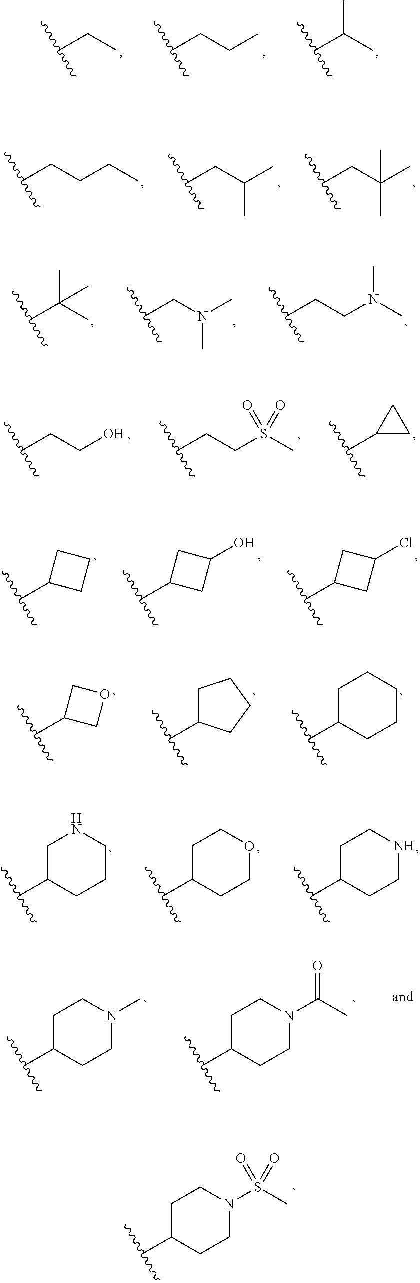

or a pharmaceutically acceptable salt or solvate thereof.

In another embodiment, Compounds of the Disclosure are compounds of Formula V:

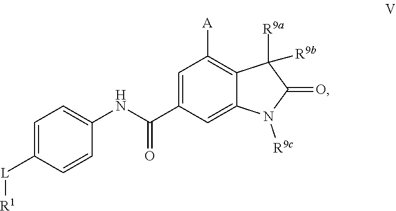

wherein R¹, R^{9a}, R^{9b}, R⁹¹, A, and L are as defined in connection with Formula I, or a pharmaceutically acceptable salt or solvate thereof.

In another embodiment, Compounds of the Disclosure are compounds of Formula V, wherein R^{9a} and R^{9b} are independently C₁-C₄ alkyl, or a pharmaceutically acceptable salt or solvate thereof.

In another embodiment, Compounds of the Disclosure are compounds of Formula V, wherein R^{9a} and R^{9b} are taken together with the carbon atom to which they are attached form an optionally substituted C₃-C₆ cycloalkyl, or a pharmaceutically acceptable salt or solvate thereof.

In another embodiment, Compounds of the Disclosure are compounds of Formula V, wherein R^{9a} and R^{9b} are taken together with the carbon atom to which they are attached form an optionally substituted 4- to 8-membered heterocyclo, or a pharmaceutically acceptable salt or solvate thereof.

In another embodiment, Compounds of the Disclosure are compounds of Formula V, wherein R^{9a} and R^{9b} are taken together with the carbon atom to which they are attached form:

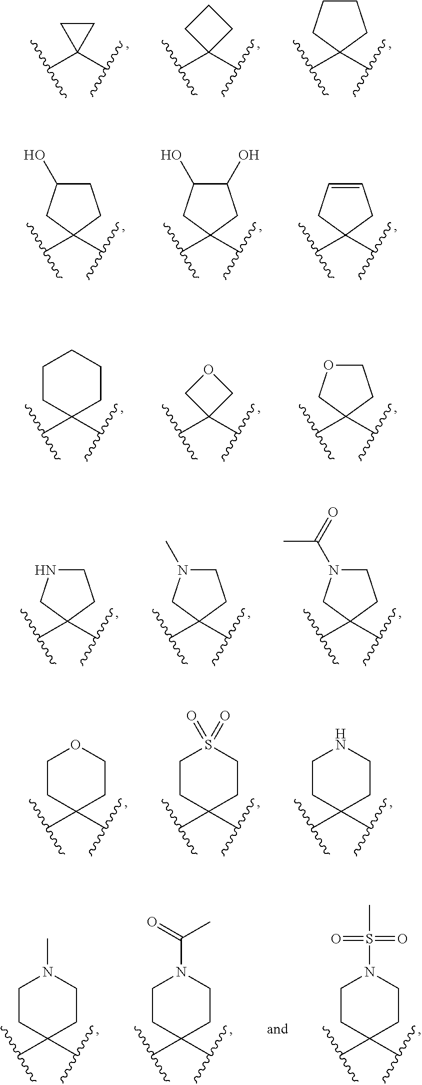

or a pharmaceutically acceptable salt or solvate thereof.

In another embodiment, Compounds of the Disclosure are compounds of Formula V, wherein R^{9c} is selected from the group consisting of hydrogen, C₁-C₆ alkyl, hydroxyalkyl, and (amino)alkyl, or a pharmaceutically acceptable salt or solvate thereof.

In another embodiment, Compounds of the Disclosure are compounds of Formula V, wherein R^{9c} is selected from the group consisting of:

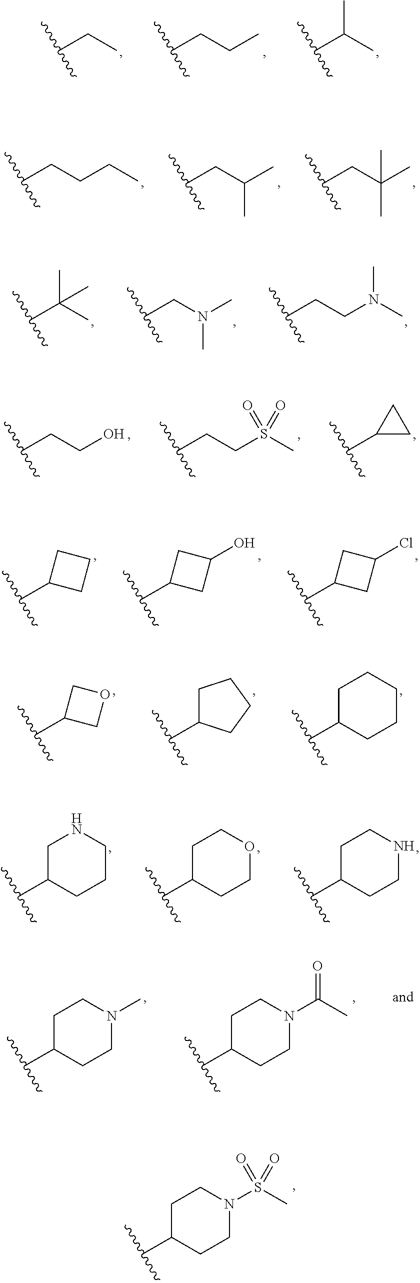

or a pharmaceutically acceptable salt or solvate thereof.

In another embodiment, Compounds of the Disclosure are compounds of Formula VI:

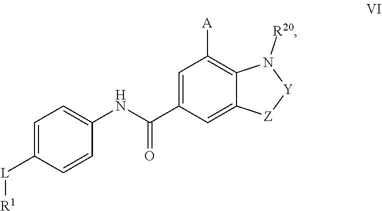

wherein:

R²⁰ is selected from the group consisting of hydrogen, C₁-C₆ alkyl, hydroxyalkyl, (amino)alkyl, (heterocyclo)alkyl, optionally substituted C₃-C₆ cycloalkyl, optionally substituted 4- to 8-membered heterocyclo,

Y is —C(R^{10b})(R^{10c})—; Z is —C(R^{10d})(R^{10e})—; or

Y is —C(═O)—; Z is —C(R^{16b})(R^{16c})—; and

R¹, R^{10b}, R^{10c}, R^{10d}, R^{10e}, R^{16b}, R^{16c} A, and L are as defined in connection with Formula I, or a pharmaceutically acceptable salt or solvate thereof.

In another embodiment, Compounds of the Disclosure are compounds of Formula VII:

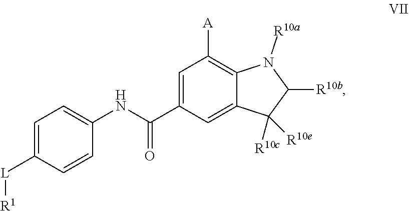

wherein R¹, R^{10a}, R^{10b}, R^{10d}, R^{10e}, A, and L are as defined in connection with Formula I, or a pharmaceutically acceptable salt or solvate thereof.

In another embodiment, Compounds of the Disclosure are compounds of Formula VII, wherein R^{10a} is selected from the group consisting of C₁-C₆ alkyl, hydroxyalkyl, (amino)alkyl, (heterocyclo)alkyl, optionally substituted C₃-C₆ cycloalkyl, —C(═O)R^{11a}, and —S(═O)₂R^{12a}, or a pharmaceutically acceptable salt or solvate thereof.

In another embodiment, Compounds of the Disclosure are compounds of Formula VII, wherein R^{10b} is selected from the group consisting of hydrogen, —CO₂H, C₁-C₄ alkyl, hydroxyalkyl, (amino)alkyl, (heterocyclo)alkyl, optionally substituted C₃-C₆ cycloalkyl, optionally substituted 4- to 8-membered heterocyclo, —(CH₂)ₘ—C(═O)N(R^{13a})(R^{13b}), and —(CH₂)ₙ—N(H)C(═O)R¹⁵; m is 0 or 1; and n is 0 or 1, or a pharmaceutically acceptable salt or solvate thereof.

In another embodiment, Compounds of the Disclosure are compounds of Formula VII, wherein R^{10b} is selected from the group consisting of

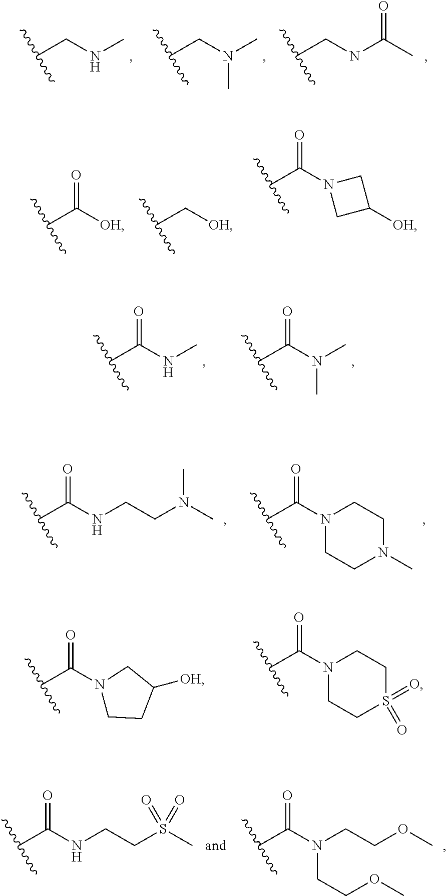

or a pharmaceutically acceptable salt or solvate thereof.

In another embodiment, Compounds of the Disclosure are compounds of Formula VII, wherein R^{10d} and R^{10e} are independently selected from the group consisting of hydrogen, C₁-C₃ alkyl, (amino)alkyl, and hydroxyalkyl, or a pharmaceutically acceptable salt or solvate thereof.

In another embodiment, Compounds of the Disclosure are compounds of Formula VIII:

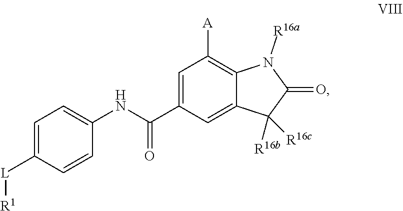

wherein R¹, R^{16a}, R^{16b}, R^{16c}, A, and L are as defined in connection with Formula I, or a pharmaceutically acceptable salt or solvate thereof.

In another embodiment, Compounds of the Disclosure are compounds of Formula VIII, wherein R^{16a} is selected from the group consisting of C₁-C₆ alkyl and C₃-C₆ cycloalkyl, or a pharmaceutically acceptable salt or solvate thereof.

In another embodiment, Compounds of the Disclosure are compounds of Formula VIII, wherein R^{16b} and R^{16c} are independently C₁-C₄ alkyl, or a pharmaceutically acceptable salt or solvate thereof.

In another embodiment, Compounds of the Disclosure are compounds of Formula IX:

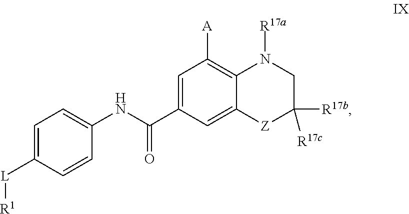

wherein R¹, R^{17a}, R^{17b}, R^{17c}, A, Z, and L are as defined in connection with Formula I, or a pharmaceutically acceptable salt or solvate thereof.

In another embodiment, Compounds of the Disclosure are compounds of Formula IX, wherein R^{17a} is C₁-C₆ alkyl, or a pharmaceutically acceptable salt or solvate thereof.

In another embodiment, Compounds of the Disclosure are compounds of Formula IX, wherein R^{17b} and R^{17c} are independently selected from the group consisting of hydrogen and C₁-C₄ alkyl, or a pharmaceutically acceptable salt or solvate thereof.

In another embodiment, Compounds of the Disclosure are compounds of Formula IX, wherein Z is —O— or —CH₂—, or a pharmaceutically acceptable salt or solvate thereof.

In another embodiment, Compounds of the Disclosure are compounds of Formula X:

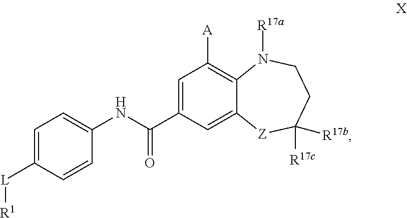

wherein R¹, R^{17a}, R^{17b}, R^{17c}, A, Z, and L are as defined in connection with Formula I, or a pharmaceutically acceptable salt or solvate thereof.

In another embodiment, Compounds of the Disclosure are compounds of Formula X, wherein R^{17a} is C₁-C₆ alkyl, or a pharmaceutically acceptable salt or solvate thereof.

In another embodiment, Compounds of the Disclosure are compounds of Formula X, wherein R^{17b} and R^{17c} are independently selected from the group consisting of hydrogen and C₁-C₄ alkyl, or a pharmaceutically acceptable salt or solvate thereof.

In another embodiment, Compounds of the Disclosure are compounds of Formula X, wherein Z is —C(R^{17e})(R^{17f})—, or a pharmaceutically acceptable salt or solvate thereof.

In another embodiment, Compounds of the Disclosure are compounds of Formula XI:

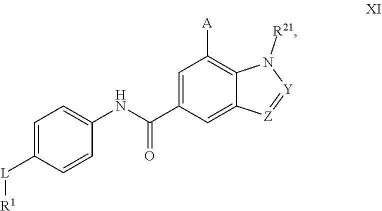

wherein:

R²¹ is selected from the group consisting of hydrogen, C₁-C₆ alkyl, and optionally substituted C₃-C₆ cycloalkyl;

Y is —C(R^{18b})═; Z is —C(R^{18c})═; or

Y is —N═; Z is —C(R^{19b})═;

R¹, R¹⁸, R^{18c}, R^{19b}, A, and L are as defined in connection with Formula I, or a pharmaceutically acceptable salt or solvate thereof.

In another embodiment, Compounds of the Disclosure are compounds of Formula XII:

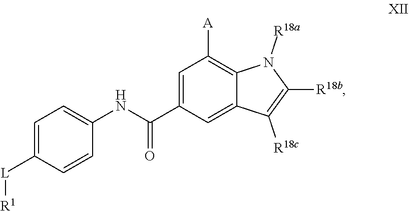

wherein R¹, R^{18a}, R^{18b}, R^{18c}, A, and L are as defined in connection with Formula I, or a pharmaceutically acceptable salt or solvate thereof.

In another embodiment, Compounds of the Disclosure are compounds of Formula XII, wherein R^{18a} is independently selected from the group consisting of hydrogen, C₁₋₄ alkyl, and C₃-C₆ cycloalkyl, or a pharmaceutically acceptable salt or solvate thereof

In another embodiment, Compounds of the Disclosure are compounds of Formula XII, wherein R^{18b} and R^{18c} are independently selected from the group consisting of hydrogen and C₁₋₄ alkyl, or a pharmaceutically acceptable salt or solvate thereof.

In another embodiment, Compounds of the Disclosure are compounds of Formula XIII:

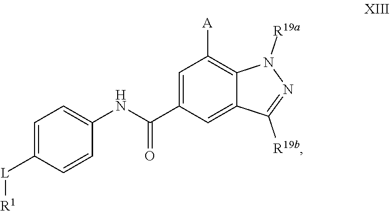

wherein R¹, R^{19a}, R^{19b}, A, and L are as defined in connection with Formula I, or a pharmaceutically acceptable salt or solvate thereof.

In another embodiment, Compounds of the Disclosure are compounds of Formula XIII, wherein R^{19a} is selected from the group consisting of hydrogen, C₁₋₄ alkyl, and C₃-C₆ cycloalkyl, or a pharmaceutically acceptable salt or solvate thereof

In another embodiment, Compounds of the Disclosure are compounds of Formula XIII, wherein R^{19b} is selected from the group consisting of hydrogen and C₁₋₄ alkyl, or a pharmaceutically acceptable salt or solvate thereof.

In another embodiment, Compounds of the Disclosure are compounds of any one of Formulae I-XIII, wherein L is —O—, or a pharmaceutically acceptable salt or solvate thereof.

In another embodiment, Compounds of the Disclosure are compounds of any one of Formulae I-XIII, wherein L is —S—, or a pharmaceutically acceptable salt or solvate thereof.

In another embodiment, Compounds of the Disclosure are compounds of any one of Formulae I-XIII, wherein R¹ is —CF₃, or a pharmaceutically acceptable salt or solvate thereof.

In another embodiment, Compounds of the Disclosure are compounds of any one of Formulae I-XIII, wherein R¹ is —CF₂Cl, or a pharmaceutically acceptable salt or solvate thereof.

In another embodiment, Compounds of the Disclosure are compounds of any one of Formulae I-XIII, wherein A is optionally substituted 5-membered heteroaryl, or a pharmaceutically acceptable salt or solvate thereof. In another embodiment, A is selected from the group consisting of:

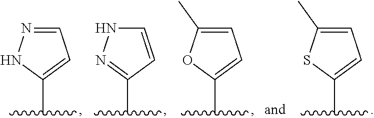

In another embodiment, A is:

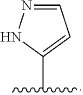 [SMILES: *c1ccn[nH]1]

In another embodiment, Compounds of the Disclosure are compounds of any one of Formulae I-XIII, wherein A is optionally substituted 6-membered heteroaryl, or a pharmaceutically acceptable salt or solvate thereof. In another embodiment, A is selected from the group consisting of:

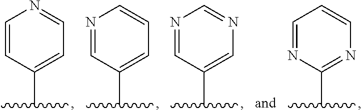

or a pharmaceutically acceptable salt or solvate thereof.

In another embodiment, Compounds of the Disclosure are any one or more of the compounds listed in Table 1, or a pharmaceutically acceptable salt or solvate thereof. The chemical names provided in Table 1 and in the Examples, below, were generated using ACDName v2015, Chemdraw® 18.1, or Chemdraw® Professional version 17.0.0.206. In the event of any ambiguity between their chemical structure and chemical name, Compounds of the Disclosure are defined by their structure.

TABLE 1

| Cpd. | | |
|---|---|---|
| No. | Structure | Name |
| III-1 | 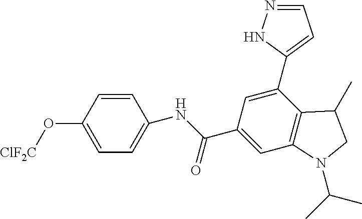 | N-(4-(chlorodifluoromethoxy)phenyl)- 1-isopropyl-3-methyl-4-(1H-pyrazol-5- yl)indoline-6-carboxamide |
| III-2 | 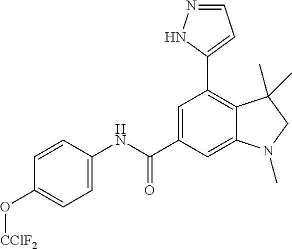 | N-(4-(chlorodifluoromethoxy)phenyl)- 1,3,3-trimethyl-4-(1H-pyrazol-5- yl)indoline-6-carboxamide |
| III-3 | 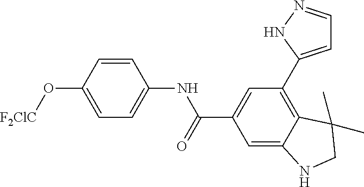 | N-(4-(chlorodifluoromethoxy)phenyl)-3,3- dimethyl-4-(1H-pyrazol-5-yl)indoline-6- carboxamide |
| III-4 | 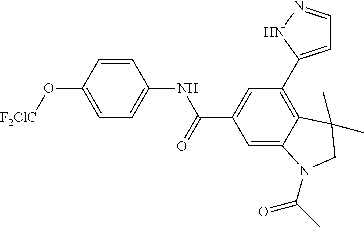 | 1-acetyl-N-(4- (chlorodifluoromethoxy)phenyl)-3,3- dimethyl-4-(1H-pyrazol-5-yl)indoline-6- carboxamide |
| III-5 | 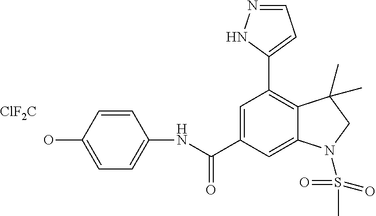 [SMILES: "ClF2C" ; CC1(C)CN(S(C)(=O)=O)c2cc(C(=O)Nc3ccc(O)cc3)cc(-c3ccn[nH]3)c21] | N-(4-(chlorodifluoromethoxy)phenyl)-3,3- dimethyl-1-(methylsulfonyl)-4-(1H- pyrazol-5-yl)indoline-6-carboxamide |
| IV-1 | 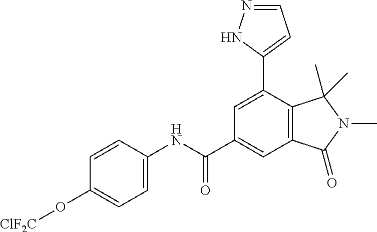 | N-(4-(chlorodifluoromethoxy)phenyl)- 1,1,2-trimethyl-3-oxo-7-(1H-pyrazol-5- yl)isoindoline-5-carboxamide |
| IV-2 | 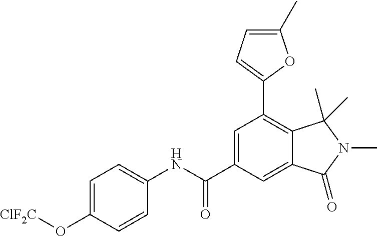 | N-(4-(chlorodifluoromethoxy)phenyl)- 1,1,2-trimethyl-7-(5-methylfuran-2-yl)-3- oxoisoindoline-5-carboxamide |
| IV-3 | 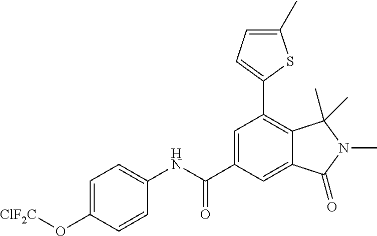 | N-(4-(chlorodifluoromethoxy)phenyl)- 1,1,2-trimethyl-7-(5-methylthiophen-2-yl)- 3-oxoisoindoline-5-carboxamide |
| IV-4 | 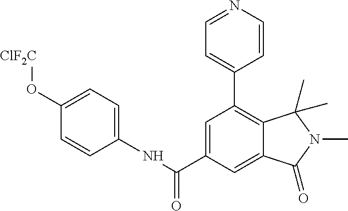 | N-(4-(chlorodifluoromethoxy)phenyl)- 1,1,2-trimethyl-3-oxo-7-(pyridin-4- yl)isoindoline-5-carboxamide |
| IV-5 | 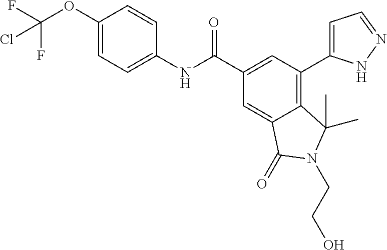 [SMILES: CC1(C)c2c(cc(C(=O)Nc3ccc(OC(F)(F)Cl)cc3)cc2-c2ccn[nH]2)C(=O)N1CCO] | N-(4-(chlorodifluoromethoxy)phenyl)-2- (2-hydroxyethyl)- 1,1-dimethyl-3-oxo-7- (1H-pyrazol-5-yl)isoindoline-5- carboxamide |
| IV-6 | 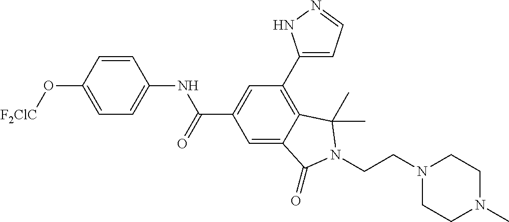 | N-(4-(chlorodifluoromethoxy)phenyl)-1,1- dimethyl-2-(2-(4-methylpiperazin-1- yl)ethyl)-3-oxo-7-(1H-pyrazol-5- yl)isoindoline-5-carboxamide |
| IV-7 | 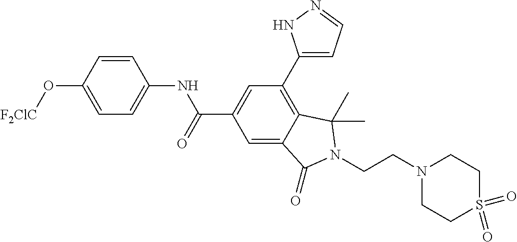 | N-(4-(chlorodifluoromethoxy)phenyl)-2- (2-(1,1-dioxidothiomorpholino)ethyl)- 1,1-dimethyl-3-oxo-7-(1H-pyrazol-5- yl)isoindoline-5-carboxamide |
| IV-8 | 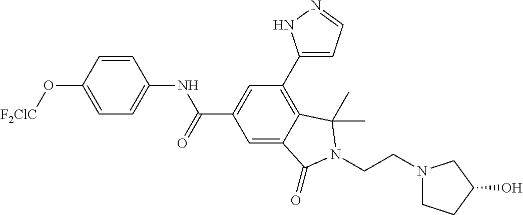 | (R)-N-(4-(chlorodifluoromethoxy)phenyl)- 2-(2-(3-hydroxypyrrolidin-1-yl)ethyl)-1,1- dimethyl-3-oxo-7-(1H-pyrazol-5- yl)isoindoline-5-carboxamide |
| IV-9 | 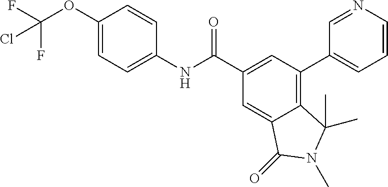 [SMILES: CN1C(=O)c2cc(C(=O)Nc3ccc(OC(F)(F)Cl)cc3)cc(-c3cccnc3)c2C1(C)C] | N-(4-(chlorodifluoromethoxy)phenyl)- 1,1,2-trimethyl-3-oxo-7-(pyridin-3- yl)isoindoline-5-carboxamide |
| V-1 | 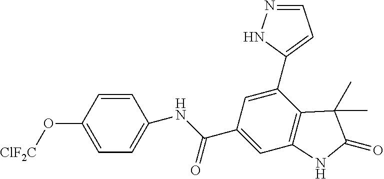 | N-(4-(chlorodifluoromethoxy)phenyl)-3,3- dimethyl-2-oxo-4-(1H-pyrazol-5- yl)indoline-6-carboxamide |
| V-2 | 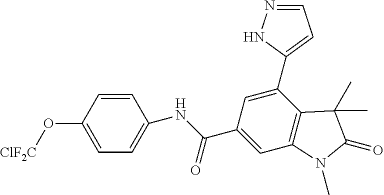 | N-(4-(chlorodifluoromethoxy)phenyl)- 1,3,3-trimethyl-2-oxo-4-(1H-pyrazol-5- yl)indoline-6-carboxamide |
| V-3 | 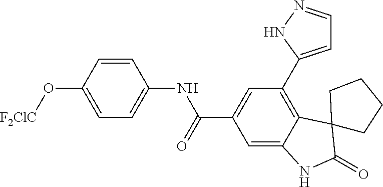 | N-(4-(chlorodifluoromethoxy)phenyl)-2′- oxo-4′-(1H-pyrazol-5- yl)spiro[cyclopentane-1,3′-indoline]-6′- carboxamide |
| V-4 | 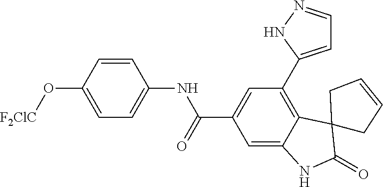 | N-(4-(chlorodifluoromethoxy)phenyl)-2′- oxo-4′-(1H-pyrazol-5- yl)spiro[cyclopentane-1,3′-indolin]-3-ene- 6′-carboxamide |
| V-5 | 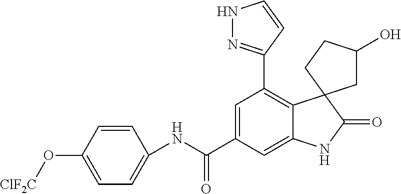 | N-(4-(chlorodifluoromethoxy)phenyl)-3- hydroxy-2′-oxo-4′-(1H-pyrazol-3- yl)spiro[cyclopentane-1,3′-indoline]-6′- carboxamide |
| V-6 | 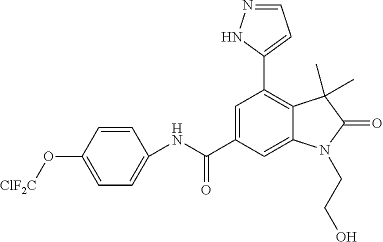 | N-(4-(chlorodifluoromethoxy)phenyl)-1-(2- hydroxyethyl)-3,3 -dimethyl-2-oxo-4-(1H- pyrazol-5-yl)indoline-6-carboxamide |
| V-7 | 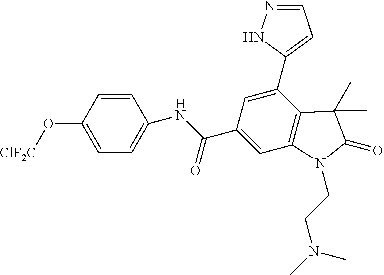 | N-(4-(chlorodifluoromethoxy)phenyl)-1-(2- (dimethylamino)ethyl)-3,3-dimethyl-2-oxo- 4-(1H-pyrazol-5-yl)indoline-6-carboxamide |
| V-8 | 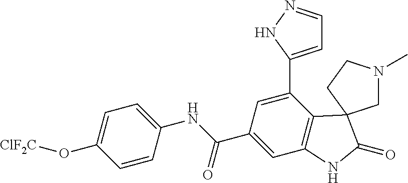 | N-(4-(chlorodifluoromethoxy)phenyl)-1′- methyl-2-oxo-4-(1H-pyrazol-5- yl)spiro[indoline-3,3′-pyrrolidine]-6- carboxamide |
| V-9 | 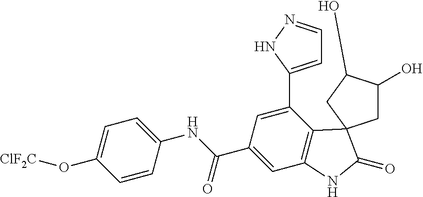 | N-(4-(chlorodifluoromethoxy)phenyl)-3,4- dihydroxy-2′-oxo-4′-(1H-pyrazol-5- yl)spiro[cyclopentane-1,3′-indoline]-6′- carboxamide |
| V-10 | 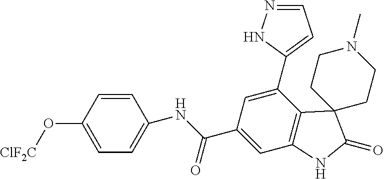 | N-(4-(chlorodifluoromethoxy)phenyl)-1′- methyl-2-oxo-4-(1H-pyrazol-5- yl)spiro[indoline-3,4′-piperidine]-6- carboxamide |
| V-11 | 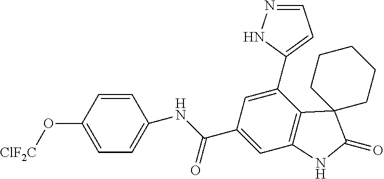 | N-(4-(chlorodifluoromethoxy)phenyl)-2′- oxo-4′-(1H-pyrazol-5-yl)spiro[cyclohexane- 1,3′-indoline]-6′-carboxamide |
| VII-1 | 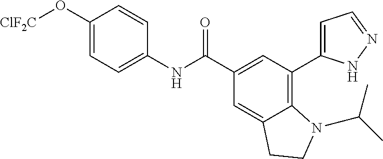 | N-(4-(chlorodifluoromethoxy)phenyl)-1- isopropyl-7-(1H-pyrazol-5-yl)indoline-5- carboxamide |
| VII-2 | 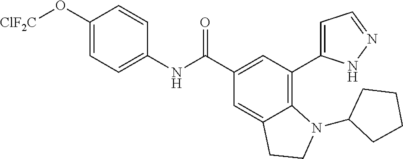 | N-(4-(chlorodifluoromethoxy)phenyl)-1- cyclopentyl-7-(1H-pyrazol-5-yl)indoline- 5-carboxamide |
| VII-3 | 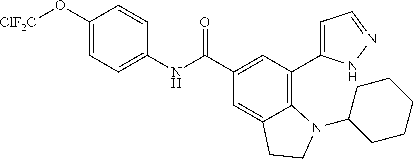 | N-(4-(chlorodifluoromethoxy)phenyl)-1- cyclohexyl-7-(1H-pyrazol-5-yl)indoline- 5-carboxamide |
| VII-4 | 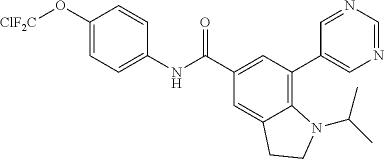 | N-(4-(chlorodifluoromethoxy)phenyl)-1- isopropyl-7-(pyrimidin-5-yl)indoline-5- carboxamide |
| VII-5 | 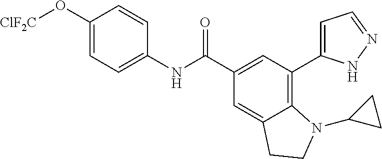 | N-(4-(chlorodifluoromethoxy)phenyl)-1- cyclopropyl-7-(1H-pyrazol-5-yl)indoline- 5-carboxamide |
| VII-6 | 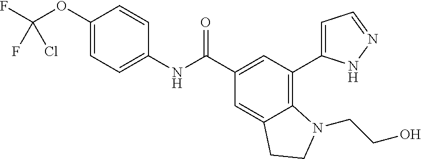 [SMILES: O=C(Nc1ccc(OC(F)(F)Cl)cc1)c1cc2c(c(-c3ccn[nH]3)c1)N(CCO)CC2] | N-(4-(chlorodifluoromethoxy)phenyl)-1-(2- hydroxyethyl)-7-(1H-pyrazol-5-yl)indoline- 5-carboxamide |
| VII-7 | 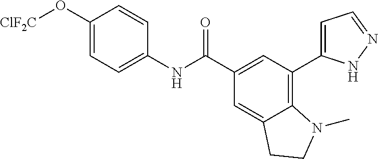 | N-(4-(chlorodifluoromethoxy)phenyl)-1- methyl-7-(1H-pyrazol-5-yl)indoline-5- carboxamide |
| VII-8 | 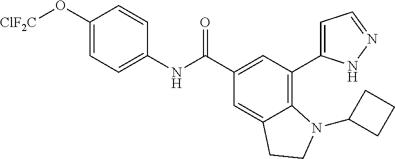 | N-(4-(chlorodifluoromethoxy)phenyl)-1- cyclobutyl-7-(1H-pyrazol-5-yl)indoline- 5-carboxamide |
| VII-9 | 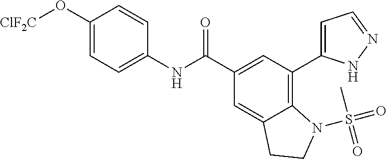 | N-(4-(chlorodifluoromethoxy)phenyl)-1- (methylsulfonyl)-7-(1H-pyrazol-5- yl)indoline-5-carboxamide |
| VII-10 | 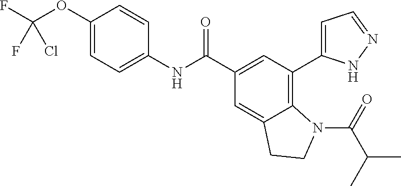 [SMILES: CC(C)C(=O)N1CCc2cc(C(=O)Nc3ccc(OC(F)(F)Cl)cc3)cc(-c3ccn[nH]3)c21] | N-(4-(chlorodifluoromethoxy)phenyl)-1- isobutyryl-7-(1H-pyrazol-5-yl)indoline- 5-carboxamide |
| VII-11 | 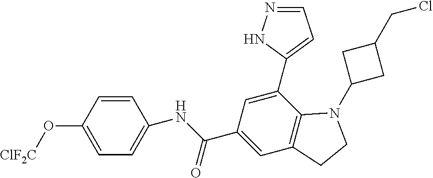 | N-(4-(chlorodifluoromethoxy)phenyl)-1-(3- (chloromethyl)cyclobutyl)-7-(1H-pyrazol-5- yl)indoline-5-carboxamide |
| VII-12 | 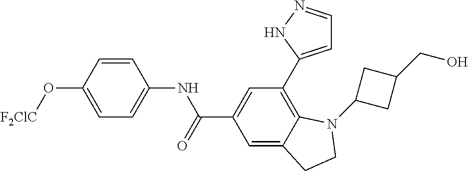 | N-(4-(chlorodifluoromethoxy)phenyl)-1-(3- (hydroxymethyl)cyclobutyl)-7-(1H-pyrazol- 5-yl)indoline-5-carboxamide |
| VII-13 | 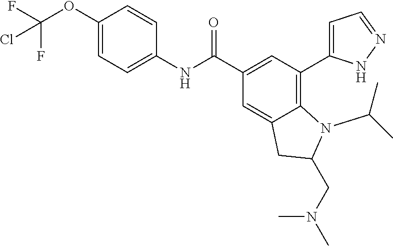 [SMILES: CC(C)N1c2c(cc(C(=O)Nc3ccc(OC(F)(F)Cl)cc3)cc2-c2ccn[nH]2)CC1CN(C)C] | N-(4-(chlorodifluoromethoxy)phenyl)-2- ((dimethylamino)methyl)-1-isopropyl-7- (1H-pyrazol-5-yl)indoline-5-carboxamide |
| VII-14 | 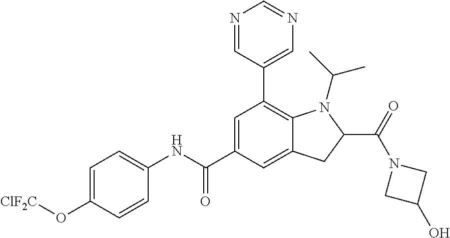 | N-(4-(chlorodifluoromethoxy)phenyl)-2-(3- hydroxyazetidine-1-carbonyl)-1-isopropyl- 7-(pyrimidin-5-yl)indoline-5-carboxamide |
| VII-15 | 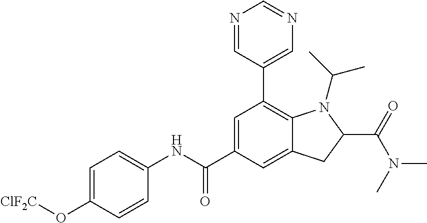 | N5-(4-(chlorodifluoromethoxy)phenyl)-1- isopropyl-N2,N2-dimethyl-7-(pyrimidin-5- yl)indoline-2,5-dicarboxamide |
| VII-16 | | N5-(4-(chlorodifluoromethoxy)phenyl)-1- isopropyl-N2-methyl-7-(pyrimidin-5- yl)indoline-2,5-dicarboxamide |
| VII-17 | 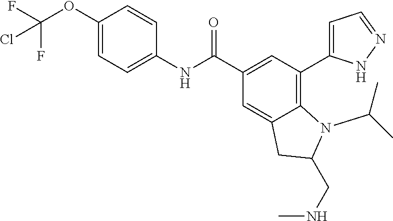 [SMILES: CNCC1Cc2cc(C(=O)Nc3ccc(OC(F)(F)Cl)cc3)cc(-c3ccn[nH]3)c2N1C(C)C] | N-(4-(chlorodifluoromethoxy)phenyl)-1- isopropyl-2-((methylamino)methyl)-7-(1H- pyrazol-5-yL)indoline-5-carboxamide |
| VII-18 | 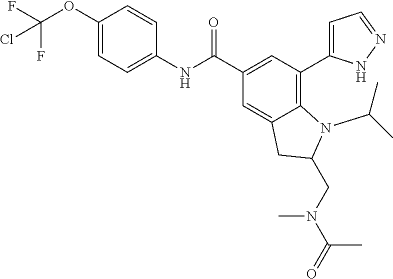 [SMILES: CC(=O)N(C)CC1Cc2cc(C(=O)Nc3ccc(OC(F)(F)Cl)cc3)cc(-c3ccn[nH]3)c2N1C(C)C] | N-(4-(chlorodifluoromethoxy)phenyl)-1- isopropyl-2-((N-methylacetamido)methyl)- 7-(1H-pyrazol-5-ypindoline-5-carboxamide |
| VII-19 | 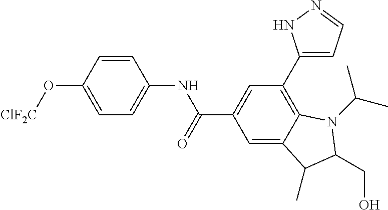 | N-(4-(chlorodifluoromethoxy)phenyl)-2- (hydroxymethyl)-1-isopropyl-3-methyl-7- (1H-pyrazol-5-yl)indoline-5-carboxamide |
| VII-20 | 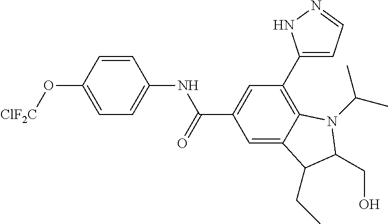 | N-(4-(chlorodifluoromethoxy)phenyl)-3- ethyl-2-(hydroxymethyl)-1-isopropyl-7- (1H-pyrazol-5-yl)indoline-5-carboxamide |
| VII-21 | 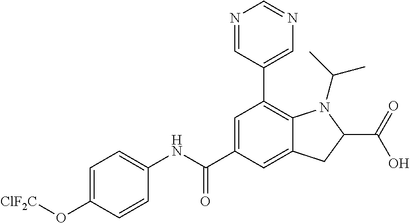 | 5-((4- (chlorodifluoromethoxy)phenyl) carbamoyl)-1-isopropyl-7-(pyrimidin- 5-yl)indoline-2-carboxylic acid |
| VII-22 | 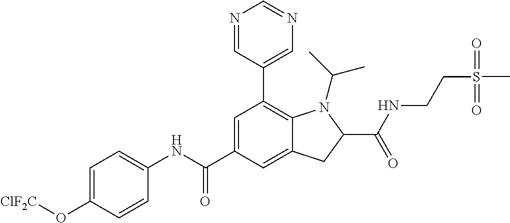 | N5-(4-(chlorodifluoromethoxy)phenyl)-1- isopropyl-N2-(2-(methylsulfonyl)ethyl)- 7-(pyrimidin-5-yl)indoline-2,5- dicarboxamide |
| VII-23 | 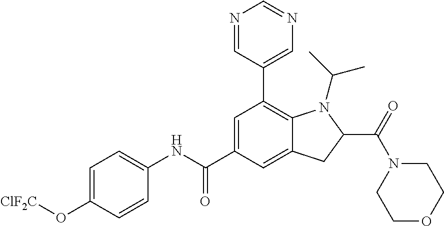 | N-(4-(chlorodifluoromethoxy)phenyl)-1- isopropyl-2-(morpholine-4-carbonyl)-7- (pyrimidin-5-yl)indoline-5-carboxamide |
| VII-24 | 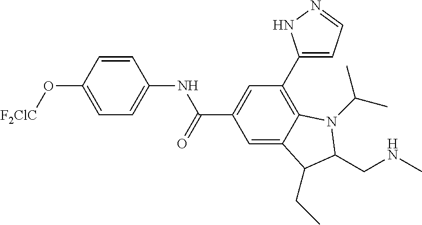 | N-(4-(chlorodifluoromethoxy)phenyl)- 3-ethyl-1-isopropyl-2- ((methylamino)methyl)-7-(1H- pyrazol-5-yl)indoline-5-carboxamide |
| VII-25 | 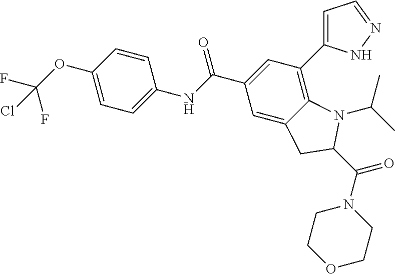 [SMILES: CC(C)N1c2c(cc(C(=O)Nc3ccc(OC(F)(F)Cl)cc3)cc2-c2ccn[nH]2)CC1C(=O)N1CCOCC1] | N-(4-(chlorodifluoromethoxy)phenyl)-1- isopropyl-2-(morpholine-4-carbonyl)-7- (1H-pyrazol-5-yl)indoline-5-carboxamide |
| VII-26 | 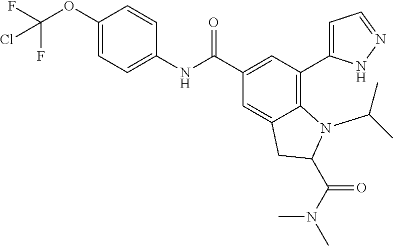 [SMILES: CC(C)N1c2c(cc(C(=O)Nc3ccc(OC(F)(F)Cl)cc3)cc2-c2ccn[nH]2)CC1C(=O)N(C)C] | N5-(4-(chlorodifluoromethoxy)phenyl)-1- isopropyl-N2,N2-dimethyl-7-(1H-pyrazol- 5-yl)indoline-2,5-dicarboxamide |
| VII-27 | 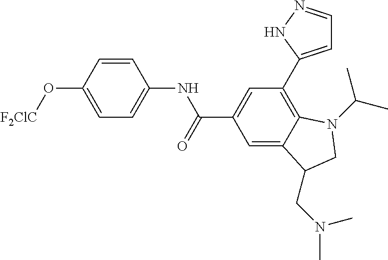 | N-(4-(chlorodifluoromethoxy)phenyl)-3- ((dimethylamino)methyl)-1-isopropyl-7- (1H-pyrazol-5-yl)indoline-5-carboxamide |
| VII-28 | 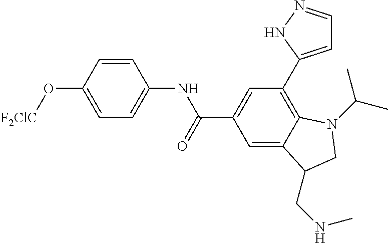 | N-(4-(chlorodifluoromethoxy)phenyl)-1- isopropyl-3-((methylamino)methyl)-7- (1H-pyrazol-5-yl)indoline-5-carboxamide |
| VII-29 | 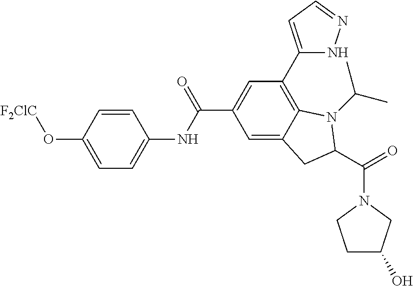 | N-(4-(chlorodifluoromethoxy)phenyl)-2- ((R)-3-hydroxypyrrolidine-1-carbonyl)-1- isopropyl-7-(1H-pyrazol-5-yl)indoline-5- carboxamide |
| VII-30 | 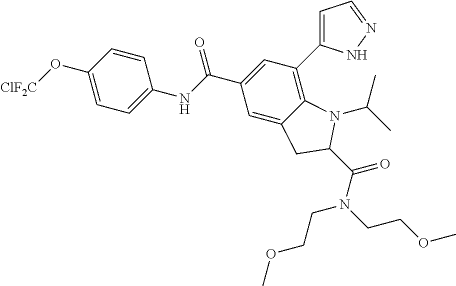 | N5-(4-(chlorodifluoromethoxy)phenyl)-1- isopropyl-N2,N2-bis(2-methoxyethyl)-7- (1H-pyrazol-5-yl)indoline-2,5- dicarboxamide |
| VII-31 | 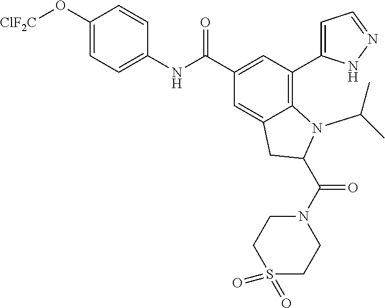 | N-(4-(chlorodifluoromethoxy)phenyl)-2- (1,1-dioxidothiomorpholine-4-carbonyl)-1- isopropyl-7-(1H-pyrazol-5-yl)indoline-5- carboxamide |
| VII-32 | 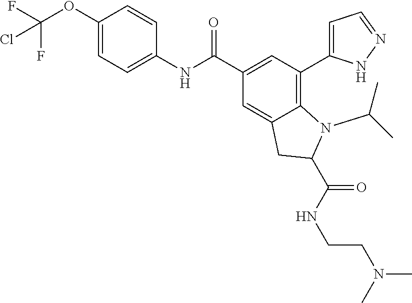 [SMILES: CC(C)N1c2c(cc(C(=O)Nc3ccc(OC(F)(F)Cl)cc3)cc2-c2ccn[nH]2)CC1C(=O)NCCN(C)C] | N5-(4-(chlorodifluoromethoxy)phenyl)- N2-(2-(dimethylamino)ethyl)-1-isopropyl- 7-(1H-pyrazol-5-yl)indoline-2,5- dicarboxamide |
| VII-33 | 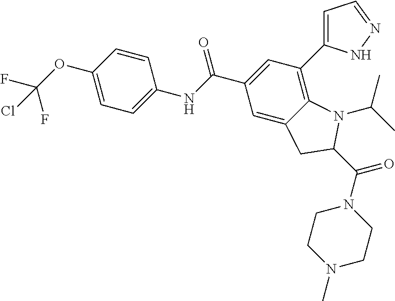 [SMILES: CC(C)N1c2c(cc(C(=O)Nc3ccc(OC(F)(F)Cl)cc3)cc2-c2ccn[nH]2)CC1C(=O)N1CCN(C)CC1] | N-(4-(chlorodifluoromethoxy)phenyl)-1- isopropyl-2-(4-methylpiperazine-1- carbonyl)-7-(1H-pyrazol-5-yl)indoline-5- carboxamide |
| VII-34 | 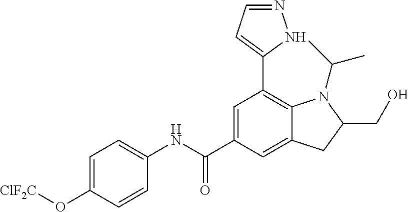 | N-(4-(chlorodifluoromethoxy)phenyl)-2- (hydroxymethyl)-1-isopropyl-7-(1H- pyrazol-5-yl)indoline-5-carboxamide |
| VII-35 | 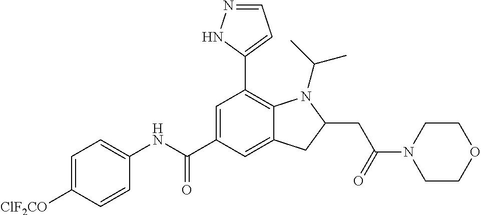 | N-(4-(chlorodifluoromethoxy)phenyl)-1- isopropyl-2-(2-morpholino-2-oxoethyl)-7- (1H-pyrazol-5-yl)indoline-5-carboxamide |
| VII-36 | 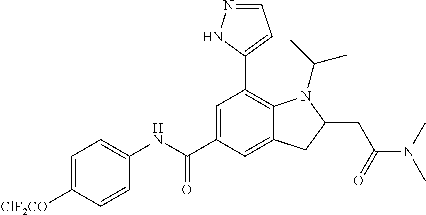 | N-(4-(chlorodifluoromethoxy)phenyl)-2- (2-(dimethylamino)-2-oxoethyl)-1- isopropyl-7-(1H-pyrazol-5-yl)indoline-5- carboxamide |
| VII-37 | 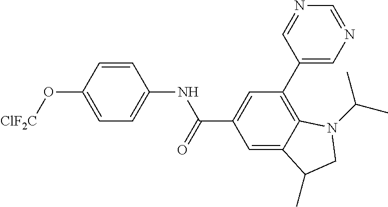 | N-(4-(chlorodifluoromethoxy)phenyl)-1- isopropyl-3-methyl-7-(pyrimidin-5- yl)indoline-5-carboxamide |
| VII-38 | 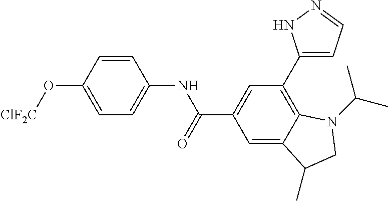 | N-(4-(chlorodifluoromethoxy)phenyl)-1- isopropyl-3-methyl-7-(1H-pyrazol-5- yl)indoline-5-carboxamide |
| VII-39 | 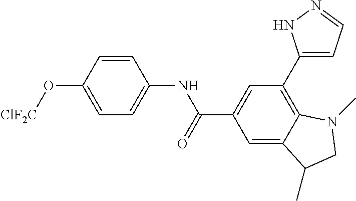 | N-(4-(chlorodifluoromethoxy)phenyl)-1,3- dimethyl-7-(1H-pyrazol-5-yl)indoline-5- carboxamide |
| VII-40 | 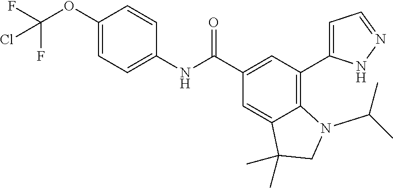 [SMILES: CC(C)N1CC(C)(C)c2cc(C(=O)Nc3ccc(OC(F)(F)Cl)cc3)cc(-c3ccn[nH]3)c21] | N-(4-(chlorodifluoromethoxy)phenyl)-1- isopropyl-3,3-dimethyl-7-(1H-pyrazol-5- yl)indoline-5-carboxamide |
| VII-41 | 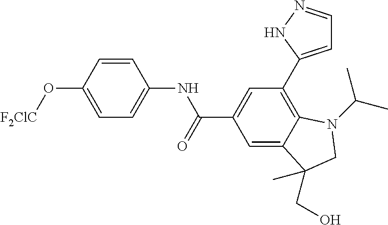 | N-(4-(chlorodifluoromethoxy)phenyl)-3- (hydroxymethyl)-1-isopropyl-3-methyl-7- (1H-pyrazol-5-yl)indoline-5-carboxamide |
| VII-42 | 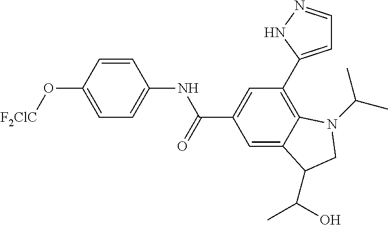 | N-(4-(chlorodifluoromethoxy)phenyl)-1,3- diisopropyl-7-(1H-pyrazol-5-yl)indoline-5- carboxamide |
| VII-43 | 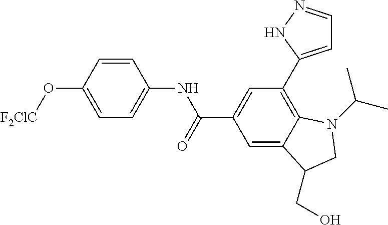 | N-(4-(chlorodifluoromethoxy)phenyl)-3- (hydroxymethyl)-1-isopropyl-7-(1H- pyrazol-5-yl)indoline-5-carboxamide |
| VII-44 | 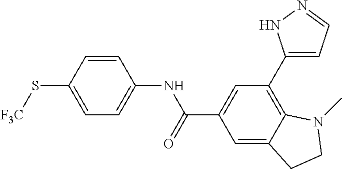 [SMILES: CN1CCc2cc(C(=O)Nc3ccc(SC(F)(F)F)cc3)cc(-c3ccn[nH]3)c21] | 1-methyl-7-(1H-pyrazol-5-yl)-N-(4- ((trifluoromethyl)thio)phenyl)indoline-5- carboxamide |
| VII-45 | 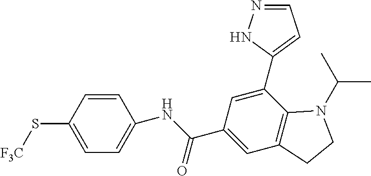 [SMILES: CC(C)N1CCc2cc(C(=O)Nc3ccc(SC(F)(F)F)cc3)cc(-c3ccn[nH]3)c21] | 1-isopropyl-7-(1H-pyrazol-5-yl)-N-(4- ((trifluoromethyl)thio)phenyl)indoline-5- carboxamide |
| VII-46 | 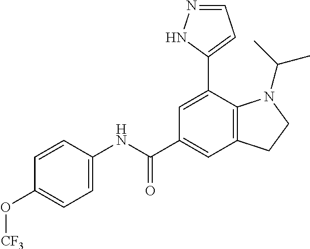 [SMILES: CC(C)N1CCc2cc(C(=O)Nc3ccc(OC(F)(F)F)cc3)cc(-c3ccn[nH]3)c21] | 1-isopropyl-7-(1H-pyrazol-5-yl)-N-(4- (trifluoromethoxy)phenyl)indoline-5- carboxamide |
| VIII-1 | 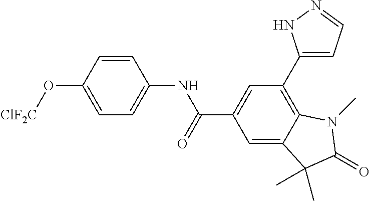 | N-(4-(chlorodifluoromethoxy)phenyl)- 1,3,3-trimethyl-2-oxo-7-(1H-pyrazol-5- yl)indoline-5-carboxamide |
| VIII-2 | 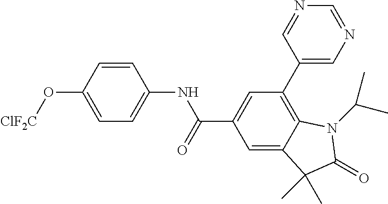 | N-(4-(chlorodifluoromethoxy)phenyl)-1- isopropyl-3,3-dimethyl-2-oxo-7- (pyrimidin-5-yl)indoline-5-carboxamide |
| VIII-3 | 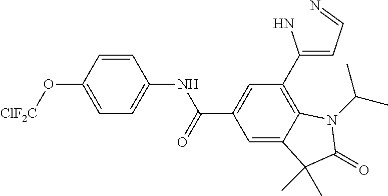 | N-(4-(chlorodifluoromethoxy)phenyl)-1- isopropyl-3,3-dimethyl-2-oxo-7-(1H- pyrazol-5-ypindoline-5-carboxamide |
| IX-1 | 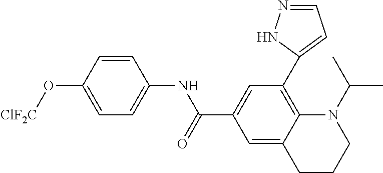 | N-(4-(chlorodifluoromethoxy)phenyl)-1- isopropyl-8-(1H-pyrazol-5-yl)-1,2,3,4- tetrahydroquinoline-6-carboxamide |
| IX-2 | 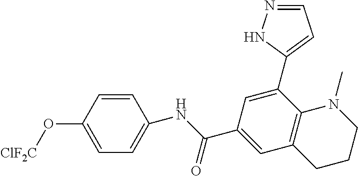 | N-(4-(chlorodifluoromethoxy)phenyl)-1- methyl-8-(1H-pyrazol-5-yl)-1,2,3,4- tetrahydroquinoline-6-carboxamide |
| IX-3 | 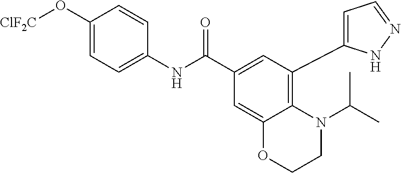 | N-(4-(chlorodifluoromethoxy)phenyl)-4- isopropyl-5-(1H-pyrazol-5-yl)-3,4- dihydro-2H-benzo[b][1,4]oxazine-7- carboxamide |
| IX-4 | 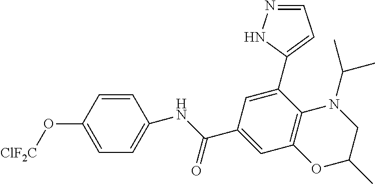 | N-(4-(chlorodifluoromethoxy)phenyl)-4- isopropyl-2-methyl-5-(1H-pyrazol-5-yl)- 3,4-dihydro-2H-benzo[b][1,4]oxazine-7- carboxamide |
| IX-5 | 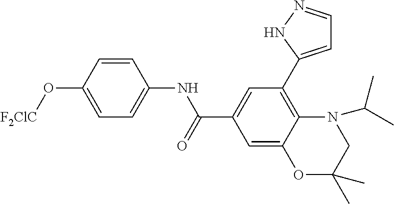 | N-(4-(chlorodifluoromethoxy)phenyl)-4- isopropyl-2,2-dimethyl-5-(1H-pyrazol-5- yl)-3,4-dihydro-2H-benzo[b][1,4]oxazine- 7-carboxamide |
| IX-6 | 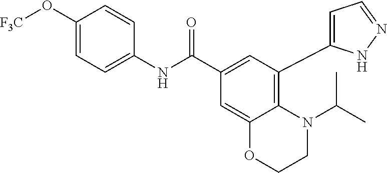 [SMILES: CC(C)N1CCOc2cc(C(=O)Nc3ccc(OC(F)(F)F)cc3)cc(-c3ccn[nH]3)c21] | 4-isopropyl-5-(1H-pyrazol-5-yl)-N-(4- (trifluoromethoxy)phenyl)-3,4-dihydro- 2H-benzo[b][1,4]oxazine-7- carboxamide |
| X-1 | 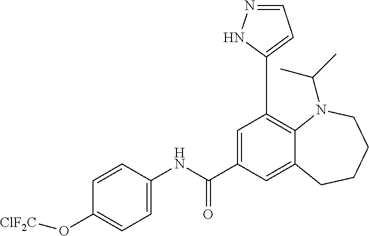 | N-(4-(chlorodifluoromethoxy)phenyl)-1- isopropyl-9-(1H-pyrazol-5-yl)-2,3,4,5- tetrahydro-1H-benzo[b]azepine-7- carboxamide |
| XII-1 | 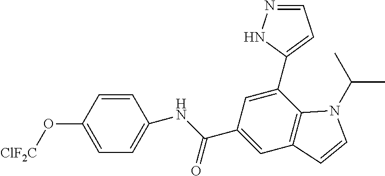 | N-(4-(chlorodifluoromethoxy)phenyl)-1- isopropyl-7-(1H-pyrazol-5-yl)-1H-indole- 5-carboxamide |
| XII-2 | 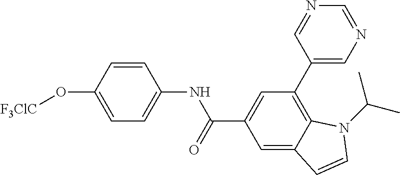 | N-(4-(chlorodifluoromethoxy)phenyl)-1- isopropyl-7-(pyrimidin-5-yl)-1H-indole-5- carboxamide |
| XII-3 | 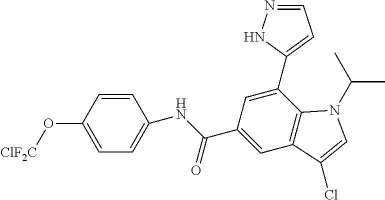 | 3-chloro-N-(4- (chlorodifluoromethoxy)phenyl)-1- isopropyl-7-(1H-pyrazol-5-yl)-1H-indole- 5-carboxamide |
| XII-4 | 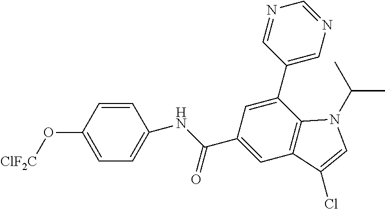 | 3-chloro-N-(4- (chlorodifluoromethoxy)phenyl)-1- isopropyl-7-(pyrimidin-5-yl)-1H-indole-5- carboxamide |
| XII-5 | 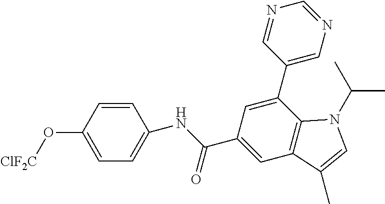 | N-(4-(chlorodifluoromethoxy)phenyl)-1- isopropyl-3-methyl-7-(pyrimidin-5-yl)-1H- indole-5-carboxamide |
| XII-6 | 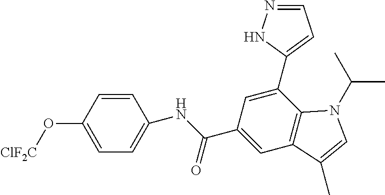 | N-(4-(chlorodifluoromethoxy)phenyl)-1- isopropyl-3-methyl-7-(1H-pyrazol-5-yl)- 1H-indole-5-carboxamide |
| XII-7 | 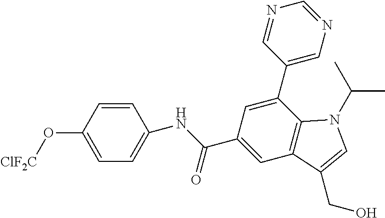 | N-(4-(chlorodifluoromethoxy)phenyl)-3- (hydroxymethyl)-1-isopropyl-7- (pyrimidin-5-yl)-1H-indole-5- carboxamide |
| XII-8 | 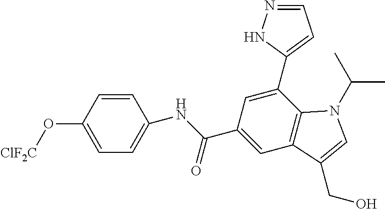 | N-(4-(chlorodifluoromethoxy)phenyl)-3- (hydroxymethyl)-1-isopropyl-7-(1H- pyrazol-5-yl)-1H-indole-5-carboxamide |
| XIII-1 | 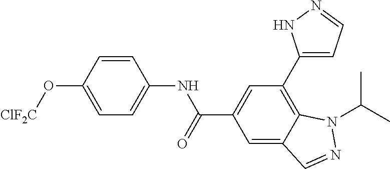 | N-(4-(chlorodifluoromethoxy)phenyl)-1- isopropyl-7-(1H-pyrazol-5-yl)-1H- indazole-5-carboxamide |
| XIV | 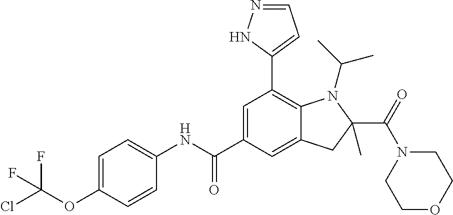 [SMILES: CC(C)N1c2c(cc(C(=O)Nc3ccc(OC(F)(F)Cl)cc3)cc2-c2ccn[nH]2)CC1(C)C(=O)N1CCOCC1] | N-(4-(chlorodifluoromethoxy)phenyl)-1- isopropyl-2-methyl-2-(morpholine-4- carbonyl)-7-(1H-pyrazol-5-yl)indoline-5- carboxamide |
| XX-2 | 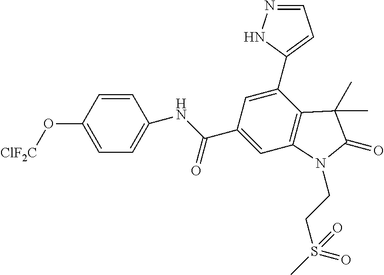 | N-(4-(chlorodifluoromethoxy)phenyl)-3,3- dimethyl-1-(2-(methylsulfonypethyl)-2- oxo-4-(1H-pyrazol-5-yl)indoline-6- carboxamide |
| XX-3 | 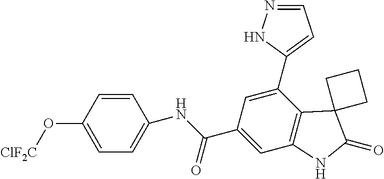 | N-(4-(chlorodifluoromethoxy)phenyl)-2′- oxo-4′-(1H-pyrazol-5-yl)spiro [cyclobutane-1,3′-indoline]-6′- carboxamide |
| XX-4 | 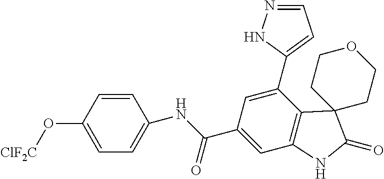 | N-(4-(chlorodifluoromethoxy)phenyl)-2- oxo-4-(1H-pyrazol-5-yl)-2′,3′,5′,6′- tetrahydrospiro[indoline-3,4′-pyran]-6- carboxamide |
| XX-5 | 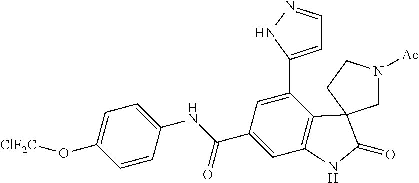 | 1′-acetyl-N-(4- (chlorodifluoromethoxy)phenyl)-2-oxo-4- (1H-pyrazol-5-yl)spiro[indoline-3,3′- pyrrolidine]-6-carboxamide |
| XX-6 | 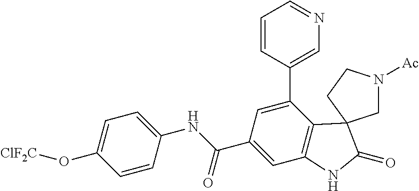 | 1′-acetyl-N-(4- (chlorodifluoromethoxy)phenyl)-2-oxo-4- (pyridin-3-yl)spiro[indoline-3,3′- pyrrolidine]-6-carboxamide |
| XX-7 | 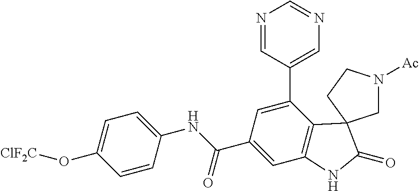 | 1′-acetyl-N-(4- (chlorodifluoromethoxy)phenyl)-2-oxo-4- (pyrimidin-5-yl)spiro[indoline-3,3′- pyrrolidine]-6-carboxamide |
| XX-8 | 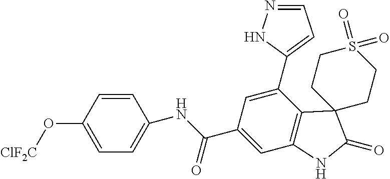 | N-(4-(chlorodifluoromethoxy)phenyl)-2- oxo-4-(1H-pyrazol-5-yl)-2′,3′,5′,6′- tetrahydrospiro[indoline-3,4′-thiopyran]-6- carboxamide 1′,1′-dioxide |
| XX-9 | 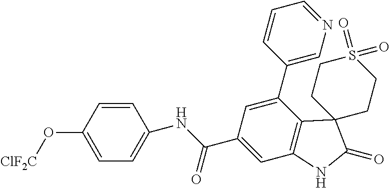 | N-(4-(chlorodifluoromethoxy)phenyl)-2- oxo-4-(pyridin-3-yl)-2′,3′,5′,6′- tetrahydrospiro[indoline-3,4′-thiopyran]-6- carboxamide 1′,1′-dioxide |
| XX-9 | 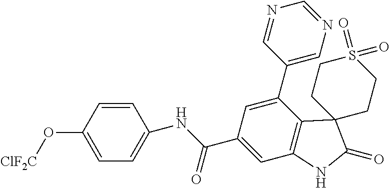 | N-(4-(chlorodifluoromethoxy)phenyl)-2- oxo-4-(pyrimidin-5-yl)-2′,3′,5′,6′- tetrahydrospiro[indoline-3,4′-thiopyran]-6- carboxamide 1′,1′-dioxide |
| XX-10 | 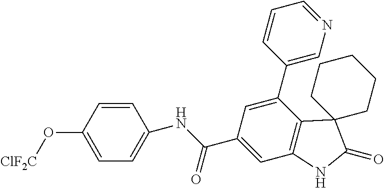 | N-(4-(chlorodifluoromethoxy)phenyl)-2′- oxo-4′-(pyridin-3-yl)spiro[cyclohexane- 1,3′-indoline]-6′-carboxamide |
| XX-11 | 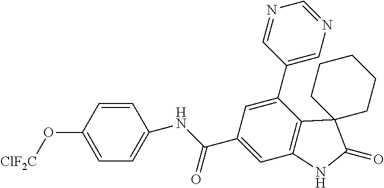 | N-(4-(chlorodifluoromethoxy)phenyl)-2′- oxo-4′-(pyrimidin-5-yl)spiro[cyclohexane- 1,3′-indoline]-6′-carboxamide |
| XX-12 | 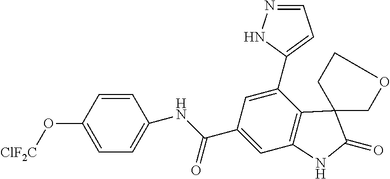 | N-(4-(chlorodifluoromethoxy)phenyl)-2′- oxo-4′-(1H-pyrazol-5-yl)-4,5-dihydro-2H- spiro[furan-3,3′-indoline]-6′-carboxamide |
| XX-13 | 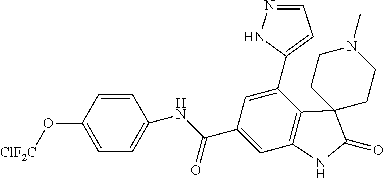 | N-(4-(chlorodifluoromethoxy)phenyl)-1′- methyl-2-oxo-4-(1H-pyrazol-5- yl)spiro[indoline-3,4′-piperidine]-6- carboxamide |
| XX-13 | 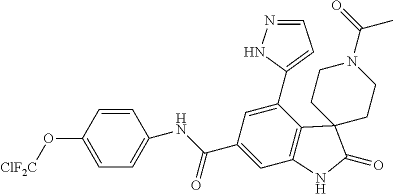 | 1′-acetyl-N-(4- (chlorodifluoromethoxy)phenyl)-2-oxo-4- (1H-pyrazol-5-yl)spiro[indoline-3,4′- piperidine]-6-carboxamide |
| XX-14 | 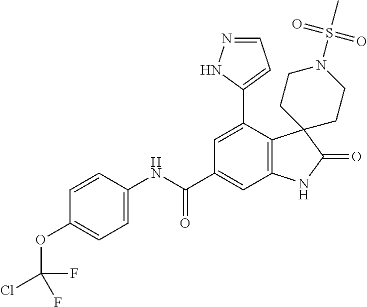 [SMILES: CS(=O)(=O)N1CCC2(CC1)C(=O)Nc1cc(C(=O)Nc3ccc(OC(F)(F)Cl)cc3)cc(-c3ccn[nH]3)c12] | N-(4-(chlorodifluoromethoxy)phenyl)-1′- (methylsulfonyl)-2-oxo-4-(1H-pyrazol-5- yl)spiro[indoline-3,4′-piperidine]-6- carboxamide |

In another embodiment, Compounds of the Disclosure are compounds of Formula I selected from group consisting of:
N-(4-(chlorodifluoromethoxy)phenyl)-3,3-dimethyl-2-oxo-4-(1H-pyrazol-5-yl)indoline-6-carboxamide;
N-(4-(chlorodifluoromethoxy)phenyl)-2′-oxo-4′-(1H-pyrazol-5-yl)spiro[cyclopentane-1,3′-indoline]-6′-carboxamide;
N-(4-(chlorodifluoromethoxy)phenyl)-1′-methyl-2-oxo-4-(1H-pyrazol-5-yl)spiro[indoline-3,3′-pyrrolidine]-6-carboxamide;
N-(4-(chlorodifluoromethoxy)phenyl)-1′-methyl-2-oxo-4-(1H-pyrazol-5-yl)spiro[indoline-3,4′-piperidine]-6-carboxamide;
N-(4-(chlorodifluoromethoxy)phenyl)-2′-oxo-4′-(1H-pyrazol-5-yl)spiro[cyclohexane-1,3′-indoline]-6′-carboxamide;
N-(4-(chlorodifluoromethoxy)phenyl)-1-cyclopropyl-7-(1H-pyrazol-5-yl)indoline-5-carboxamide;
N-(4-(chlorodifluoromethoxy)phenyl)-1-isobutyryl-7-(1H-pyrazol-5-yl)indoline-5-carboxamide;
N5-(4-(chlorodifluoromethoxy)phenyl)-1-isopropyl-N2,N2-bis(2-methoxyethyl)-7-(1H-pyrazol-5-yl)indoline-2,5-dicarboxamide;
N-(4-(chlorodifluoromethoxy)phenyl)-1-isopropyl-2-(2-morpholino-2-oxoethyl)-7-(1H-pyrazol-5-yl)indoline-5-carboxamide;
N-(4-(chlorodifluoromethoxy)phenyl)-2-(2-(dimethylamino)-2-oxoethyl)-1-isopropyl-7-(1H-pyrazol-5-yl)indoline-5-carboxamide; and
1-isopropyl-7-(1H-pyrazol-5-yl)-N-(4-((trifluoromethyl)thio)phenyl)indoline-5-carboxamide, or a pharmaceutically acceptable salt or solvate thereof.

In another embodiment, the disclosure provides a pharmaceutical composition comprising a Compound of the Disclosure and a pharmaceutically acceptable carrier.

In another embodiment, Compounds of the Disclosure are enantiomerically enriched, e.g., the enantiomeric excess or “ee” of the compound is about 5% or more as measured by chiral HPLC. In another embodiment, the ee is about 10%. In another embodiment, the ee is about 20%. In another embodiment, the ee is about 30%. In another embodiment, the ee is about 40%. In another embodiment, the ee is about 50%. In another embodiment, the ee is about 60%. In another embodiment, the ee is about 70%. In another embodiment, the ee is about 80%. In another embodiment, the ee is about 85%. In another embodiment, the ee is about 90%. In another embodiment, the ee is about 91%. In another embodiment, the ee is about 92%. In another embodiment, the ee is about 93%. In another embodiment, the ee is about 94%. In another embodiment, the ee is about 95%. In another embodiment, the ee is about 96%. In another embodiment, the ee is about 97%. In another embodiment, the ee is about 98%. In another embodiment, the ee is about 99%.

The present disclosure encompasses the preparation and use of salts of Compounds of the Disclosure. As used herein, the pharmaceutical “pharmaceutically acceptable salt” refers to salts or zwitterionic forms of Compounds of the Disclosure. Salts of Compounds of the Disclosure can be prepared during the final isolation and purification of the compounds or separately by reacting the compound with a suitable acid. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

### II. Intermediates of the Disclosure

The disclosure also provides synthetic intermediates, collectively referred to as “Intermediates of the Disclosure,” that can be used to prepare Compounds of the Disclosure.

### III. Methods of Preparing Compounds and Intermediates of the Disclosure

The disclosure also provides methods of preparing Compounds of the Disclosure and/or Intermediates of the Disclosure.

### IV. Methods of Treating Disease with Compounds of the Disclosure

Compounds of the Disclosure inhibit BCR-ABL and are thus useful in the treatment or prevention of a variety of diseases and conditions. In particular, Compounds of the Disclosure are useful in methods of treating or preventing a disease or condition wherein inhibition of BCR-ABL provides a benefit. These diseases and conditions include cancers, e.g., metastatic invasive carcinomas, proliferative diseases, viral infections, e.g., pox and Ebola viruses. These diseases and conditions also include diseases or disorders associated with abnormally activated kinase activity of wild-type ABL1, including non-malignant diseases or disorders include CNS diseases, e.g., neurodegenerative diseases, e.g., Alzheimer's disease and Parkinson's diseases, muscular dystrophies, autoimmune diseases, inflammatory diseases, viral infections, and prion diseases.

In one embodiment, the cancer is referred to as a “BCR-ABL driven cancer.” BCR-ABL driven cancers are known in the art. The therapeutic methods of this disclosure comprise administering a therapeutically effective amount of a Compound of the Disclosure to a subject, e.g., human, in need thereof. The present methods also encompass optionally administering a second therapeutic agent to the subject in addition to the Compound of the Disclosure. The second therapeutic agent is selected from drugs known as useful in treating the disease or condition afflicting the subject in need thereof, e.g., a chemotherapeutic agent, e.g., an ATP-competitive BCR-ABL inhibitor, and/or radiation known as useful in treating a particular cancer.

The present disclosure provides Compounds of the Disclosure as BCR-ABL inhibitors for the treatment of diseases and conditions wherein inhibition of BCR-ABL has a beneficial effect. Compounds of the Disclosure typically have a half maximal inhibitory concentration (IC₅₀) for inhibiting BCR-ABL of less than 100 μM. In other embodiments, the IC₅₀ for inhibiting BCR-ABL is less than 50 μM, less than 25 μM, and less than 5 μM, less than about 1 μM, less than about 0.5 μM, less than about 0.1 μM, less than about 0.05 μM, or less than about 0.01 μM. In one embodiment, the present rative diseases, muscular dystrophies, autoimmune, diseases, inflammatory diseases, v condition wherein inhibition of BCR-ABL provides a benefit comprising administering a therapeutically effective amount of a Compound of the Disclosure to an individual in need thereof.

In another embodiment, the present disclosure relates to a method of treating an individual suffering from a disease or condition wherein inhibition of BCR-ABL provides a benefit, the method comprising administering a therapeutically effective amount of a Compound of the Disclosure.

Since Compounds of the Disclosure are inhibitors of BCR-ABL protein, a number of diseases and conditions mediated by BCR-ABL can be treated by employing these compounds. The present disclosure is thus directed generally to a method for treating a condition or disorder responsive to BCR-ABL inhibition in a subject, e.g., a human subject, suffering from, or at risk of suffering from, the condition or disorder, the method comprising administering to the subject an effective amount of one or more Compounds of the Disclosure.

In another embodiment, the present disclosure is directed to a method of inhibiting BCR-ABL in a subject in need thereof, said method comprising administering to the subject an effective amount of at least one Compound of the Disclosure.

The methods of the present disclosure can be accomplished by administering a Compound of the Disclosure as the neat compound or as a pharmaceutical composition. Administration of a pharmaceutical composition, or neat compound of a Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered. Further provided are kits comprising a Compound of the Disclosure and, optionally, a second therapeutic agent, packaged separately or together, and an insert having instructions for using these active agents.

In one embodiment, a Compound of the Disclosure is administered in conjunction with a second therapeutic agent useful in the treatment of a disease or condition wherein inhibition of BCR-ABL provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered from a single composition or two separate compositions.

The second therapeutic agent is administered in an amount to provide its desired therapeutic effect. The effective dosage range for each second therapeutic agent is known in the art, and the second therapeutic agent is administered to an individual in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more dose of the second therapeutic agent can be administered. The Compound of the Disclosure therefore can be used in conjunction with one or more second therapeutic agents, for example, but not limited to, anticancer agents.

Diseases and conditions treatable by the Compounds of the Disclosure and methods of the present disclosure include, but are not limited to, cancer and other proliferative disorders, neurogenerative disorders, muscular dystrophies, autoimmune diseases, inflammatory diseases, viral infections, and prion diseases. In one embodiment, a human subject is treated with a Compound of the Disclosure, or a pharmaceutical composition comprising a Compound of the Disclosure, wherein the compound is administered in an amount sufficient to inhibit BCR-ABL protein in the subject.

In another aspect, the present disclosure provides a method of treating cancer in a subject comprising administering a therapeutically effective amount of a Compound of the Disclosure. While not being limited to a specific mechanism, in some embodiments, Compounds of the Disclosure treat cancer by inhibiting BCR-ABL. Examples of treatable cancers include, but are not limited to, any one or more of the cancers of Table 3.

TABLE 3

| | | | |
|---|---|---|---|
| adrenal cancer | acinic cell | acoustic neuroma | acral lentigious |
| | carcinoma | | melanoma |
| acrospiroma | acute eosinophilic | acute erythroid | acute |
| | leukemia | leukemia | lymphoblastic |
| | | | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressiveNK-cell |
| neoplasm | carcinoma | leukemia/lymphoma | leukemia |
| AIDS-related | alveolar | alveolar soft part | ameloblastic |
| lymphoma | rhabdomyosarcoma | sarcoma | fibroma |
| anaplastic large | anaplastic thyroid | angioimmunoblastic | angiomyolipoma |
| cell lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell | biliary tract |
| prolymphocytic | | carcinoma | cancer |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell | craniopharyngioma |
| | papilloma | sarcoma of | |
| | | the kidney | |
| cutaneous T-cell | cervical cancer | colorectal cancer | Degos disease |
| lymphoma | | | |
| desmoplastic | diffuse large B-cell | dysembryoplastic | dysgerminoma |
| small round | lymphoma | neuroepithelial | |
| cell tumor | | tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular | follicular thyroid | ganglioneuroma | gastrointestinal |
| lymphoma | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological | hepatoblastoma |
| cancer | | cancer | |
| hepatosplenic T-cell | Hodgkin's | non-Hodgkin's | invasive lobular |
| lymphoma | lymphoma | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic |
| | leukemia | leukemia | lymphocytic |
| | | | leukemia |
| liver cancer | small cell lung | non-small cell lung | MALT lymphoma |
| | cancer | cancer | |
| malignant fibrous | malignant peripheral | malignant triton | mantle cell |
| histiocytoma | nerve sheath tumor | tumor | lymphoma |
| marginal zone B- | mast cell leukemia | mediastinal germ | medullary |
| cell lymphoma | | cell tumor | carcinoma of the |
| | | | breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid | myxoma | myxosarcoma | nasopharyngeal |
| liposarcoma | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell |
| carcinoma | | tumors | carcinoma |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial |
| | | | carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In another embodiment, the cancer is a solid tumor. In another embodiment, the cancer a hematological cancer. Exemplary hematological cancers include, but are not limited to, the cancers listed in Table 4. In another embodiment, the hematological cancer is acute lymphocytic leukemia, chronic lymphocytic leukemia (including B-cell chronic lymphocytic leukemia), or acute myeloid leukemia. In another embodiment, the hematological cancer is chronic myeloid leukemia

TABLE 4

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | chronic myeloid leukemia |

In another embodiment, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In another embodiment, the cancer is selected from the group consisting of acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

In another embodiment, the present disclosure provides methods of treating a benign proliferative disorder, such as, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

In another embodiment, the present disclosure provides methods of treating neurodegenerative diseases comprising administration of an effective amount of a Compound of the Disclosure to a subject in need of such treatment. Exemplary non-limiting neurodegenerative diseases include Alzheimer's disease, multiple sclerosis, Parkinson's disease, amyotrophic lateral sclerosis, and certain lysosomal storage disorders.

In another embodiment, the present disclosure provides methods of treating muscular dystrophies comprising administration of an effective amount of a Compound of the Disclosure to a subject in need of such treatment. Exemplary non-limiting muscular dystrophies include Myotonic, Duchenne, Becker, Limb-girdle, Facioscapulohumeral, Congenital, Oculopharyngeal, Distal, and Emery-Dreifuss muscular dystrophies.

In another embodiment, the present disclosure provides methods of treating infectious and noninfectious inflammatory events, and autoimmune and other inflammatory diseases comprising administration of an effective amount of a Compound of the Disclosure to a subject in need of such treatment. Examples of autoimmune and inflammatory diseases, disorders, and syndromes treated using the compounds and methods described herein include inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, Type I diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituatarism, Guillain-Barre syndrome, Behcet's disease, scleracierma, mycosis fungoides, acute inflammatory responses (such as acute respiratory distress syndrome and ischemia/reperfusion injury), and Graves' disease.

In another embodiment, the present disclosure provides a method of treating systemic inflammatory response syndromes, such as LPS-induced endotoxic shock and/or bacteria-induced sepsis by administration of an effective amount of a Compound of the Disclosure to a mammal, in particular a human in need of such treatment.

In another embodiment, the present disclosure provides a method for treating viral infections and diseases. Examples of viral infections and diseases treated using the compounds and methods described herein include episome-based DNA viruses including, but not limited to, human papillomavirus, Herpesvirus, Epstein-Barr virus, human immunodeficiency virus, hepatitis B virus, and hepatitis C virus.

In another embodiment, prion diseases or disorders comprising administration of an effective amount of a Compound of the Disclosure to a subject in need of such treatment. Exemplary non-limiting prion diseases or disorders include Creutzfeldt-Jakob disease, Gerstmann-Straussler-Scheinker syndrome, fatal familial insomnia, and kuru.

In another embodiment, the present disclosure provides therapeutic method of modulating protein methylation, gene expression, cell proliferation, cell differentiation and/or apoptosis in vivo in diseases mentioned above, in particular cancer, inflammatory disease, and/or viral disease is provided by administering a therapeutically effective amount of a Compound of the Disclosure to a subject in need of such therapy.

In another embodiment, the present disclosure provides a method of regulating endogenous or heterologous promoter activity by contacting a cell with a Compound of the Disclosure.

In methods of the present disclosure, a therapeutically effective amount of a Compound of the Disclosure, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the subject, and ultimately is determined by the attendant physician. Dosage amounts and intervals can be adjusted individually to provide plasma levels of the Compound of the Disclosure that are sufficient to maintain the desired therapeutic effects. The desired dose can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual subject, which can vary with the age, weight, and response of the particular subject.

Compounds of the Disclosure typically are administered in admixture with a pharmaceutical carrier to give a pharmaceutical composition selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington's Pharmaceutical Sciences, Mack Publishing Co., Easton, Pa., 19th ed. 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

The disclosure provides the following particular embodiments in connection with treating a disease in a subject.

Embodiment I. A method of treating a subject, the method comprising administering to the subject a therapeutically effective amount of a Compound of the Disclosure, wherein the subject has cancer, a neurodegenerative disorder, muscular dystrophy, an autoimmune disease, an inflammatory disease, a viral infection, or a prion disease.

Embodiment II. The method Embodiment I, wherein the subject has cancer.

Embodiment III. The method of Embodiment II, wherein the cancer is any one or more of the cancers of Table 3.

Embodiment IV. The method of Embodiment II, wherein the cancer is selected from the group consisting of acute monocytic leukemia, acute myelogenous leukemia, chronic myelogenous leukemia, chronic lymphocytic leukemia mixed lineage leukemia, NUT midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment V. The method of Embodiment II, wherein the cancer is any one or more of the cancers of Table 4

Embodiment VI. The method of any one of Embodiments I-V further comprising administering a therapeutically effective amount of a second therapeutic agent useful in the treatment of the disease or condition, e.g., an immune checkpoint inhibitor or other anticancer agent.

Embodiment VII. The method of any one of Embodiments I-VI, wherein the Compound of the Disclosure is a compound of any one of Formulae I-X, or a pharmaceutically acceptable salt or solvate thereof.

Embodiment VIII. The method of Embodiment VII, wherein the Compound of the Disclosure is a compound of Formula V, or a pharmaceutically acceptable salt or solvate thereof.

Embodiment IX. A pharmaceutical composition comprising a Compound of the Disclosure and a pharmaceutically acceptable excipient for use in treating cancer, a neurodegenerative disorder, muscular dystrophy, an autoimmune disease, an inflammatory disease, a viral infection, or a prion disease.

Embodiment X. The pharmaceutical composition of Embodiment IX for use in treating cancer.

Embodiment XI. The pharmaceutical composition of Embodiment X, wherein the cancer is any one or more of the cancers of Table 3.

Embodiment XII. The pharmaceutical composition of Embodiment X, wherein the cancer is selected from the group consisting of acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment XIII. The pharmaceutical composition of Embodiment X, wherein the cancer is any one or more of the cancers of Table 4.

Embodiment XIV. The pharmaceutical composition of any one of Embodiments IX-XIII, wherein the Compound of the Disclosure is a compound of any one of Formulae I-X, or a pharmaceutically acceptable salt or solvate thereof.

Embodiment XV. The pharmaceutical composition of Embodiment XIV, wherein the Compound of the Disclosure is a compound of Formula V, or a pharmaceutically acceptable salt or solvate thereof.

Embodiment XVI. A Compound of the Disclosure for use in treatment of cancer, a neurodegenerative disorder, muscular dystrophy, an autoimmune disease, an inflammatory disease, a viral infection, or a prion disease.

Embodiment XVII. The compound of Embodiment XVI for use in treating cancer.

Embodiment XVIII. The compound of Embodiment XVII, wherein the cancer is any one or more of the cancers of Table 3.

Embodiment XIX. The compound of Embodiment XVII, wherein the cancer is selected from the group consisting of acute monocytic leukemia, acute myelogenous leukemia, chronic myelogenous leukemia, chronic lymphocytic leukemia mixed lineage leukemia, NUT midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment XX. The compound of Embodiment XVII, wherein the cancer is any one or more of the cancers of Table 4.

Embodiment XXI. The compound of any one of Embodiments XVI-XX, wherein the Compound of the Disclosure is a compound of any one of Formulae I-X, or a pharmaceutically acceptable salt or solvate thereof.

Embodiment XXII. The compound of Embodiment XXI, wherein the Compound of the Disclosure is a compound of Formula V, or a pharmaceutically acceptable salt or solvate thereof.

Embodiment XXIII. Use of a Compound of the Disclosure for the manufacture of a medicament for treatment of cancer, a neurodegenerative disorder, muscular dystrophy, an autoimmune disease, an inflammatory disease, a viral infection, or a prion disease.

Embodiment XXIV. The use of Embodiment XXIII for the treatment of cancer.

Embodiment XXV. The use of Embodiment XXIV, wherein the cancer is any one or more of the cancers of Table 3.

Embodiment XXVI. The use of Embodiment XXIII, wherein the cancer is selected from the group consisting of acute monocytic leukemia, acute myelogenous leukemia, chronic myelogenous leukemia, chronic lymphocytic leukemia mixed lineage leukemia, NUT midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

Embodiment XXVII. The use of Embodiment XXIV, wherein the cancer is any one or more of the cancers of Table 4.

Embodiment XXVIII. The use of any one of Embodiments XXIII-XXVII, wherein the Compound of the Disclosure is a compound of any one of Formulae I-X, or a pharmaceutically acceptable salt or solvate thereof.

Embodiment XXIX. The use of Embodiment XXI, wherein the Compound of the Disclosure is a compound of Formula V, or a pharmaceutically acceptable salt or solvate thereof.

Embodiment XXX. A method of inhibiting BCR-ABL protein within a cell of a subject in need thereof, the method comprising administering to the subject a compound of any one of Formulae I-X, or a pharmaceutically acceptable salt or solvate thereof.

Embodiment XXXI. A method of inhibiting BCR-ABL protein within a cell of a subject in need thereof, the method comprising administering to the subject a compound of Formula V, or a pharmaceutically acceptable salt or solvate thereof.

### V. Kits of the Disclosure

In another embodiment, the present disclosure provides kits which comprise a Compound of the Disclosure (or a composition comprising a Compound of the Disclosure) packaged in a manner that facilitates their use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure, e.g., the method of any one of Embodiments I-VI. In one embodiment, the compound or composition is packaged in a unit dosage form. The kit further can include a device suitable for administering the composition according to the intended route of administration.

### VI. Definitions

The term “a disease or condition wherein inhibition of BCR-ABL provides a benefit” and the like pertains to a disease or condition in which BCR-ABL is important or necessary, e.g., for the onset, progress, expression of that disease or condition, or a disease or a condition which is known to be treated by an BCR-ABL inhibitor. Examples of such conditions include, but are not limited to, a cancer, a neurodegenerative disorder, muscular dystrophy, an autoimmune disease, an inflammatory disease, a viral infection, or a prion disease. One of ordinary skill in the art is readily able to determine whether a Compound of the Disclosure treats a disease or condition mediated by a BCR-ABL inhibitor for any particular cell type, for example, by assays which conveniently can be used to assess the activity of particular compounds. See, e.g., Yue and Turkson, Expert Opinion Invest Drugs 18:45-56 (2009).

The term “BCR-ABL” refers to the fusion gene formed when pieces of chromosomes 9 and 22 break off and trade places. The ABL gene from chromosome 9 joins to the BCR gene on chromosome 22, to form the BCR-ABL fusion gene. The changed chromosome 22 with the fusion gene on it is called the Philadelphia chromosome. The BCR-ABL fusion gene is found in patients having cancer. For example, the BCR-ABL fusion gene most patients with chronic myelogenous leukemia (CML), and in some patients with acute lymphoblastic leukemia (ALL) or acute myelogenous leukemia (AML). This fusion gene encodes the chimeric BCR-ABL protein.

The term “second therapeutic agent” refers to a therapeutic agent different from a Compound of the Disclosure and that is known to treat the disease or condition of interest. For example when a cancer is the disease or condition of interest, the second therapeutic agent can be a known chemotherapeutic drug, like taxol, or radiation, for example.

The term “disease” or “condition” denotes disturbances and/or anomalies that are regarded as being pathological conditions or functions, and that can manifest themselves in the form of particular signs, symptoms, and/or malfunctions. As demonstrated below, Compounds of the Disclosure are inhibitors of BCR-ABL and can be used in treating or preventing diseases and conditions wherein inhibition of BCR-ABL provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and subject to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to a subject in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour (h), 2 h, 4 h, 6 h, 12 h, 24 h, 48 h, 72 h, 96 h, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 h, 4 h, 6 h, 12 h, 24 h, 48 h, 72 h, 96 h, 1 week, 2 weeks, 3 weeks, 4 weeks, 5 weeks, 6 weeks, 8 weeks, or 12 weeks after) the administration of a second therapeutic agent treatment modality (e.g., radiotherapy), to a subject in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 h apart, 2 h to 3 h apart, 3 h to 4 h apart, 4 h to 5 h apart, 5 h to 6 h apart, 6 h to 7 h apart, 7 h to 8 h apart, 8 h to 9 h apart, 9 h to 10 h apart, 10 h to 11 h apart, 11 h to 12 h apart, no more than 24 h apart or no more than 48 h apart. In one embodiment, the components of the combination therapies are administered at about 1 minute to about 24 h apart.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a})C(═O)R^{56b}, —N(R^{56c})S(═O)₂R^{56d}, —C(═O)R⁵⁷, —S(═O)R^{56e}, or —S(═O)₂R⁵⁸; wherein:

R^{56a} is hydrogen or alkyl;

R^{56b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;

R^{56c} is hydrogen or alkyl;

R^{56d} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;

R^{56e} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;

R⁵⁷ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl; and

R⁵⁸ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In one embodiment, the alkynyl is a C₂-C₆ alkynyl. In another embodiment, the alkynyl is a C₂-C₄alkynyl. In another embodiment, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)C≡CH.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In one embodiment, the haloalkyl group is a C₁-C₆ haloalkyl. In another embodiment, the haloalkyl group is a C₁-C₄ haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In one embodiment, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In one embodiment, the alkoxy is a C₁-C₆ alkoxy. In another embodiment, the alkoxy is a C₁-C₄ alkoxy. In another embodiment, the alkyl is a C₁-C₆ alkyl. In another embodiment, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any interior position of the aliphatic hydrocarbon chain so long as each —O—, —N(H)—, —N(C₁-C₄ alkyl)-, and —S— group is separated by at least two —CH₂— groups. In one embodiment, one —CH₂— group is replaced with one —O— group. In another embodiment, two —CH₂— groups are replaced with two —O— groups. In another embodiment, three —CH₂— groups are replaced with three —O— groups. In another embodiment, four —CH₂— groups are replaced with four —O— groups. Non-limiting exemplary heteroalkyl groups include —CH₂OCH₃, —CH₂OCH₂CH₂CH₃, —CH₂CH₂CH₂OCH₃, —CH₂CH₂OCH₂CH₂OCH₂CH₃, —CH₂CH₂OCH₂CH₂OCH₂CH₂OCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, and R⁵⁸ are as defined in connection with the term “optionally substituted alkyl” and R⁵⁹ is (hydroxy)alkyl or (amino)alkyl. The term optionally substituted cycloalkyl also includes cycloalkyl groups having fused optionally substituted aryl or optionally substituted heteroaryl groups such as

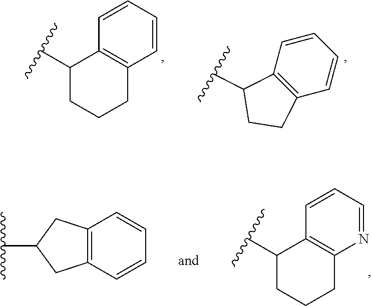 [SMILES: *C1CCCc2ccccc21 ; *C1CCc2ccccc21 ; *C1Cc2ccccc2C1 ; *C1CCCc2ncccc21]

Non-limiting exemplary optionally substituted cycloalkyl groups include:

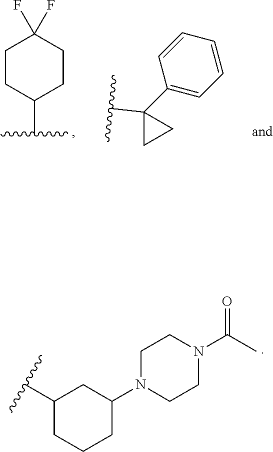 [SMILES: *C1CCC(F)(F)CC1 ; *C1(c2ccccc2)CC1 ; *C1CCCC(N2CCN(C(C)=O)CC2)C1]

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

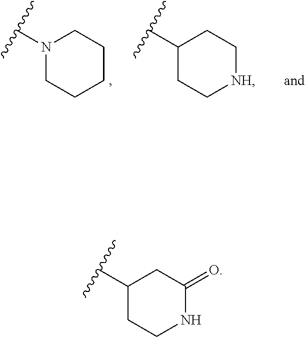 [SMILES: *N1CCCCC1 ; *C1CCNCC1 ; *C1CCNC(=O)C1]

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.” Substitution may occur on any available carbon or nitrogen atom of the heterocyclo group. Non-limiting exemplary optionally substituted heterocyclo groups include:

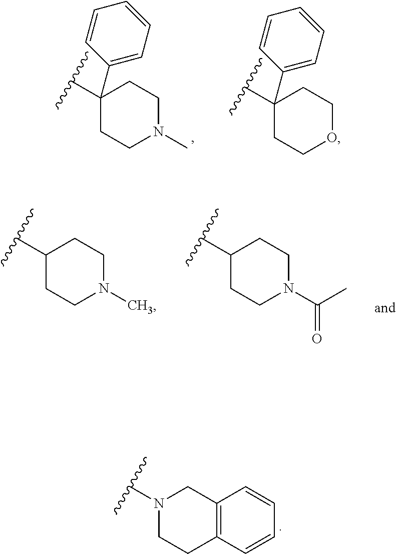

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R⁵⁶°, R⁵⁷, R⁵⁸, and R⁵⁹ are as defined in connection with the term “optionally substituted cycloalkyl.”

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, O-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., TH-pyrrol-2-yl and TH-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted heteroaryl has two substituents. In another embodiment, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “(cyano)alkyl” as used herein by itself or as part of another group refers to an alkyl substituted with one, two, or three cyano groups. In one embodiment, the alkyl is substituted with one cyano group. In another embodiment, the alkyl is a C₁-C₆ alkyl In another embodiment, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. In another embodiment, the alkyl is substituted with one optionally substituted cycloalkyl group. In another embodiment, the alkyl is substituted with two optionally substituted cycloalkyl groups. Non-limiting exemplary (cycloalkyl)alkyl groups include:

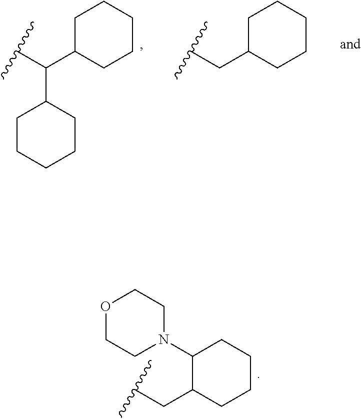 [SMILES: *C(C1CCCCC1)C1CCCCC1 ; *CC1CCCCC1 ; *CC1CCCCC1N1CCOCC1]

The term “sulfonamido” as used herein by itself or as part of another group refers to a radical of the formula —SO₂NR^{50a}R^{50b}, wherein R^{50a} and R^{50b} are each independently hydrogen, alkyl, optionally substituted cycloalkyl, optionally substituted heterocyclo, optionally substituted aryl, or optionally substituted heteroaryl; or R^{50a} and R^{50b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “ureido” as used herein by itself or as part of another group refers to a radical of the formula —NR^{51a}—C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R^{51c} taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R^{53c} are each independently hydrogen, alkyl, optionally substituted cycloalkyl, optionally substituted heterocyclo, optionally substituted aryl, or optionally substituted heteroaryl; or R^{52b} and R^{52c} taken together with the nitrogen to which they are attached form a 4- to 8-membered optionally substituted heterocyclo group; and R⁵³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom. Non-limiting exemplary (heterocyclo)alkyl groups include:

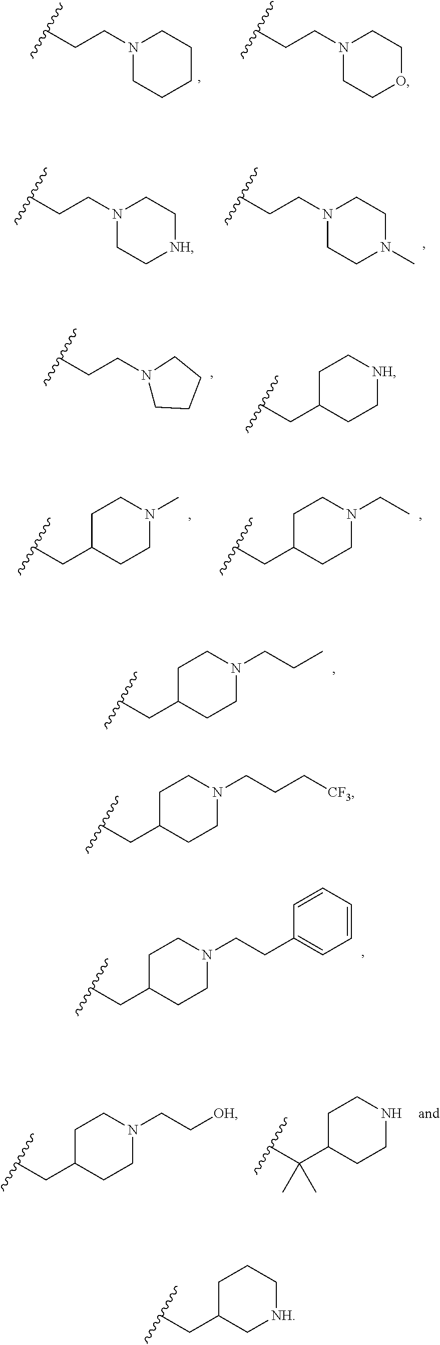

The term “carbamate” as used herein by itself or as part of another group refers to a radical of the formula —NR^{54a}—C(═O)—OR^{54b}, wherein R^{54a} is hydrogen or alkyl, and R^{54b} is hydrogen, alkyl, optionally substituted cycloalkyl, optionally substituted heterocyclo, optionally substituted aryl, or optionally substituted heteroaryl. A non-limiting exemplary carbamate group is —NH—(C═O)—OtBu.

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl. Non-limiting exemplary (heteroaryl)alkyl groups include:

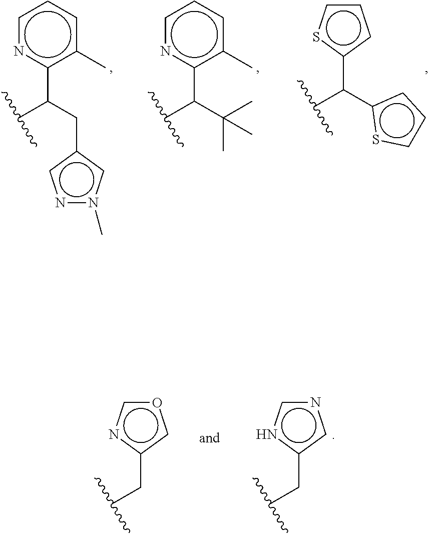

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “amido” as used herein by itself or as part of another group refers to a radical of formula —C(═O)NR^{60a}R^{60b}, wherein R^{60a} and R^{60b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{60a} and R^{60b} taken together with the nitrogen to which they are attached from a 4- to 8-membered optionally substituted heterocyclo group. In one embodiment, R^{60a} and R^{60b} are each independently hydrogen or C₁-C₆ alkyl.

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

In another embodiment, the amino is an “alkylamino,” i.e., an amino group wherein R^{55a} is C₁₋₆ alkyl and R^{55b} is hydrogen. In one embodiment, R^{55a} is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R^{55a} and R^{55b} are each independently C₁₋₆ alkyl. In one embodiment, R^{55a} and R^{55b} are each independently C₁-C₄ alkyl. Non-limiting exemplary dialkylamino groups include —N(CH₃)₂ and —N(CH₃)CH₂CH(CH₃)₂.

In another embodiment, the amino is a “hydroxyalkylamino,” i.e., an amino group wherein R^{55a} is (hydroxyl)alkyl and R^{55b} is hydrogen or C₁-C₄ alkyl.

In another embodiment, the amino is a “cycloalkylamino,” i.e., an amino group wherein R^{55a} is optionally substituted cycloalkyl and R^{55b} is hydrogen or C₁-C₄ alkyl.

In another embodiment, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂, and —N(CH₃)CH₂Ph.

In another embodiment, the amino is a “(cycloalkyl)alkylamino,” i.e., an amino group wherein R^{55a} is (cycloalkyl)alkyl and R^{55b} is hydrogen or C₁-C₄ alkyl. Non-limiting exemplary (cycloalkyl)alkylamino groups include:

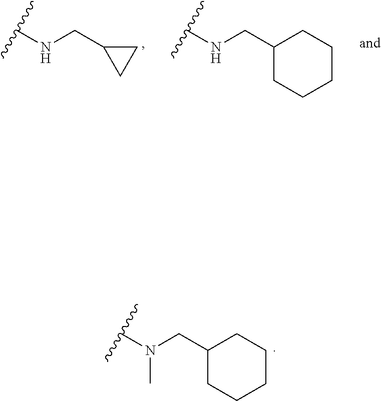

In another embodiment, the amino is a “(heterocyclo)alkylamino,” i.e., an amino group wherein R^{55a} is (heterocyclo)alkyl and R^{55b} is hydrogen or C₁-C₄ alkyl. Non-limiting exemplary (heterocyclo)alkylamino groups include:

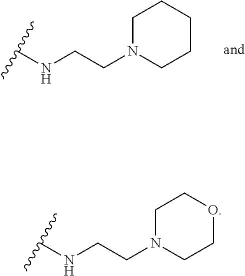 [SMILES: *NCCN1CCCCC1 ; *NCCN1CCOCC1]

The term “(amino)alkyl” as used herein by itself or as part of another group refers to an alkyl substituted with one amino group. In one embodiment, the amino group is —NH₂. In one embodiment, the amino group is an alkylamino. In another embodiment, the amino group is a dialkylamino. In another embodiment, the alkyl is a C₁-C₆ alkyl. In another embodiment, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a composition wherein substantially all of the atoms at a position within the Compound of the Disclosure are replaced by an atom having a different atomic mass or mass number. In another embodiment, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

Compounds of the Disclosure may contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number±10%. Thus, “about 10” means 9 to 11.

### EXAMPLES

### General Synthetic Schemes

Unless otherwise stated, starting materials and reagents used in preparing these compounds are either available from commercial suppliers such as Aldrich Chemical Co., or are prepared by methods known to those skilled in the art following procedures set forth in references such as Fieser and Fieser's Reagents for Organic Synthesis, Volumes 1-7 (John Wiley and sons, 1991); Rodd's Chemistry of Carbon Compounds, Volumes 1-5 and Supplementals (Elservier Science Publishers, 1989); Organic Reactions, Volumes 1-40 (John Wiley and Sons, 1991), March's Advanced Organic Chemistry, (John Wiley and Sons, 5^{th} Edition), and Larock's Comprehensive Organic Transformations (VCH Publishers Inc., 1989). These synthetic methods are merely illustrative of some methods by which the Compounds and Intermediates of the Disclosure can be synthesized, and various modifications to these methods can be made in view of this disclosure. The starting materials and the intermediates, and the final products for the reaction may be isolated and purified if desired using conventional techniques, including but no limited to filtration, distillation, crystallization, chromatography and the like. Such materials may be characterized using conventional means, including physical constants and spectral data. In the reactions described, it may be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry” (John Wiley and Sons, 1991).

Unless specified to the contrary, the reactions described herein take place at atmospheric pressure over a temperature range from about −78° C. to about 150° C., more preferably from about 0° C. to about 125° C., and most preferably at about room (or ambient) temperature, e.g., about 22° C.

Compounds of the Disclosure and Intermediates of the Disclosure can be prepared according to General Scheme 1, wherein R¹, R^{2a}, R^{2b}, R^{2c}, R^{2d}, R³, R^{4a}, R^{4b}, A, L, X, Y, Z, and [symbol] are as defined in the specification, ‘halo’ represents, e.g., chloro, bromo or iodo, and “alkyl” represents, e.g., methyl, ethyl, isopropyl or tert-butyl group.

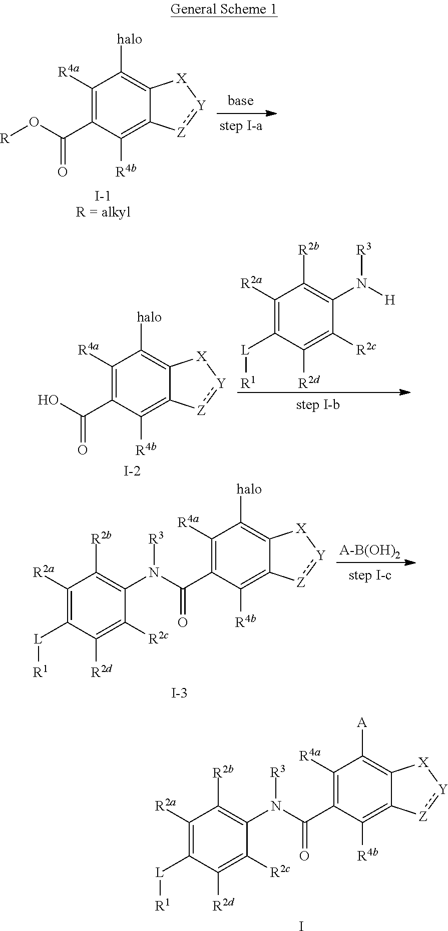

Step I-a: An intermediate of formula (I-2) can be prepared by hydrolyzing the ester from an intermediate of formula (I-1) in the presence of suitable solvent (for example tetrahydrofuran, 1,4-dioxane, methanol, acetonitrile, ethanol, and the like) and a suitable base (for example sodium hydroxide, potassium hydroxide, and the like). The reaction takes place from about 0° C. to reflux and can take up to about 2 to 24 hours to complete. The intermediate of formula (I-1) can be purchased from commercial sources or prepared according to the representative examples below.

Step I-b: Intermediate of formula (I-2) is turned to acid chloride, then reacted with compound of formula (I-4) to form intermediate of formula (I-3) in the presence of a suitable organic base such as triethyl amine or diisopropylethylamine. The intermediate of formula (I-3) can also be prepared by coupling of intermediate of formula (I-2) with intermediate of formula (I-4) in the presence of a suitable coupling reagent such as HATU, EDC, DCC, CDI, HBTU, and the like, in the presence of a suitable base such as triethyl amine, diisopropylethylamine, DMAP and the like, and in the presence of a suitable solvent such as dichloromethane or tetrahydrofuran. The reaction takes place from about 0° C. to room temperature and can take up to about 2 to 24 hours to complete.

Step I-c: A compound of the Formula I can be prepared by reacting the intermediate of formula (I-3) with a aromatic boronic acid of formula A-B(OH)₂ in the presence of a suitable catalyst (for example Pd(dppf)Cl₂, Pd(OAc)₂, Pd₂(dba)₃, Pd(PPh₃)₄, and the like), and in the presence of a suitable ligand (for example triphenyl phosphine, tricyclohexyl phosphine, BINAP, and the like), and in the presence of a suitable base (for example potassium carbonate, cesium carbonate, potassium hydroxide, and the like), and in the presence of a suitable solvent (for example tetrahydrofuran, 1,4-dioxane, toluene, ethanol, dimethyl ether, and the like). The reaction takes place at atmospheric pressure or in a microwave reactor from about room temperature to 150° C. and can take up to about 1 to 10 hours to complete.

### Example 1—Synthesis of Compounds of the Disclosure

### Example III-1

### Synthesis of N-(4-(chlorodifluoromethoxy)phenyl)-1-isopropyl-3-methyl-4-(1H-pyrazol-5-yl)indoline-6-carboxamide (Cpd. No. III-1)

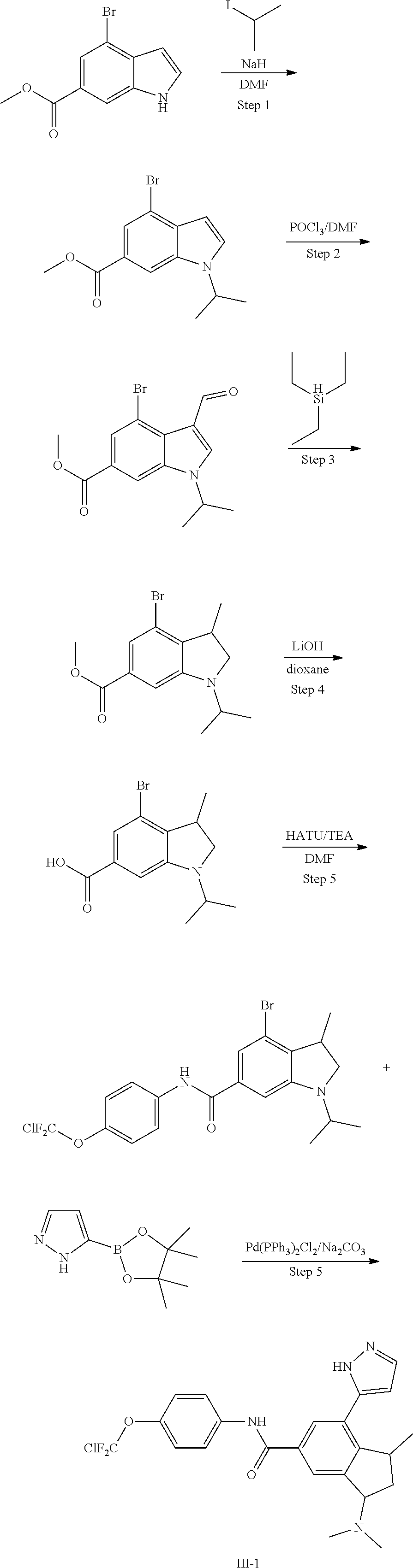

### Step 1: Synthesis of methyl 4-bromo-1-isopropyl-1H-indole-6-carboxylate

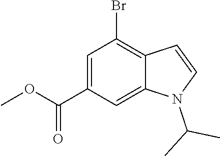 [SMILES: COC(=O)c1cc(Br)c2ccn(C(C)C)c2c1]

To a suspension of NaH (0.378 g, 15.74 mmol) N,N-dimethylformamide (10 mL), was added methyl 4-bromo-1H-indole-6-carboxylate (2 g, 7.87 mmol). The mixture was stirred at room temperature for 10 min, then was added 2-iodopropane (2.68 g, 15.74 mmol) and stirred for another 3 h. The mixture was concentrated under reduced pressure to give a crude product that was eluted through a silica gel column (ethyl acetate/hexane from 10% to 40%) to give methyl 4-bromo-1-isopropyl-1H-indole-6-carboxylate (1.7 g, 72.9%).

### Step 2: Synthesis of methyl 4-bromo-3-formyl-1-isopropyl-1H-indole-6-carboxylate

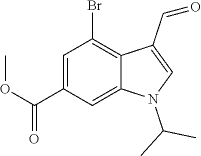 [SMILES: COC(=O)c1cc(Br)c2c(C=O)cn(C(C)C)c2c1]

N,N-dimethylformamide (1.5 mL) was added to a 100 mL two-necked-round-bottomed flask, cooled to 0° C. To the mixture was dropped phosphoryl trichloride (647 mg, 4.22 mmol) over 10 min, stirred for 15 min, then was added a solution of methyl 4-bromo-1-isopropyl-1H-indole-6-carboxylate (500 mg, 1.688 mmol) in 4 mL N,N dimethylformamide. The reaction mixture was stirred for 40 min at 10° C., then warmed up to 35° C. and stirred for another 40 min. Water (20 mL) was added to the reaction mixture followed by extraction with ethyl acetate (20 mL×3). The organic layers were combined and concentrated under reduced pressure, and the residue was purified by silica gel column chromatography (ethyl acetate/hexane from 10% to 40%) to give methyl 4-bromo-3-formyl-1-isopropyl-1H-indole-6-carboxylate (315 mg, 71.9%) as a white solid.

### Step 3: Synthesis of methyl 4-bromo-1-isopropyl-3-methylindoline-6-carboxylate

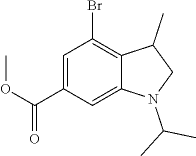 [SMILES: COC(=O)c1cc(Br)c2c(c1)N(C(C)C)CC2C]

methyl 4-bromo-3-formyl-1-isopropyl-1H-indole-6-carboxylate (200 mg, 0.617 mmol) was dissolved in 5.0 mL trifluoroacetic acid. Triethylsilane (215 mg, 1.851 mmol) was added to the reaction mixture, and the mixture was stirred at 60° C. for 3 h. Water (10 mL) was added to the reaction mixture, followed by extraction with ethyl acetate (10 mL×3). The combined organic layers were dried over Na₂SO₄, filtered and concentrated to give a crude product that was eluted through a silica gel column (ethyl acetate/hexane from 10% to 50%) to give methyl 4-bromo-1-isopropyl-3-methylindoline-6-carboxylate (577.0 mg, 93%). MS: 312.0 (M+H)⁺.

### Step 4: Synthesis of 4-bromo-1-isopropyl-3-methylindoline-6-carboxylic acid

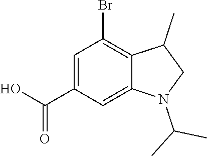 [SMILES: CC1CN(C(C)C)c2cc(C(=O)O)cc(Br)c21]

Lithium hydroxide (2N, 5.0 mmol) was added to a solution of methyl 4-bromo-1-isopropyl-3-methylindoline-6-carboxylate (180.0 mg, 0.577 mmol) in dioxane (3.0 mL). The mixture was stirred at 40° C. for overnight. The reaction mixture was concentrated, then acidified with 1 N HCl (20.0 mL). The precipitate was collected and washed with water and hexane, then dried under vacuum to give 7-bromo-1-isopropylindoline-5-carboxylic acid (160 mg, 93.0%) as a white solid. MS: 299.0 (M+H)⁺.

### Step 5: Synthesis of 4-bromo-N-(4-(chlorodifluoromethoxy)phenyl)-1-isopropyl-3-methylindoline-6-carboxamide

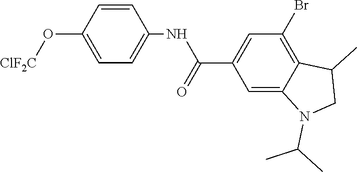

4-(chlorodifluoromethoxy)aniline (150.0 mg, 0.775 mmol), diisopropylethyl amine (125.0 mg, 0.969 mmol) and 2-(7-azabenzotriazol-1-yl)-N,N,N′,N′-tetramethyluronium hexafluorophosphate (368.0 mg, 0.969 mmol) were added to a solution of 4-bromo-1-isopropyl-3-methylindoline-6-carboxylic acid (193.0 mg, 0.464 mmol) in N,N-dimethylformamide (6.0 mL). The mixture was stirred at 45° C. for 5 h. The reaction mixture was diluted with dichloromethane (10.0 mL), then washed with water (30.0 mL) and brine (30.0 mL). The organic layer was dried over Na₂SO₄, filtered and concentrated to give a crude product that was eluted through a silica gel column (ethyl acetate/hexane from 0% to 35%) to give 4-bromo-N-(4-(chlorodifluoromethoxy)phenyl)-1-isopropyl-3-methylindoline-6-carboxamide (186.0 mg, 60.8%) as a white solid. MS: 474.9 (M+H)⁺.

### Step 6: Synthesis of N-(4-(chlorodifluoromethoxy)phenyl)-1-isopropyl-3-methyl-4-(1H-pyrazol-5-yl)indoline-6-carboxamide (Cpd. No. III-1)

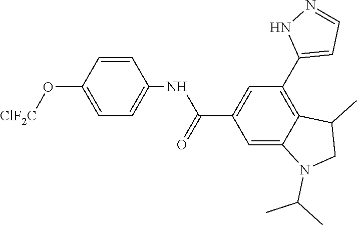

5-(4,4,5,5-tetramethyl-1,3,2-dioxaborolan-2-yl)-1H-pyrazole (63.9 mg, 0.329 mmol) and Pd(PPh3)₂Cl₂ (19.26 mg, 0.027 mmol) were added to a solution of 4-bromo-N-(4-(chlorodifluoromethoxy)phenyl)-1-isopropyl-3-methylindoline-6-carboxamide (130.0 mg, 0.247 mmol) in dimethoxyethane/water/EtOH (0.15 mL/0.3 L/1.5 mL. The mixture was purged with nitrogen and the reaction mixture was stirred at 110° C. under MW for 2 h. The reaction mixture was diluted with diethyl ether (20.0 mL), then washed with water (20.0 mL) and brine (20.0 mL). The organic layer was dried over Na₂SO₄, filtered and concentrated to give a crude product that was purified using preparative HPLC to give N-(4-(chlorodifluoromethoxy)phenyl)-1-isopropyl-3-methyl-4-(1H-pyrazol-5-yl)indoline-6-carboxamide (45 mg, 35.6%) as a white solid. 1H NMR (400 MHz, DMSO-d₆) δ 13.03 (s, 1H), 10.34 (s, 1H), 7.99-7.92 (m, 2H), 7.89-7.83 (m, 1H), 7.56-7.52 (m, 1H), 7.41-7.39 (m, 2H), 6.94-6.91 (m, 1H), 6.77-6.75 (m, 1H), 4.06-3.95 (m, 1H), 3.95-3.84 (m, 1H), 3.59-3.49 (m, 1H), 3.20-3.13 (m, 1H), 1.30 (d, J=6.1 Hz, 6H), 1.15 (d, J=7.4 Hz, 3H). MS: 461.0 (M+H)⁺.

### Example III-2

### Synthesis of N-(4-(chlorodifluoromethoxy)phenyl)-1,3,3-trimethyl-4-(1H-pyrazol-5-yl)indoline-6-carboxamide (Cpd. No. III-2)

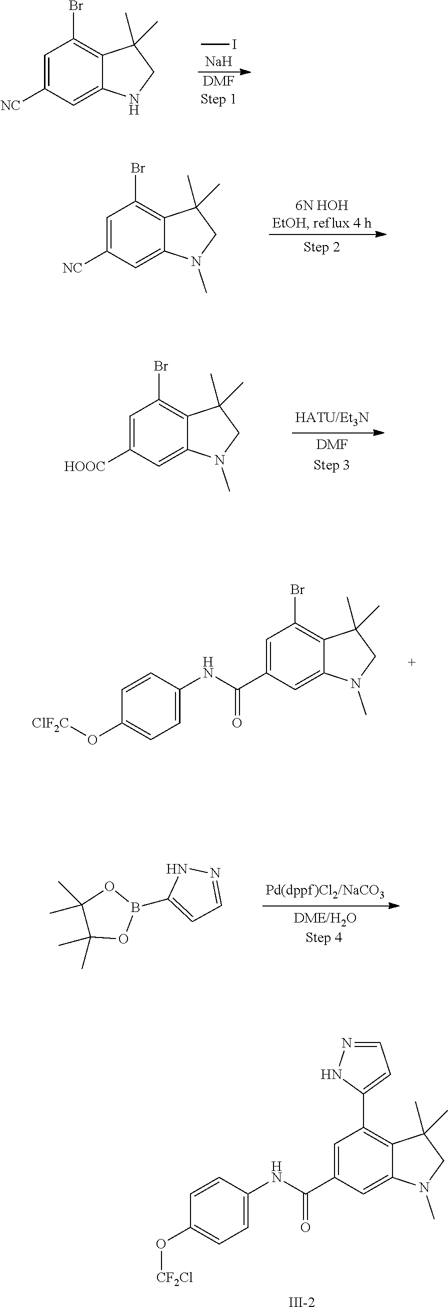

### Step 1: Synthesis of 4-bromo-1,3,3-trimethylindoline-6-carbonitrile

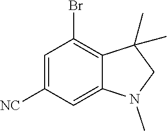

In a 50 mL round-bottomed flask, NaH (19.11 mg, 0.796 mmol) was dissolved in N,N-dimethylformamide (5 mL) under nitrogen, 4-bromo-3,3-dimethylindoline-6-carbonitrile (200 mg, 0.796 mmol) in 3 mL N,N-dimethylformamide was added to the reaction mixture and stirred for 10 min, then iodomethane (226 mg, 1.593 mmol) was added to the reaction mixture. The mixture was stirred for 3 h at 30° C., then cooled to 0° C. The reaction was quenched with aqueous NH₄Cl, followed by extraction with ethyl acetate (15 mL×3). The combined organic layers were dried over Na₂SO₄, filtered and concentrated to give a crude product that was purified by silica gel column chromatography (ethyl acetate/hexane from 10% to 40%) to give 4-bromo-1,3,3-trimethylindoline-6-carbonitrile (150 mg, 71.0%). MS: 266.02 (M+H)⁺.

### Step 2: Synthesis of 4-bromo-1,3,3-trimethylindoline-6-carboxylic acid

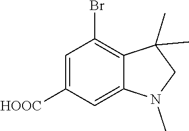 [SMILES: CN1CC(C)(C)c2c(Br)cc(C(=O)O)cc21]

Essentially the same protocol of Step 4 in EXAMPLE 3 to afford 4-bromo-1,3,3-trimethylindoline-6-carboxylic acid. MS: 284.02 (M+H)⁺.

### Step 3: Synthesis of 4-bromo-N-(4-(chlorodifluoromethoxy)phenyl)-1,3,3-trimethylindoline-6-carboxamide

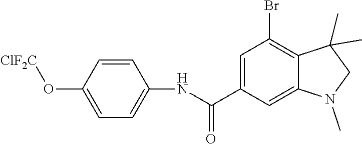

Essentially the same protocol of Step 5 in EXAMPLE 3 to afford 4-bromo-N-(4-(chlorodifluoromethoxy)phenyl)-1,3,3-trimethylindoline-6-carboxamide. MS: 459.02 (M+H)⁺.

### Step 4: Synthesis of N-(4-(chlorodifluoromethoxy)phenyl)-1,3,3-trimethyl-4-(1H-pyrazol-5-yl)indoline-6-carboxamide

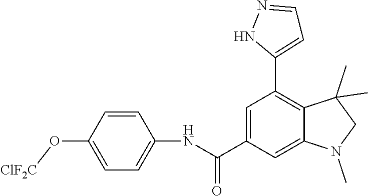

Essentially the same protocol of the preparation of Cpd. No. III-3 in EXAMPLE III-3 was used to afford Cpd. No. III-2 as a white solid. ¹H NMR (400 MHz, DMSO-d₆) δ 12.98 (s, 1H), 10.31 (s, 1H), 7.94-7.83 (m, 3H), 7.37 (d, J=8.6 Hz, 2H), 7.26 (s, 1H), 7.04-7.01 (m, 1H), 6.45-6.42 (m, 1H), 3.06 (s, 2H), 2.81 (s, 3H), 1.26 (s, 6H).

### Example III-3

### Synthesis of N-(4-(chlorodifluoromethoxy)phenyl)-3,3-dimethyl-4-(1H-pyrazol-5-yl)indoline-6-carboxamide (Cpd. No. III-3)

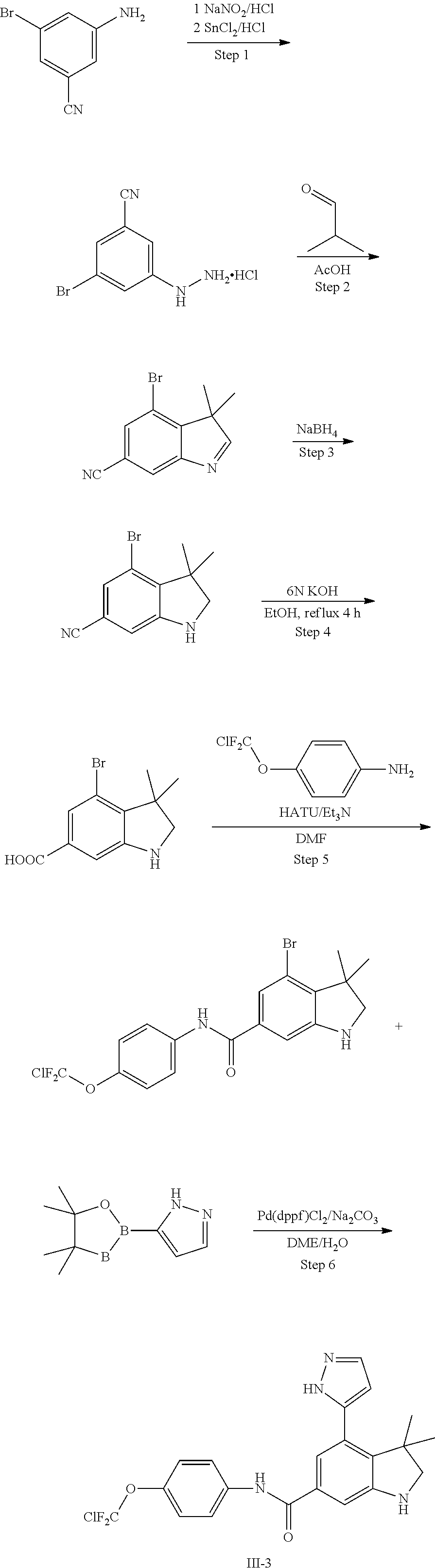

### Step 1: Synthesis of 3-bromo-5-hydrazinylbenzonitrile hydrochloride

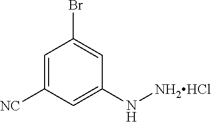

In a 500 mL three-necked-round-bottomed flask, to a solution of 3-amino-5-bromobenzonitrile (12.7 g, 65.2 mmol) in HCl (150 mL) was added a solution of sodium nitrite (6.75 g, 98 mmol) in water (220 mL) dropwise at −5° C. The reaction mixture was stirred at −5° C. for 1 h, then was added tin(II) chloride (30.9 g, 163 mmol) in HCl (135 mL). The mixture was stirred at room temperature for another 1 h, then the resulting precipitate was collected to afford methyl 3-bromo-5-hydrazinylbenzoate hydrochloride (13 g, 72.2%). MS: 213.90 (M+H)⁺.

### Step 2: 4-bromo-3,3-dimethyl-3H-indole-6-carbonitrile

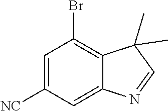

In a 50 mL round-bottomed flask, to a solution of 3-bromo-5-hydrazinylbenzonitrile hydrochloride (1.75 g, 7.12 mmol) in acetic acid (10 mL) under nitrogen, was added isobutyraldehyde (1.28 g, 17.76 mmol). The reaction mixture was stirred at 50° C. for 2 h, then was poured to 150 mL aqueous NaHCO₃, followed by extraction with dichloromethane (100 mL×3). The combined organic layers were dried over Na₂SO₄, and concentrated to give a crude product that was used in the next step without purification. MS: 249.99 (M+H)⁺.

### Step 3: Synthesis of 4-bromo-3,3-dimethylindoline-6-carbonitrile

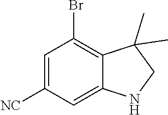

To a solution of 4-bromo-3,3-dimethyl-3H-indole-6-carbonitrile in 12 mL tetrahydrofuran was added NaBH₄ (404 mg, 10.64 mmol) at 0° C. After stirring for 20 min, water (10 mL) was added to quench the reaction. The mixture was extracted with ethyl acetate (10 mL×3). The combined organic layers were dried over Na₂SO₄, filtered and concentrated to give a crude product that was eluted through a silica gel column (ethyl acetate/hexane from 10% to 50%) to give 4-bromo-3,3-dimethylindoline-6-carbonitrile (400 mg, 19.82%). MS: 252.00, 253.90 (M+H)⁺.

### Step 4: Synthesis of 4-bromo-3,3-dimethylindoline-6-carboxylic acid

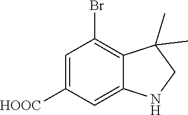

In a 100 mL round-bottomed flask, 4-bromo-3,3-dimethylindoline-6-carbonitrile (200 mg, 0.796 mmol) was dissolved in EtOH (10 mL) under nitrogen, 3 mL 6N KOH (1120 mg, 19.96 mmol) was added to the reaction mixture, the reaction was heated to reflux for overnight. The reaction mixture was acidified with TN HCl and extracted with ethyl acetate (10 mL×3). The resulting organic layers were combined, dried over Na₂SO₄, filtered and concentrated to give a crude product that was eluted through a silica gel column (ethyl acetate/hexane from 10% to 80%) to give 4-bromo-3,3-dimethylindoline-6-carboxylic acid (150 mg, 69.7%) as a colorless oil. MS: 252.00, 253.90 (M+H)⁺.

### Step 5: Synthesis of 4-bromo-N-(4-(chlorodifluoromethoxy)phenyl)-3,3-dimethylindoline-6-carboxamide

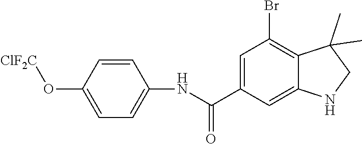

In a 50 mL round-bottomed flask, to a solution of 4-bromo-3,3-dimethylindoline-6-carboxylic acid (200 mg, 0.740 mmol) in N,N-dimethylformamide (3 mL), was added TEA (150 mg, 1.481 mmol) and 2-(7-azabenzotriazol-1-yl)-N,N,N′,N′-tetramethyluronium hexafluorophosphate (422 mg, 1.111 mmol). The reaction mixture was stirred for 10 min, then was added 4-(chlorodifluoromethoxy)aniline (215 mg, 1.111 mmol). After the addition, the mixture was stirred for overnight at room temperature, then quenched with water (20 mL), followed by extraction with ethyl acetate (10 mL×3). The combined organic layers were concentrated to give a crude product that was eluted through a silica gel column (ethyl acetate/hexane from 0% to 20%) to give 4-bromo-N-(4-(chlorodifluoromethoxy) phenyl)-3,3-dimethylindoline-6-carboxamide (100 mg, 30.3%). MS: 445.90, 447.90 (M+H)⁺.

### Step 6: Synthesis of N-(4-(chlorodifluoromethoxy)phenyl)-3,3-dimethyl-4-(1H-pyrazol-5-yl)indoline-6-carboxamide (Cpd. No. III-3)

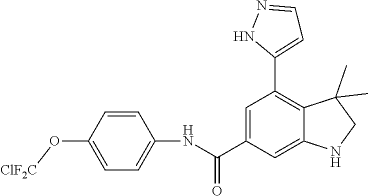

5-(4,4,5,5-tetramethyl-1,3,2-dioxaborolan-2-yl)-1H-pyrazole (69.7 mg, 0.359 mmol), Na₂CO₃ (76.0 mg, 0.718 mmol) and PdCl₂(dppf)-CH₂Cl₂ adduct (16.8 mg, 0.024 mmol) were added to a solution of 4-bromo-N-(4-(chlorodifluoromethoxy)phenyl)-3,3-dimethylindoline-6-carboxamide (110.0 mg, 0.239 mmol) in dimethoxyethane (2 mL) and water (0.4 ml). The mixture was purged with nitrogen and stirred at 110° C. under MW for 2 h. The reaction mixture was diluted with diethyl ether (20.0 mL), then washed with water (20.0 mL) and brine (20.0 mL). The organic layer was dried over Na₂SO₄, filtered and concentrated to give a crude product that was purified using preparative HPLC to give N-(4-(chlorodifluoromethoxy)phenyl)-3,3-dimethyl-4-(1H-pyrazol-5-yl)indoline-6-carboxamide (30.0 mg, 28.1%) as a white solid. ¹H NMR (400 MHz, chloroform-d) δ 7.97 (s, 1H), 7.75-7.73 (m, 1H), 7.69 (d, J=8.7 Hz, 2H), 7.57-7.51 (m, 1H), 7.27-7.19 (m, 2H), 7.11 (s, 1H), 7.05-6.98 (m, 1H), 6.55-6.49 (m, 1H), 3.36 (s, 2H), 1.25 (s, 6H). MS: 434.00 (M+H)⁺.

### Example III-4

### Synthesis of 1-acetyl-N-(4-(chlorodifluoromethoxy)phenyl)-3,3-dimethyl-4-(1H-pyrazol-5-yl)indoline-6-carboxamide (Cpd. No. III-4)

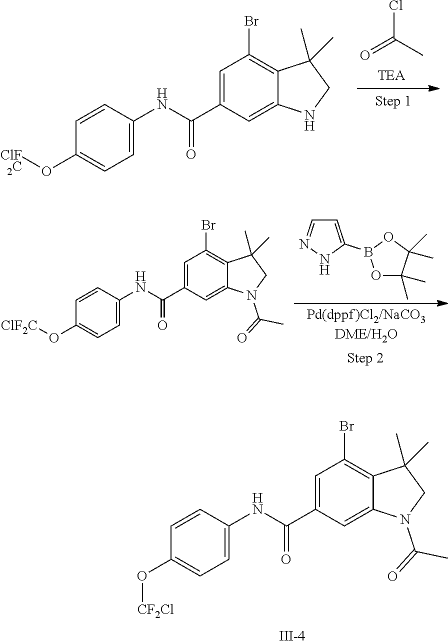

### Step 1: Synthesis of 1-acetyl-4-bromo-N-(4-(chlorodifluoromethoxy)phenyl)-3,3-dimethylindoline-6-carboxamide

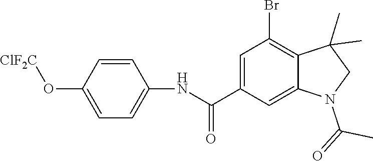

In a 50 mL round-bottomed flask, to a solution of 4-bromo-N-(4-(chlorodifluoromethoxy) phenyl)-3,3-dimethylindoline-6-carboxamide (100 mg, 0.224 mmol) in dichloromethane (5 mL) was added TEA (45.4 mg, 0.449 mmol). After being cooled to 0° C., the mixture was added acetyl chloride (21.13 mg, 0.269 mmol) and stirred for another 1 h at room temperature. Water (10 mL) was added to the reaction mixture followed by extraction with ethyl acetate (15 mL×3). The combined organic layers were concentrated to give a crude product that was purified through a silica gel column (ethyl acetate/hexane from 10% to 70%) to give 1-acetyl-4-bromo-N-(4-(chlorodifluoromethoxy)phenyl)-3,3-dimethylindoline-6-carboxamide (70 mg, 64.0%). ¹H NMR (400 MHz, chloroform-d) δ 8.69 (s, 1H), 8.14 (s, 1H), 7.88 (s, 1H), 7.76-7.69 (m, 2H), 7.28-7.23 (m, 2H), 3.88 (s, 2H), 2.29 (s, 3H), 1.58 (s, 6H), MS: 487.90, 489.90 (M+H)⁺.

### Step 2: Synthesis of 1-acetyl-N-(4-(chlorodifluoromethoxy)phenyl)-3,3-dimethyl-4-(1H-pyrazol-5-yl)indoline-6-carboxamide

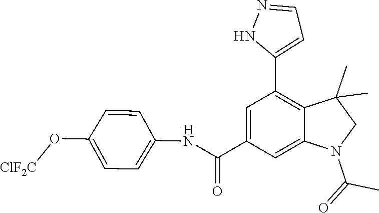

Essentially the same protocol of the preparation of Cpd. No. III-3 in Example III-3 was used to afford Cpd. No. III-4 as a white solid. ¹H NMR (400 MHz, chloroform-d) δ 8.76 (s, 1H), 8.31 (s, 1H), 7.76 (d, J=8.8 Hz, 2H), 7.71-7.68 (m, 3H), 7.27 (s, 1H), 6.50 (s, 1H), 3.81 (s, 2H), 2.28 (s, 3H), 1.31 (s, 6H). MS: 434.00 (M+H)⁺.

### Example III-5

### Synthesis of N-(4-(chlorodifluoromethoxy)phenyl)-3,3-dimethyl-1-(methylsulfonyl)-4-(1H-pyrazol-5-yl)indoline-6-carboxamide (Cpd. No. III-5)

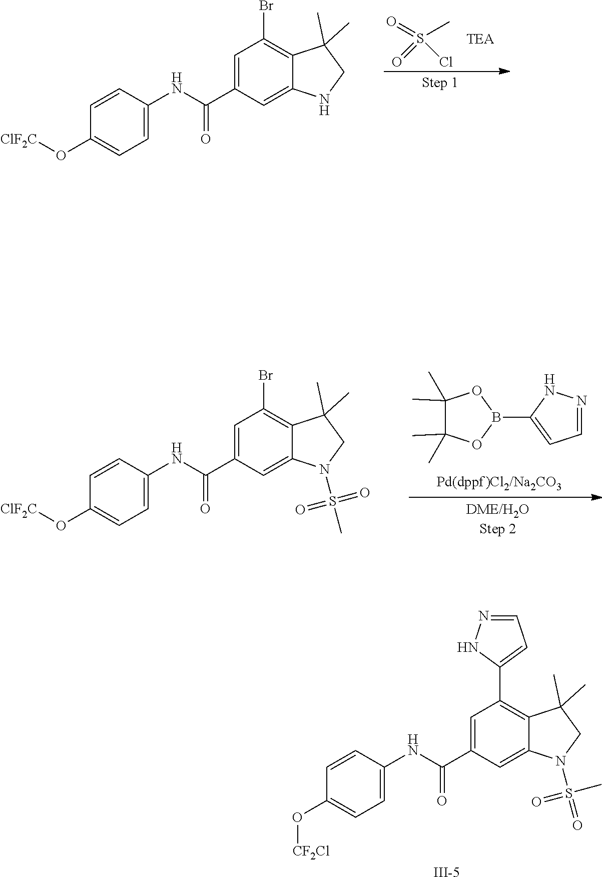

### Step 1: Synthesis of 4-bromo-N-(4-(chlorodifluoromethoxy)phenyl)-3,3-dimethyl-1-(methylsulfonyl)indoline-6-carboxamide

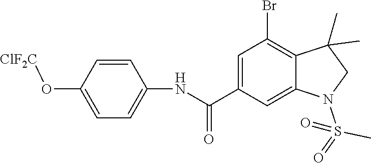

In a 50 mL round-bottomed flask, 4-bromo-N-(4-(chlorodifluoromethoxy) phenyl)-3,3-dimethylindoline-6-carboxamide (70 mg, 0.157 mmol) and TEA (31.8 mg, 0.314 mmol) were dissolved in dichloromethane (5 mL) under nitrogen. To the resulting solution was added methanesulfonyl chloride (27.0 mg, 0.236 mmol). After being stirred for 1 h at room temperature, the mixture was quenched with water (10 mL), followed by extraction with ethyl acetate (10 mL×3). The combined organic layers were concentrated to give a crude product that was purified by silica gel column chromotography (ethyl acetate/hexane from 10% to 70%) to give 4-bromo-N-(4-(chlorodifluoromethoxy)phenyl)-3,3-dimethyl-1-(methylsulfonyl)indoline-6-carboxamide (40 mg, 48.6%). MS: 523.85, 525.80 (M+H)⁺.

### Step 2: Synthesis of N-(4-(chlorodifluoromethoxy)phenyl)-3,3-dimethyl-1-(methylsulfonyl)-4-(1H-pyrazol-5-yl)indoline-6-carboxamide

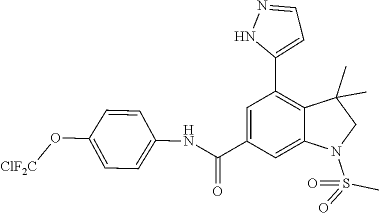

Essentially the same protocol of the preparation of Cpd. No. III-3 in EXAMPLE III-3 was used to afford Cpd. No. III-5 as a white solid. ¹H NMR (400 MHz, DMSO-d₆) δ 10.46 (s, 1H), 7.87 (d, J=8.6 Hz, 2H), 7.81 (s, 1H), 7.63 (d, J=1.6 Hz, 1H), 7.36-7.33 (m, 3H), 6.50 (d, J=2.3 Hz, 1H), 3.66 (s, 2H), 3.13 (s, 3H), 1.28 (s, 6H).

### Example IV-1

### Synthesis of N-(4-(chlorodifluoromethoxy)phenyl)-1,1,2-trimethyl-3-oxo-7-(1H-pyrazol-5-yl)isoindoline-5-carboxamide (Cpd. No. IV-1)

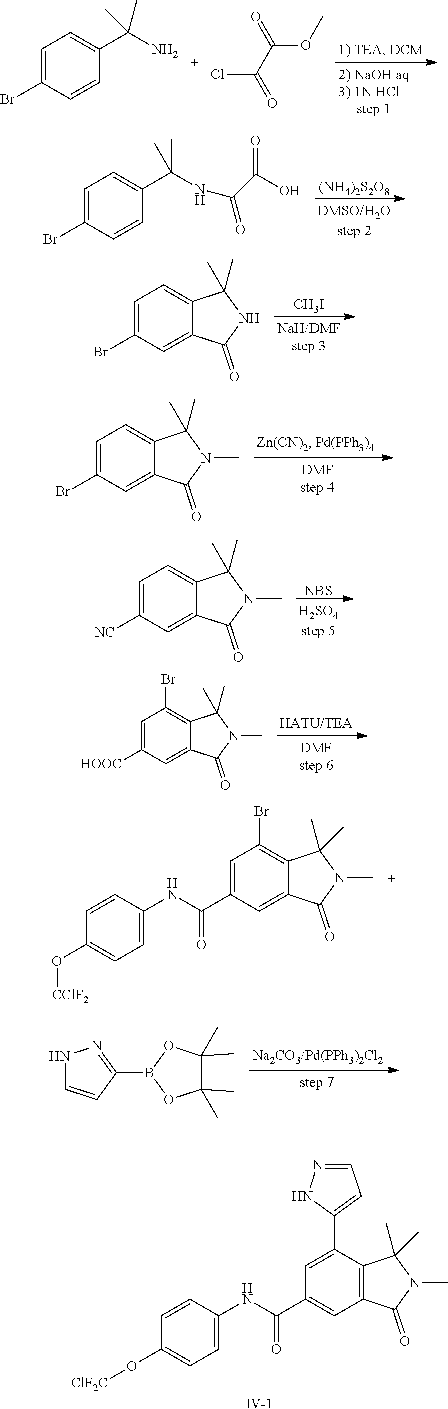

### Step 1: Synthesis of 2-((2-(4-bromophenyl)propan-2-yl)amino)-2-oxoacetic acid

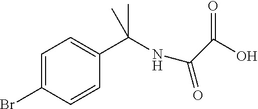 [SMILES: CC(C)(NC(=O)C(=O)O)c1ccc(Br)cc1]

In a nitrogen flushed 100 mL three-necked round-bottomed flask 2-(4-bromophenyl)propan-2-amine (1 g, 4.67 mmol) was dissolved in dichloromethane (10 mL) under nitrogen to give a colorless solution. Triethylamine (0.945 g, 9.34 mmol) was added to the solution, then methyl 2-chloro-2-oxoacetate (0.629 g, 5.14 mmol) was dropped to the reaction mixture at −10° C. After addition, the mixture was warmed up to 25° C. and stirred for 1 h, then quenched with 10% aqueous HCl followed by extraction with CH₂Cl₂ (50 mL×3). The combined organic layers were dried over Na₂SO₄ and concentrated to syrup. The residue was dissolved in methanol and stirred with NaOH solution for 30 min at 25° C. The resulting suspension was neutralized with HCl solution, then extracted with ethyl acetate. The combined organic layers were dried over Na₂SO₄ and concentrated to give 2-((2-(4-bromophenyl)propan-2-yl)amino)-2-oxoacetic acid (1.36 g, 100%) as a pale yellow oil. It was used in next step without purification. ¹H NMR (400 MHz, chloroform-d) δ 7.60 (s, 1H), 7.54-7.46 (m, 2H), 7.30-7.22 (m, 2H), 1.76 (s, 6H). MS: 386.0 (M+H)⁺.

### Step 2: Synthesis of 6-bromo-3,3-dimethylisoindolin-1-one

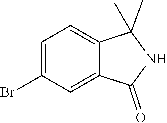

In an oven-dried 50 mL round-bottomed flask 2-((2-(4-bromophenyl)propan-2-yl)amino)-2-oxoacetic acid (286 mg, 1.000 mmol) and ammonium persulfate (456 mg, 1.999 mmol) was dissolved in DMSO (10 mL) and water (0.500 mL) under nitrogen to give a yellow suspension. The mixture was stirred at 100° C. for 4 h. After being cooled to room temperature, the mixture was added water (30 mL), followed by extraction with ethyl acetate. The combined organic layers were dried over Na₂SO₄, filtered and concentrated to give a crude product that was purified by silica gel chromatography (ethyl acetate/hexane from 60% to 80%) to give 6-bromo-3,3-dimethylisoindolin-1-one (130 mg, 54.2%) as a yellow solid. ¹H NMR (400 MHz, chloroform-d) δ 7.96 (dd, J=1.9, 0.5 Hz, 1H), 7.70 (dd, J=8.1, 1.9 Hz, 1H), 7.30 (dd, J=8.1, 0.6 Hz, 1H), 6.37 (s, 1H), 1.57 (s, 6H). MS: 240.0 (M+H)⁺.

### Step 3: Synthesis of 6-bromo-2,3,3-trimethylisoindolin-1-one

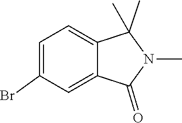

In an oven-dried 100 mL round-bottomed flask 6-bromo-3,3-dimethylisoindolin-1-one (1.5 g, 6.25 mmol) was dissolved in N,N-dimethylformamide (20 mL) under nitrogen to give a yellow solution. NaH (0.180 g, 7.50 mmol) was added to the solution. After being stirred at room temperature for 0.5 h, the mixture was added iodomethane (1.330 g, 9.37 mmol). After being stirred at room temperature for 1 h, the mixture was quenched with water (30 mL), followed by extraction with ethyl acetate. The combined organic layers were dried over Na₂SO₄, filtered and concentrated to give the title intermediate (1.40 g, 88%) as a pale yellow oil. It was used in next step without purification. ¹H NMR (400 MHz, chloroform-d) δ 7.98 (d, J=1.8 Hz, 1H), 7.67 (dd, J=8.0, 1.8 Hz, 1H), 7.31 (d, J=8.0 Hz, 1H), 3.04 (s, 3H), 1.47 (s, 6H). MS: 254.1 (M+H)⁺.

### Step 4: Synthesis of 1,1,2-trimethyl-3-oxoisoindoline-5-carbonitrile

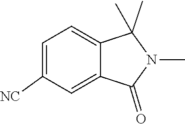

In an oven-dried 25 mL round-bottomed flask 6-bromo-2,3,3-trimethylisoindolin-1-one (700 mg, 2.75 mmol) was dissolved in N,N-dimethylformamide (10 mL) under nitrogen to give a yellow solution. Dicyanozinc (323 mg, 2.75 mmol) and Pd(PPh₃)₄ (318 mg, 0.275 mmol) were added to the solution. The mixture was stirred at 110° C. for 16 h. Water (50 mL) was added to the reaction mixture followed by extraction with ethyl acetate (50 mL×3). The combined organic layers were dried over Na₂SO₄, filtered and concentrated to give a crude product that was eluted through a silica gel column (ethyl acetate/hexane from 65% to 75%) to give 1,1,2-trimethyl-3-oxoisoindoline-5-carbonitrile (613 mg, 111%) as a yellow solid. ¹H NMR (400 MHz, chloroform-d) δ 8.13 (s, 1H), 7.84 (d, J=7.8 Hz, 1H), 7.56 (d, J=7.9 Hz, 1H), 3.07 (s, 3H), 1.51 (s, 6H). MS: 201.1 (M+H)⁺.

### Step 5: Synthesis of 7-bromo-1,1,2-trimethyl-3-oxoisoindoline-5-carboxylic acid

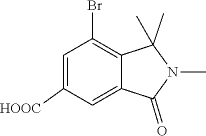 [SMILES: CN1C(=O)c2cc(C(=O)O)cc(Br)c2C1(C)C]

In an oven-dried 50 mL round-bottomed flask, 1,1,2-trimethyl-3-oxoisoindoline-5-carbonitrile (613 mg, 3.06 mmol) was dissolved in H₂SO₄ (8 mL) under nitrogen to give a yellow solution. N-Bromosuccinimide (599 mg, 3.37 mmol) was added to the reaction mixture. The mixture was stirred at 100° C. overnight. Water (4 mL) was added to the mixture, then it was stirred at 100° C. for 3 h. Water (50 mL) was added to the reaction mixture, followed by extraction with ethyl acetate (100 mL×3). The combined organic layers were dried over Na₂SO₄, filtered and concentrated to give a crude product that was eluted through a silica gel column (ethyl acetate/hexane from 80% to 100%) to give 7-bromo-1,1,2-trimethyl-3-oxoisoindoline-5-carboxylic acid (1.06 g, 116%) as a yellow solid. ¹H NMR (400 MHz, DMSO-d₆) δ 8.27 (d, J=1.4 Hz, 1H), 8.22 (d, J=1.4 Hz, 1H), 2.96 (s, 3H), 1.59 (s, 6H). MS: 298.0 (M+H)⁺.

### Step 6: Synthesis of 7-bromo-N-(4-(chlorodifluoromethoxy)phenyl)-1,1,2-trimethyl-3-oxoisoindoline-5-carboxamide

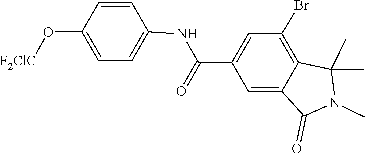

In an oven-dried 100 mL round-bottomed flask, 7-bromo-1,1,2-trimethyl-3-oxoisoindoline-5-carboxylic acid (400 mg, 1.342 mmol) was dissolved in N,N-dimethylformamide (10 mL) under nitrogen to give a yellow solution. To the solution were added triethylamine (204 mg, 2.013 mmol), 2-(3H-[1,2,3]triazolo[4,5-b]pyridin-3-yl)-1,1,3,3-tetramethylisouronium hexafluorophosphate(V) (765 mg, 2.013 mmol) and 4-(chlorodifluoromethoxy)aniline (312 mg, 1.610 mmol). The mixture was stirred for overnight at room temperature. Water (50 mL) was added to the mixture followed by extraction with ethyl acetate (50 mL×3). The combined organic layers were dried over Na₂SO₄, filtered and concentrated to give a crude product that was eluted through a silica gel column chromotagraph (ethyl acetate/hexane from 50% to 80%) to give 7-bromo-N-(4-(chlorodifluoromethoxy)phenyl)-1,1,2-trimethyl-3-oxoisoindoline-5-carboxamide (240 mg, 37.8%) as a yellow solid. MS: 473.0 (M+H)⁺.

### Step 7: Synthesis of N-(4-(chlorodifluoromethoxy)phenyl)-1,1,2-trimethyl-3-oxo-7-(1H-pyrazol-5-yl)isoindoline-5-carboxamide (Cpd. No. IV-1)

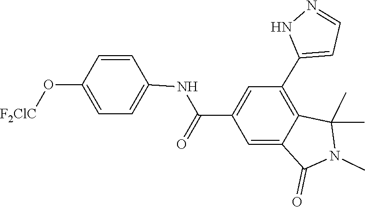

In a 10 mL microwave tube, 7-bromo-N-(4-(chlorodifluoromethoxy)phenyl)-1,1,2-trimethyl-3-oxoisoindoline-5-carboxamide (240 mg, 0.507 mmol) and 5-(4,4,5,5-tetramethyl-1,3,2-dioxaborolan-2-yl)-1H-pyrazole (147 mg, 0.760 mmol) were suspended in dimethoxyethane (1.5 mL) and water (0.5 mL) under nitrogen to give a yellow suspension. To the mixture were added Na₂CO₃ (161 mg, 1.520 mmol) and Pd(PPh₃)₂Cl₂ (37.1 mg, 0.051 mmol). The mixture was stirred at 120° C. under MW irradiation for 2 h. Then the mixture was eluted through a silica gel column (ethyl acetate/hexane from 80% to 100%) to give N-(4-(chlorodifluoromethoxy)phenyl)-1,1,2-trimethyl-3-oxo-7-(1H-pyrazol-5-yl)isoindoline-5-carboxamide (60 mg, 25.7%) as a white solid. ¹H NMR (400 MHz, DMSO-d₆) δ 13.27 (s, 1H), 10.70 (s, 1H), 8.34 (d, J=1.7 Hz, 1H), 8.29 (d, J=1.7 Hz, 1H), 8.02-7.93 (m, 3H), 7.46-7.36 (m, 2H), 6.74 (d, J=2.1 Hz, 1H), 3.00 (s, 3H), 1.61 (s, 6H). MS: 461.1 (M+H)⁺.

### Example IV-2

### Synthesis of N-(4-(chlorodifluoromethoxy)phenyl)-1,1,2-trimethyl-7-(5-methylfuran-2-yl)-3-oxoisoindoline-5-carboxamide (Cpd. No. IV-2)

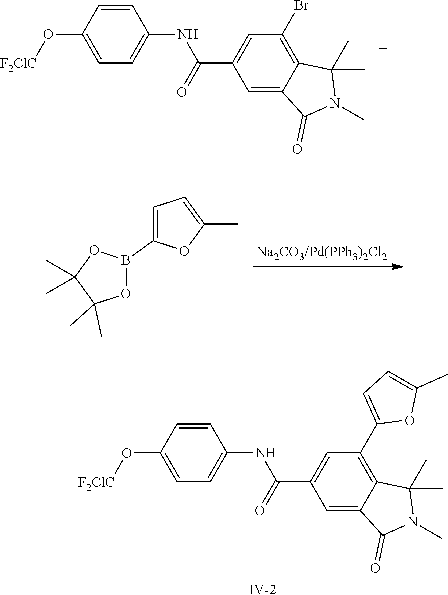

Essentially the same protocol of the preparation of Cpd. No. IV-1 in Example IV-1 was used to afford Cpd. No. IV-2 (30.0 mg) as a white solid. ¹H NMR (400 MHz, DMSO-d₆) δ 10.66 (s, 1H), 8.31 (d, J=1.6 Hz, 1H), 8.28 (d, J=1.7 Hz, 1H), 7.94 (d, J=9.0 Hz, 2H), 7.39 (d, J=8.7 Hz, 2H), 6.95 (d, J=3.3 Hz, 1H), 6.39-6.33 (m, 1H), 3.00 (s, 3H), 2.43 (s, 3H), 1.57 (s, 6H). MS: 475.1 (M+H)⁺.

### Example IV-3

### Synthesis of N-(4-(chlorodifluoromethoxy)phenyl)-1,1,2-trimethyl-7-(5-methylthiophen-2-yl)-3-oxoisoindoline-5-carboxamide (Cpd. No. IV-3)

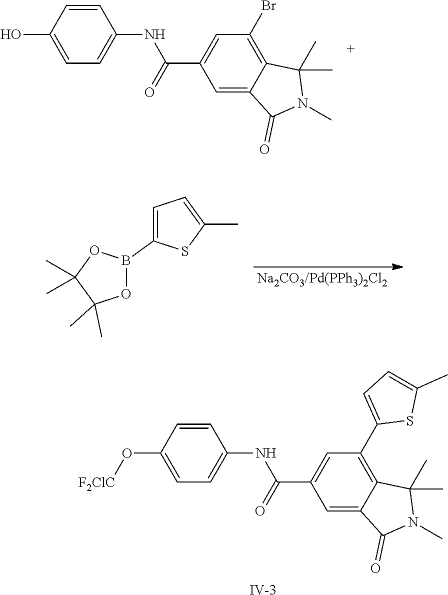

Essentially the same protocol of the preparation of Cpd. No. IV-1 in Example IV-1 was used to afford Cpd. No. IV-3 (20.0 mg) as a white solid. ¹H NMR (400 MHz, DMSO-d₆) δ 10.64 (s, 1H), 8.40 (d, J=1.8 Hz, 1H), 8.06 (d, J=1.7 Hz, 1H), 7.96-7.89 (m, 2H), 7.37 (d, J=8.7 Hz, 2H), 7.05 (d, J=3.5 Hz, 1H), 6.95-6.89 (m, 1H), 2.95 (s, 3H), 2.54 (s, 3H), 1.41 (s, 6H). MS: 491.0 (M+H)⁺.

### Example IV-4

### Synthesis of N-(4-(chlorodifluoromethoxy)phenyl)-1,1,2-trimethyl-3-oxo-7-(pyridin-4-yl)isoindoline-5-carboxamide (Cpd. No. IV-4)

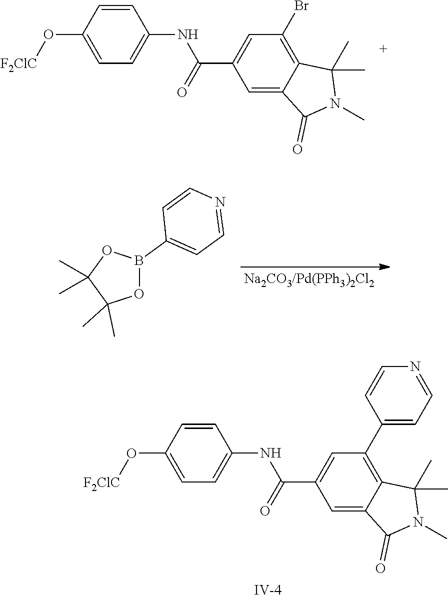

Essentially the same protocol of the preparation of Cpd. No. IV-1 in EXAMPLE IV-1 was used to afford Cpd. No. IV-4 (12.0 mg) as a white solid. ¹H NMR (400 MHz, DMSO-d₆) δ 10.64 (s, 1H), 8.78 (d, J=5.1 Hz, 2H), 8.46 (d, J=1.7 Hz, 1H), 8.00-7.89 (m, 3H), 7.62-7.55 (m, 2H), 7.38 (d, J=8.7 Hz, 2H), 2.94 (s, 3H), 1.27 (s, 6H). MS: 472.1 (M+H)⁺.

### Example IV-5

### Synthesis of N-(4-(chlorodifluoromethoxy)phenyl)-2-(2-hydroxyethyl)-1,1-dimethyl-3-oxo-7-(1H-pyrazol-5-yl)isoindoline-5-carboxamide (Cpd. No. IV-5)

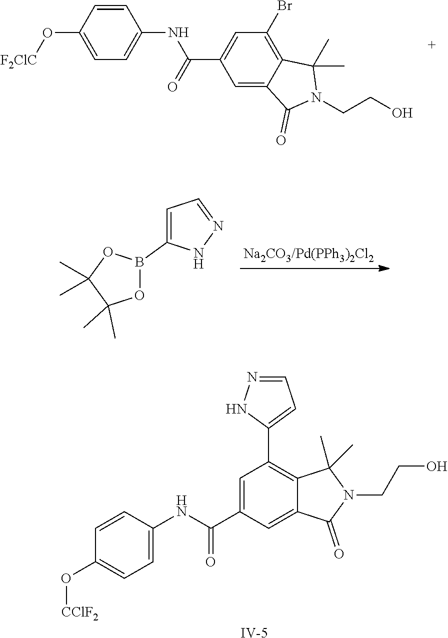

Essentially the same protocol of the step 7 in EXAMPLE IV-6 was used to afford Cpd. No. IV-5 (4.8 mg) as a white solid. ¹H NMR (400 MHz, DMSO-d₆) δ 10.67 (s, 1H), 8.34 (d, J=1.7 Hz, 1H), 8.26 (d, J=1.7 Hz, 1H), 7.96-7.90 (m, 3H), 7.40 (d, J=8.8 Hz, 2H), 6.70 (d, J=2.3 Hz, 1H), 3.67 (t, J=6.9 Hz, 2H), 3.53 (t, J=6.9 Hz, 2H), 1.60 (s, 6H). MS: 490.9 (M+H)⁺.

### Example IV-6

### Synthesis of N-(4-(chlorodifluoromethoxy)phenyl)-1,1-dimethyl-2-(2-(4-methylpiperazin-1-yl)ethyl)-3-oxo-7-(1H-pyrazol-5-yl)isoindoline-5-carboxamide (Cpd. No. IV-6)

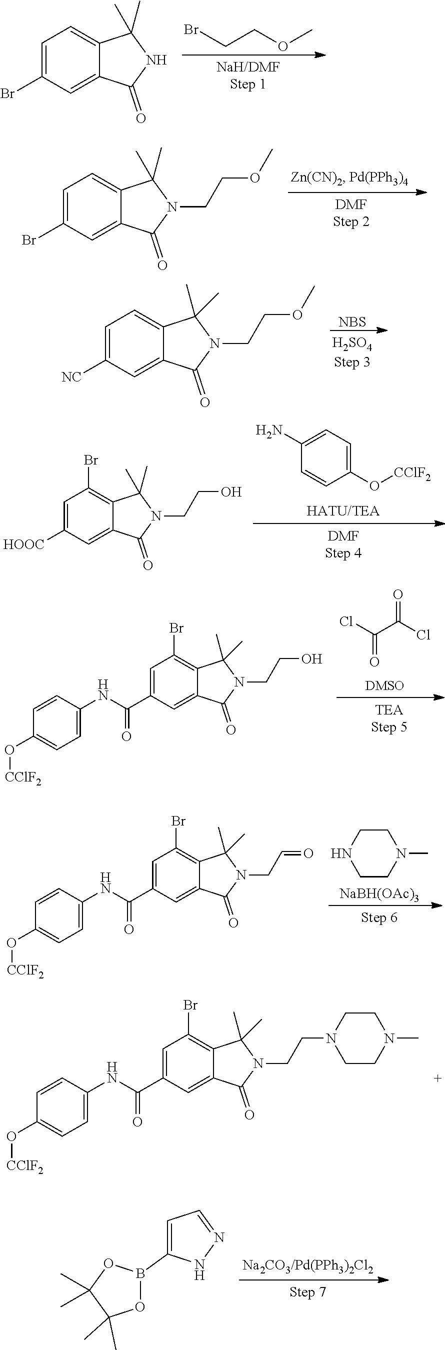

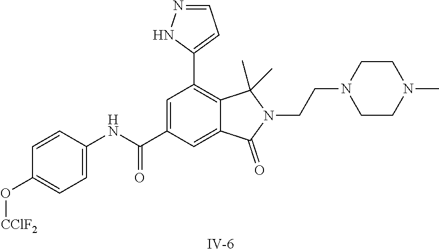

### Step 1: Synthesis of 6-bromo-2-(2-methoxyethyl)-3,3-dimethylisoindolin-1-one

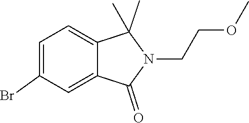 [SMILES: COCCN1C(=O)c2cc(Br)ccc2C1(C)C]

In an oven-dried 25 mL round-bottomed flask, 6-bromo-3,3-dimethylisoindolin-1-one (4.77 g, 19.87 mmol) was dissolved in N,N-dimethylformamide (40 mL) under nitrogen to give a yellow solution. NaH (1.192 g, 49.7 mmol) was added to the reaction mixture. It was stirred at room temperature for 0.5 h. The mixture was cooled to 0° C. under an ice/water bath, then was added 1-bromo-2-methoxyethane (27.6 g, 199 mmol) dropwise. The mixture was stirred at 50° C. for 2 h, then quenched with water (50 mL) after cooling, followed by extraction with ethyl acetate (40 mL×3). The combined organic layers were dried over Na₂SO₄, filtered and concentrated to give a crude product that was purified by silica gel column (ethyl acetate/hexane from 20% to 35%) to give 6-bromo-2-(2-methoxyethyl)-3,3-dimethylisoindolin-1-one (5.12 g, 86%) as a yellow oil. MS: 297.9 (M+H)⁺.

### Step 2: Synthesis of 2-(2-methoxyethyl)-1,1-dimethyl-3-oxoisoindoline-5-carbonitrile

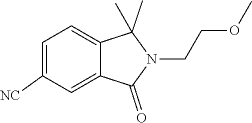 [SMILES: COCCN1C(=O)c2cc(C#N)ccc2C1(C)C]

In an oven-dried 25 mL round-bottomed flask, 6-bromo-2-(2-methoxyethyl)-3,3-dimethylisoindolin-1-one (5.12 g, 17.17 mmol) was dissolved in N,N-dimethylformamide (40 mL) under nitrogen to give a yellow solution. Dicyanozine (2.016 g, 17.17 mmol) and Pd(PPh₃)₄ (1.984 g, 1.717 mmol) were added to the mixture. The reaction mixture was stirred at 110° C. for overnight. After being cooled to room temperature, the mixture was quenched with water (50 mL), followed by extraction with ethyl acetate (40 mL×3). The combined organic layers were dried over Na₂SO₄, filtered and concentrated to give a crude product that was purified by silica gel column (ethyl acetate/hexane from 65% to 75%) to give 2-(2-methoxyethyl)-1,1-dimethyl-3-oxoisoindoline-5-carbonitrile (4.56 g, 109%) as a yellow solid. MS: 245.1 (M+H)⁺.

### Step 3: Synthesis of 7-bromo-2-(2-hydroxyethyl)-1,1-dimethyl-3-oxoisoindoline-5-carboxylic acid

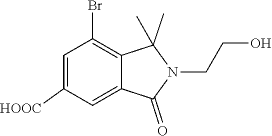 [SMILES: CC1(C)c2c(Br)cc(C(=O)O)cc2C(=O)N1CCO]

In an oven-dried 50 mL round-bottomed flask, 2-(2-methoxyethyl)-1,1-dimethyl-3-oxoisoindoline-5-carbonitrile (3.56 g, 14.57 mmol) was dissolved in H₂SO₄ (15 mL) under nitrogen to give a color solution. N-Bromosuccinimide (2.85 g, 16.03 mmol) was added to the reaction mixture. The reaction mixture was stirred at 100° C. for overnight. 5 mL water was added to the mixture, then it was stirred at 100° C. for 8 h. Cooled to room temperature, water was added to the mixture followed by extraction with ethyl acetate (20 mL×6). The combined organic layers were dried over Na₂SO₄, filtered and concentrated to give a crude product that was purified by silica gel column chromatography (ethyl acetate/hexane from 80% to 100%) to give 7-bromo-2-(2-hydroxyethyl)-1,1-dimethyl-3-oxoisoindoline-5-carboxylic acid (1.06 g, 21.76%) as a yellow oil. MS: 327.9 (M+H)⁺.

### Step 4: 7-bromo-N-(4-(chlorodifluoromethoxy)phenyl)-2-(2-hydroxyethyl)-1,1-dimethyl-3-oxoisoindoline-5-carboxamide

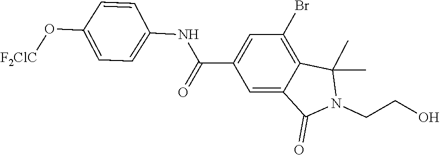

In an oven-dried 100 mL round-bottomed flask, 7-bromo-2-(2-hydroxyethyl)-1,1-dimethyl-3-oxoisoindoline-5-carboxylic acid (1.04 g, 3.17 mmol) was dissolved in N,N-dimethylformamide (10 mL) under nitrogen to give a yellow solution. Triethylamine (0.481 g, 4.75 mmol), 2-(3H-[1,2,3]triazolo[4,5-b]pyridin-3-yl)-1,1,3,3-tetramethylisouronium hexafluorophosphate (1.808 g, 4.75 mmol) and 4-(chlorodifluoromethoxy)aniline (0.736 g, 3.80 mmol) were added to the mixture. The mixture was stirred for overnight at room temperature. Water (20 mL) was added to the mixture followed by extraction with ethyl acetate (20 mL×3). The combined organic layers were dried over Na₂SO₄, filtered and concentrated to give a crude product that was purified by silica gel column chromatography (ethyl acetate/hexane from 50% to 80%) to give 7-bromo-N-(4-(chlorodifluoromethoxy)phenyl)-2-(2-hydroxyethyl)-1,1-dimethyl-3-oxoisoindoline-5-carboxamide (1.05 g, 65.8%) as a yellow solid. MS: 502.9 (M+H)⁺.

### Step 5: Synthesis of 7-bromo-N-(4-(chlorodifluoromethoxy)phenyl)-1,1-dimethyl-3-oxo-2-(2-oxoethyl)isoindoline-5-carboxamide

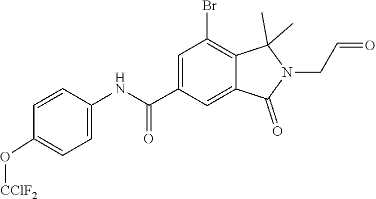

In a nitrogen flushed 25 mL two-necked round-bottomed flask, oxalyl dichloride (136 mg, 1.072 mmol) was dissolved in dichloromethane (5 mL) under nitrogen to give a colorless solution. The mixture was cooled to −78° C. with a dry ice/acetone bath, then it was added dimethylsulfoxide (168 mg, 2.144 mmol) dropwise over 10 min. The mixture was stirred for 15 min at the same temperature, followed by dropping a solution of 7-bromo-N-(4-(chlorodifluoromethoxy)phenyl)-2-(2-hydroxyethyl)-1,1-dimethyl-3-oxoisoindoline-5-carboxamide (360 mg, 0.715 mmol) in dichloromethane (5 mL) over 10 min. The mixture was maintained at −78° C. and stirred for another 1 h, then quenched with triethylamine (362 mg, 3.57 mmol), followed by warmed up to room temperature and stirred for another 30 min. The mixture was diluted with saturated aqueous NH₄Cl (10 mL), followed by extraction with dichloromethane (20 mL×3). The combined organic layers were dried over Na₂SO₄, filtered and concentrated to give a crude product that was purified by silica gel column chromatography (ethyl acetate/hexane from 50% to 100%) to give 7-bromo-N-(4-(chlorodifluoromethoxy)phenyl)-1,1-dimethyl-3-oxo-2-(2-oxoethyl)isoindoline-5-carboxamide (120 mg, 33.5%) as a yellow solid. MS: 501.0 (M+H)⁺.

### Step 6: Synthesis of 7-bromo-N-(4-(chlorodifluoromethoxy)phenyl)-1,1-dimethyl-2-(2-(4-methylpiperazin-1-yl)ethyl)-3-oxoisoindoline-5-carboxamide

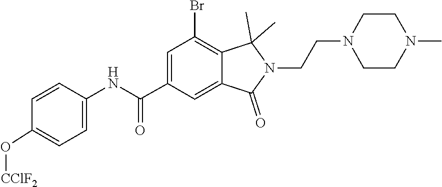

In a nitrogen flushed 25 mL two-necked round-bottomed flask, 7-bromo-N-(4-(chlorodifluoromethoxy)phenyl)-1,1-dimethyl-3-oxo-2-(2-oxoethyl)isoindoline-5-carboxamide (70 mg, 0.140 mmol) and 1-methylpiperazine (28.0 mg, 0.279 mmol) were dissolved in dichloromethane (5 mL) under nitrogen to give a yellow solution. NaBH(OAc)₃ (89 mg, 0.419 mmol) was added to the mixture. The mixture was stirred for overnight at room temperature, then concentrated to give a crude product that was purified by silica gel column chromatography (methol/ethyl acetate from 0% to 20%) to give 7-bromo-N-(4-(chlorodifluoromethoxy)phenyl)-1,1-dimethyl-2-(2-(4-methylpiperazin-1-yl)ethyl)-3-oxoisoindoline-5-carboxamide (67 mg, 82%) as a yellow oil. MS: 585.1 (M+H)⁺.

### Step 7: N-(4-(chlorodifluoromethoxy)phenyl)-1,1-dimethyl-2-(2-(4-methylpiperazin-1-yl)ethyl)-3-oxo-7-(1H-pyrazol-5-yl)isoindoline-5-carboxamide (Cpd. No. IV-6)

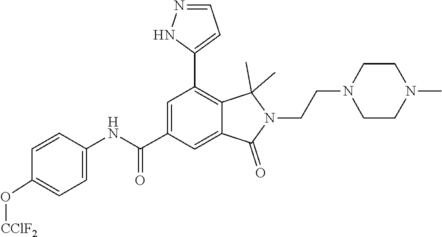

In a 10 mL microwave tube, 7-bromo-N-(4-(chlorodifluoromethoxy)phenyl)-1,1-dimethyl-2-(2-(4-methylpiperazin-1-yl)ethyl)-3-oxoisoindoline-5-carboxamide (67 mg, 0.114 mmol) and 5-(4,4,5,5-tetramethyl-1,3,2-dioxaborolan-2-yl)-1H-pyrazole (33.3 mg, 0.172 mmol) were dissolved in dimethoxyethane (1.0 mL) and water (0.3 mL) under nitrogen to give a yellow suspension. Na₂CO₃ (36.4 mg, 0.343 mmol) and Pd(PPh₃)₂Cl₂ (1.12 mg, 0.001 mmol) were added to the reaction mixture. Exchange nitrogen three times. It was stirred 2 h at 120° C. under MW reactor. The crude product was purified by silica gel column chromatography (ethyl acetate/hexane from 80% to 100%) to give N-(4-(chlorodifluoromethoxy)phenyl)-1,1-dimethyl-2-(2-(4-methylpiperazin-1-yl)ethyl)-3-oxo-7-(1H-pyrazol-5-yl)isoindoline-5-carboxamide (6 mg, 9.2%) as a white solid. ¹H NMR (400 MHz, DMSO-d₆) δ 10.65 (s, 1H), 8.32 (d, J=1.8 Hz, 1H), 8.26 (d, J=1.8 Hz, 1H), 7.98-7.89 (m, 3H), 7.38 (d, J=8.7 Hz, 2H), 6.69 (d, J=2.3 Hz, 1H), 3.66-3.59 (m, 2H), 3.47-3.35 (m, 2H), 3.34-3.20 (m, 2H), 3.10-2.95 (m, 2H), 2.83-2.74 (m, 4H), 2.79 (s, 3H), 1.60 (s, 6H), MS: 573.2 (M+H)⁺.

### Example IV-7

### Synthesis of N-(4-(chlorodifluoromethoxy)phenyl)-2-(2-(1,1-dioxidothiomorpholino)ethyl)-1,1-dimethyl-3-oxo-7-(1H-pyrazol-5-yl)isoindoline-5-carboxamide (Cpd. No. IV-7)

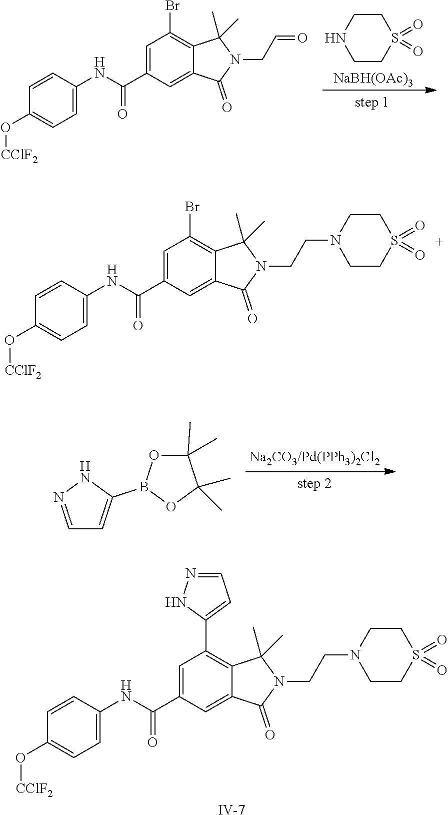

Essentially the same protocol of the preparation of Cpd. No. IV-6 in Example IV-6 was used to afford Cpd. No. IV-7 (6.0 mg) as a white solid. ¹H NMR (400 MHz, DMSO-d₆) δ 10.65 (s, 1H), 8.32 (d, J=1.8 Hz, 1H), 8.26 (d, J=1.8 Hz, 1H), 7.99-7.90 (m, 3H), 7.38 (d, J=8.6 Hz, 2H), 6.69 (d, J=2.3 Hz, 1H), 3.62-3.50 (m, 2H), 3.34-3.13 (m, 8H), 3.04-2.92 (m, 2H), 1.60 (s, 6H). MS: 608.1 (M+H)⁺.

### Example IV-8

### Synthesis of (R)—N-(4-(chlorodifluoromethoxy)phenyl)-2-(2-(3-hydroxypyrrolidin-1-yl)ethyl)-1,1-dimethyl-3-oxo-7-(1H-pyrazol-5-yl)isoindoline-5-carboxamide (Cpd. No. IV-8)

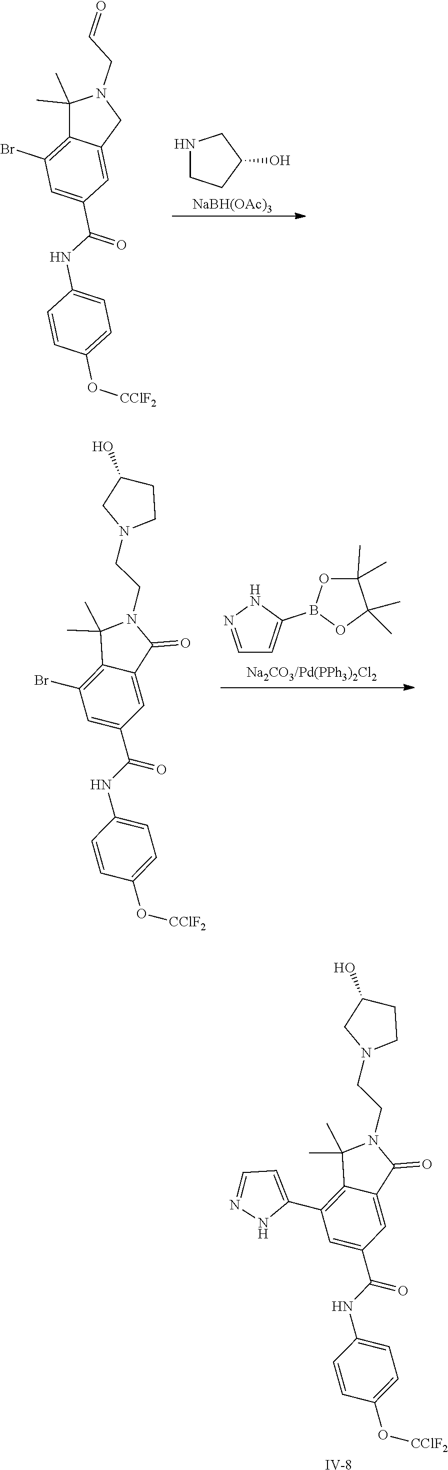

Essentially the same protocol of the preparation of Cpd. No. IV-6 in Example IV-6 was used to afford Cpd. No. IV-8 (6.0 mg) as a white solid. ¹H NMR (400 MHz, DMSO-d₆) δ 13.26 (s, 1H), 10.67 (s, 1H), 8.38-8.28 (m, 2H), 7.98-7.90 (m, 3H), 7.39 (d, J=8.7 Hz, 2H), 6.71 (d, J=2.3 Hz, 1H), 5.52 (s, 1H), 4.48-4.41 (m, 1H), 3.83-3.79 (m, 2H), 3.43-3.38 (m, 2H), 3.22-3.17 (m, 2H), 2.29 (s, 2H), 2.04-1.95 (m, 2H), 1.63 (s, 6H). MS: 560.2 (M+H)⁺.

### Example IV-9

### Synthesis of N-(4-(chlorodifluoromethoxy)phenyl)-1,1,2-trimethyl-3-oxo-7-(pyridin-3-yl)isoindoline-5-carboxamide (Cpd. No. IV-9)

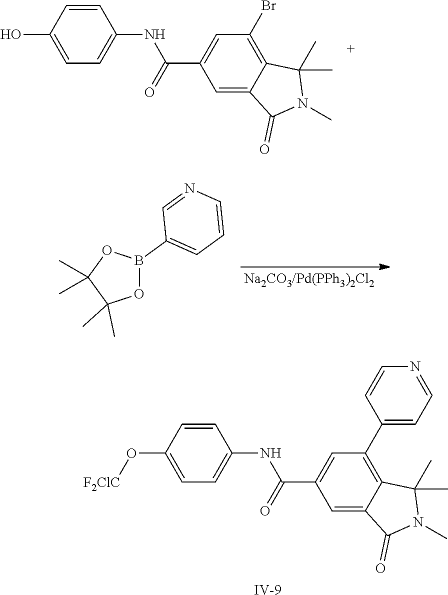

Essentially the same protocol of the preparation of Cpd. No. IV-1 in Example IV-1 was used to afford Cpd. No. IV-9 (190.0 mg) as a white solid. ¹H NMR (400 MHz, DMSO-d₆) δ 10.65 (s, 1H), 8.76 (d, J=4.2 Hz, 1H), 8.69 (s, 1H), 8.46 (d, J=1.7 Hz, 1H), 8.03 (d, J=1.7 Hz, 1H), 8.00-7.90 (m, 3H), 7.62 (dd, J=7.8, 4.9 Hz, 1H), 7.39 (d, J=8.8 Hz, 2H), 2.95 (s, 3H), 1.26 (s, 6H). MS: 472.1 (M+H)⁺.

### Example V-1

### Synthesis of N-(4-(chlorodifluoromethoxy)phenyl)-3,3-dimethyl-2-oxo-4-(1H-pyrazol-5-yl)indoline-6-carboxamide (Cpd. No. V-1)

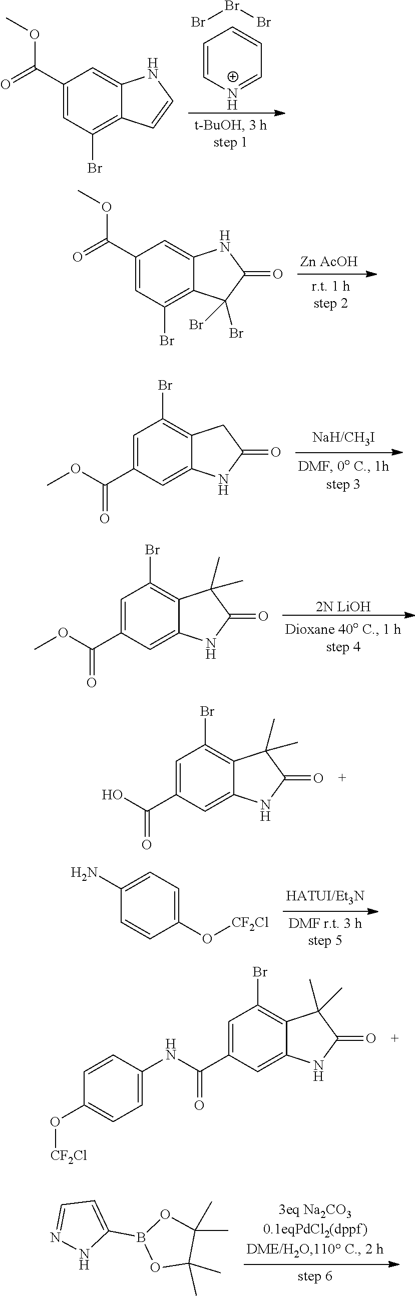

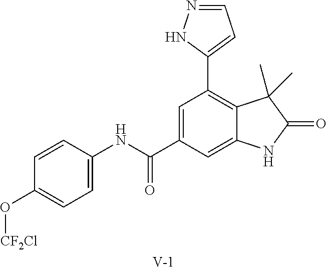

### Step 1: Synthesis of methyl 3,3,4-tribromo-2-oxoindoline-6-carboxylate

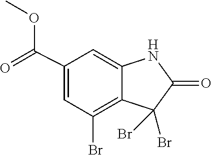 [SMILES: COC(=O)c1cc(Br)c2c(c1)NC(=O)C2(Br)Br]

In a 25 mL round-bottomed flask, to a solution of methyl 4-bromo-1H-indole-6-carboxylate (2.0 g, 7.87 mmol) in Butan-1-ol (30 mL), was added pyridinium tribromide (10.07 g, 31.5 mmol), Followed by being stirred at 40° C. for 3 h. Water (30 mL) was added to the mixture followed by extraction with ethyl acetate (25 mL×3). The combined organic layers were dried over Na₂SO₄, filtered and concentrated to give a crude product that was eluted through a silica gel column (ethyl acetate/hexane from 2% to 10%) to give methyl 3,3,4-tribromo-2-oxoindoline-5-carboxylate (3.3 g, 98%). MS: 427.70, 429.70 (M+H)⁺.

### Step 2: Synthesis of methyl 4-bromo-2-oxoindoline-6-carboxylate

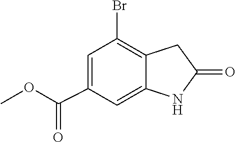

In a 100 mL round-bottomed flask, to a solution of methyl 3,3,4-tribromo-2-oxoindoline-6-carboxylate (2.0 g, 4.67 mmol) in AcOH (6 mL), was added zinc (1.22 g, 18.7 mmol), then the mixture was stirred for 1.5 h at room temperature. After the reaction was completed, the mixture was filtered and concentrated, then diluted with water, followed by extraction with ethyl acetate (15 mL×3). The combined organic layers were washed with aqueous NaCl, dried over Na₂SO₄, filtered and concentrated to give crude product (1.2 g. 95%), which was used in the next step without purification. MS: 271.0, 273.00 (M+H)⁺.

### Step 3: Synthesis of methyl 4-bromo-3,3-dimethyl-2-oxoindoline-6-carboxylate

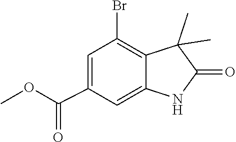 [SMILES: COC(=O)c1cc(Br)c2c(c1)NC(=O)C2(C)C]

In a 100 mL three-necked-round-bottomed flask, to the suspension of sodium hydride (85 mg, 3.55 mmol) in tetrahydrofuran (5 mL), was added methyl-4-bromo-2-oxoindoline-6-carboxylate (480 mg, 1.777 mmol) in 5 mL tetrahydrofuran at 0° C. After being stirred for 10 min, iodomethane (252 mg, 1.777 mmol) was added to the mixture and the mixture stirred for another 1 h, then quenched with 10 mL aqueous NH₄Cl slowly, followed by extraction with ethyl acetate (15 mL×3). The combined organic layers were concentrated to give a crude product that was eluted through a silica gel column (ethyl acetate/hexane from 10% to 40%) to give methyl 4-bromo-3,3-dimethyl-2-oxoindoline-6-carboxylate (337 mg, 63.6%). ¹H NMR (400 MHz, chloroform-d) δ 7.90 (d, J=1.3 Hz, 1H), 7.53 (d, J=1.4 Hz, 1H), 3.95 (s, 3H), 1.58 (s, 6H). MS: 298.95, 300.95 (M+H)⁺.

### Step 4: Synthesis of 4-bromo-3,3-dimethyl-2-oxoindoline-6-carboxylic acid

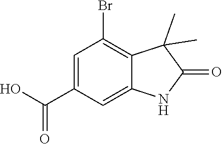 [SMILES: CC1(C)C(=O)Nc2cc(C(=O)O)cc(Br)c21]

In a 25 mL round-bottomed flask, to a solution of methyl 4-bromo-3,3-dimethyl-2-oxoindoline-6-carboxylate (170.0 mg, 0.57 mmol) in 1,4-dioxane (3 mL), was added 2 mL 2N lithium hydroxide. The mixture was stirred for overnight at 50° C. The mixture was acidified with 1N HCl, extracted with ethyl acetate. The resulting organic layers were combined and concentrated to give a crude product that was purified by silica gel column chromatography (ethyl acetate/hexane from 5% to 40%) to give 4-bromo-3,3-dimethyl-2-oxoindoline-6-carboxylic acid (160.0 mg, 99%) as a colorless oil. MS: 284.90, 286.90 (M+H)⁺.

### Step 5: Synthesis of 4-bromo-N-(4-(chlorodifluoromethoxy)phenyl)-3,3-dimethyl-2-oxoindoline-6-carboxamide

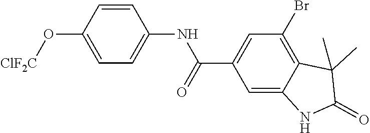

In a 25 mL round-bottomed flask, 4-bromo-3,3-dimethyl-2-oxoindoline-6-carboxylic acid (138.0 mg, 0.713 mmol) was dissolved in N,N-dimethylformamide (5.0 mL), diisopropylethyl amine (115.0 mg, 0.891 mmol) and 2-(7-azabenzotriazol-1-yl)-N,N,N′,N′-tetramethyluronium hexafluorophosphate (339.0 mg, 0.891 mmol) were added to the mixture, stirred for 10 min, 4-(chlorodifluoromethoxy)aniline (138.0 mg, 0.713 mmol) was added to the mixture, it was stirred for overnight, water was added to the mixture followed by extraction with ethyl acetate. The combined organic layers were concentrated to give a crude product that was eluted through a silica gel column (ethyl acetate/hexane from 5% to 40%) to give 4-bromo-N-(4-(chlorodifluoromethoxy)phenyl)-3,3-dimethyl-2-oxoindoline-6-carboxamide (223 mg, 72%). MS: 459.90, 461.90 (M+H)⁺.

### Step 6: Synthesis of N-(4-(chlorodifluoromethoxy)phenyl)-3,3-dimethyl-2-oxo-4-(1H-pyrazol-5-yl)indoline-6-carboxamide (Cpd. No. V-1)

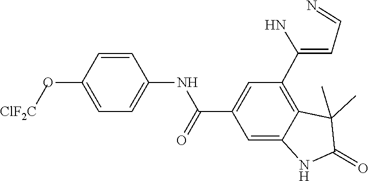

In a 5 mL tube, to a solution of 4-bromo-N-(4-(chlorodifluoromethoxy)phenyl)-3,3-dimethyl-2-oxoindoline-6-carboxamide (110.0 mg, 0.239 mmol) in dimethoxyethane (1.5 mL), EtOH (0.15 mL) and water (0.3 mL) was added 5-(4,4,5,5-tetramethyl-1,3,2-dioxaborolan-2-yl)-1H-pyrazole (69.7 mg, 0.359 mmol), Pd(PPh₃)₂Cl₂ (16.8 mg, 0.024 mmol) and Na₂CO₃ (76.0 mg, 0.718 mmol). The mixture was stirred at 110° C. under MW for 1.5 h, then quenched with water, followed by extraction with ethyl acetate. The combined organic layers were concentrated to give a crude product that was eluted through a silica gel column (ethyl acetate/hexane from 5% to 40%) to give N-(4-(chlorodifluoromethoxy)phenyl)-3,3-dimethyl-2-oxo-4-(1H-pyrazol-5-yl)indoline-6-carboxamide (30.0 mg, 28.1%). ¹H NMR (400 MHz, DMSO-d₆) δ 13.11 (s, 1H), 10.66 (s, 1H), 10.49 (s, 1H), 7.93-7.86 (m, 3H), 7.72 (s, 1H), 7.40-7.33 (m, 3H), 6.67 (s, 1H), 1.40 (s, 6H). MS: 447.00 (M+H)⁺.

### Example V-2

### Synthesis of N-(4-(chlorodifluoromethoxy)phenyl)-1,3,3-trimethyl-2-oxo-4-(1H-pyrazol-5-yl)indoline-6-carboxamide (Cpd. No. V-2)

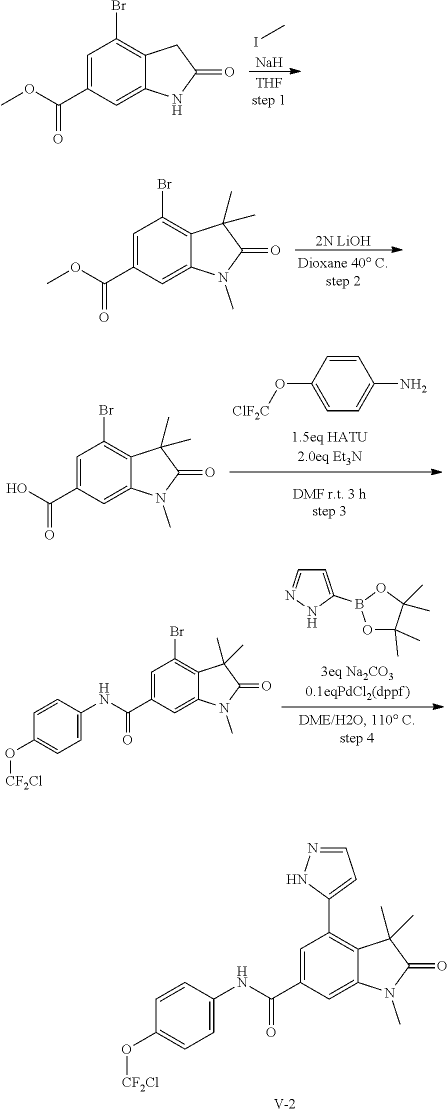

### Step 1: Synthesis of methyl 4-bromo-1,3,3-trimethyl-2-oxoindoline-6-carboxylate

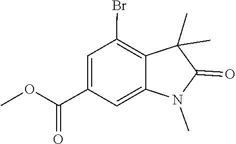 [SMILES: COC(=O)c1cc(Br)c2c(c1)N(C)C(=O)C2(C)C]

In a 100 mL two-necked-round-bottomed flask, to a suspension of sodium hydride (133 mg, 3.33 mmol) in tetrahydrofuran (5 mL) was added methyl 4-bromo-2-oxoindoline-6-carboxylate (300 mg, 1.111 mmol) in 4 mL tetrahydrofuran at 0° C. After being stirred for 10 min, the mixture was added iodomethane (788 mg, 5.55 mmol) and stirred for another 1 h, then quenched with 10 mL aqueous NH₄Cl, followed by extraction with ethyl acetate (15 mL×3). The combined organic layers were concentrated to give a crude product that was eluted through a silica gel column (ethyl acetate/hexane from 10% to 40%) to give methyl 4-bromo-1,3,3-trimethyl-2-oxoindoline-6-carboxylate (280 mg, 81%). ¹H NMR (400 MHz, DMSO-d₆) δ 13.37 (s, 1H), 7.73 (d, J=1.3 Hz, 1H), 7.50 (d, J=1.3 Hz, 1H), 3.20 (s, 3H), 1.42 (s, 6H). MS: 312.95, 314.95 (M+H)⁺.

### Step 2: Synthesis of 4-bromo-1,3,3-trimethyl-2-oxoindoline-6-carboxylic acid

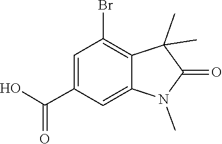 [SMILES: CN1C(=O)C(C)(C)c2c(Br)cc(C(=O)O)cc21]

Essentially the same protocol of Step 4 in EXAMPLE V-1 to afford 4-bromo-1,3,3-trimethyl-2-oxoindoline-6-carboxylic acid. MS: 299.00 (M+H)⁺.

### Step 3: Synthesis of 4-bromo-N-(4-(chlorodifluoromethoxy)phenyl)-1,3,3-trimethyl-2-oxoindoline-6-carboxamide

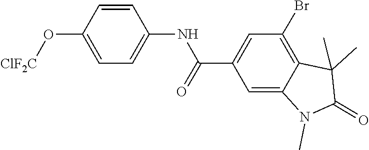

Essentially the same protocol of Step 5 in EXAMPLE V-1 to afford 4-bromo-N-(4-(chlorodifluoromethoxy)phenyl)-1,3,3-trimethyl-2-oxoindoline-6-carboxamide. MS: 474.00 (M+H)⁺.

### Step 4: Synthesis of N-(4-(chlorodifluoromethoxy)phenyl)-1,3,3-trimethyl-2-oxo-4-(1H-pyrazol-5-yl)indoline-6-carboxamide

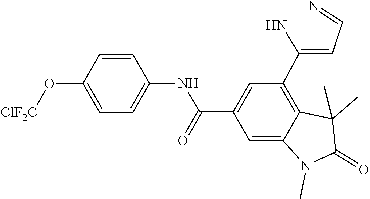

Essentially the same protocol of the preparation of Cpd. No. V-1 in EXAMPLE V-1 was used to afford Cpd. No. V-2 (19.5 mg) as a white solid. ¹H NMR (400 MHz, DMSO-d₆) δ 13.14 (s, 1H), 10.49 (s, 1H), 7.96-7.87 (m, 3H), 7.81 (d, J=1.6 Hz, 1H), 7.53 (d, J=1.6 Hz, 1H), 7.39 (d, J=8.7 Hz, 2H), 6.69 (t, J=2.1 Hz, 1H), 3.25 (s, 3H), 1.43 (s, 6H). MS: 461.00 (M+H)⁺.

### Example V-3

### Synthesis of N-(4-(chlorodifluoromethoxy)phenyl)-2′-oxo-4′-(1H-pyrazol-5-yl)spiro[cyclopentane-1,3′-indoline]-6′-carboxamide (Cpd. No. V-3)

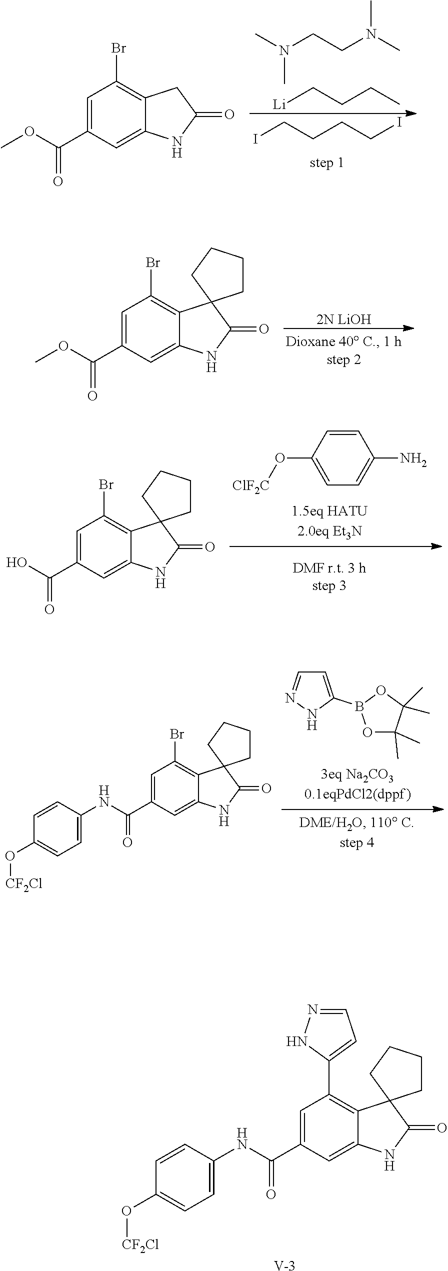

### Step 1: Synthesis of methyl 4′-bromo-2′-oxospiro[cyclopentane-1,3′-indoline]-6′-carboxylate

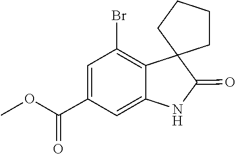 [SMILES: COC(=O)c1cc(Br)c2c(c1)NC(=O)C21CCCC1]

In an oven-dried 50 mL round-bottomed flask, to a solution of methyl 4-bromo-2-oxoindoline-6-carboxylate (200 mg, 0.741 mmol) in tetrahydrofuran (2 mL) was added N1,N1,N2,N2-tetramethylethane-1,2-diamine (172 mg, 1.481 mmol) and butyllithium (11.9 mg, 0.186 mmol) dropwise under nitrogen at −78° C., the mixture was stirred at −78° C. for 1 h, then was added 1,4-diiodobutane (1147 mg, 3.70 mmol) dropwise over 20 min. After the addition of materials, the mixture was warmed up to room temperature and stirred for overnight, then quenched with saturated aqueous NH₄Cl (10 mL), followed by extraction with ethyl acetate (20 mL×3). The combined organic layers were concentrated to give a crude product that was eluted through a silica gel column (ethyl acetate/hexane from 0 to 50%) to give methyl 4′-bromo-2′-oxospiro[cyclopentane-1,3′-indoline]-6′-carboxylate (120 mg, 50.0%). ¹H NMR (400 MHz, chloroform-d) δ 7.90 (d, J=1.5 Hz, 1H), 7.46 (d, J=1.6 Hz, 1H), 3.94 (s, 3H), 2.44-2.35 (m, 2H), 2.19-2.10 (m, 4H), 2.09-2.00 (m, 2H). MS: 323.90, 325.90 (M+H)⁺.

### Step 2: Synthesis of 4′-bromo-2′-oxospiro[cyclopentane-1,3′-indoline]-6′-carboxylic acid

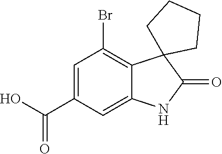 [SMILES: O=C(O)c1cc(Br)c2c(c1)NC(=O)C21CCCC1]

Essentially the same protocol of Step 4 in EXAMPLE V-1 MS: 311.00 (M+H)⁺.

### Step 3: Synthesis of 4′-bromo-N-(4-(chlorodifluoromethoxy)phenyl)-2′-oxospiro[cyclopentane-1,3′-indoline]-6′-carboxamide

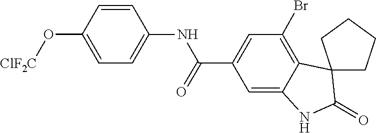

Essentially the same protocol of Step 5 in EXAMPLE V-1 MS: 486.00 (M+H)⁺.

### Step 4: Synthesis of N-(4-(chlorodifluoromethoxy)phenyl)-2′-oxo-4′-(1H-pyrazol-5-yl)spiro[cyclopentane-1,3′-indoline]-6′-carboxamide

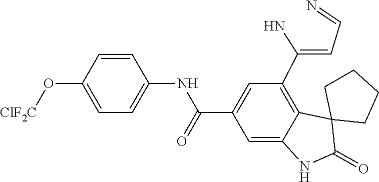

Essentially the same protocol of the preparation of Cpd. No. V-1 in Example V-1 was used to afford Cpd. No. V-3 (19.5 mg) as a white solid. ¹H NMR (400 MHz, DMSO-d₆) δ 13.13 (s, 1H), 10.54 (s, 1H), 10.49 (s, 1H), 7.96-7.88 (m, 3H), 7.70-7.66 (m, 1H), 7.42-7.34 (m, 3H), 6.64-6.58 (m, 1H), 2.36-2.28 (m, 2H), 1.91-1.86 (m, 2H), 1.85-1.76 (m, 2H), 1.73-1.68 (m, 2H). MS: 473.00 (M+H)⁺.

### Example V-4

### N-(4-(chlorodifluoromethoxy)phenyl)-2′-oxo-4′-(1H-pyrazol-5-yl)spiro[cyclopentane-1,3′-indolin]-3-ene-6′-carboxamide (Cpd. No. V-4)

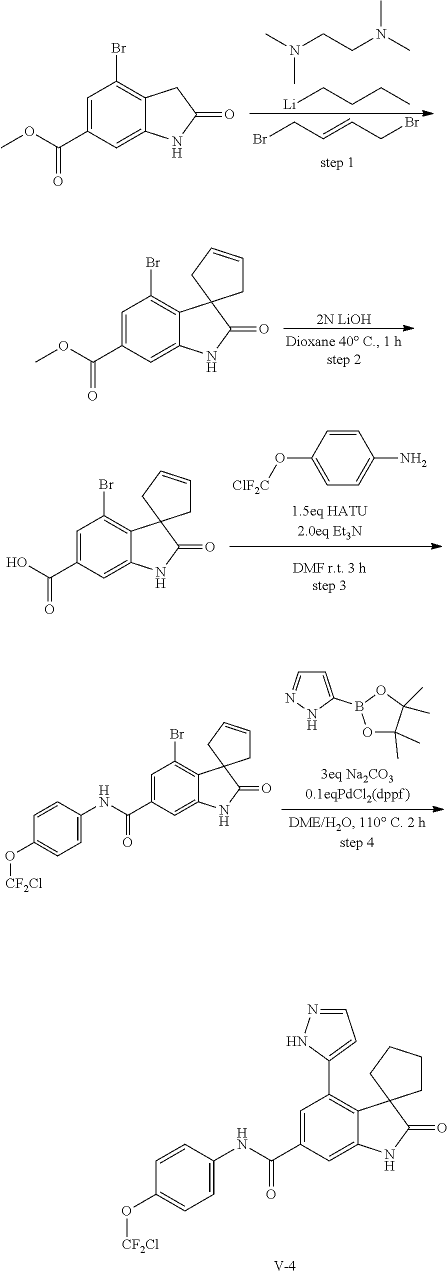

Essentially the same protocol of the preparation of Cpd. No. V-3 in EXAMPLE V-3 was used to afford Cpd. No. V-4 (19.5 mg) as a white solid. ¹H NMR (400 MHz, DMSO-d₆) δ 13.07 (s, 1H), 10.59 (s, 1H), 10.53 (s, 1H), 7.93 (d, J=8.6 Hz, 2H), 7.85 (d, J=9.3 Hz, 2H), 7.43-7.34 (m, 3H), 6.59 (s, 1H), 5.71 (s, 2H), 3.01-2.92 (m, 2H), 2.74-2.69 (m, 1H), 2.63-2.58 (m, 1H). MS: 470.95 (M+H)⁺.

### Example V-5

### Synthesis of N-(4-(chlorodifluoromethoxy)phenyl)-3-hydroxy-2′-oxo-4′-(1H-pyrazol-5-yl)spiro[cyclopentane-1,3′-indoline]-6′-carboxamide (Cpd. No. V-5)

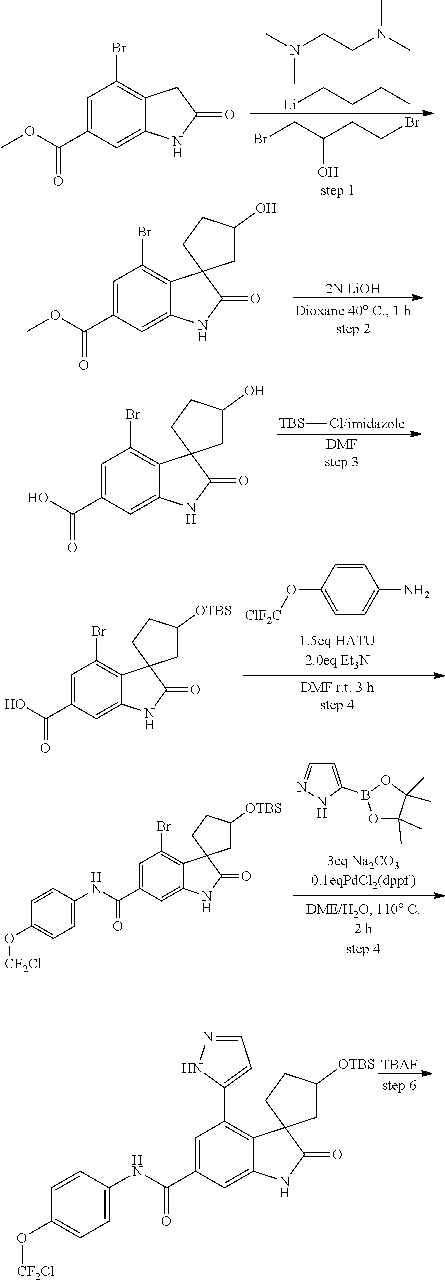

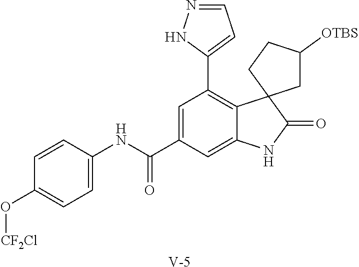

### Step 1: Synthesis of methyl 4′-bromo-3-hydroxy-2′-oxospiro[cyclopentane-1,3′-indoline]-6′-carboxylate

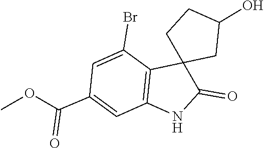 [SMILES: COC(=O)c1cc(Br)c2c(c1)NC(=O)C21CCC(O)C1]

In a 50 mL round-bottomed flask, methyl 4-bromo-2-oxoindoline-6-carboxylate (300 mg, 1.111 mmol) was dissolved in tetrahydrofuran (2 mL) under nitrogen, N1,N1,N2,N2-tetramethylethane-1,2-diamine (258 mg, 2.222 mmol) was added to the mixture at −78° C., then butyllithium (50 mg, 0.781 mmol) was added to the mixture dropwise over 10 min. After adding the materials, the mixture was stirred for 1 h at −78° C., was added 1,4-dibromobutan-2-ol (1288 mg, 5.55 mmol) dropwise over 10 min, and stirred for overnight at room temperature, then quenched with saturated aqueous NH₄Cl (10 mL), followed by extraction with ethyl acetate (20 mL×3). The combined organic layers were dried over Na₂SO₄, filtered and concentrated to give a crude product that was eluted through a silica gel column (ethyl acetate/hexane from 0 to 50%) to give methyl 4′-bromo-3-hydroxy-2′-oxospiro [cyclopentane-1,3′-indoline]-6′-carboxylate (80 mg, 21.17%). MS: 340.90 (M+H)⁺.

### Step 2: Synthesis of 4′-bromo-3-hydroxy-2′-oxospiro[cyclopentane-1,3′-indoline]-6′-carboxylic acid

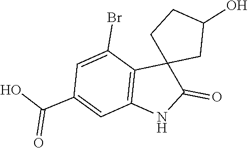 [SMILES: O=C(O)c1cc(Br)c2c(c1)NC(=O)C21CCC(O)C1]

In a 25 mL round-bottomed flask, to a solution of methyl 4′-bromo-3-hydroxy-2′-oxospiro [cyclopentane-1,3′-indoline]-6′-carboxylate (80 mg, 0.235 mmol) in 1,4-dioxane (3 mL) was added 2 N LiOH (2 mL). The reaction mixture was stirred at 50° C. for overnight, then acidified with 1N HCl to pH=4, followed by extraction with ethyl acetate (15 mL×3). The combined organic layers were concentrated to give a crude product that was eluted through a silica gel column (ethyl acetate/hexane from 0% to 90%) to give 4′-bromo-3-hydroxy-2′-oxospiro[cyclopentane-1,3′-indoline]-6′-carboxylic acid (45 mg, 58.7%). MS: 325.90 (M+H)⁺.

### Step 3: Synthesis of 4′-bromo-3-((tert-butyldimethylsilyl)oxy)-2′-oxospiro[cyclopentane-1,3′-indoline]-6′-carboxylic acid

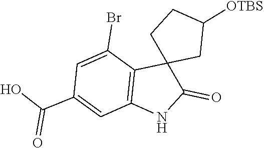 [SMILES: CC(C)(C)[Si](C)(C)OC1CCC2(C1)C(=O)Nc1cc(C(=O)O)cc(Br)c12]

In a 25 mL round-bottomed flask, to a solution of 4′-bromo-3-hydroxy-2′-oxospiro [cyclopentane-1,3′-indoline]-6′-carboxylic acid (55 mg, 0.169 mmol) in N,N-dimethylformamide (5 mL) was added imidazole (22.96 mg, 0.337 mmol) and TBS-Cl (38.1 mg, 0.253 mmol). The mixture was stirred at room temperature for overnight, then quenched with water (10 mL), followed by extraction with ethyl acetate (10 mL×3). The combined organic layers were concentrated to give a crude product that was purified by silica gel column chromatography (ethyl acetate/hexane from 0% to 70%) to give 4′-bromo-3-((tert-butyldimethylsilyl)oxy)-2′-oxospiro[cyclopentane-1,3′-indoline]-6′-carboxylic acid (68 mg, 92%) as a solid. MS: 440.90 (M+H)⁺.

### Step 4: Synthesis of 4′-bromo-3-((tert-butyldimethylsilyl)oxy)-N-(4-(chlorodifluoromethoxy)phenyl)-2′-oxospiro[cyclopentane-1,3′-indoline]-6′-carboxamide

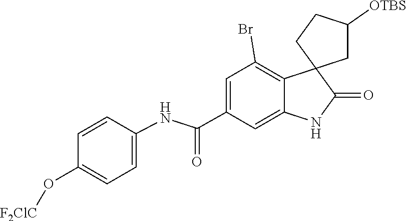

In an oven-dried 50 mL round-bottomed flask, to a solution of 4′-bromo-3-((tert-butyldimethylsilyl)oxy)-2′-oxospiro[cyclopentane-1,3′-indoline]-6′-carboxylic acid (68 mg, 0.154 mmol) in N,N-dimethylformamide (5 mL) was added 4-(chlorodifluoromethoxy)aniline (35.9 mg, 0.185 mmol), 2-(7-azabenzotriazol-1-yl)-N,N,N′,N′-tetramethyluronium hexafluorophosphate (88 mg, 0.232 mmol) and DIEA (29.9 mg, 0.232 mmol), stirred for overnight, then quenched with water (10 mL), followed by extraction with ethyl acetate (10 mL×3). The combined organic layers were dried over Na₂SO₄, filtered and concentrated to give a crude product that was eluted through a silica gel column (ethyl acetate/hexane from 0% to 80%) to give 4′-bromo-3-((tert-butyldimethylsilyl)oxy)-N-(4-(chlorodifluoromethoxy)phenyl)-2′-oxospiro[cyclopentane-1,3′-indoline]-6′-carboxamide (66 mg, 69.4%). MS: 616.90 (M+H)⁺.

### Step 5: Synthesis of 3-((tert-butyldimethylsilyl)oxy)-N-(4-(chlorodifluoromethoxy)phenyl)-2′-oxo-4′-(1H-pyrazol-5-yl)spiro[cyclopentane-1,3′-indoline]-6′-carboxamide

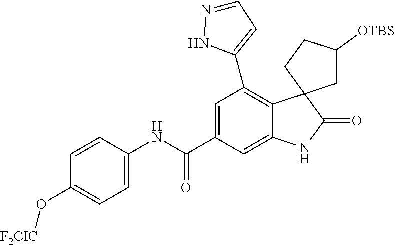

Essentially the same protocol of the preparation of Cpd. No. V-1 in Example V-1 was used to afford 3-((tert-butyldimethylsilyl)oxy)-N-(4-(chlorodifluoromethoxy)phenyl)-2′-oxo-4′-(1H-pyrazol-5-yl)spiro[cyclopentane-1,3′-indoline]-6′-carboxamide.

### Step 6: Synthesis of N-(4-(chlorodifluoromethoxy)phenyl)-3-hydroxy-2′-oxo-4′-(1H-pyrazol-5-yl)spiro[cyclopentane-1,3′-indoline]-6′-carboxamide (Cpd. No. V-5)

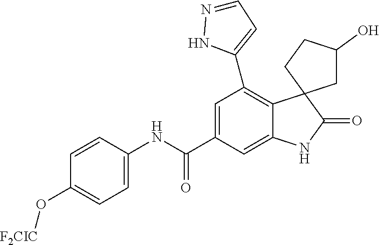

In a 100 mL round-bottomed flask, to a solution of 3-((tert-butyldimethylsilyl)oxy)-N-(4-(chlorodifluoromethoxy)phenyl)-2′-oxo-4′-(1H-pyrazol-5-yl)spiro[cyclopentane-1,3′-indoline]-6′-carboxamide (60 mg, 0.099 mmol) in tetrahydrofuran (5 mL) was added tetrabutyl ammonium fluoride (52.0 mg, 0.199 mmol). The mixture was stirred for 2 h at room temperature. The solvent was removed to give a crude product that was eluted through a silica gel column (ethyl acetate/hexane from 10% to 50%) to give N-(4-(chlorodifluoromethoxy)phenyl)-3-hydroxy-2′-oxo-4′-(1H-pyrazol-5-yl) spiro [cyclopentane-1,3′-indoline]-6′-carboxamide (15 mg, 30.8%) as a light yellow solid. ¹H NMR (400 MHz, DMSO-d₆) δ 13.12 (s, 1H), 10.71 (s, 1H), 10.45 (s, 1H), 7.93-7.86 (m, 3H), 7.67 (s, 1H), 7.39-7.34 (m, 3H), 6.60 (s, 1H), 2.33-2.19 (m, 2H), 2.04-1.97 (m, 1H), 1.91-1.86 (m, 4H).

### Example V-6

### Synthesis of N-(4-(chlorodifluoromethoxy)phenyl)-1-(2-hydroxyethyl)-3,3-dimethyl-2-oxo-4-(1H-pyrazol-5-yl)indoline-6-carboxamide (Cpd. No. V-6)

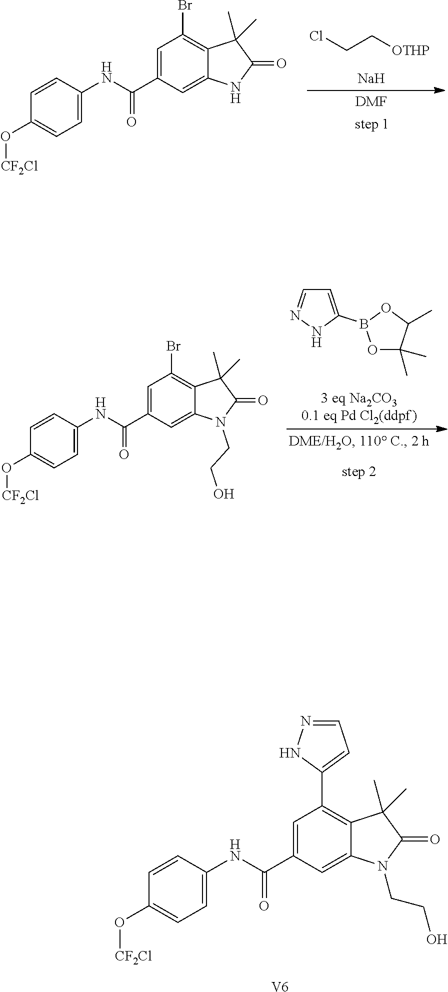

### Step 1: Synthesis of 4-bromo-N-(4-(chlorodifluoromethoxy)phenyl)-1-(2-hydroxyethyl)-3,3-dimethyl-2-oxoindoline-6-carboxamide

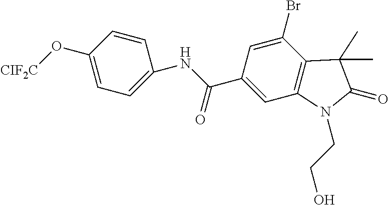

In a 100 mL round-bottomed flask, to a suspension of sodium hydride (8.35 mg, 0.348 mmol) in N,N-dimethylformamide (5 mL), was added 4-bromo-N-(4-(chlorodifluoromethoxy)phenyl)-3,3-dimethyl-2-oxoindoline-6-carboxamide in 1 mL N,N-dimethylformamide and 2-(2-chloroethoxy)tetrahydro-2H-pyran (43.0 mg, 0.261 mmol). The mixture was stirred for 2 h, then quenched with 10 mL NH₄Cl, followed by extraction with ethyl acetate (15 mL×3). The combined organic layers were dried over Na₂SO₄, filtered and concentrated to give a crude product that was used in the next step without purification as a yellow oil. ¹H NMR (400 MHz, chloroform-d) δ 8.14 (s, 1H), 8.04 (s, 1H), 7.75-7.70 (m, 2H), 7.66 (d, J=1.4 Hz, 1H), 7.60 (d, J=1.4 Hz, 1H), 7.30-7.24 (m, 2H), 4.04-3.93 (m, 2H), 3.79-3.64 (m, 2H), 1.57 (s, 3H), 1.55 (s, 3H). MS: 504.00 (M+H)⁺.

### Step 2: Synthesis of N-(4-(chlorodifluoromethoxy)phenyl)-1-(2-hydroxyethyl)-3,3-dimethyl-2-oxo-4-(1H-pyrazol-5-yl)indoline-6-carboxamide (Cpd. No. V-6)

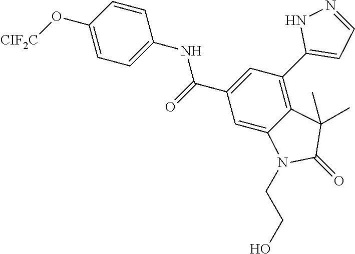

Essentially the same protocol of the preparation of Cpd. No. V-1 in Example V-1 was used to afford Cpd. No. V-6 (5.0 mg) as a white solid. ¹H NMR (400 MHz, DMSO-d₆) δ 10.50 (s, 1H), 7.95-7.87 (m, 3H), 7.77 (s, 1H), 7.63 (s, 1H), 7.40 (d, J=8.6 Hz, 2H), 6.67 (d, J=2.2 Hz, 1H), 3.86 (t, J=6.0 Hz, 2H), 3.67 (t, J=5.9 Hz, 2H), 1.41 (s, 6H). MS: 492.00 (M+H)⁺.

### Example V-7

### Synthesis of N-(4-(chlorodifluoromethoxy)phenyl)-1-(2-(dimethylamino)ethyl)-3,3-dimethyl-2-oxo-4-(1H-pyrazol-5-yl)indoline-6-carboxamide (Cpd. No. V-7)

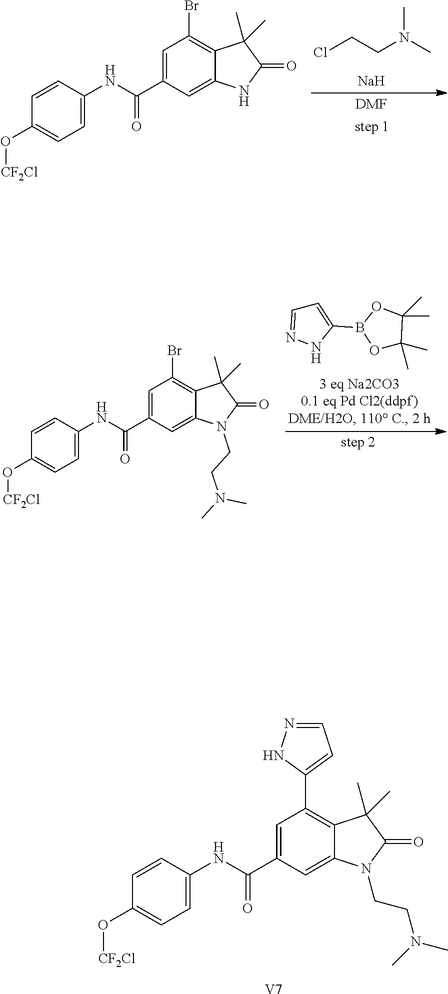

### Step 1: Synthesis of 4-bromo-N-(4-(chlorodifluoromethoxy)phenyl)-1-(2-(dimethylamino)ethyl)-3,3-dimethyl-2-oxoindoline-6-carboxamide

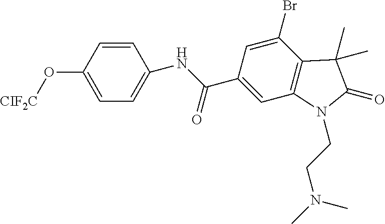

In a 100 mL round-bottomed flask, a suspension of sodium hydride (8.35 mg, 0.348 mmol) in N,N-dimethylformamide (3 mL) was added a solution of 4-bromo-N-(4-(chlorodifluoromethoxy)phenyl)-3,3-dimethyl-2-oxoindoline-6-carboxamide in 3 mL N,N-dimethylformamide and 2-chloro-N,N-dimethylethan-1-amine (28.1 mg, 0.261 mmol). The mixture was stirred at 50° C. for overnight, then quenched with 10 mL aqueous NH₄Cl, followed by extraction with ethyl acetate (15 mL×3). The combined organic layers were dried over Na₂SO₄, filtered and concentrated to give a crude product that was used in the next step without purification as a yellow oil. ¹H NMR (400 MHz, DMSO-d₆) δ 10.53 (s, 1H), 7.93-7.85 (m, 2H), 7.81 (d, J=1.3 Hz, 1H), 7.63-7.58 (m, 1H), 7.39 (d, J=8.7 Hz, 2H), 3.88 (t, J=6.5 Hz, 2H), 2.62-2.56 (m, 2H), 2.25 (s, 6H), 1.44 (s, 6H). MS: 531.90 (M+H)⁺.

### Step 2: Synthesis of N-(4-(chlorodifluoromethoxy)phenyl)-1-(2-(dimethylamino)ethyl)-3,3-dimethyl-2-oxo-4-(1H-pyrazol-5-yl)indoline-6-carboxamide (Cpd. No. V-7)

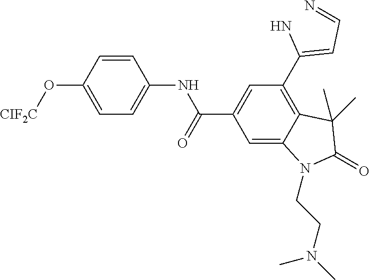

Essentially the same protocol of the preparation of Cpd. No. V-1 in EXAMPLE V-1 was used to afford Cpd. No. V-7 (15.0 mg) as a white solid. ¹H NMR (400 MHz, DMSO-d₆) δ 13.19 (s, 1H), 10.69 (s, 1H), 8.02 (d, J=8.7 Hz, 2H), 7.91 (s, 1H), 7.83 (s, 1H), 7.79 (s, 1H), 7.40 (d, J=8.8 Hz, 2H), 6.70 (s, 1H), 4.09-4.04 (m, 2H), 3.07-3.02 (m, 2H), 2.58 (s, 6H), 1.44 (s, 6H).

### Example V-8

### N-(4-(chlorodifluoromethoxy)phenyl)-1′-methyl-2-oxo-4-(1H-pyrazol-3-yl)spiro[indoline-3,3′-pyrrolidine]-6-carboxamide (Cpd. No. V-8)

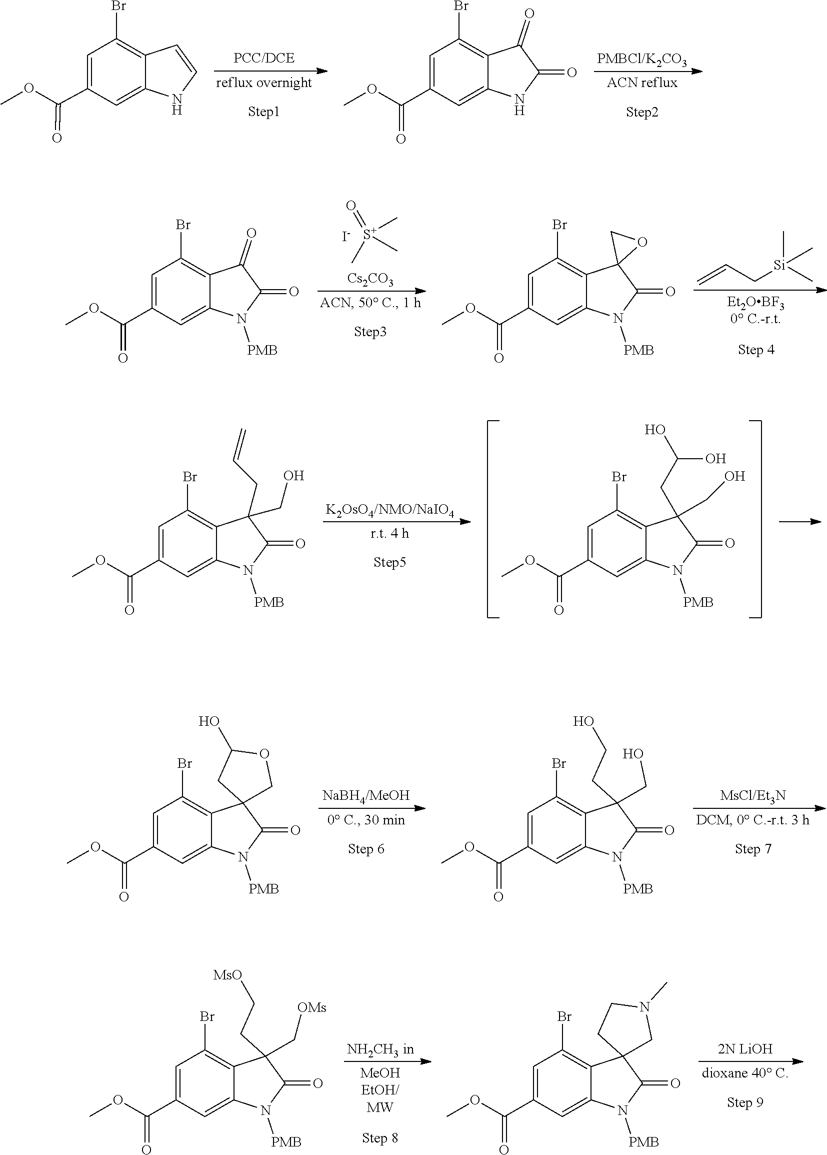

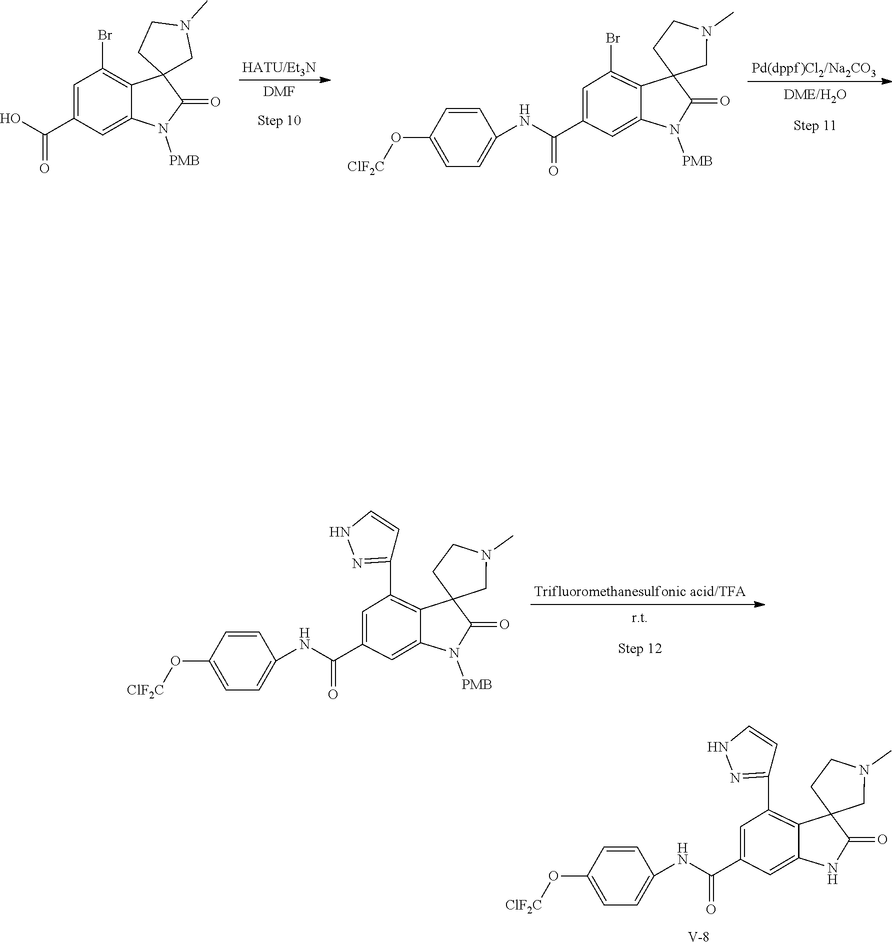

### Step 1: Synthesis of methyl 4-bromo-2,3-dioxoindoline-6-carboxylate

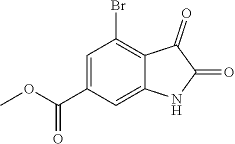 [SMILES: COC(=O)c1cc(Br)c2c(c1)NC(=O)C2=O]

In a 250 mL round-bottomed flask, to a solution of methyl 4-bromo-1H-indole-6-carboxylate (10.0 g, 39.4 mmol) in 1,2-Dichloroethane (150 ml), was added PCC (21.21 g, 98 mmol). The mixture was stirred for overnight at 80° C., then the mixture was cooled to room temperature, filtered and concentrated. The crude product was eluted through a silica gel column (ethyl acetate/hexane from 10% to 100%) to give methyl 4-bromo-2,3-dioxoindoline-6-carboxylate (4.5 g, 40.3%) as a red solid. ¹H NMR (400 MHz, DMSO-d₆) δ 11.34 (s, 1H), 7.66 (d, J=1.2 Hz, 1H), 7.29 (d, J=1.2 Hz, 1H), 3.89 (s, 3H).

### Step 2: Synthesis of methyl 4-bromo-1-(4-methoxybenzyl)-2,3-dioxoindoline-6-carboxylate

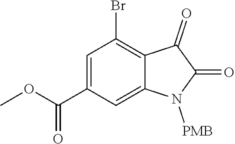

In a 100 mL round-bottomed flask, to a solution of methyl 4-bromo-2,3-dioxoindoline-6-carboxylate (1.5 g, 5.28 mmol) in acetonitrile (20 ml), were added K₂CO₃ (1.460 g, 10.56 mmol) and 1-(chloromethyl)-4-methoxybenzene (0.992 g, 6.34 mmol). The mixture was stirred for 2 h at 80° C., then evaporated to remove acetonitrile. The resulting residue was eluted through a silica gel column (ethyl acetate/hexane from 10% to 40%) to give methyl 4-bromo-1-(4-methoxybenzyl)-2,3-dioxoindoline-6-carboxylate (1.6 g, 75.0%) as a red solid. MS: 405.10 (M+1)⁺.

### Step 3: Synthesis of methyl 4-bromo-1-(4-methoxybenzyl)-2-oxospiro[indoline-3,2′-oxirane]-6-carboxylate

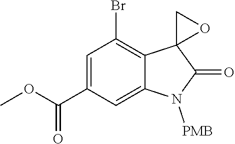

To a suspension of dimethylmethanesulfinic iodide (174 mg, 0.792 mmol) and Cs₂CO₃ (516 mg, 1.583 mmol) in dry CH₃CN (10 ml) was added a solution of methyl 4-bromo-1-(4-methoxybenzyl)-2,3-dioxoindoline-6-carboxylate (320 mg, 0.792 mmol) in acetonitrile (10 mL) dropwise at 50° C. over 10 min. The reaction was stirred at the same temperature until it was completed, then the reaction mixture was filtered and the filtrate was evaporated to dryness. The crude product was eluted through a silica gel column (ethyl acetate/hexane from 0% to 20%) to give methyl 4-bromo-1-(4-methoxybenzyl)-2-oxospiro[indoline-3,2′-oxirane]-6-carboxylate (150 mg, 45.3%). ¹H NMR (400 MHz, chloroform-d) δ 7.87 (d, J=1.3 Hz, 1H), 7.47 (d, J=1.3 Hz, 1H), 7.33-7.26 (m, 2H), 6.93-6.84 (m, 2H), 4.94 (s, 2H), 4.24 (d, J=7.1 Hz, 1H), 3.94 (s, 3H), 3.80 (s, 3H), 3.57 (d, J=7.1 Hz, 1H). Ms: 419.10 (M+1)⁺.

### Step 4: Synthesis of methyl 3-allyl-4-bromo-3-(hydroxymethyl)-1-(4-methoxybenzyl)-2-oxoindoline-6-carboxylate

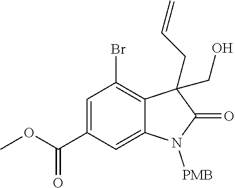

In a 25 mL round-bottomed flask, to a solution of methyl 4-bromo-1-(4-methoxybenzyl)-2-oxospiro[indoline-3,2′-oxirane]-6-carboxylate (100 mg, 0.239 mmol) and allyltrimethylsilane (54.6 mg, 0.478 mmol) in dichloromethane (5 ml), was added BF₃.EtO₂ (67.9 mg, 0.478 mmol) at 0° C. The mixture was stirred for 2 h at room temperature, quenched with 1N HCl, followed by extraction with ethyl acetate (15 ml×3). The combined organic layers were concentrated. The crude product was eluted through a silica gel column (ethyl acetate/hexane from 10% to 30%) to give methyl 3-allyl-4-bromo-3-(hydroxymethyl)-1-(4-methoxybenzyl)-2-oxoindoline-6-carboxylate (30 mg, 27.3%). MS: 461.20 (M+H)⁺.

### Step 5: Synthesis of methyl 4′-bromo-5-hydroxy-1′-(4-methoxybenzyl)-2′-oxo-4,5-dihydro-2H-spiro[furan-3,3′-indoline]-6′-carboxylate

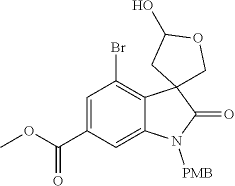

In a 100 mL round-bottomed flask, to a solution of methyl 3-allyl-4-bromo-3-(hydroxymethyl)-1-(4-methoxybenzyl)-2-oxoindoline-6-carboxylate (60 mg, 0.130 mmol) in tetrahydrofuran (5 ml), were added N-methylmorpholine oxide (30.5 mg, 0.261 mmol), potassium osmate dihydrate (2.401 mg, 6.52 μmol) and sodium periodate (112 mg, 0.521 mmol). The mixture was stirred for 3 h at room temperature, quenched with water (30 ml), followed by extraction with ethyl acetate (20 ml×3). The combined organic layers were dried over Na₂SO₄, filtered and concentrated to give the crude product. The crude product was eluted through a silica gel column (ethyl acetate/hexane from 5% to 35%) to give methyl 4′-bromo-5-hydroxy-1′-(4-methoxybenzyl)-2′-oxo-4,5-dihydro-2H-spiro[furan-3,3′-indoline]-6′-carboxylate (55 mg, 91.67%) as a colorless oil. MS: 448.00 (M+H—H₂O)⁺.

### Step 6: Synthesis of methyl 4-bromo-3-(2-hydroxyethyl)-3-(hydroxymethyl)-1-(4-methoxybenzyl)-2-oxoindoline-6-carboxylate

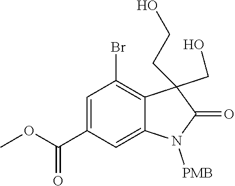

In a 100 mL round-bottomed flask, to a solution of methyl 4′-bromo-5-hydroxy-1′-(4-methoxybenzyl)-2′-oxo-4,5-dihydro-2H-spiro[furan-3,3′-indoline]-6′-carboxylate (300 mg, 0.649 mmol) in methanol (10 ml) was added NaBH₄ (49.1 mg, 1.298 mmol) at 0° C. The mixture was stirred for 0.5 h at room temperature, quenched with water (10 ml), followed by extraction with ethyl acetate (10 ml×3). The combined organic layers were dried over Na₂SO₄, filtered and concentrated to give the crude product (240 mg) without further purification.

### Step 7: Synthesis of methyl 4-bromo-1-(4-methoxybenzyl)-3-(2-((methylsulfonyl)oxy)ethyl)-3-(((methylsulfonyl)oxy)methyl)-2-oxoindoline-6-carboxylate

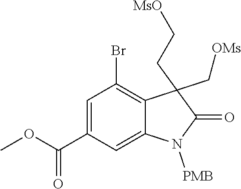

In a 100 mL round-bottomed flask, to a solution of methyl 4-bromo-3-(2-hydroxyethyl)-3-(hydroxymethyl)-1-(4-methoxybenzyl)-2-oxoindoline-6-carboxylate (240 mg, 0.517 mmol) and triethyl amine (235 mg, 2.326 mmol) in dichloromethane (5 mL) was added methanesulfonyl chloride (296 mg, 2.58 mmol) dropwise at 0° C. The mixture was stirred for 2 h at room temperature, quenched with water (10 ml), followed by extraction with ethyl acetate (10 ml×3). The combined organic layers were dried over Na₂SO₄, filtered and concentrated to give the crude product (167 mg) without further purification. MS: 621.02 (M+1)⁺.

### Step 8: Synthesis of methyl 4-bromo-1-(4-methoxybenzyl)-1′-methyl-2-oxospiro[indoline-3,3′-pyrrolidine]-6-carboxylate

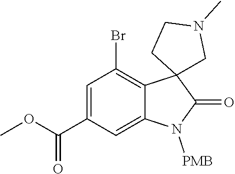

In an oven-dried 5 mL microwave tube, methyl 4-bromo-1-(4-methoxybenzyl)-3-(2-((methylsulfonyl)oxy)ethyl)-3-(((methylsulfonyl)oxy)methyl)-2-oxoindoline-6-carboxylate (167 mg, 0.269 mmol), methylamine (55.7 mg, 0.538 mmol), and triethylamine (109 mg, 1.077 mmol) were dissolved in ethanol (3 ml) under nitrogen. The mixture was stirred at 105° C. for 4 h under MW, then evaporated to remove ethanol. The residue was eluted through a silica gel column (ethyl acetate/hexane from 10% to 100%) to give methyl 4-bromo-1-(4-methoxybenzyl)-1′-methyl-2-oxospiro[indoline-3,3′-pyrrolidine]-6-carboxylate (50 mg, 40.4%). MS: 460.08 (M+1)⁺.

### Step 9: Synthesis of 4-bromo-1-(4-methoxybenzyl)-1′-methyl-2-oxospiro[indoline-3,3′-pyrrolidine]-6-carboxylic acid

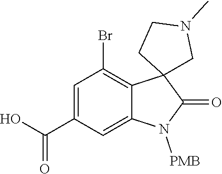

To a solution of methyl 4-bromo-1-(4-methoxybenzyl)-1′-methyl-2-oxospiro[indoline-3,3′-pyrrolidine]-6-carboxylate (50 mg, 0.109 mmol) in dioxane (3 mL), was added KOH (61.1 mg, 1.089 mmol). The mixture was stirred for 2 h, then was acidified with 1N HCl (2 ml). After being evaporated to removed water, the resulting residue was purified by silica gel column chromatography (methanol/dichloromethane from 10% to 50%) to give 4-bromo-1-(4-methoxybenzyl)-1′-methyl-2-oxospiro[indoline-3,3′-pyrrolidine]-6-carboxylic acid (23 mg, 47.4%) as a white solid. MS: 446.07 (M+H)⁺.

### Step 10: Synthesis of 4-bromo-N-(4-(chlorodifluoromethoxy)phenyl)-1-(4-methoxybenzyl)-1′-methyl-2-oxospiro[indoline-3,3′-pyrrolidine]-6-carboxamide

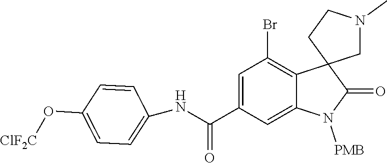

To a solution of 4-bromo-1-(4-methoxybenzyl)-1′-methyl-2-oxospiro[indoline-3,3′-pyrrolidine]-6-carboxylic acid (35 mg, 0.079 mmol) in N,N-dimethylformamide (3 ml), were added triethylamine (15.91 mg, 0.157 mmol) and 2-(7-azabenzotriazol-1-yl)-N,N,N′,N′-tetramethyluronium hexafluorophosphate (59.8 mg, 0.157 mmol). The mixture was stirred for 10 min at room temperature, then was added 4-(chlorodifluoromethoxy)aniline (18.26 mg, 0.094 mmol) and stirred overnight. The reaction mixture was diluted with ethyl acetate (20 ml), washed with water (10 ml) and brine (10 ml). The organic layer was concentrated, and the crude product was eluted through a silica gel column (ethyl acetate/hexane from 0% to 30%) to give 4-bromo-N-(4-(chlorodifluoromethoxy) phenyl)-1-(4-methoxybenzyl)-1′-methyl-2-oxospiro[indoline-3,3′-pyrrolidine]-6-carboxamide (30 mg, 61.5%). MS: 621.90 (M+H)⁺.

### Step 11: Synthesis of N-(4-(chlorodifluoromethoxy)phenyl)-1-(4-methoxybenzyl)-1′-methyl-2-oxo-4-(1H-pyrazol-3-yl)spiro[indoline-3,3′-pyrrolidine]-6-carboxamide

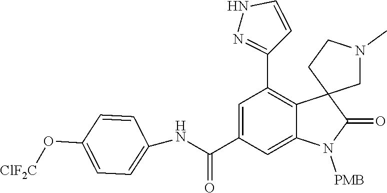

To a solution of 4-bromo-N-(4-(chlorodifluoromethoxy)phenyl)-1-(4-methoxybenzyl)-1′-methyl-2-oxospiro[indoline-3,3′-pyrrolidine]-6-carboxamide (30 mg, 0.048 mmol), 5-(4,4,5,5-tetramethyl-1,3,2-dioxaborolan-2-yl)-1H-pyrazole (14.06 mg, 0.072 mmol) in dimethoxyethane (2 ml) and water (0.4 ml), were added PdCl₂(dppf)-CH₂Cl₂ adduct (3.95 mg, 4.83 μmol) and Na₂CO₃ (10.24 mg, 0.097 mmol). The mixture was stirred at 110° C. under MW for 1.5 h, then quenched with water (10 ml), followed by extraction with ethyl acetate (10 ml×3). The combined organic layers were concentrated, and the resulting crude product was eluted through a silica gel column (ethyl acetate/hexane from 5% to 100%, then with methanol/dichloromethane from 1% to 20%) to give N-(4-(chlorodifluoromethoxy)phenyl)-1-(4-methoxybenzyl)-1′-methyl-2-oxo-4-(1H-pyrazol-5-yl)spiro[indoline-3,3′-pyrrolidine]-6-carboxamide (12 mg, 40.8%). MS: 609.00 (M+H)⁺.

### Step 12: Synthesis of N-(4-(chlorodifluoromethoxy)phenyl)-1′-methyl-2-oxo-4-(1H-pyrazol-3-yl) spiro[indoline-3,3′-pyrrolidine]-6-carboxamide

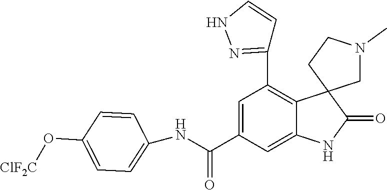

To a solution of 4-bromo-N-(4-(chlorodifluoromethoxy)phenyl)-1-(4-methoxybenzyl)-1′-methyl-2-oxospiro[indoline-3,3′-pyrrolidine]-6-carboxamide (12 mg, 0.019 mmol) in trifluoroacetic acid was added 0.5 mL trifluoromethanesulfonic acid at −10° C. The mixture was warmed to room temperature and stirred at the same temperature for 3 h, then concentrated to removed trifluoroacetic acid. The resulting residue was purified with preparative HPLC to give N-(4-(chlorodifluoromethoxy)phenyl)-1′-methyl-2-oxo-4-(1H-pyrazol-5-yl)spiro[indoline-3,3′-pyrrolidine]-6-carboxamide (2 mg, 21.21%). MS: 488.95 (M+H)⁺.

### Example V-9

### Synthesis of N-(4-(chlorodifluoromethoxy)phenyl)-3,4-dihydroxy-2′-oxo-4′-(1H-pyrazol-5-yl)spiro[cyclopentane-1,3′-indoline]-6′-carboxamide (Cpd. No. V-9)

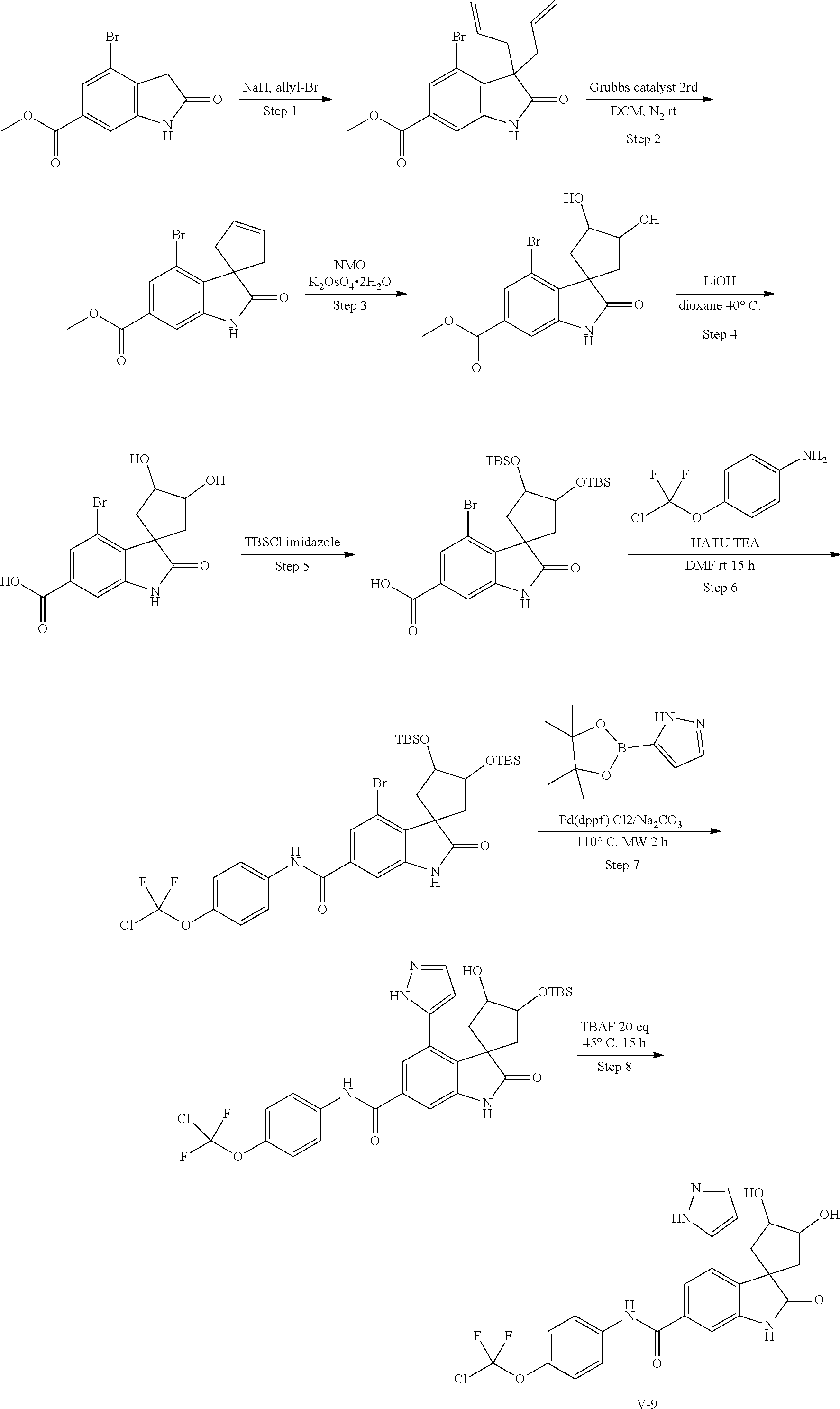

### Step 1: Synthesis of methyl 3,3-diallyl-4-bromo-2-oxoindoline-6-carboxylate

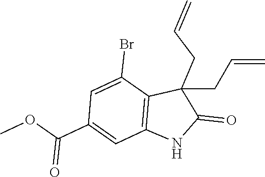 [SMILES: C=CCC1(CC=C)C(=O)Nc2cc(C(=O)OC)cc(Br)c21]

In a 100 mL round-bottomed flask, to a suspension of sodium hydride (118 mg, 2.96 mmol) in N,N-dimethylformamide (5 ml), was added the solution of methyl 4-bromo-2-oxoindoline-6-carboxylate (400 mg, 1.481 mmol) in N,N-dimethylformamide (3 ml). After being stirred for 15 min, the mixture was added 3-bromoprop-1-ene (358 mg, 2.96 mmol). The mixture was stirred for another 2 h at room temperature, then quenched with saturated NH₄Cl aqueous solution (10 ml), followed by extraction with ethyl acetate (20 ml×3). The combined organic layers were dried over Na₂SO₄, filtered and concentrated to give the crude product, which was eluted through a silica gel column (ethyl acetate/hexane from 5% to 50%) to give methyl 3,3-diallyl-4-bromo-2-oxoindoline-6-carboxylate (450 mg, 87%). ¹H NMR (400 MHz, chloroform-d) δ 8.51 (s, 1H), 7.89 (d, J=1.3 Hz, 1H), 7.48 (d, J=1.3 Hz, 1H), 5.41-5.26 (m, 2H), 5.09-4.97 (m, 2H), 4.87-4.78 (m, 2H), 3.94 (s, 3H), 3.13 (dd, J=13.5, 7.4 Hz, 2H), 2.64 (dd, J=13.5, 7.3 Hz, 2H).

### Step 2: Synthesis of methyl 4′-bromo-2′-oxospiro[cyclopentane-1,3′-indolin]-3-ene-6′-carboxylate

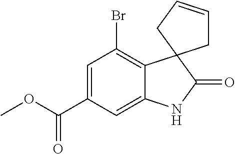 [SMILES: COC(=O)c1cc(Br)c2c(c1)NC(=O)C21CC=CC1]

To a solution of methyl 3,3-diallyl-4-bromo-2-oxoindoline-6-carboxylate (90 mg, 0.257 mmol) in dichloromethane (10 ml) under nitrogen, was added Grubbs ii catalyst (43.6 mg, 0.051 mmol). The reaction mixture was stirred for 4 h at room temperature, then was concentrated. The resulting residue was eluted through a silica gel column (ethyl acetate/hexane from 0% to 50%) to give methyl 4′-bromo-2′-oxospiro[cyclopentane-1,3′-indolin]-3-ene-6′-carboxylate (40 mg, 48.3%). MS: 323.00 (M+H)⁺.

### Step 3: Synthesis of methyl 4′-bromo-3,4-dihydroxy-2′-oxospiro[cyclopentane-1,3′-indoline]-6′-carboxylate

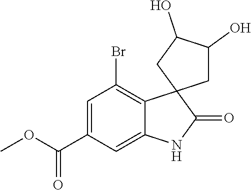 [SMILES: COC(=O)c1cc(Br)c2c(c1)NC(=O)C21CC(O)C(O)C1]

In a 50 mL round-bottomed flask, to a solution of methyl 4′-bromo-2′-oxospiro[cyclopentane-1,3′-indolin]-3-ene-6′-carboxylate (120 mg, 0.372 mmol) and N-methylmorpholine oxide (87 mg, 0.745 mmol) in acetone (5 mL) was added a solution of potassium osmate (6.19 mg, 0.019 mmol) in water (0.500 mL) at 0° C. The reaction mixture was stirred at the same temperature for overnight, then diluted with water (10 mL), followed by extraction with ethyl acetate (10 mL×3). The combined organic layers were washed with Na₂S₂O₃ aqueous solution, concentrated to afford crude product, which was eluted through a silica gel column (ethyl acetate/hexane from 0% to 80%) to give methyl 4′-bromo-3,4-dihydroxy-2′-oxospiro[cyclopentane-1,3′-indoline]-6′-carboxylate (100 mg, 75%). MS: 357.00 (M+H)⁺.

### Step 4: Synthesis of 4′-bromo-3,4-dihydroxy-2′-oxospiro[cyclopentane-1,3′-indoline]-6′-carboxylic acid

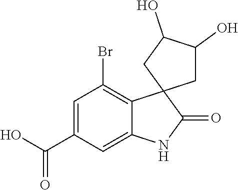 [SMILES: O=C(O)c1cc(Br)c2c(c1)NC(=O)C21CC(O)C(O)C1]

To a solution of methyl 4′-bromo-3,4-dihydroxy-2′-oxospiro[cyclopentane-1,3′-indoline]-6′-carboxylate (95 mg, 0.267 mmol) in dioxane (5 ml), was added lithium hydroxide (2N, 2 mL). The reaction mixture was stirred for 3 h at 40° C., then acidified with HCl (1N, 5 ml) to adjust pH=2, followed by extraction with ethyl acetate (10 ml×6). The combined organic layers were dried over Na₂SO₄, filtered and concentrated to give crude product 78 mg. MS: 342.99 (M+H)⁺.

### Step 5: Synthesis of 4′-bromo-3,4-bis((tert-butyldimethylsilyl)oxy)-2′-oxospiro[cyclopentane-1,3′-indoline]-6′-carboxylic acid

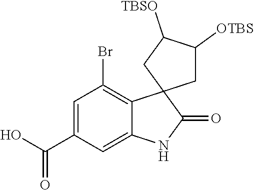 [SMILES: CC(C)(C)[Si](C)(C)OC1CC2(CC1O[Si](C)(C)C(C)(C)C)C(=O)Nc1cc(C(=O)O)cc(Br)c12]

To a solution of 4′-bromo-3,4-dihydroxy-2′-oxospiro[cyclopentane-1,3′-indoline]-6′-carboxylic acid (78 mg, 0.228 mmol) in N,N-dimethylformamide (5 ml), were added imidazole (78 mg, 1.140 mmol) and tert-butylchlorodimethylsilane (172 mg, 1.140 mmol). The reaction mixture was stirred at room temperature for 36 h, then quenched with water (5 ml), followed by extraction with ethyl acetate (10 ml×6). The combined organic layers were dried over Na₂SO₄, filtered and concentrated. The resulting residue was eluted (methanol/ethyl acetate from 0% to 20%) to give 4′-bromo-3,4-bis((tert-butyldimethylsilyl)oxy)-2′-oxospiro [cyclopentane-1,3′-indoline]-6′-carboxylic acid (79 mg, 60.7%). MS: 571.16 (M+H)⁺.

### Step 6: Synthesis of 4′-bromo-3,4-bis((tert-butyldimethylsilyl)oxy)-N-(4-(chlorodifluoromethoxy) phenyl)-2′-oxospiro[cyclopentane-1,3′-indoline]-6′-carboxamide

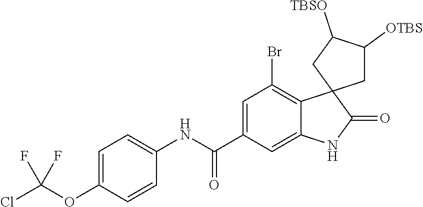 [SMILES: CC(C)(C)[Si](C)(C)OC1CC2(CC1O[Si](C)(C)C(C)(C)C)C(=O)Nc1cc(C(=O)Nc3ccc(OC(F)(F)Cl)cc3)cc(Br)c12]

Essentially the same protocol of step 5 in EXAMPLE V-1 to give 4′-bromo-3,4-bis((tert-butyldimethylsilyl)oxy)-N-(4-(chlorodifluoromethoxy)phenyl)-2′-oxospiro[cyclopentane-1,3′-indoline]-6′-carboxamide (78 mg, 76%) as a white solid. MS: 746.16 (M+H).

### Step 7: Synthesis of 3-((tert-butyldimethylsilyl)oxy)-N-(4-(chlorodifluoromethoxy)-phenyl)-4-hydroxy-2′-oxo-4′-(1H-pyrazol-5-yl)spiro[cyclopentane-1,3′-indoline]-6′-carboxamide

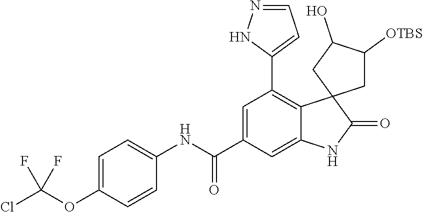 [SMILES: CC(C)(C)[Si](C)(C)OC1CC2(CC1O)C(=O)Nc1cc(C(=O)Nc3ccc(OC(F)(F)Cl)cc3)cc(-c3ccn[nH]3)c12]

4′-bromo-3,4-bis((tert-butyldimethylsilyl)oxy)-N-(4-(chlorodifluoromethoxy) phenyl)-2′-oxospiro[cyclopentane-1,3′-indoline]-6′-carboxamide (43 mg, 0.058 mmol) 5-(4,4,5,5-tetramethyl-1,3,2-dioxaborolan-2-yl)-1H-pyrazole (16.77 mg, 0.086 mmol), sodium carbonate (12.21 mg, 0.115 mmol), PdCl₂(dppf) (4.71 mg, 5.76 μmol) were dissolved in dimethoxyethane (2 mL) and water (0.4 ml). The mixture was stirred at 110° C. for 2 h under MW, then quenched with water (10 mL), followed by extraction with ethyl acetate (10 ml×3). The combined organic layers were concentrated to give crude product which was eluted through a silica gel column (ethyl acetate/hexane from 5% to 80%) to give 3,4-bis((tert-butyldimethylsilyl)oxy)-N-(4-(chlorodifluoromethoxy)phenyl)-2′-oxo-4′-(1H-pyrazol-5-yl)spiro[cyclopentane-1,3′-indoline]-6′-carboxamide (28 mg, 66.3%) as a white solid.

### Step 8: Synthesis of N-(4-(chlorodifluoromethoxy)phenyl)-3,4-dihydroxy-2′-oxo-4′-(1H-pyrazol-5-yl) spiro[cyclopentane-1,3′-indoline]-6′-carboxamide (Cpd. No. V-9)

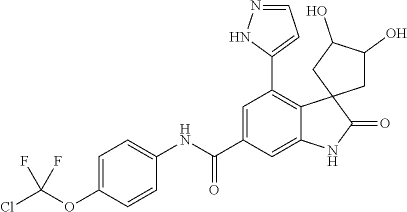 [SMILES: O=C(Nc1ccc(OC(F)(F)Cl)cc1)c1cc2c(c(-c3ccn[nH]3)c1)C1(CC(O)C(O)C1)C(=O)N2]

To a solution of 3-((tert-butyldimethylsilyl)oxy)-N-(4-(chlorodifluoromethoxy)-phenyl)-4-hydroxy-2′-oxo-4′-(1H-pyrazol-5-yl)spiro[cyclopentane-1,3′-indoline]-6′-carboxamide (28 mg, 0.045 mmol) in tetrahydrofuran (3 ml), was added tetrabutyl ammonium fluoride (236 mg, 0.904 mmol). The reaction mixture was heated at 45° C. for overnight, then purified by preparative HPLC to give N-(4-(chlorodifluoromethoxy)phenyl)-3,4-dihydroxy-2′-oxo-4′-(1H-pyrazol-5-yl)spiro[cyclopentane-1,3′-indoline]-6′-carboxamide (3 mg, 13.2%). Ms: 506.10 (M+H)⁺.

### Example V-10

### Synthesis of N-(4-(chlorodifluoromethoxy)phenyl)-1′-methyl-2-oxo-4-(1H-pyrazol-5-yl)spiro[indoline-3,4′-piperidine]-6-carboxamide (Cpd. No. V-10)

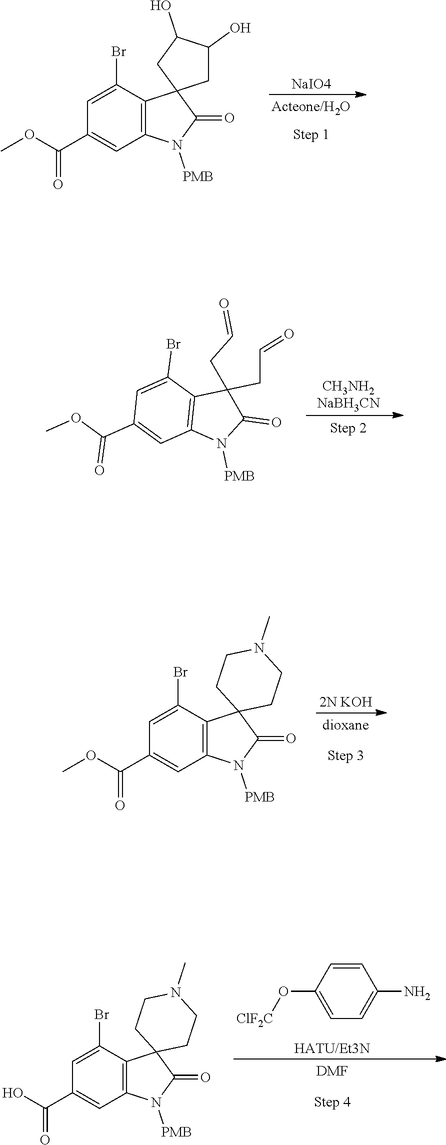

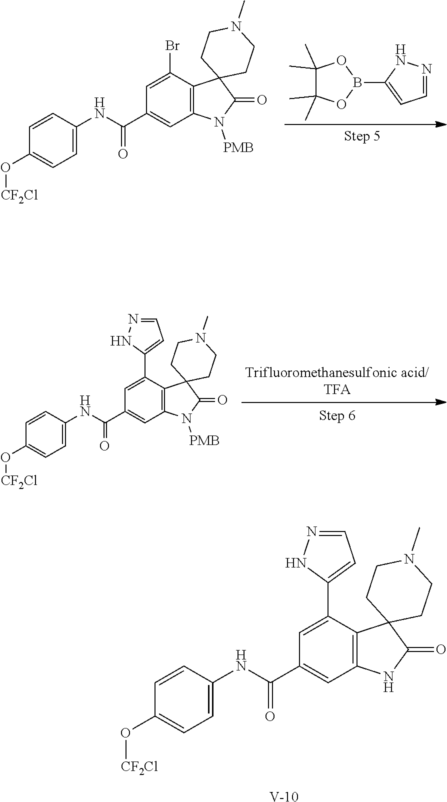

### Step 1: Synthesis of methyl 4-bromo-1-(4-methoxybenzyl)-2-oxo-3,3-bis(2-oxoethyl)-indoline-6-carboxylate

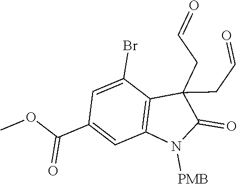

In a 100 mL round-bottomed flask, to a solution of methyl 4′-bromo-3,4-dihydroxy-1′-(4-methoxybenzyl)-2′-oxospiro[cyclopentane-1,3′-indoline]-6′-carboxylate (223 mg, 0.468 mmol) in tetrahydrofuran (5 ml), was added sodium periodate (200 mg, 0.936 mmol). The mixture was stirred for 3 h at room temperature, then diluted with water (30 ml), followed by extraction with ethyl acetate (20 ml×3). The combined organic layers were dried over Na₂SO₄, filtered and concentrated to give methyl 4-bromo-1-(4-methoxybenzyl)-2-oxo-3,3-bis(2-oxoethyl)indoline-6-carboxylate (207 mg, 93%) without further purification. MS: 475.05 (M+H)⁺.

### Step 2: Synthesis of methyl 4-bromo-1-(4-methoxybenzyl)-1′-methyl-2-oxospiro[indoline-3,4′-piperidine]-6-carboxylate

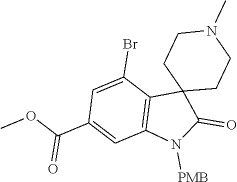

To a solution of methyl 4-bromo-1-(4-methoxybenzyl)-2-oxo-3,3-bis(2-oxoethyl)indoline-6-carboxylate (207 mg, 0.436 mmol) in methanol (10 ml), were added sodium cyanotrihydroborate (54.9 mg, 0.873 mmol), methylamine (20.33 mg, 0.655 mmol), acetic acid (2.62 mg, 0.044 mmol) under nitrogen. The mixture was stirred for 2 h at room temperature, then eluted through a silica gel column (ethyl acetate/hexane from 10% to 100%) to give methyl 4-bromo-1-(4-methoxybenzyl)-1′-methyl-2-oxospiro[indoline-3,4′-piperidine]-6-carboxylate (160 mg, 77%) as a colorless oil. ¹H NMR (400 MHz, DMSO-d₆) δ 7.72 (d, J=1.4 Hz, 1H), 7.37 (d, J=1.4 Hz, 1H), 7.24-7.16 (m, 2H), 6.95-6.86 (m, 2H), 4.90 (s, 2H), 3.83 (s, 3H), 3.72 (s, 3H), 2.94-2.71 (m, 4H), 2.42 (s, 3H), 1.94-1.89 (m, 2H), 1.71-1.53 (m, 2H). MS: 474.10 (M+H)⁺.

### Step 3: Synthesis of 4-bromo-1-(4-methoxybenzyl)-1′-methyl-2-oxospiro[indoline-3,4′-piperidine]-6-carboxylic acid

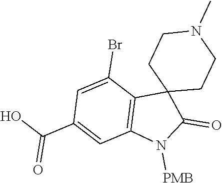

In a nitrogen flushed 25 mL round-bottomed flask, to a solution of methyl 4-bromo-1-(4-methoxybenzyl)-1′-methyl-2-oxospiro[indoline-3,4′-piperidine]-6-carboxylate (160 mg, 0.338 mmol) in 1,4-dioxane (3 mL), was added 2N LiOH (2 mL). The mixture was stirred at 50° C. for overnight, then acidified with HCl (1N, 2 mL). After evaporating to remove water, the resulting residue was purified by silica gel column chromatography (methanol/dichloromethane from 10% to 50%) to give 4-bromo-1-(4-methoxybenzyl)-1′-methyl-2-oxospiro[indoline-3,4′-piperidine]-6-carboxylic acid (150 mg, 97%). MS: 460.08 (M+H)⁺.

### Step 4: Synthesis of 4-bromo-N-(4-(chlorodifluoromethoxy)phenyl)-1-(4-methoxybenzyl)-1′-methyl-2-oxospiro[indoline-3,4′-piperidine]-6-carboxamide

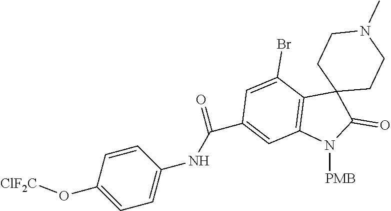

Essentially the same protocol in the step 5 of EXAMPLE V-1 to give 4-bromo-N-(4-(chlorodifluoromethoxy) phenyl)-1-(4-methoxybenzyl)-1′-methyl-2-oxospiro[indoline-3,4′-piperidine]-6-carboxamide (100 mg, 55.7%). MS: 635.08 (M+H)⁺.

### Step 5: Synthesis of N-(4-(chlorodifluoromethoxy)phenyl)-1-(4-methoxybenzyl)-1′-methyl-2-oxo-4-(1H-pyrazol-5-yl)spiro[indoline-3,4′-piperidine]-6-carboxamide

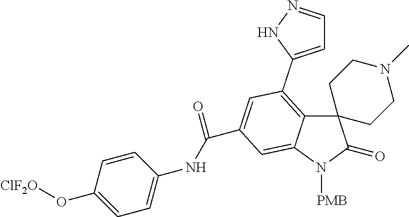

4-bromo-N-(4-(chlorodifluoromethoxy)phenyl)-1-(4-methoxybenzyl)-1′-methyl-2-oxospiro[indoline-3,4′-piperidine]-6-carboxamide (105 mg, 0.165 mmol) Na₂CO₃ (52.6 mg, 0.496 mmol), 5-(4,4,5,5-tetramethyl-1,3,2-dioxaborolan-2-yl)-1H-pyrazole (48.1 mg, 0.248 mmol) and PdCl₂(dppf)-CH₂Cl₂ adduct (13.51 mg, 0.017 mmol) were dissolved in dimethoxyethane (2 mL) and water (0.4 mL). The mixture was stirred for at 110° C. for 2 h under microwave, then quenched with water (10 mL), followed by extraction with ethyl acetate (10 ml×3). The combined organic layers were concentrated to afford crude product, which was eluted through a silica gel column (ethyl acetate/hexane from 5% to 100%) to give N-(4-(chlorodifluoromethoxy)phenyl)-1-(4-methoxybenzyl)-1′-methyl-2-oxo-4-(1H-pyrazol-5-yl)spiro[indoline-3,4′-piperidine]-6-carboxamide (28.5 mg, 27.7%). MS: 622.20 (M+H)⁺.

### Step 6: Synthesis of N-(4-(chlorodifluoromethoxy)phenyl)-1′-methyl-2-oxo-4-(1H-pyrazol-5-yl)spiro[indoline-3,4′-piperidine]-6-carboxamide (Cpd. No. V-10)

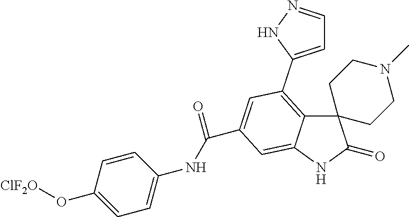

To a solution of N-(4-(chlorodifluoromethoxy)phenyl)-1-(4-methoxybenzyl)-1′-methyl-2-oxo-4-(1H-pyrazol-5-yl)spiro[indoline-3,4′-piperidine]-6-carboxamide (28.5 mg, 0.046 mmol) in trifluoroacetic acid (3 mL) was added trifluoromethanesulfonic acid (5 mL) at 0° C. The mixture was stirred for overnight at room temperature, then evaporate in vacuo to remove trifluoroacetic acid. The resulting residue was purified with preparative HPLC to give N-(4-(chlorodifluoromethoxy)phenyl)-1′-methyl-2-oxo-4-(1H-pyrazol-5-yl)spiro[indoline-3,4′-piperidine]-6-carboxamide (5 mg, 6.5%). MS: 503.13 (M+H)⁺.

### Example V-11

### Synthesis of N-(4-(chlorodifluoromethoxy)phenyl)-2′-oxo-4′-(1H-pyrazol-5-yl)spiro[cyclohexane-1,3′-indoline]-6′-carboxamide (Cpd. No. V-11)

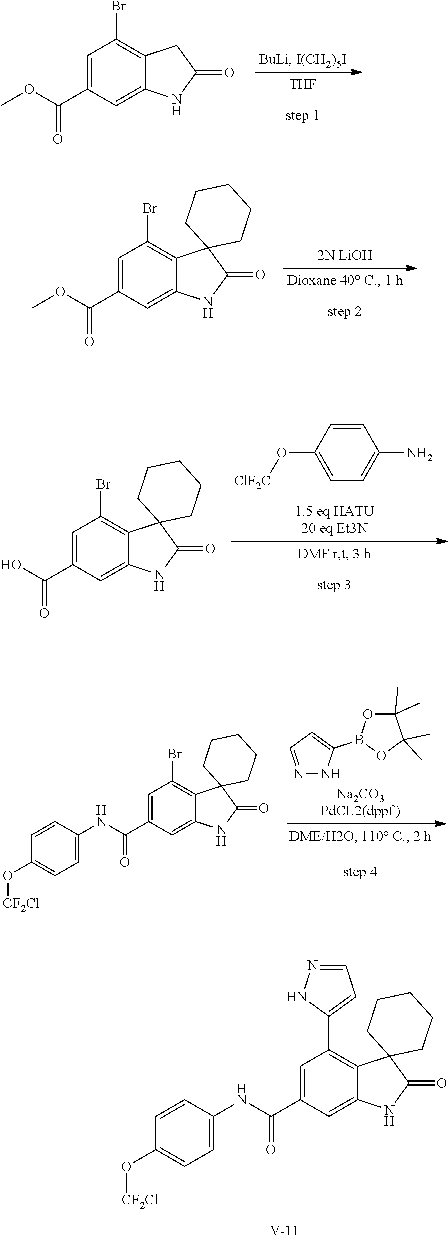

### Step 1: Synthesis of methyl 4′-bromo-2′-oxospiro[cyclohexane-1,3′-indoline]-6′-carboxylate

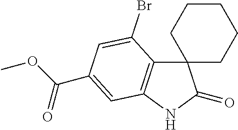 [SMILES: COC(=O)c1cc(Br)c2c(c1)NC(=O)C21CCCCC1]

In a 50 mL round-bottomed flask, to a solution of methyl 4-bromo-2-oxoindoline-6-carboxylate (150 mg, 0.555 mmol) in tetrahydrofuran (2 ml), was added N1,N1,N2,N2-tetramethylethane-1,2-diamine (129 mg, 1.111 mmol) under nitrogen. After being cooled to cooled to −78° C., the mixture was dropped butyllithium (1N in hexane, 50 mg, 0.781 mmol) over 10 min and stirred for 1 h at −78° C., then was dropped 1,5-diiodopentane (374 mg, 1.11 mmol) over 10 min and warmed up to room temperature. The mixture was stirred for overnight, then quenched with saturated NH₄Cl aqueous solution (10 ml), followed by extraction with ethyl acetate (20 ml×3). The combined organic layers were concentrated to give crude product which was eluted through a silica gel column (ethyl acetate/hexane from 0% to 50%) to give methyl 4′-bromo-2′-oxospiro [cyclohexane-1,3′-indoline]-6′-carboxylate (50 mg, 26.6%). MS: 337.90 (M+H)⁺.

### Step 2: Synthesis of 4′-bromo-2′-oxospiro[cyclohexane-1,3′-indoline]-6′-carboxylic acid

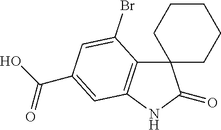 [SMILES: O=C(O)c1cc(Br)c2c(c1)NC(=O)C21CCCCC1]

Essentially the same protocol used in the step 2 of EXAMPLE V-3 to afford 4′-bromo-2′-oxospiro[cyclohexane-1,3′-indoline]-6′-carboxylic acid. MS: 324.90 (M+H)⁺.

### Step 3: synthesis 4′-bromo-N-(4-(chlorodifluoromethoxy)phenyl)-2′-oxospiro [cyclohexane-1,3′-indoline]-6′-carboxamide

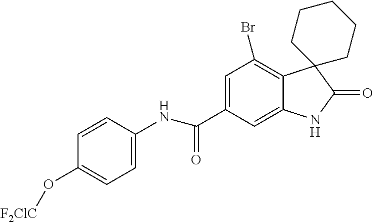

Essentially the same protocol used in the step 3 of EXAMPLE V-3 to afford 4′-bromo-N-(4-(chlorodifluoromethoxy)phenyl)-2′-oxospiro [cyclohexane-1,3′-indoline]-6′-carboxamide. MS: 500.90 (M+H)⁺.

### Step 4: Synthesis of N-(4-(chlorodifluoromethoxy)phenyl)-2′-oxo-4′-(1H-pyrazol-5-yl) spiro [cyclohexane-1,3′-indoline]-6′-carboxamide (Cpd. No. V-11)

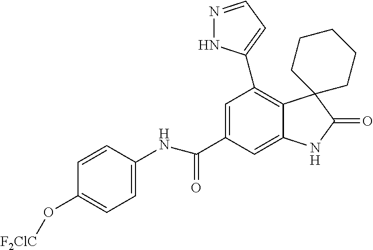

Essentially the same protocol used in the step 4 of EXAMPLE V-3 to afford N-(4-(chlorodifluoromethoxy)phenyl)-2′-oxo-4′-(1H-pyrazol-5-yl) spiro[cyclohexane-1,3′-indoline]-6′-carboxamide. ¹H NMR (400 MHz, DMSO-d₆) δ 13.12 (s, 1H), 10.48 (s, 1H), 7.95-7.86 (m, 3H), 7.61 (s, 1H), 7.41-7.32 (m, 3H), 6.57 (s, 1H), 2.19-1.98 (m, 3H), 1.69-1.44 (m, 5H), 1.34-1.13 (m, 2H). MS: 487.00 (M+H)⁺.

### Example VII-1

### Synthesis of N-(4-(chlorodifluoromethoxy)phenyl)-1-isopropyl-7-(1H-pyrazol-5-yl)indoline-5-carboxamide (Cpd. No. VII-1)

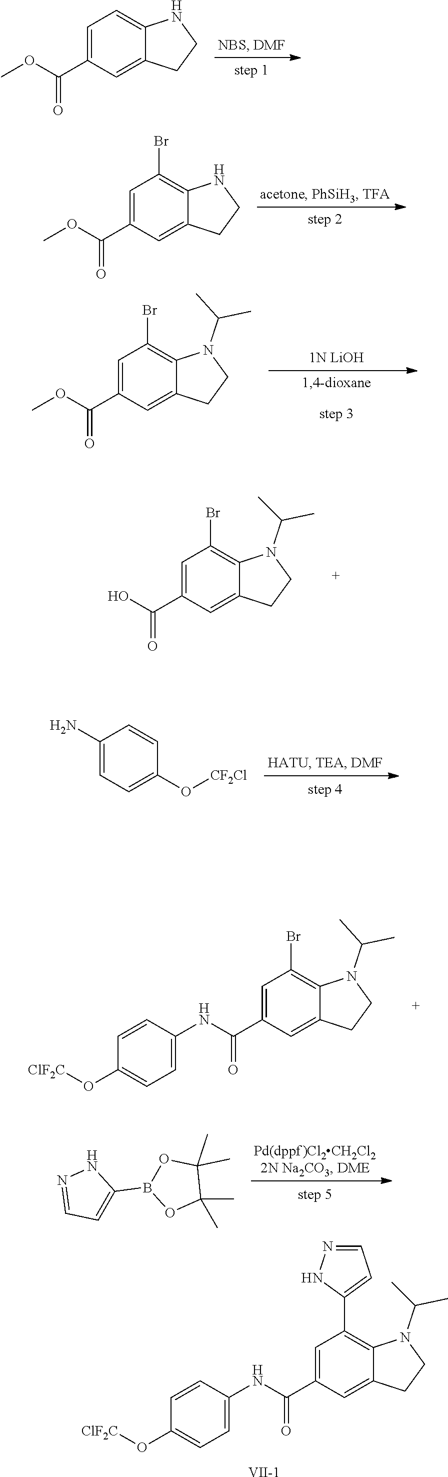

### Step 1: Synthesis of Methyl 7-bromoindoline-5-carboxylate

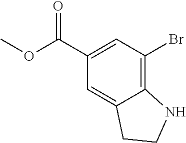

To a solution of methyl indoline-5-carboxylate (1.50 g, 8.46 mmol) in dioxane/CH₃COOH (5.0 mL/2.0 mL) was added a solution of N-bromosuccinimide (1.81 g, 10.16 mmol) in dioxane (30.0 mL) dropwise at 0° C. The mixture was stirred at 25° C. for 2 h, then concentrated. The resulting residue was basified with 2 N NaOH (5.0 mL), diluted with water (100.0 mL), extracted with ethyl acetate (30 mL×3). The combined organic layers were dried over Na₂SO₄, filtered and concentrated to give a crude product that was eluted through a silica gel column (ethyl acetate/hexane from 0% to 30%) to give methyl 7-bromoindoline-5-carboxylate (1.50 g, 69.2%) as a white solid. MS: 256.1 (M+H)⁺.

### Step 2: Synthesis of Methyl 7-bromo-1-isopropylindoline-5-carboxylate

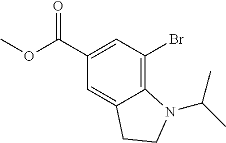 [SMILES: COC(=O)c1cc(Br)c2c(c1)CCN2C(C)C]

Methyl 7-bromoindoline-5-carboxylate (1.00 g, 3.90 mmol) and phenylsilane (4.23 g, 39.0 mmol) were dissolved in trifluoroacetic acid (10 mL) and acetone (10 mL). The mixture was stirred at room temperature for 2 h, then concentrated. The residue was basified with 2N Na₂CO₃ (15.0 mL), diluted with water (100.0 mL), extracted with ethyl acetate (30 mL×3), the combined organic layers were dried over Na₂SO₄, filtered and concentrated to give a crude product that was eluted through a silica gel column (ethyl acetate/hexane from 0% to 35%) to give methyl 7-bromo-1-isopropylindoline-5-carboxylate (1.00 g, 86.0%) as a yellow oil. MS: 298.2 (M+H)⁺.

### Step 3: Synthesis of 7-bromo-1-isopropylindoline-5-carboxylic acid

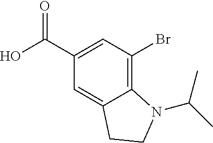 [SMILES: CC(C)N1CCc2cc(C(=O)O)cc(Br)c21]

Lithium hydroxide (1.0 N, 6.71 mmol) was added to a solution of methyl 7-bromo-1-isopropylindoline-5-carboxylate in dioxane (15.0 mL). The mixture was stirred at 40° C. for 5 h, then concentrated and acidified with 1N HCl (20.0 mL). The precipitate was filtered and the filtrate cake was washed with water and hexane, and dried under vacuum to give 7-bromo-1-isopropylindoline-5-carboxylic acid (0.90 g, 94.0%) as white solid. MS: 284.1 (M+H)⁺.

### Step 4: Synthesis of 7-bromo-N-(4-(chlorodifluoromethoxy) phenyl)-1-isopropylindoline-5-carboxamide

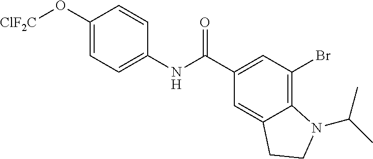

4-(chlorodifluoromethoxy)aniline (204.0 mg, 1.06 mmol), triethylamine (142.0 mg, 1.41 mmol) and 2-(7-azabenzotriazol-1-yl)-N,N,N′,N′-tetramethyluronium hexafluorophosphate (535.0 mg, 1.41 mmol) were added to a solution of 7-bromo-1-isopropylindoline-5-carboxylic acid (200.0 mg, 0.70 mmol) in N,N-dimethylformamide (6.0 mL). The mixture was stirred at 45° C. for 5 h. Then the reaction mixture was diluted with ethyl acetate (50.0 mL) and washed with water (50.0 mL) and brine (50.0 mL). The organic layer was dried over Na₂SO₄, filtered and concentrated to give a crude product that was eluted through a silica gel column (ethyl acetate/hexane from 0% to 35%) to give 7-bromo-N-(4-(chlorodifluoromethoxy)phenyl)-1-isopropylindoline-5-carboxamide (160.0 mg, 49.4%) as a white solid. MS: 459.1 (M+H)⁺.

### Step 5: Synthesis of N-(4-(chlorodifluoromethoxy) phenyl)-1-isopropyl-7-(1H-pyrazol-5-yl) indoline-5-carboxamide (Cpd. No. VII-1)

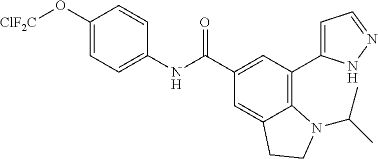

5-(4,4,5,5-tetramethyl-1,3,2-dioxaborolan-2-yl)-1H-pyrazole (67.5 mg, 0.35 mmol) and PdCl₂(dppf)-CH₂Cl₂ adduct (28.5 mg, 0.035 mmol) were added to a solution of 7-bromo-N-(4-(chlorodifluoromethoxy) phenyl)-1-isopropylindoline-5-carboxamide (80.0 mg, 0.17 mmol) in dimethoxyethane (9 mL)/2N Na₂CO₃ (3 mL). The mixture was purged with nitrogen, then stirred at 100° C. under MW for 0.5 h. The mixture was diluted with diethyl ether (20.0 mL) and washed with water (20.0 mL) and brine (20.0 mL). The organic layer was dried over Na₂SO₄, filtered and concentrated to give a crude product that was purified using preparative HPLC to give N-(4-(chlorodifluoromethoxy)phenyl)-1-isopropyl-7-(1H-pyrazol-5-yl)indoline-5-carboxamide (40.5 mg, 52.1%) as a white solid. ¹H NMR (400 MHz, DMSO-d₆) δ 13.21-12.75 (m, 1H), 10.07 (s, 1H), 7.87 (d, J=9.0 Hz, 2H), 7.83-7.46 (m, 3H), 7.31 (d, J=9.0 Hz, 2H), 6.38 (s, 1H), 3.70 (m, 1H), 3.47 (t, J=8.6 Hz, 2H), 3.01 (t, J=8.6 Hz, 2H), 0.88 (d, J=6.4 Hz, 6H). MS: 447.1 (M+H)⁺.

### Example VII-2

### Synthesis of N-(4-(chlorodifluoromethoxy) phenyl)-1-cyclopentyl-7-(1H-pyrazol-5-yl) indoline-5-carboxamide (Cpd. No. VII-2)

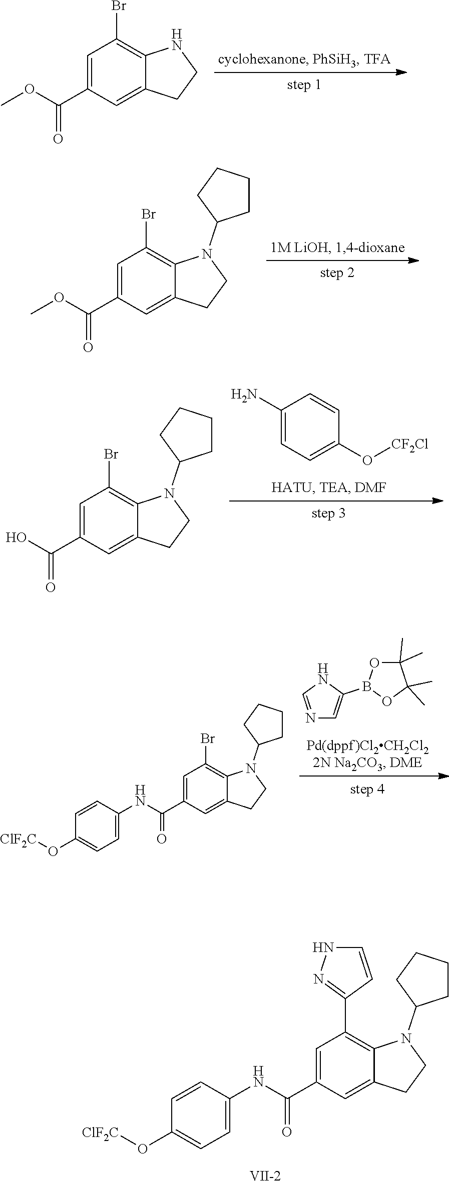

Essentially the same protocol of the preparation of Cpd. No. VII-1 in Example VII-1 was used to afford Cpd. No. VII-2 (21.6 mg) as a white solid. ¹H NMR (400 MHz, DMSO-d₆) δ 13.17-12.71 (m, 1H), 10.04 (s, 1H), 7.86 (d, J=9.0 Hz, 2H), 7.82-7.46 (m, 3H), 7.30 (d, J=9.0 Hz, 2H), 6.42-6.33 (m, 1H), 3.91-3.78 (m, 1H), 3.50 (t, J=8.6 Hz, 2H), 3.02 (t, J=8.6 Hz, 2H), 1.60-1.34 (m, 6H), 1.27-1.12 (m, 2H). MS: 473.2 (M+H)⁺.

### Example VII-3

### Synthesis of N-(4-(chlorodifluoromethoxy) phenyl)-1-cyclohexyl-7-(1H-pyrazol-5-yl) indoline-5-carboxamide (Cpd. No. VII-3)

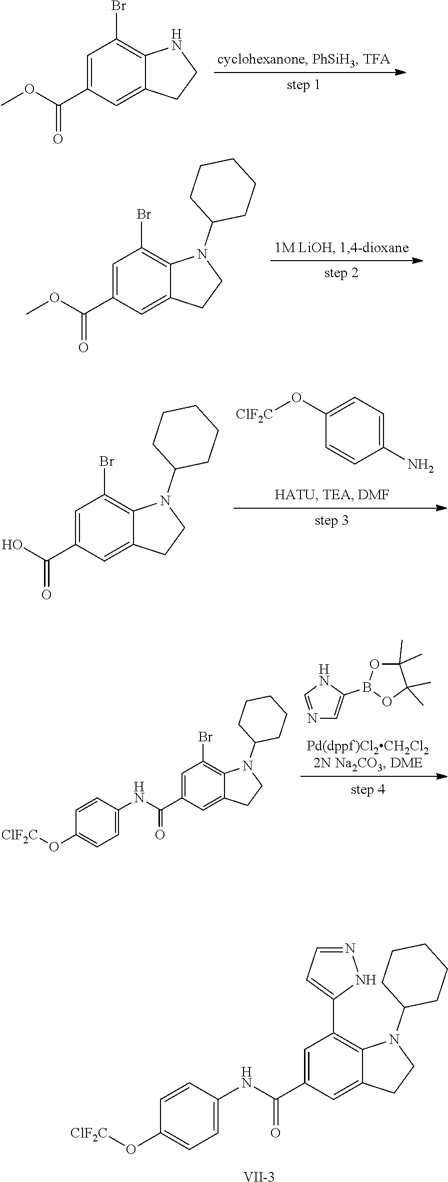

Essentially the same protocol of the preparation of Cpd. No. VII-1 in Example VII-1 was used to afford Cpd. No. VII-3 (26.0 mg) as a white solid. ¹H NMR (400 MHz, DMSO-d₆) δ 13.01-12.98 (m, 1H), 10.03 (s, 1H), 7.86 (d, J=9.0 Hz, 2H), 7.73-7.54 (m, 3H), 7.30 (d, J=9.0 Hz, 2H), 6.34 (s, 1H), 3.48 (t, J=8.6 Hz, 2H), 2.99 (t, J=8.6 Hz, 2H), 1.65-1.37 (m, 5H), 1.34-1.16 (m, 3H), 0.99-0.90 (m, 1H), 0.76-0.66 (m, 2H). MS: 487.1 (M+H)⁺.

### Example VII-4

### Synthesis of N-(4-(chlorodifluoromethoxy) phenyl)-1-isopropyl-7-(pyrimidin-5-yl)indoline-5-carboxamide (Cpd. No. VII-4)

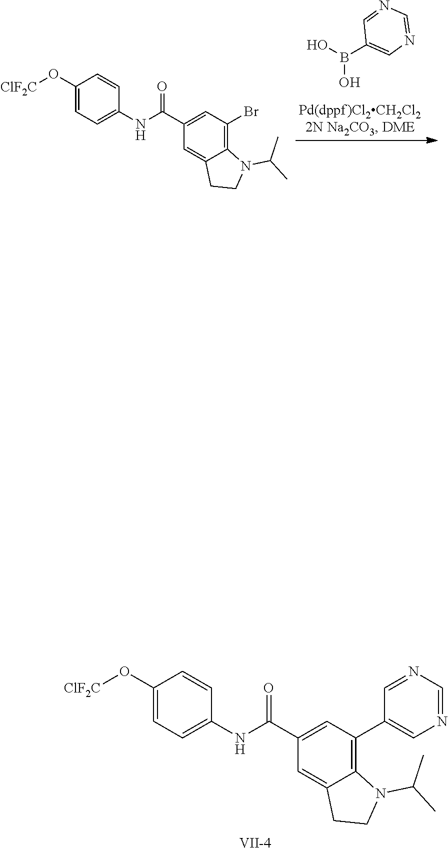

Essentially the same protocol of the preparation of Cpd. No. VII-1 in Example VII-1 was used to afford Cpd. No. VII-4 (23.0 mg) as a white solid. ¹H NMR (400 MHz, DMSO-d₆) δ 10.06 (s, 1H), 9.21 (s, 1H), 8.93 (s, 2H), 7.85 (d, J=8.8 Hz, 2H), 7.74 (s, 1H), 7.63 (s, 1H), 7.32 (d, J=8.8 Hz, 2H), 3.51 (t, J=8.6 Hz, 2H), 3.29-3.20 (m, 1H), 3.05 (t, J=8.6 Hz, 2H), 0.90-0.88 (m, 6H). MS: 459.1 (M+H)⁺.

### Example VII-5

### Synthesis of N-(4-(chlorodifluoromethoxy) phenyl)-1-cyclopropyl-7-(1H-pyrazol-5-yl) indoline-5-carboxamide (Cpd. No. VII-5)

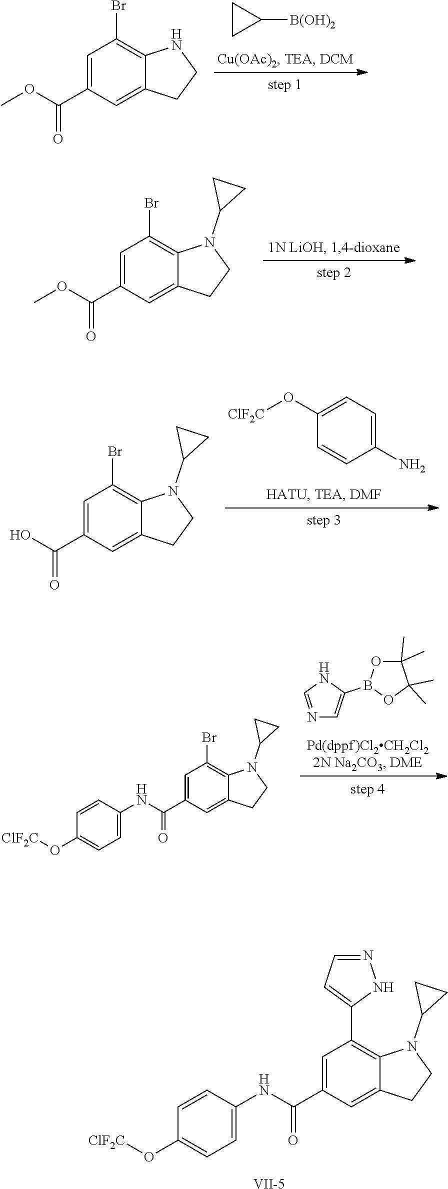

Essentially the same protocol of the preparation of Cpd. No. VII-1 in Example VII-1 was used to afford Cpd. No. VII-5 (20.0 mg) as a white solid. ¹H NMR (400 MHz, DMSO-d₆) δ 12.99-12.66 (m, 1H), 10.06 (s, 1H), 7.87 (d, J=9.0 Hz, 2H), 7.79-7.43 (m, 3H), 7.30 (d, J=9.0 Hz, 2H), 6.34 (s, 1H), 3.52 (t, J=8.6 Hz, 2H), 2.96 (t, J=8.6 Hz, 2H), 2.37-2.18 (m, 1H), 0.34-0.27 (m, 2H), 0.07-0.06 (m, 2H). MS: 445.2 (M+H)⁺.

### Example VII-6

### Synthesis of N-(4-(chlorodifluoromethoxy) phenyl)-1-(2-hydroxyethyl)-7-(1H-pyrazol-5-yl) indoline-5-carboxamide (Cpd. No. VII-6)

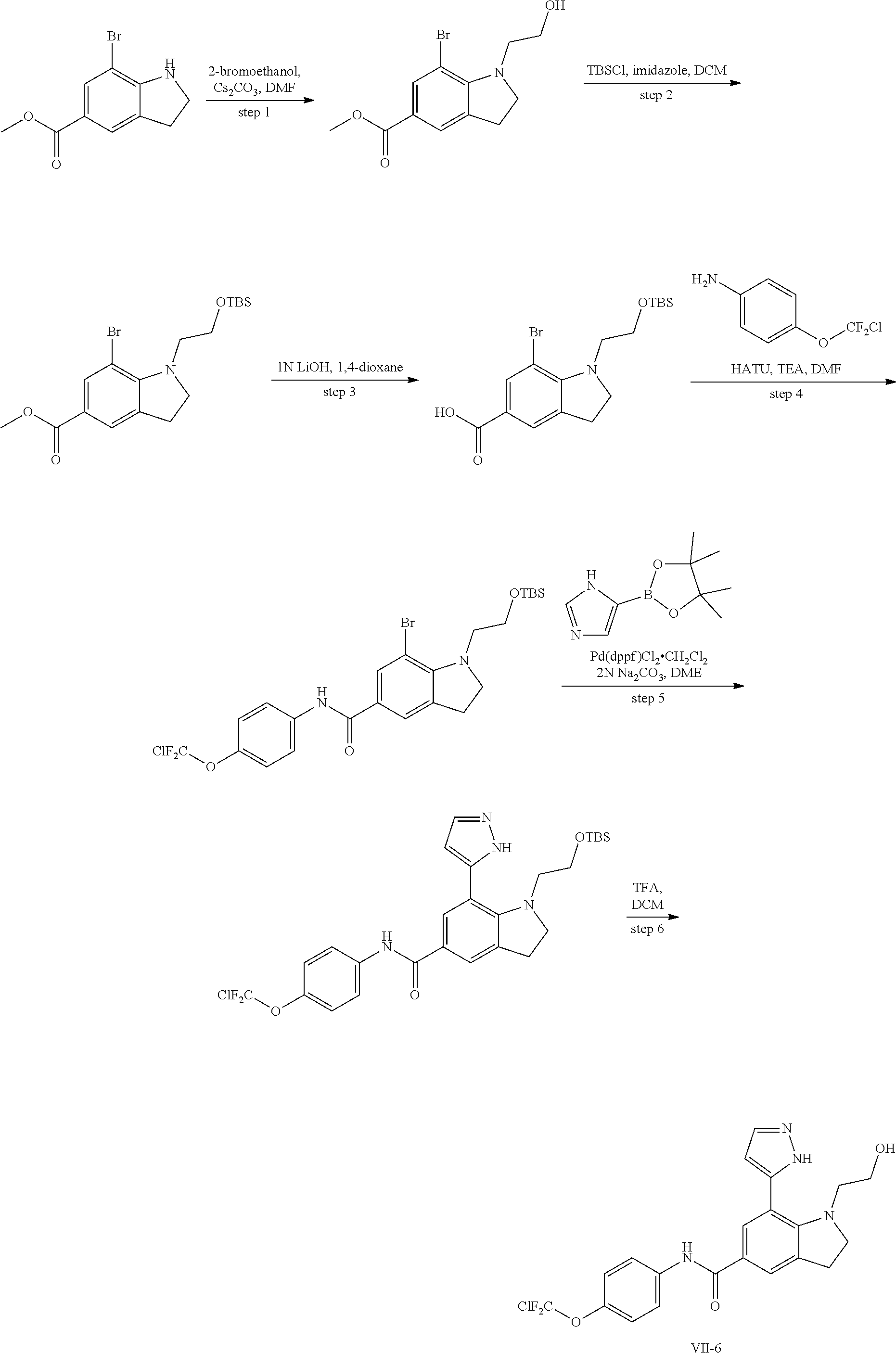

Essentially the same protocol of the preparation of Cpd. No. VII-1 in Example VII-1 was used to afford Cpd. No. VII-6 (6.0 mg) as a white solid. ¹H NMR (400 MHz, DMSO-d₆) δ 10.06 (s, 1H), 7.87 (d, J=9.0 Hz, 2H), 7.76-7.62 (m, 3H), 7.30 (d, J=9.0 Hz, 2H), 7.25-6.94 (m, 1H), 6.39 (d, J=4.0 Hz, 1H), 3.67-3.62 (m, 2H), 3.43-3.37 (m, 1H), 3.35-3.31 (m, 2H), 3.04 (t, J=8.0 Hz, 2H), 2.95 (t, J=8.0 Hz, 2H). MS: 449.1 (M+H)⁺.

### Example VII-7

### Synthesis of N-(4-(chlorodifluoromethoxy)phenyl)-1-methyl-7-(1H-pyrazol-5-yl)indoline-5-carboxamide (Cpd. No. VII-7)

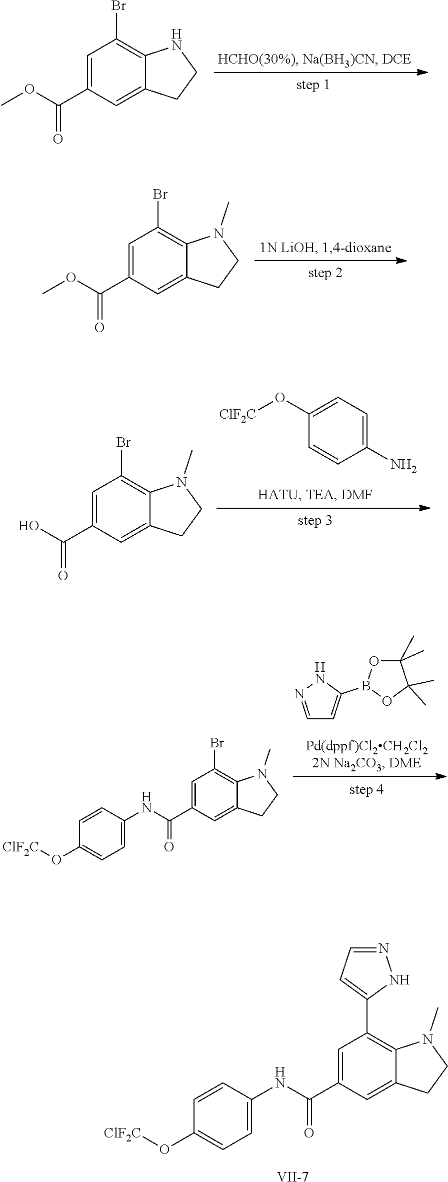

Essentially the same protocol of the preparation of Cpd. No. VII-1 in Example VII-1 was used to afford Cpd. No. VII-7 (20.0 mg) as a white solid. ¹H NMR (400 MHz, DMSO-d₆) δ 10.11 (s, 1H), 7.88 (d, J=9.2 Hz, 2H), 7.73-7.71 (m, 2H), 7.31 (d, J=9.2 Hz, 2H), 7.25-6.95 (m, 1H), 6.41 (d, J=1.8 Hz, 1H), 3.47 (t, J=8.6 Hz, 2H), 3.04 (t, J=8.6 Hz, 2H), 2.48 (s, 3H). MS: 419.1 (M+H)⁺.

### Example VII-8

### Synthesis of N-(4-(chlorodifluoromethoxy) phenyl)-1-cyclobutyl-7-(1H-pyrazol-5-yl) indoline-5-carboxamide (Cpd. No. VII-8)

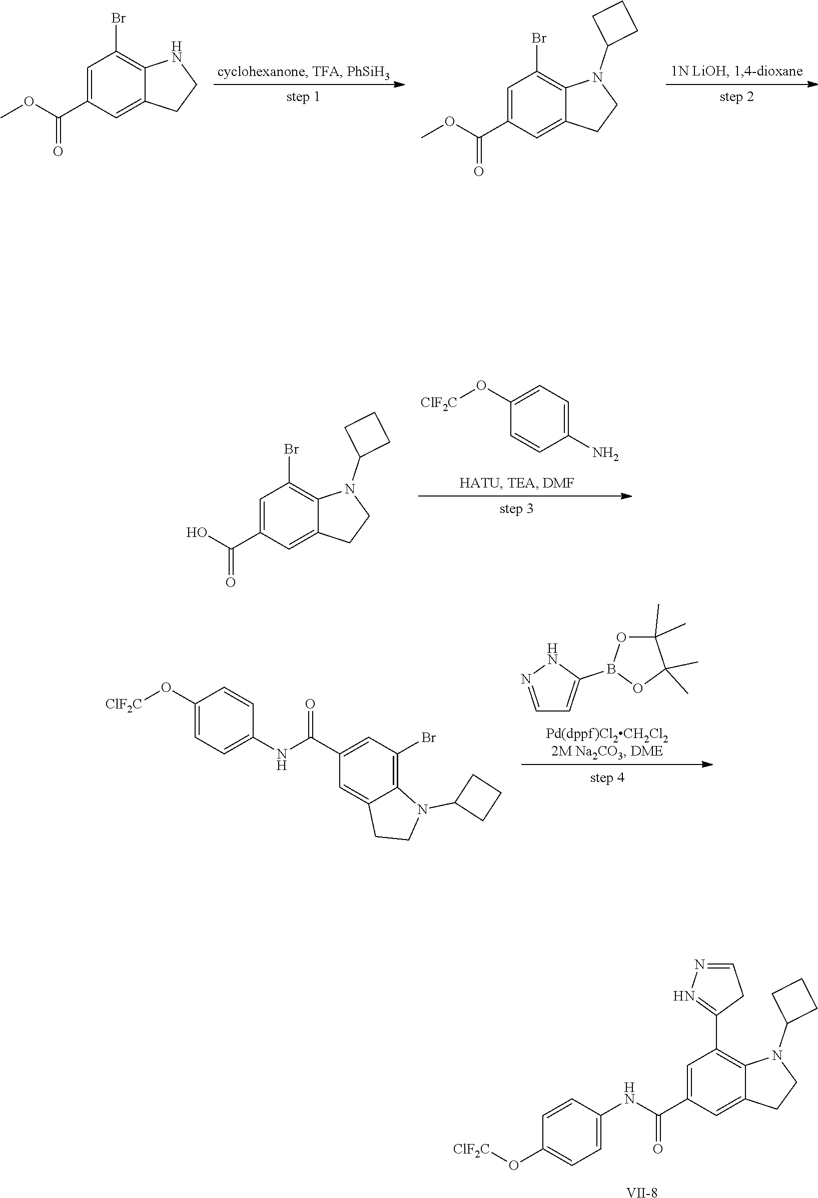

Essentially the same protocol of the preparation of Cpd. No. VII-1 in EXAMPLE VII-1 was used to afford Cpd. No. VII-8 (60.0 mg) as a white solid. ¹H NMR (400 MHz, DMSO-d₆) δ 13.02-12.99 (m, 1H), 10.05 (s, 1H), 7.87-7.85 (m, 3H), 7.74-7.55 (m, 2H), 7.31-7.29 (m, 2H), 6.41-6.30 (m, 1H), 3.65-3.61 (m, 2H), 3.03-3.02 (m, 2H), 2.08-2.03 (m, 2H), 1.66-1.63 (m, 2H), 1.53-1.43 (m, 1H), 1.30-1.11 (m, 2H). MS: 459.2 (M+H)⁺.

### Example VII-9

### Synthesis of N-(4-(chlorodifluoromethoxy) phenyl)-1-(methylsulfonyl)-7-(1H-pyrazol-5-yl) indoline-5-carboxamide (Cpd. No. VII-9)

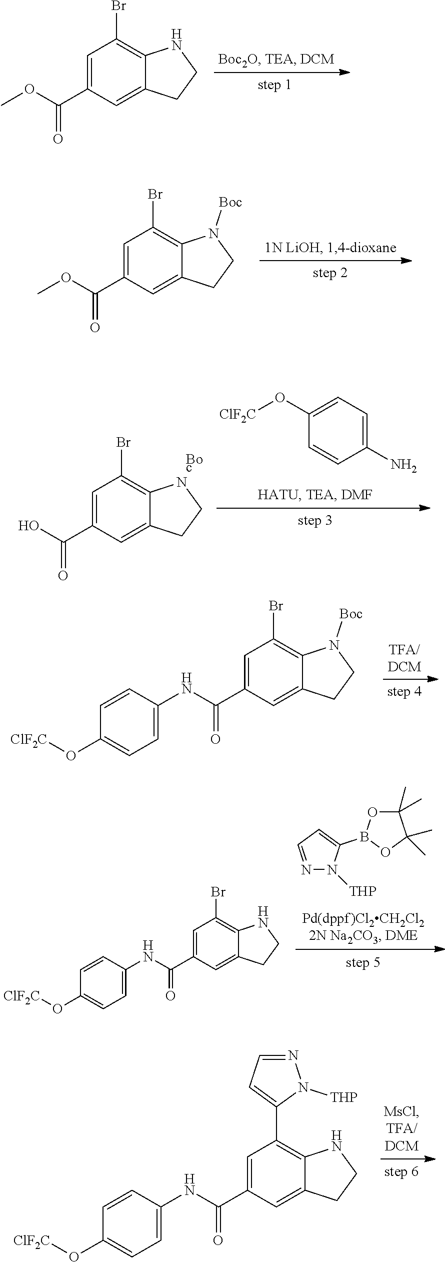

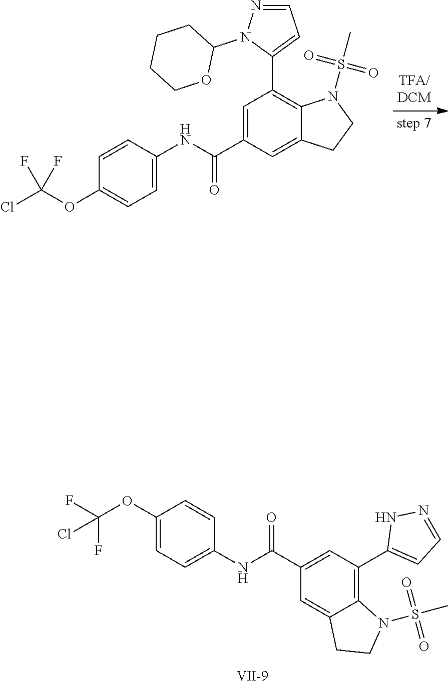 [SMILES: CS(=O)(=O)N1CCc2cc(C(=O)Nc3ccc(OC(F)(F)Cl)cc3)cc(-c3ccnn3C3CCCCO3)c21 ; CS(=O)(=O)N1CCc2cc(C(=O)Nc3ccc(OC(F)(F)Cl)cc3)cc(-c3ccn[nH]3)c21 ; "VII-9"]

### Step 1: Synthesis of 1-tert-butyl 5-methyl 7-bromoindoline-1,5-dicarboxylate

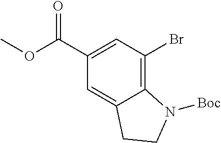 [SMILES: COC(=O)c1cc(Br)c2c(c1)CCN2C(=O)OC(C)(C)C]

N,N-dimethylpyridin-4-amine (0.72 g, 5.86 mmol) and di-tert-butyl dicarbonate (1.28 g, 5.86 mmol) were added to a solution of methyl 7-bromoindoline-5-carboxylate (1.00 g, 3.96 mmol) in dichloromethane (50.0 mL). The mixture was stirred at room temperature for 12 h. The reaction mixture was diluted with dichloromethane (50.0 mL), then washed with water (60.0 mL) and brine (60.0 mL). The organic layer was dried over Na₂SO₄, filtered and concentrated to give a crude product that was eluted through a silica gel column (ethyl acetate/hexane from 0% to 30%) to give 1-(tert-butyl) 5-methyl 7-bromoindoline-1,5-dicarboxylate (1.26 g, 91.0%) as a white solid. MS: 356.1 (M+H)⁺.

### Step 2: Synthesis of 7-bromo-1-(tert-butoxycarbonyl)indoline-5-carboxylic acid

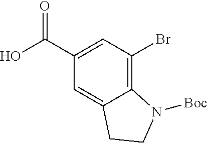 [SMILES: CC(C)(C)OC(=O)N1CCc2cc(C(=O)O)cc(Br)c21]

Lithium hydroxide (7.0 mmol, 2N, 3.5 mL) was added to a solution of 1-tert-butyl 5-methyl 7-bromoindoline-1,5-dicarboxylate (1.26 g, 3.5 mmol) in 1,4-dioxane (21.0 mL). The mixture was stirred at 50° C. for 5 h. The mixture was concentrated, then acidified with TN HCl. The resulting precipitate was collected and washed with water and hexane, dried by vacuum to give product (0.90 g, 74.4%) as a white solid. MS: 342.1 (M+H)⁺.

### Step 3: Synthesis of tert-butyl-7-bromo-5-((4-(chlorodifluoromethoxy)phenyl) carbamoyl) indoline-1-carboxylate

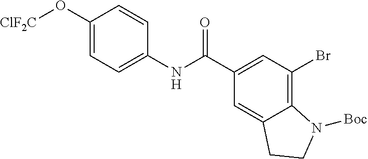

4-(chlorodifluoromethoxy)aniline (1.02 g, 5.26 mmol), triethylamine (0.53 g, 5.26 mmol) and 2-(7-azabenzotriazol-1-yl)-N,N,N′,N′-tetramethyluronium hexafluorophosphate (2.00 g, 5.26 mmol) were added to a solution of 7-bromo-1-(tert-butoxycarbonyl)indoline-5-carboxylic acid (0.90 g, 2.63 mmol) in N,N-dimethylformamide (20.0 mL). The mixture was stirred at 45° C. for 5 h. The mixture was diluted with ethyl acetate (100.0 mL) and washed with water (100.0 mL) and saturated aqueous NaCl (100.0 mL). The organic layer was dried over Na₂SO₄, filtered and concentrated to give a crude product that was eluted through a silica gel column (ethyl acetate/hexane from 0% to 30%) to give tert-butyl-7-bromo-5-((4-(chlorodifluoromethoxy) phenyl)carbamoyl) indoline-1-carboxylate (0.88 g, 64.6%) as a white solid. MS: 519.1 (M+H)⁺.

### Step 4: Synthesis of 7-bromo-N-(4-(chlorodifluoromethoxy)phenyl)indoline-5-carboxamide)

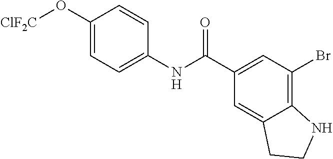

In a nitrogen flushed 25 mL round-bottomed flask tert-butyl 7-bromo-5-((4-(chlorodifluoromethoxy)phenyl)carbamoyl)indoline-1-carboxylate (0.88 g, 1.70 mmol) and trifluoroacetic acid (2.0 mL) was dissolved in dichloromethane (10.0 mL). The mixture was stirred at room temperature for 1 h, then concentrated to give a crude product that was eluted through a silica gel column (ethyl acetate/hexane from 0% to 70%) to give 7-bromo-N-(4-(chlorodifluoromethoxy)phenyl)indoline-5-carboxamide (0.65 g, 92.0%) as a yellow oil. MS: 419.1 (M+H)⁺.

### Step 5: Synthesis of N-(4-(chlorodifluoromethoxy)phenyl)-7-(1-(tetrahydro-2H-pyran-2-yl)-1H-pyrazol-5-yl)indoline-5-carboxamide

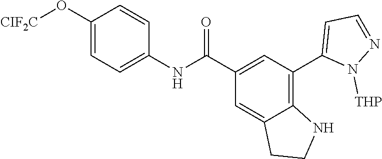

1-(tetrahydro-2H-pyran-2-yl)-5-(4,4,5,5-tetramethyl-1,3,2-dioxaborolan-2-yl)-1H-pyrazole (0.86 g, 3.11 mmol) and PdCl₂(dppf)-CH₂Cl₂ adduct (0.25 g, 0.31 mmol) were added to a solution of 7-bromo-N-(4-(chlorodifluoromethoxy)phenyl)indoline-5-carboxamide (0.65 g, 1.56 mmol) in dimethoxyethane/2N Na₂CO₃ (3:1, 10 mL). The mixture was purged with nitrogen, then stirred at 100° C. under MW for 0.5 h. The reaction mixture was diluted with ethyl acetate (100.0 mL), washed with water (100.0 mL) and saturated aqueous NaCl (100.0 mL). The organic layer was dried over Na₂SO₄, filtered and concentrated to give a crude product that was eluted through a silica gel column (ethyl acetate/hexane from 0% to 45%) to give N-(4-(chlorodifluoromethoxy)phenyl)-7-(1-(tetrahydro-2H-pyran-2-yl)-1H-pyrazol-5-yl)indoline-5-carboxamide (0.60 g, 79.0%) as a yellow solid. MS: 489.2 (M+H)⁺.

### Step 6: Synthesis of N-(4-(chlorodifluoromethoxy)phenyl)-1-(methylsulfonyl)-7-(1-(tetrahydro-2H-pyran-2-yl)-1H-pyrazol-5-yl)indoline-5-carboxamide

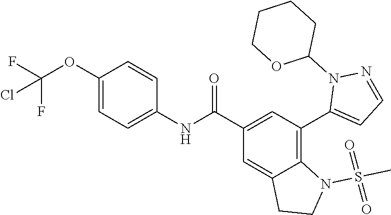 [SMILES: CS(=O)(=O)N1CCc2cc(C(=O)Nc3ccc(OC(F)(F)Cl)cc3)cc(-c3ccnn3C3CCCCO3)c21]

Diisopropylethyl amine (55.5 mg, 0.43 mmol) and methanesulfonyl chloride (49.2 mg, 0.43 mmol) were added to a solution of N-(4-(chlorodifluoromethoxy)phenyl)-7-(1-(tetrahydro-2H-pyran-2-yl)-1H-pyrazol-5-yl)indoline-5-carboxamide (70.0 mg, 0.14 mmol) in dichloromethane (2.0 mL). The mixture was stirred at room temperature for 12 h. The mixture was diluted with dichloromethane (20.0 mL), washed with water (20.0 mL), brine (20.0 mL). The organic layer was dried over Na₂SO₄, filtered and concentrated to give a crude product that was eluted through a silica gel column (ethyl acetate/hexane from 0 to 45%) to give N-(4-(chlorodifluoromethoxy)phenyl)-1-(methylsulfonyl)-7-(1-(tetrahydro-2H-pyran-2-yl)-1H-pyrazol-5-yl)indoline-5-carboxamide (40.0 mg, 49.3%) as a yellow solid. MS: 567.1 (M+H)⁺.

### Step 7: Synthesis of N-(4-(chlorodifluoromethoxy)phenyl)-1-(methylsulfonyl)-7-(1H-pyrazol-5-yl)indoline-5-carboxamide (Cpd. No. VII-9)

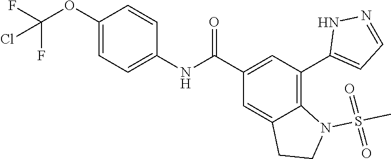 [SMILES: CS(=O)(=O)N1CCc2cc(C(=O)Nc3ccc(OC(F)(F)Cl)cc3)cc(-c3ccn[nH]3)c21]

N-(4-(chlorodifluoromethoxy)phenyl)-1-(methylsulfonyl)-7-(1-(tetrahydro-2H-pyran-2-yl)-1H-pyrazol-5-yl)indoline-5-carboxamide (40.0 mg, 0.07 mmol) was dissolved in dichloromethane/trifluoroacetic acid (5:1). The mixture was stirred at room temperature for 2 h. The mixture was concentrated to give a crude product that was purified using preparative HPLC to give N-(4-(chlorodifluoromethoxy)phenyl)-1-(methylsulfonyl)-7-(1H-pyrazol-5-yl)indoline-5-carboxamide (6.6 mg, 19.3%) as a white solid. ¹H NMR (400 MHz, DMSO-d₆) δ 10.51 (s, 1H), 8.12 (s, 1H), 7.91-7.86 (m, 3H), 7.69 (s, 1H), 7.36 (d, J=9.0 Hz, 2H), 6.67 (d, J=1.6 Hz, 1H), 4.14 (t, J=7.2 Hz, 2H), 3.15 (t, J=7.2 Hz, 2H), 2.98 (s, 3H). MS: 482.1 (M+H)⁺.

### Example VII-10

### Synthesis of N-(4-(chlorodifluoromethoxy) phenyl)-1-isobutyryl-7-(1H-pyrazol-5-yl) indoline-5-carboxamide (Cpd. No. VII-10)

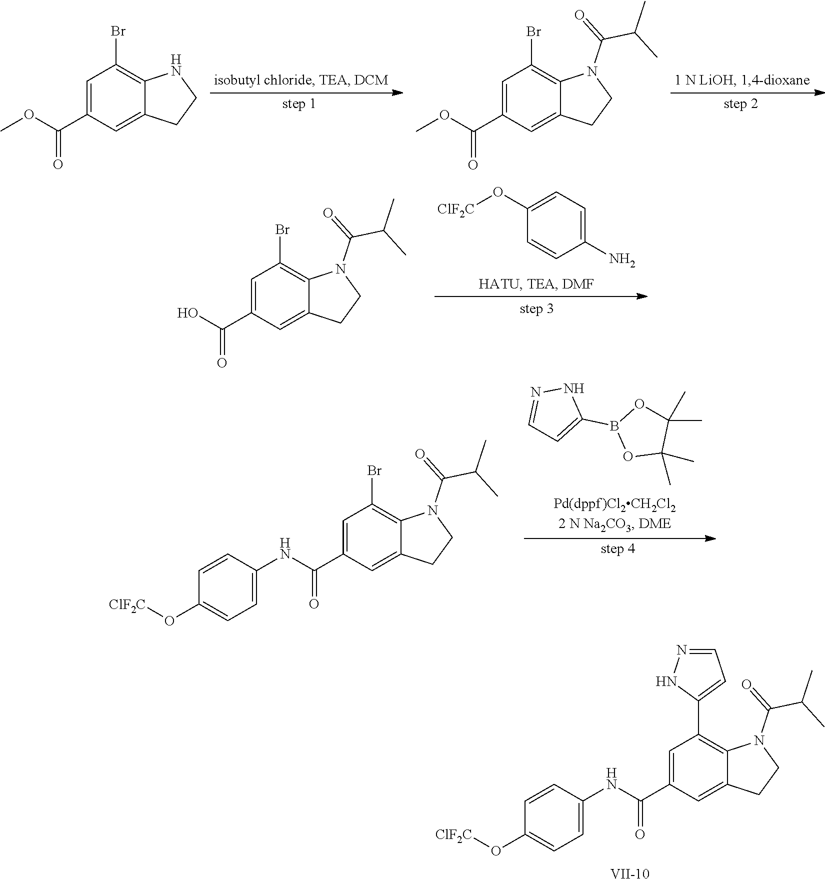

Essentially the same protocol of the preparation of Cpd. No. VII-1 in Example VII-1 was used to afford Cpd. No. VII-10 (20.0 mg) as a white solid. ¹H NMR (400 MHz, DMSO-d₆) δ 10.44 (s, 1H), 8.06 (s, 1H), 7.91 (d, J=9.0 Hz, 2H), 7.83 (s, 1H), 7.68 (s., 1H), 7.35 (d, J=9.0 Hz, 2H), 6.39 (s., 1H), 6.06 (s., 1H), 4.21 (t, J=7.0 Hz, 2H), 3.10 (t, J=7.0 Hz, 2H), 2.80-2.77 (m, 1H), 0.89-0.82 (m, 6H). MS: 475.1 (M+H)⁺.

### Example VII-11

### Synthesis of N-(4-(chlorodifluoromethoxy)phenyl)-1-(3-(chloromethyl)cyclobutyl)-7-(1H-pyrazol-3-yl)indoline-5-carboxamide (Cpd. No. VII-11)

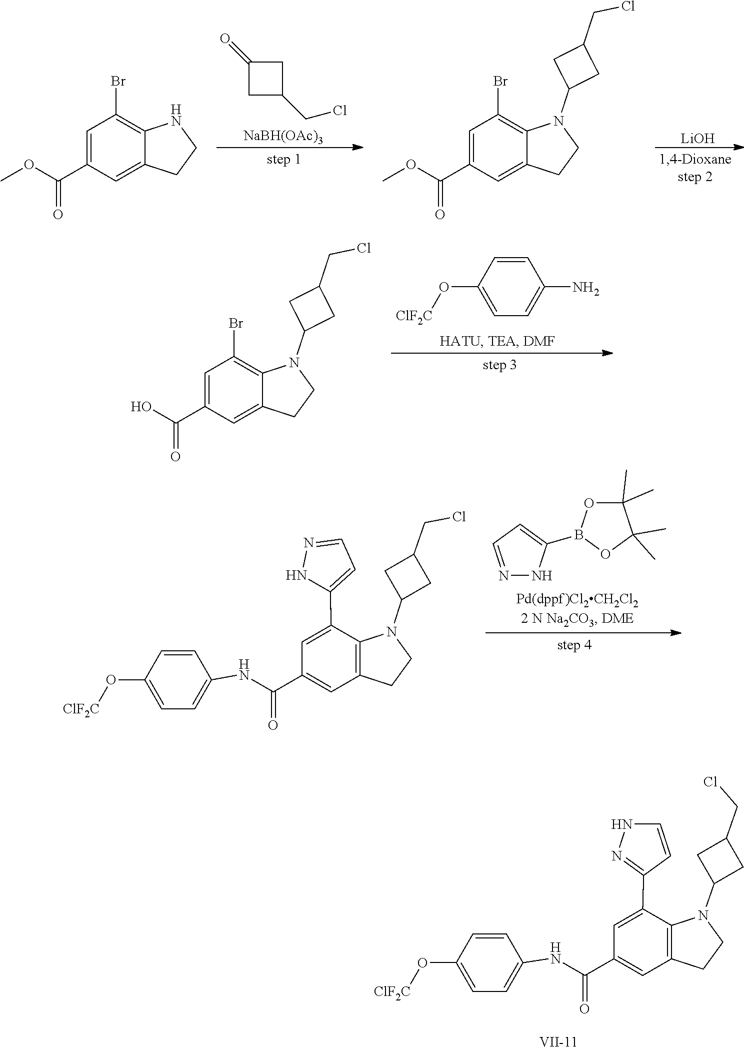

Essentially the same protocol of the preparation of Cpd. No. VII-1 in Example VII-1 was used to afford Cpd. No. VII-11 (110.0 mg) as a white solid. ¹H NMR (400 MHz, DMSO-d₆) δ 10.09 (s, 1H), 7.94-7.82 (m, 2H), 7.76 (d, J=2.0 Hz, 1H), 7.71-7.66 (m, 2H), 7.31 (d, J=8.7 Hz, 2H), 6.40 (d, J=2.0 Hz, 1H), 3.85-3.70 (m, 1H), 3.65 (t, J=8.7 Hz, 2H), 3.60 (d, J=5.0 Hz, 2H), 3.03 (t, J=8.6 Hz, 2H), 1.84-1.81 (m, 5H). MS: 507.1 (M+H)⁺.

### Example VII-12

### Synthesis of N-(4-(chlorodifluoromethoxy)phenyl)-1-(3-(hydroxymethyl)cyclobutyl)-7-(1H-pyrazol-3-yl)indoline-5-carboxamide (Cpd. No. VII-12)

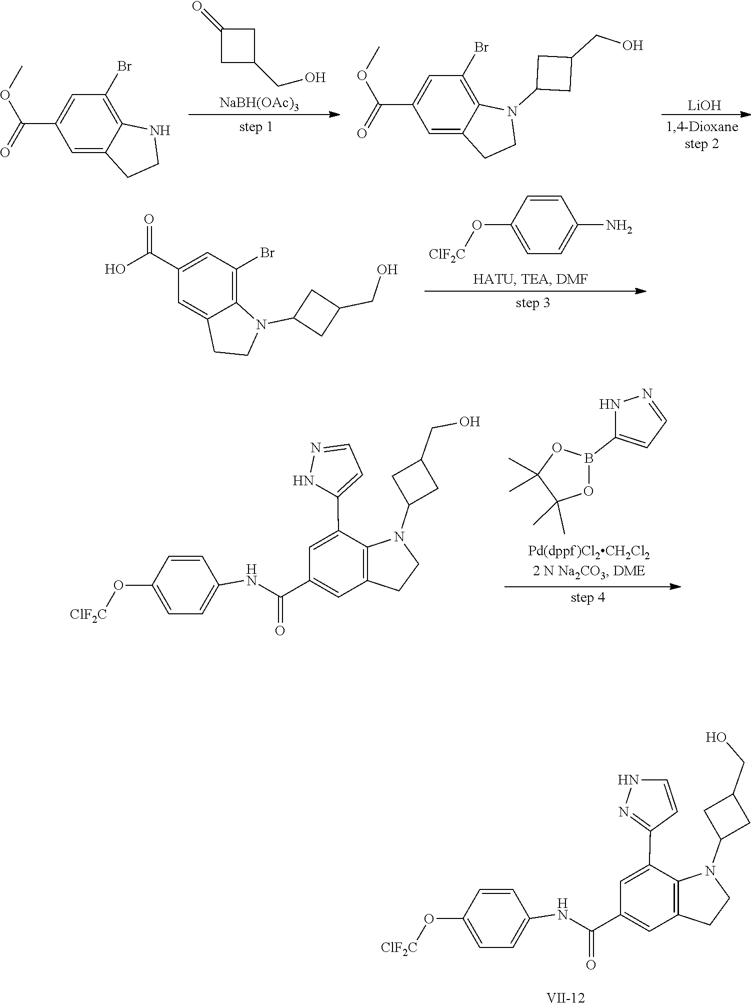

Essentially the same protocol of the preparation of Cpd. No. VII-1 in Example VII-1 was used to afford Cpd. No. VII-12 (80.0 mg) as a white solid. ¹H NMR (400 MHz, DMSO-d₆) δ 12.92 (s, 1H), 10.07 (s, 1H), 7.90-7.84 (m, 2H), 7.73 (s, 1H), 7.69-7.63 (m, 2H), 7.31 (d, J=8.7 Hz, 2H), 6.38 (d, J=2.0 Hz, 1H), 4.62-4.28 (m, 1H), 3.76 (s, 1H), 3.61 (t, J=8.7 Hz, 2H), 3.26 (d, J=5.1 Hz, 2H), 3.02 (t, J=8.6 Hz, 2H), 1.87-1.76 (m, 2H), 1.73-1.55 (m, 3H). MS: 489.1 (M+H)⁺.

### Example VII-13

### Synthesis of N-(4-(chlorodifluoromethoxy)phenyl)-2-((dimethylamino)methyl)-1-isopropyl-7-(1H-pyrazol-5-yl)indoline-5-carboxamide (Cpd. No. VII-13)

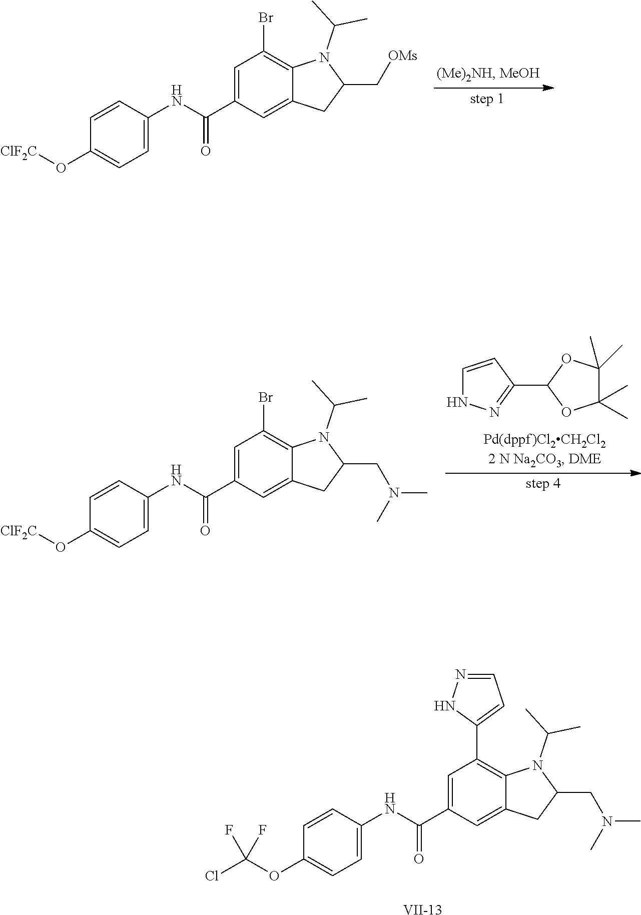

Essentially the same protocol of the preparation of Cpd. No. VII-17 in Example VII-17 was used to afford Cpd. No. VII-13 (30.0 mg) as a white solid. ¹H NMR (400 MHz, DMSO-d₆) δ 13.32-12.75 (m, 1H), 10.23 (s, 1H), 8.00 (s, 1H), 7.88 (d, J=9.0 Hz, 2H), 7.84-7.52 (m, 2H), 7.33 (d, J=9.0 Hz, 2H), 6.69-6.40 (m, 1H), 3.92-3.88 (m, 1H), 3.57-3.54 (m, 1H), 3.19-3.12 (m, 1H), 2.97-2.93 (m, 1H), 2.38-2.29 (m, 1H), 2.27-2.21 (m, 6H), 2.17-2.15 (m., 1H), 1.11-1.10 (m, 3H), 0.81-0.80 (m, 3H). MS: 504.1 (M+H)⁺.

### Example VII-14

### Synthesis of N-(4-(chlorodifluoromethoxy)phenyl)-2-(3-hydroxyazetidine-1-carbonyl)-1-isopropyl-7-(pyrimidin-5-yl)indoline-5-carboxamide (Cpd. No. VII-14)

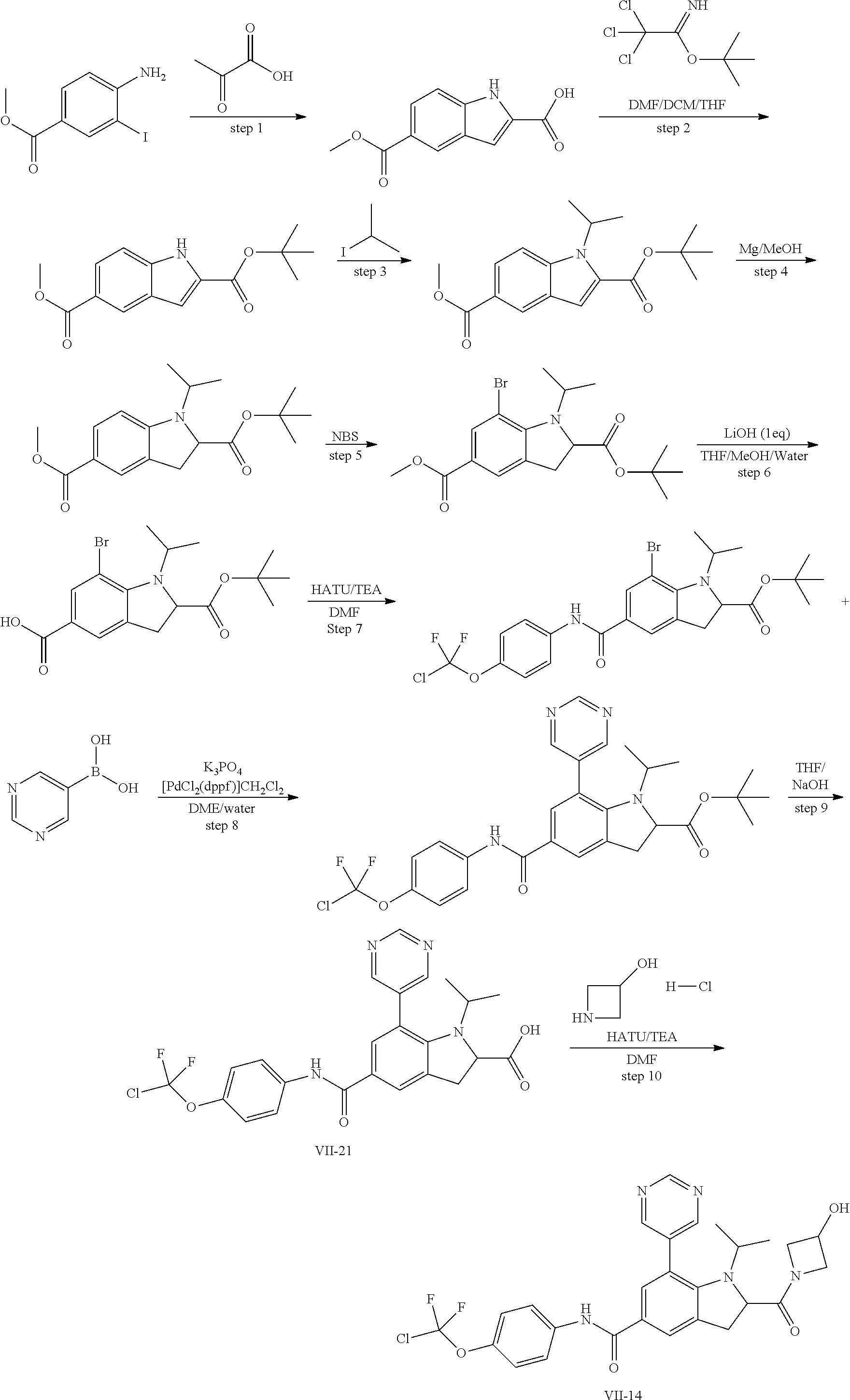

### Step 1: Synthesis of 5-(methoxycarbonyl)-1H-indole-2-carboxylic acid

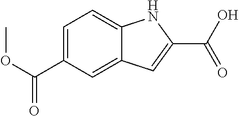 [SMILES: COC(=O)c1ccc2[nH]c(C(=O)O)cc2c1]

Under Ar, a mixture of 4-amino-3-iodo-benzoic acid methyl ester (25 g, 90.2 mmol), 2-oxo-propionic acid (13 mL, 184 mmol), 1,4-diaza-bicyclo[2.2.2]octane (20.4 g, 184 mmol) and Pd(OAc)₂ (0.5 g, 2.22 mmol) in N,N-dimethylformamide (100 mL) was stirred at 100° C. for 10 h. The solvent was removed in vacuo to give a residue which was slurried in water (200 mL), and the precipitate was obtained to give 5-(methoxycarbonyl)-1H-indole-2-carboxylic acid (17 g, 86%) as a gray solid. MS: 220.3 (M+H)⁺.

### Step 2: Synthesis of 2-(tert-butyl) 5-methyl 1H-indole-2,5-dicarboxylate

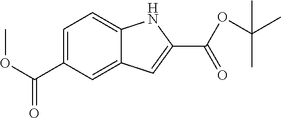 [SMILES: COC(=O)c1ccc2[nH]c(C(=O)OC(C)(C)C)cc2c1]

Under Ar, to a suspension of 5-(methoxycarbonyl)-1H-indole-2-carboxylic acid (17 g, 78 mmol) in N,N-dimethylformamide (25 mL)/tetrahydrofuran (1 L) were added tert-butyl 2,2,2-trichloroacetimidate (136 g, 620 mmol) in 7 portions and BF₃OEt₂ (7.7 g, 54.3 mmol)(7 portions) at room temperature, then the mixture was stirred at room temperature for 36 h. The mixture was quenched with saturated NaHCO₃ solution, which was partitioned between ethyl acetate/water. The organic layer was concentrated in vacuo to give a light yellow crude product, which was eluted through a silica gel column (hexane/ethyl acetate=5/1) to give 2-(tert-butyl) 5-methyl 1H-indole-2,5-dicarboxylate (18 g, 84%) as a light yellow solid. MS: 276.3 (M+H)⁺.

### Step 3: Synthesis of 2-(tert-butyl) 5-methyl 1-isopropyl-1H-indole-2,5-dicarboxylate

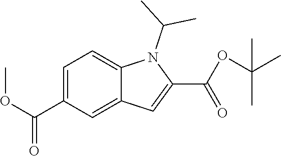 [SMILES: COC(=O)c1ccc2c(c1)cc(C(=O)OC(C)(C)C)n2C(C)C]

2-Iodopropane (14.82 g, 87 mmol) and Cs₂CO₃ (28.4 g, 87 mmol) were added to a solution of 2-tert-butyl 5-methyl 1H-indole-2,5-dicarboxylate (6.0 g, 21.79 mmol) in N,N-dimethylformamide (80.0 mL). The mixture was stirred at 70° C. for 12 h. The mixture was diluted with ethyl acetate (200 mL), washed with water (100 mL) and brine (150 mL). The organic layer was dried over Na₂SO₄, filtered and concentrated to give a crude product that was eluted through a silica gel column (ethyl acetate/hexane from 0% to 15%) to give 2-(tert-butyl) 5-methyl 1-isopropyl-1H-indole-2,5-dicarboxylate (6.1 g, 88%) as a yellow solid. MS: 318.2 (M+H)⁺.

### Step 4: Synthesis of 2-tert-butyl 5-methyl 1-isopropylindoline-2,5-dicarboxylate

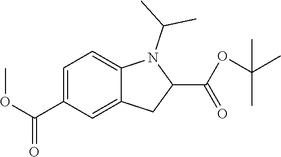 [SMILES: COC(=O)c1ccc2c(c1)CC(C(=O)OC(C)(C)C)N2C(C)C]

Magnesium (0.444 g, 18.27 mmol) was added to a solution of 2-(tert-butyl) 5-methyl 1-isopropyl-1H-indole-2,5-dicarboxylate (2.9 g, 9.15 mmol) in methanol (9.0 mL). The mixture was stirred at room temperature for 12 h. Then the mixture was quenched with NH₄HCl aqueous solution and diluted with ethyl acetate (100 mL), washed with water (100 mL) and brine (100 mL). The organic layer was dried over Na₂SO₄, filtered and concentrated to give a crude product that was eluted through a silica gel column (ethyl acetate/hexane from 0% to 30%) to give 2-(tert-butyl) 5-methyl 1-isopropylindoline-2,5-dicarboxylate (1.5 g, 51.4%) as a yellow oil. MS: 320.2 (M+H)⁺.

### Step 5: Synthesis of 2-tert-butyl 5-methyl 7-bromo-1-isopropylindoline-2,5-dicarboxylate

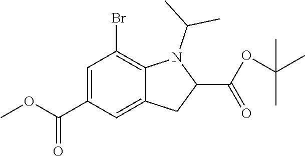 [SMILES: COC(=O)c1cc(Br)c2c(c1)CC(C(=O)OC(C)(C)C)N2C(C)C]

N-(12-boranylidene)thiohydroxylamine (0.267 g, 4.70 mmol) was added to a solution of 2-(tert-butyl) 5-methyl 1-isopropylindoline-2,5-dicarboxylate (1.5 g, 4.7 mmol) in 1,4-dioxane (30.0 mL) at 0° C. The mixture was stirred at room temperature for 2 h. The mixture was quenched with NaHCO₃ aqueous solution, the mixture was diluted with ethyl acetate (80 mL), washed with water (80 mL) and brine (80 mL). The organic layer was dried over Na₂SO₄, filtered and concentrated to give a crude product that was eluted through a silica gel column (ethyl acetate/hexane from 0% to 30%) to give 2-(tert-butyl) 5-methyl 7-bromo-1-isopropylindoline-2,5-dicarboxylate (1.5 g, 80%) as a yellow oil. MS: 398.1 (M+H)⁺.

### Step 6: Synthesis of 7-bromo-2-(tert-butoxycarbonyl)-1-isopropylindoline-5-carboxylic acid

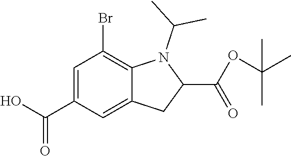 [SMILES: CC(C)N1c2c(Br)cc(C(=O)O)cc2CC1C(=O)OC(C)(C)C]

Under Ar, to a solution of 2-(tert-butyl) 5-methyl 7-bromo-1-isopropylindoline-2,5-dicarboxylate (1.1 g, 2.76 mmol) in methanol (2 mL)/tetrahydrofuran (8.00 mL) were added LiOH solution (2N, 2.7 mL, 5.43 mmol) and stirred at room temperature for 6 h. The mixture was acidified with HCl to pH=4˜5, then extracted with ethyl acetate (30 mL×2). The combined organic layers were washed with brine, dried over Na₂SO₄, filtered and concentrated in vacuo to give the title compound, which was obtained without purification as colorless solid and used in the next step (1 g, crude). MS: 382.3 (M+H)⁺.

### Step 7: Synthesis of 7-bromo-2-(tert-butoxycarbonyl)-1-isopropylindoline-5-carboxylic acid

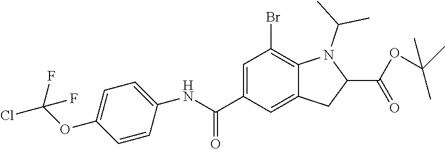 [SMILES: CC(C)N1c2c(Br)cc(C(=O)Nc3ccc(OC(F)(F)Cl)cc3)cc2CC1C(=O)OC(C)(C)C]

Under Ar, a mixture of 7-bromo-2-(tert-butoxycarbonyl)-1-isopropylindoline-5-carboxylic acid (1 g, 2.60 mmol), 4-(chlorodifluoromethoxy)aniline (1.008 g, 5.20 mmol), Et₃N (0.790 g, 7.81 mmol) and 2-(7-azabenzotriazol-1-yl)-N,N,N′,N′-tetramethyluronium hexafluorophosphate (1.979 g, 5.20 mmol) in N,N-dimethylformamide (10 mL) was stirred at room temperature overnight. The mixture was partitioned between ethyl acetate/water. The organic layer was concentrated in vacuo to give a yellow oil that was eluted through a silica gel column (hexane/ethyl acetate=5/1) to give tert-butyl 7-bromo-5-((4-(chlorodifluoromethoxy)phenyl)carbamoyl)-1-isopropylindoline-2-carboxylate (900 mg, 61.8%) as a light yellow solid. MS: 561.3 (M+H)⁺.

### Step 8: Synthesis of tert-butyl 5-((4-(chlorodifluoromethoxy)phenyl)carbamoyl)-1-isopropyl-7-(pyrimidin-5-yl)indoline-2-carboxylate

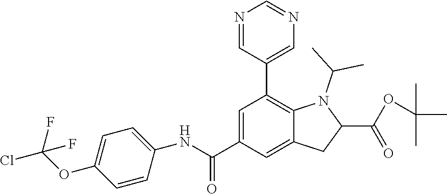 [SMILES: CC(C)N1c2c(cc(C(=O)Nc3ccc(OC(F)(F)Cl)cc3)cc2-c2cncnc2)CC1C(=O)OC(C)(C)C]

Under Ar, a mixture of tert-butyl 7-bromo-5-((4-(chlorodifluoromethoxy)phenyl)carbamoyl)-1-isopropylindoline-2-carboxylate (200 mg, 0.357 mmol), pyrimidin-5-ylboronic acid (133 mg, 1.072 mmol), K₃PO₄ (303 mg, 1.429 mmol) and [PdCl₂(dppf)]CH₂Cl₂ (29.2 mg, 0.036 mmol) in dimethoxyethane (4 mL)/water (1 mL) was stirred at 110° C. under microwave for 2.5 h. The mixture was partitioned between ethyl acetate/water. The organic layer was concentrated in vacuo to give a black oil, which was eluted through a silica gel column (hexane/ethyl acetate=1/1) to give tert-butyl 5-((4-(chlorodifluoromethoxy)phenyl)carbamoyl)-1-isopropyl-7-(pyrimidin-5-yl)indoline-2-carboxylate (180 mg, 90%) as a light yellow solid. MS: 559.4 (M+H)⁺.

### Step 9: Synthesis of 5-((4-(chlorodifluoromethoxy)phenyl)carbamoyl)-1-isopropyl-7-(pyrimidin-5-yl)indoline-2-carboxylic acid (Cpd. No. VII-21)

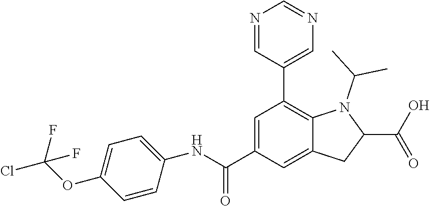 [SMILES: CC(C)N1c2c(cc(C(=O)Nc3ccc(OC(F)(F)Cl)cc3)cc2-c2cncnc2)CC1C(=O)O]

Under Ar, a mixture of tert-butyl 5-((4-(chlorodifluoromethoxy)phenyl)carbamoyl)-1-isopropyl-7-(pyrimidin-5-yl)indoline-2-carboxylate (150 mg, 0.268 mmol) in tetrahydrofuran (8 mL)/NaOH (2N, 2 mL) was stirred at 80° C. for 8 h. The mixture was acidified with HCl to pH=5-6, and partitioned between ethyl acetate/water. The organic layer was washed with brine, dried over Na₂SO₄, filtered and concentrated in vacuo to give 5-((4-(chlorodifluoromethoxy)phenyl)carbamoyl)-1-isopropyl-7-(pyrimidin-5-yl)indoline-2-carboxylic acid (120 mg, 89%) as a white solid. ¹H NMR (400 MHz, DMSO-d₆) δ 10.18 (s, 1H), 9.19 (s, 1H), 9.12 (s, 2H), 7.86 (d, J=9.0 Hz, 2H), 7.82 (s, 1H), 7.78 (s, 1H), 7.34 (d, J=8.7 Hz, 2H), 4.44 (dd, J=10.9, 3.6 Hz, 1H), 3.50 (dd, J=16.7, 10.9 Hz, 1H), 3.25 (t, J=6.6 Hz, 1H), 3.09 (dd, J=16.7, 3.5 Hz, 1H), 1.00 (d, J=6.8 Hz, 3H), 0.81 (d, J=6.4 Hz, 3H). MS: 503.3 (M+H)⁺.

### Step 10: N-(4-(chlorodifluoromethoxy)phenyl)-2-(3-hydroxyazetidine-1-carbonyl)-1-isopropyl-7-(pyrimidin-5-yl)indoline-5-carboxamide (Cpd. No. VII-14)

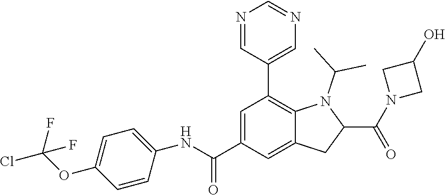 [SMILES: CC(C)N1c2c(cc(C(=O)Nc3ccc(OC(F)(F)Cl)cc3)cc2-c2cncnc2)CC1C(=O)N1CC(O)C1]

Under Ar, to a solution of 5-((4-(chlorodifluoromethoxy)phenyl)carbamoyl)-1-isopropyl-7-(pyrimidin-5-yl)indoline-2-carboxylic acid (20 mg, 0.040 mmol), 2-(7-azabenzotriazol-1-yl)-N,N,N′,N′-tetramethyluronium hexafluorophosphate (37.8 mg, 0.099 mmol), azetidin-3-ol hydrochloride (8.71 mg, 0.080 mmol) in N,N-dimethylformamide (1 mL) was added Et₃N (16.10 mg, 0.159 mmol) at room temperature. The mixture was stirred at room temperature for over night, then partitioned between ethyl acetate/water. The organic layer was concentrated in vacuo to give a light yellow oil, which was purified using preparative HPLC to give N-(4-(chlorodifluoromethoxy)phenyl)-2-(3-hydroxyazetidine-1-carbonyl)-1-isopropyl-7-(pyrimidin-5-yl)indoline-5-carboxamide (11 mg, 49.6%) as a white solid. ¹H NMR (400 MHz, DMSO-d₆) δ 10.15 (s, 1H), 9.21 (d, J=0.8 Hz, 1H), 9.06 (d, J=2.8 Hz, 2H), 7.88-7.82 (m, 2H), 7.75 (d, J=1.9 Hz, 1H), 7.72 (d, J=5.2 Hz, 1H), 7.34 (d, J=8.7 Hz, 2H), 5.83-5.78 (m, 1H), 4.58-4.34 (m, 3H), 4.17-4.08 (m, 1H), 4.07-3.88 (m, 1H), 3.69-3.46 (m, 2H), 3.32-3.22 (m, 1H), 2.99-2.89 (m, 1H), 0.94 (d, J=6.8 Hz, 3H), 0.79 (d, J=6.4 Hz, 3H). MS: 558.4 (M+H)⁺.

### Example VII-15

### Synthesis of N5-(4-(chlorodifluoromethoxy)phenyl)-1-isopropyl-N2,N2-dimethyl-7-(pyrimidin-5-yl)indoline-2,5-dicarboxamide (Cpd. No. VII-15)

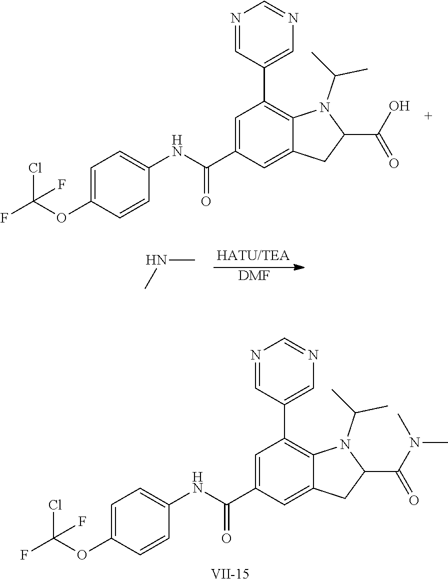

Essentially the same protocol of the preparation of Cpd. No. VII-14 in Example VII-14 was used to afford Cpd. No. VII-15 (6.3 mg) as a white solid. ¹H NMR (400 MHz, DMSO-d₆) δ 10.13 (s, 1H), 9.19 (s, 1H), 9.17 (s, 2H), 7.90-7.83 (m, 2H), 7.78 (d, J=1.8 Hz, 1H), 7.69 (d, J=1.7 Hz, 1H), 7.34 (d, J=8.7 Hz, 2H), 4.86 (dd, J=10.9, 3.9 Hz, 1H), 3.61 (dd, J=16.6, 10.9 Hz, 1H), 3.33-3.24 (m, 1H), 3.13 (s, 3H), 2.88 (s, 3H), 2.81 (dd, J=16.7, 3.8 Hz, 1H), 0.90 (d, J=6.8 Hz, 3H), 0.86 (d, J=6.4 Hz, 3H). MS: 530.4 (M+H)⁺.

### Example VII-16

### Synthesis of N5-(4-(chlorodifluoromethoxy)phenyl)-1-isopropyl-N2-methyl-7-(pyrimidin-5-yl)indoline-2,5-dicarboxamide (Cpd. No. VII-16)

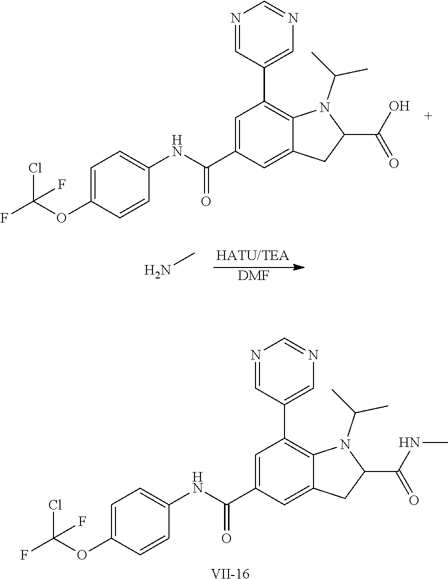

Essentially the same protocol of the preparation of Cpd. No. VII-14 in Example VII-14 was used to afford Cpd. No. VII-16 (7.7 mg) as a white solid. ¹H NMR (400 MHz, DMSO-d₆) δ 10.19 (s, 1H), 9.24 (s, 1H), 9.22 (s, 2H), 7.90-7.83 (m, 2H), 7.81-7.76 (m, 3H), 7.35 (d, J=8.7 Hz, 2H), 4.31 (dd, J=11.3, 5.1 Hz, 1H), 3.56 (dd, J=16.9, 11.4 Hz, 1H), 3.33-3.26 (m, 1H), 3.03 (dd, J=16.9, 5.1 Hz, 1H), 2.69 (d, J=4.6 Hz, 3H), 0.98 (d, J=6.8 Hz, 3H), 0.74 (d, J=6.4 Hz, 3H). MS: 516.4 (M+H)⁺.

### Example VII-17

### Synthesis of N-(4-(chlorodifluoromethoxy)phenyl)-1-isopropyl-2-((methylamino)methyl)-7-(1H-pyrazol-5-yl) indoline-5-carboxamide (Cpd. No. VII-17)

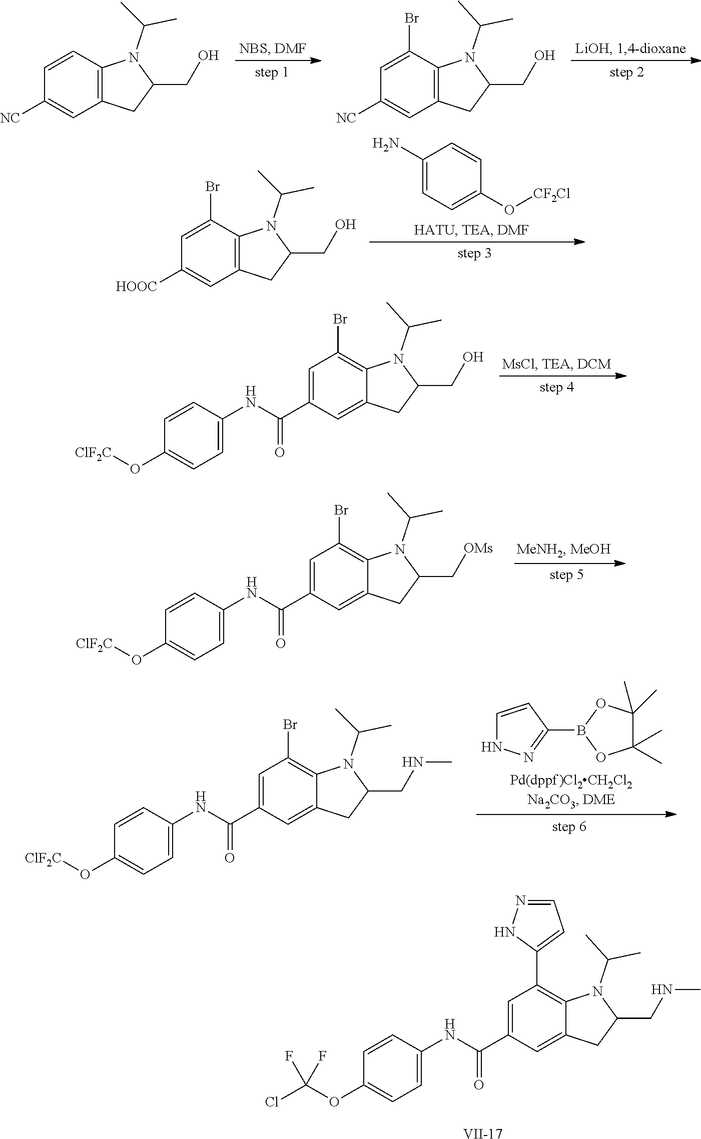

### Step 1: 7-bromo-2-(hydroxymethyl)-1-isopropylindoline-5-carbonitrile

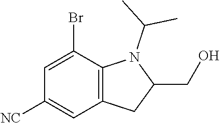 [SMILES: CC(C)N1c2c(Br)cc(C#N)cc2CC1CO]

N-Bromosuccinimide (3.70 g, 20.81 mmol) was added to a solution of 2-(hydroxymethyl)-1-isopropylindoline-5-carbonitrile (3.0 g, 13.87 mmol) in 1,4-dioxane (80.0 mL). The mixture was stirred at room temperature for 3 h. The mixture was concentrated and diluted with ethyl acetate (150 mL), washed with water (150 mL) and brine (150 mL). The organic layer was dried over Na₂SO₄, filtered and concentrated to give a crude product that was eluted through a silica gel column (ethyl acetate/hexane from 0% to 60%) to give 7-bromo-2-(hydroxymethyl)-1-isopropylindoline-5-carbonitrile (3.3 g, 81%) as a yellow oil. MS: 295.1 (M+H)⁺.

### Step 2: Synthesis of 7-bromo-2-(hydroxymethyl)-1-isopropylindoline-5-carboxylic acid

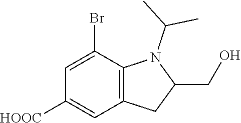 [SMILES: CC(C)N1c2c(Br)cc(C(=O)O)cc2CC1CO]

Lithium hydroxide (1N, 40.0 mmol) was added to a solution of 7-bromo-2-(hydroxymethyl)-1-isopropylindoline-5-carbonitrile (2.0 g, 6.78 mmol) in 1,4-dioxane. The mixture was stirred at 88° C. for 16 h. The mixture was concentrated, acidified with 1N HCl. The resulting precipitate was collected, washed with water and hexane, then dried under vacuum to give product 2.0 g as a white solid. MS: 314.1 (M+H)⁺.

### Step 3: Synthesis of 7-bromo-N-(4-(chlorodifluoromethoxy)phenyl)-2-(hydroxymethyl)-1-isopropylindoline-5-carboxamide

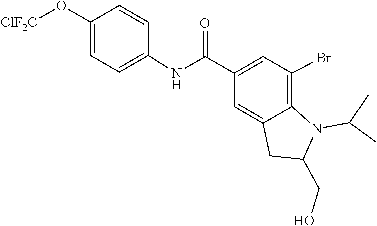

4-(Chlorodifluoromethoxy)aniline (2.095 g, 10.82 mmol), triethylamine (0.821 g, 8.12 mmol) and 2-(7-azabenzotriazol-1-yl)-N,N,N′,N′-tetramethyluronium hexafluorophosphate (2.469 g, 6.49 mmol) were added to a solution of 7-bromo-2-(hydroxymethyl)-1-isopropylindoline-5-carboxylic acid (1.7 g, 5.41 mmol) in N,N-dimethylformamide (30.0 mL). The mixture was stirred at room temperature for 16 h. The reaction mixture was diluted with ethyl acetate (80 mL), washed with water (80 mL) and brine (80 mL). The organic layer was dried over Na₂SO₄, filtered and concentrated to give a crude product that was eluted through a silica gel column (ethyl acetate/hexane from 0% to 100%) to give 7-bromo-N-(4-(chlorodifluoromethoxy)phenyl)-2-(hydroxymethyl)-1-isopropylindoline-5-carboxamide (1.96 g, 74.0%) as a white solid. MS: 491.2 (M+H)⁺.

### Step 4: Synthesis of (7-bromo-5-((4-(chlorodifluoromethoxy)phenyl)carbamoyl)-1-isopropylindolin-2-yl)methyl methanesulfonate

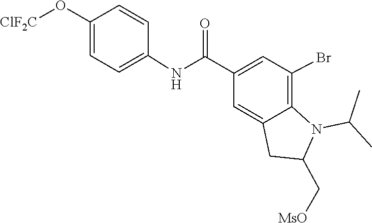

To a solution of triethylamine (31.0 mg, 0.306 mmol) and 7-bromo-N-(4-(chlorodifluoromethoxy)phenyl)-2-(hydroxymethyl)-1-isopropylindoline-5-carboxamide (100.0 mg, 0.2 mmol) in dichloromethane (10 mL) was dropped methanesulfonyl chloride (28.1 mg, 0.245 mmol). The mixture was stirred at room temperature for 2 h, then diluted with dichloromethane (20 mL), washed with water (20 mL) and brine (20 mL). The organic layer was dried over Na₂SO₄, filtered and concentrated to give crude product (100 mg), which was used in the next step without purification. MS: 569.1;

### Step 5: Synthesis of 7-bromo-N-(4-(chlorodifluoromethoxy)phenyl)-1-isopropyl-2-((methylamino)methyl)indoline-5-carboxamide

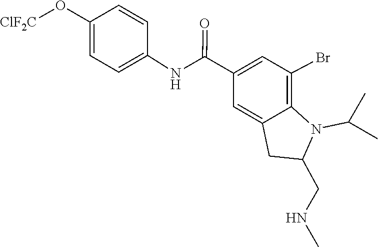

Methanamine (2N in methanol, 17.61 mmol) was added to a solution of (7-bromo-5-((4-(chlorodifluoromethoxy)phenyl)carbamoyl)-1-isopropylindolin-2-yl)methyl methanesulfonate (1.0 g, 1.76 mmol) in methanol (5.0 mL). The mixture was heated to 60° C. in sealing tube for 12 h. The mixture was cooled to room temperature, concentrated to give a crude product that was eluted through a silica gel column (ethyl acetate/hexane from 0 to 80%) to give 7-bromo-N-(4-(chlorodifluoromethoxy)phenyl)-1-isopropyl-2-((methylamino)methyl)indoline-5-carboxamide (0.66 g, 74.5%) as a yellow oil. MS: 504.1 (M+H)⁺.

### Step 6: Synthesis of N-(4-(chlorodifluoromethoxy)phenyl)-1-isopropyl-2-((methylamino)methyl)-7-(1H-pyrazol-5-yl)indoline-5-carboxamide (Cpd. No. VII-17)

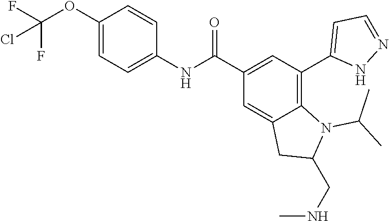 [SMILES: CNCC1Cc2cc(C(=O)Nc3ccc(OC(F)(F)Cl)cc3)cc(-c3ccn[nH]3)c2N1C(C)C]

5-(4,4,5,5-Tetramethyl-1,3,2-dioxaborolan-2-yl)-1H-pyrazole (93 mg, 0.477 mmol) and PdCl₂(dppf)-CH₂Cl₂ adduct (46.6 mg, 0.064 mmol) were added to a solution of 7-bromo-N-(4-(chlorodifluoromethoxy)phenyl)-1-isopropyl-2 ((methylamino)methyl) indoline-5-carboxamide (160 mg, 0.31 mmol) in DCE/2N Na₂CO₃ (0.9 mL/0.3 mL). The mixture was filled with nitrogen three times and the mixture was heated to 100° C. The mixture was diluted with ethyl acetate (30 mL), washed with water (30 mL) and brine (30 mL). The organic layer was dried over Na₂SO₄, filtered and concentrated to give a crude product that was purified using preparative HPLC to give N-(4-(chlorodifluoromethoxy)phenyl)-1-isopropyl-2-((methylamino)methyl)-7-(1H-pyrazol-5-yl)indoline-5-carboxamide (26 mg, 16.68%) as a white solid. ¹H NMR (400 MHz, DMSO-d₆) δ 12.90 (s, 1H), 10.52-9.93 (m, 1H), 7.99-7.96 (m, 1H), 7.88 (d, J=9.0 Hz, 2H), 7.82-7.56 (m, 2H), 7.33 (d, J=9.0 Hz, 2H), 6.64 (s, 1H), 3.89-3.87 (m, 1H), 3.52-3.49 (m, 1H), 3.26-3.13 (m, 2H), 2.82-2.78 (m, 1H), 2.57-2.53 (m, 2H), 2.36 (s, 3H), 1.13-1.12 (m, 3H), 0.79-0.77 (m, 3H). MS: 490.1 (M+H)⁺.

### Example VII-18

### Synthesis of N-(4-(chlorodifluoromethoxy)phenyl)-1-isopropyl-2-((N-methylacetamido)methyl)-7-(1H-pyrazol-5-yl)indoline-5-carboxamide (Cpd. No. VII-18)

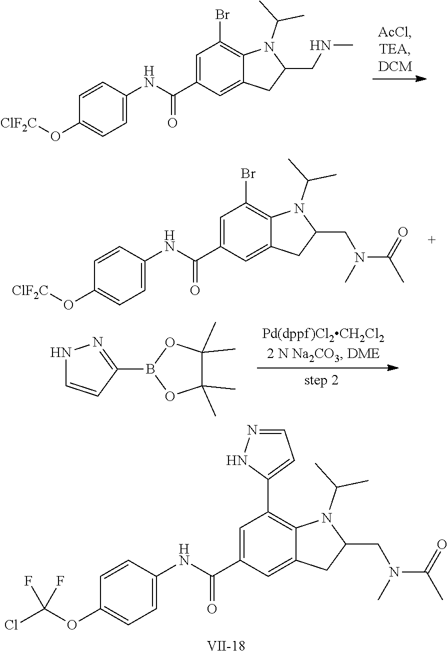

Essentially the same protocol of the preparation of Cpd. No. VII-17 in Example VII-17 was used to afford Cpd. No. VII-18 (40.0 mg) as a white solid. ¹H NMR (400 MHz, DMSO-d₆) δ 12.97 (s., 1H), 10.44-10.08 (m, 1H), 8.27-8.01 (m, 1H), 7.89 (d, J=9.0 Hz, 2H), 7.85-7.57 (m, 2H), 7.33 (d, J=9.0 Hz, 2H), 6.74-6.48 (m, 1H), 4.04-4.02 (m, 1H), 3.55-3.51 (m, 1H), 3.32-3.13 (m, 3H), 3.10-2.91 (m, 3H), 2.66-2.55 (m, 1H), 2.12-1.89 (m, 3H), 1.14-0.96 (m, 3H), 0.82-0.69 (m, 3H). MS: 532.1 (M+H)⁺.

### Example VII-19

### Synthesis of N-(4-(chlorodifluoromethoxy)phenyl)-2-(hydroxymethyl)-1-isopropyl-3-methyl-7-(1H-pyrazol-5-yl)indoline-5-carboxamide (Cpd. No. VII-19)

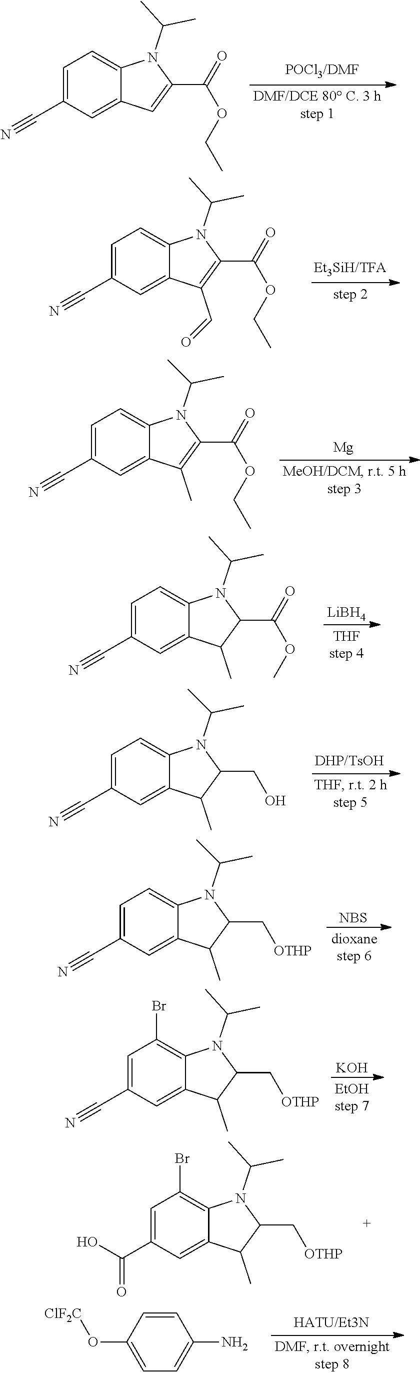

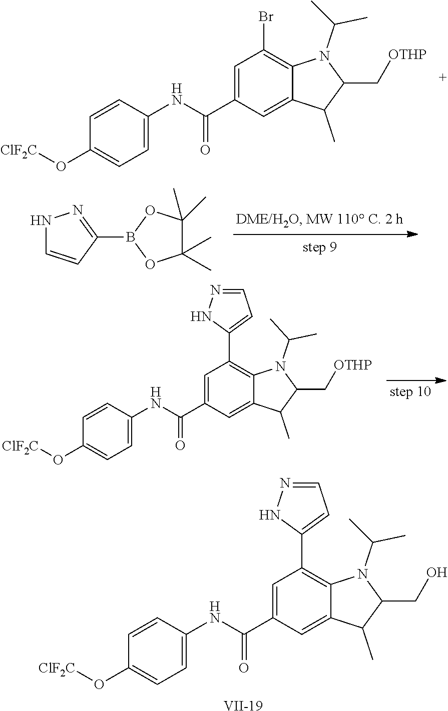

### Step 1: Synthesis of ethyl 5-cyano-3-formyl-1-isopropyl-1H-indole-2-carboxylate

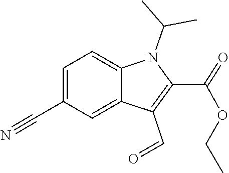 [SMILES: CCOC(=O)c1c(C=O)c2cc(C#N)ccc2n1C(C)C]

To N,N-dimethylformamide (10 mL) at 0° C., was added phosphorus oxychloride (0.629 g, 4.10 mmol) under nitrogen. The mixture was stirred at 0° C. for 30 min, then was added ethyl 5-bromo-1H-indole-2-carboxylate (1.0 g, 3.73 mmol). The mixture was stirred at the same temperature for 0.5 h, concentrated and diluted with ethyl acetate. The organic layer was washed with saturated NaHCO₃ solution, then was dried over Na₂SO₄, filtered and concentrated to give a crude product that was eluted through a silica gel column (ethyl acetate/hexane (10% to 50%) to give ethyl 5-bromo-3-formyl-1H-indole-2-carboxylate (0.4 g, 36.2%). MS: 285.12 (M+H)⁺.

### Step 2: Synthesis of ethyl 5-cyano-1-isopropyl-3-methyl-1H-indole-2-carboxylate

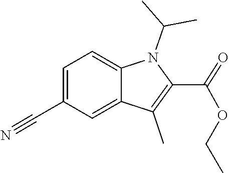 [SMILES: CCOC(=O)c1c(C)c2cc(C#N)ccc2n1C(C)C]

In a 100 mL round-bottomed flask, to a solution of ethyl 5-cyano-3-formyl-1-isopropyl-1H-indole-2-carboxylate (4.3 g, 15.12 mmol) in trifluoroacetic acid (5 mL) was added triethylsilane (5.28 g, 45.4 mmol). The mixture was stirred at 60° C. for 3 h, then evaporated to remove trifluoroacetic acid, followed by partitioned between water and ethyl acetate. The organic layer was washed with saturated NaHCO₃ solution, dried over Na₂SO₄, filtered and concentrated to give a crude product that was eluted through a silica gel column (eluted with ethyl acetate/hexane from 10% to 50%) to give ethyl 5-cyano-1-isopropyl-3-methyl-1H-indole-2-carboxylate (3.5 g, 86%). MS: 271.14 (M+H)⁺.

### Step 3-10: Synthesis of N-(4-(chlorodifluoromethoxy)phenyl)-2-(hydroxymethyl)-1-isopropyl-3-methyl-7-(1H-pyrazol-5-yl)indoline-5-carboxamide (Cpd. No. VII-19)

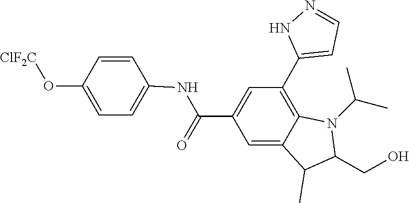

Essentially the same protocol of the preparation of Cpd. No. VII-34 in Example VII-34 was used to afford Cpd. No. VII-19 (85.0 mg) as a white solid. ¹H NMR (400 MHz, DMSO-d₆) δ 10.17 (s, 1H), 7.90 (d, J=8.9 Hz, 2H), 7.84-7.79 (m, 1H), 7.68-7.65 (m, 1H), 7.62-7.51 (m, 1H), 7.35 (d, J=8.7 Hz, 2H), 6.55 (s, 1H), 4.83-4.78 (m, 1H), 3.57-3.44 (m, 1H), 3.44-3.29 (m, 2H), 3.16-3.02 (m, 1H), 1.27 (d, J=7.2 Hz, 3H), 1.09 (d, J=6.8 Hz, 3H), 0.80 (d, J=6.4 Hz, 3H).

### Example VII-20

### Synthesis of N-(4-(chlorodifluoromethoxy)phenyl)-3-ethyl-2-(hydroxymethyl)-1-isopropyl-7-(1H-pyrazol-5-yl)indoline-5-carboxamide (Cpd. No. VII-20)

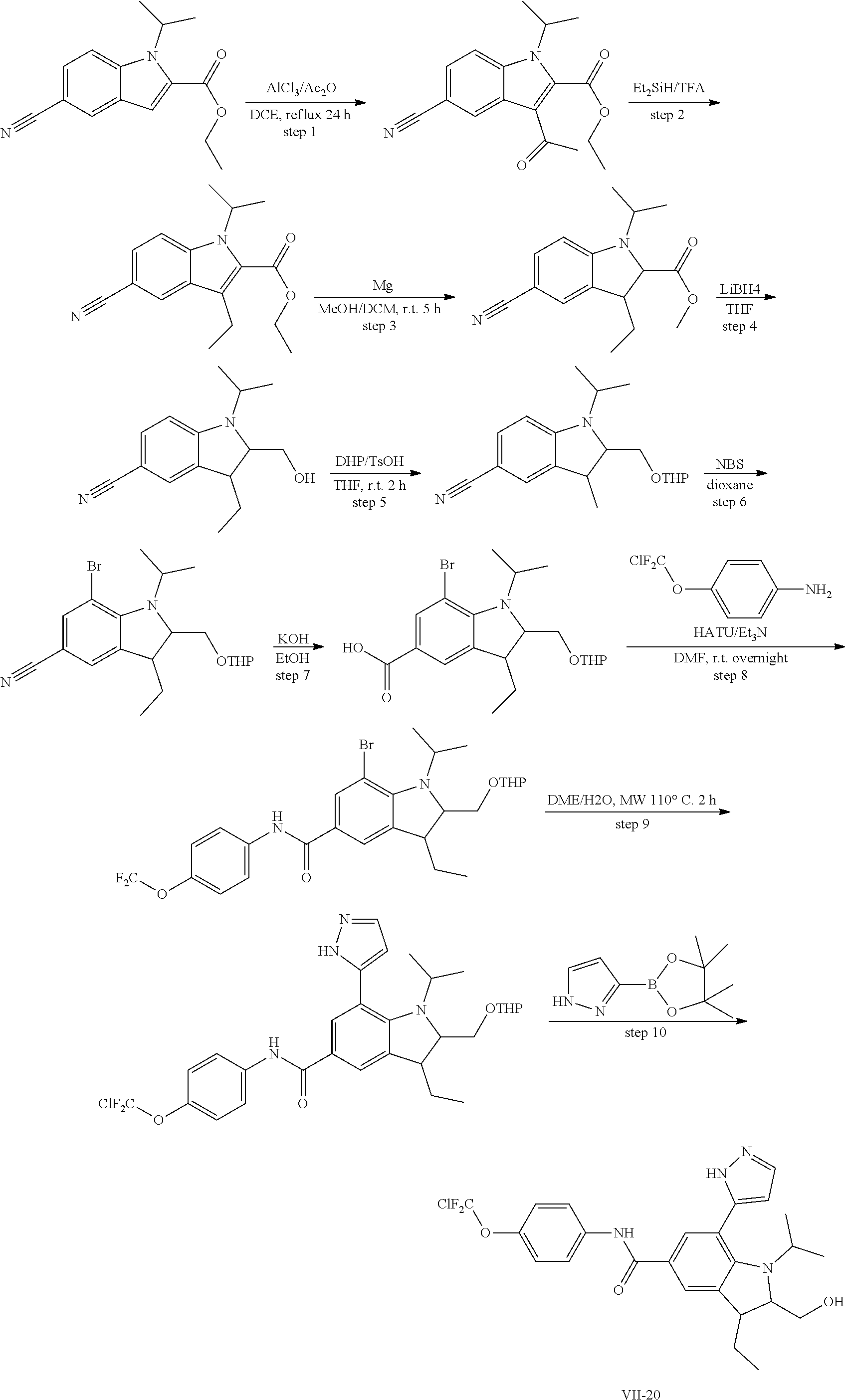

### Step 1: Synthesis of ethyl 3-acetyl-5-cyano-1-isopropyl-1H-indole-2-carboxylate

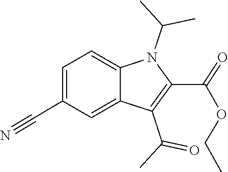 [SMILES: CCOC(=O)c1c(C(C)=O)c2cc(C#N)ccc2n1C(C)C]

In a nitrogen flushed 25 mL two-necked round-bottomed flask, to a suspension of aluminum trichloride (6.50 g, 48.8 mmol) in DCE (20 mL) in ice/water bath, was added acetic anhydride (4.98 g, 48.8 mmol). After being stirred for 5 min in ice/water bath, the mixture was added a solution of ethyl 5-cyano-1-isopropyl-1H-indole-2-carboxylate (2.5 g, 9.75 mmol) in DCE dropwise. The mixture was refluxed for 24 h, then quenched with water (50 mL), followed by extraction with ethyl acetate (30 mL×3). The combined organic layers were washed with brine and dried over Na₂SO₄, filtered and concentrated to give a crude product that was eluted through a silica gel column (ethyl acetate/hexane from 10% to 30%) to give ethyl 3-acetyl-5-cyano-1-isopropyl-1H-indole-2-carboxylate (2.3 g, 79%) as a yellow solid. ¹H NMR (400 MHz, chloroform-d) δ 8.57 (dd, J=1.6, 0.7 Hz, 1H), 7.65 (dd, J=8.8, 0.8 Hz, 1H), 7.56 (dd, J=8.7, 1.6 Hz, 1H), 4.85-4.69 (m, 1H), 4.55 (q, J=7.2 Hz, 2H), 2.59 (s, 3H), 1.69 (d, J=7.0 Hz, 6H), 1.48 (t, J=7.2 Hz, 3H).

### Step 2: Synthesis of thyl 5-cyano-3-ethyl-1-isopropyl-1H-indole-2-carboxylate

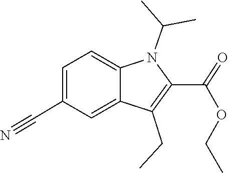 [SMILES: CCOC(=O)c1c(CC)c2cc(C#N)ccc2n1C(C)C]

Under nitrogen, to a solution of ethyl 3-acetyl-5-cyano-1-isopropyl-1H-indole-2-carboxylate (2.5 g, 8.38 mmol) in trifluoroacetic acid (6 mL) was added triethylsilane (3.90 g, 33.5 mmol) in a 100 mL round-bottomed flask. The reaction mixture was stirred at 60° C. for 3 h, then evaporated to remove trifluoroacetic acid, followed by partition between water and ethyl acetate. The organic layer was washed with saturated aqueous NaHCO₃ (30 mL×3), dried over Na₂SO₄, filtered and concentrated to give ethyl 5-cyano-3-ethyl-1-isopropyl-1H-indole-2-carboxylate (1.96 g, 82%), which was pure enough for the next step. ¹H NMR (400 MHz, chloroform-d) δ 8.06 (d, J=1.5 Hz, 1H), 7.63 (d, J=8.8 Hz, 1H), 7.49 (d, J=8.8, 1.6 Hz, 1H), 5.52-5.40 (m, 1H), 4.46 (q, J=7.1 Hz, 2H), 3.02 (q, J=7.5 Hz, 2H), 1.65 (d, J=7.0 Hz, 6H), 1.47 (t, J=7.1 Hz, 3H), 1.27 (t, J=7.4 Hz, 3H).

### Step 3-10: Synthesis of N-(4-(chlorodifluoromethoxy)phenyl)-3-ethyl-2-(hydroxymethyl)-1-isopropyl-7-(1H-pyrazol-5-yl)indoline-5-carboxamide (Cpd. No. VII-20)

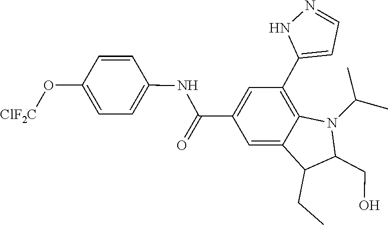

Essentially the same protocol of the preparation of Cpd. No. VII-34 in Example VII-34 was used to afford Cpd. No. VII-20 (15.0 mg) as a white solid. ¹H NMR (400 MHz, DMSO-d₆) δ 12.92 (s, 1H), 10.16 (s, 1H), 7.92-7.87 (m, 2H), 7.80-7.82 (m, 2H), 7.70 (d, J=1.9 Hz, 1H), 7.35 (d, J=8.7 Hz, 2H), 6.53 (s, 1H), 4.79-4.74 (m, 1H), 3.55-3.46 (m, 1H), 3.48-3.43 (m, 1H), 3.28-3.23 (m, 1H), 2.92-2.83 (m, 1H), 1.78-1.63 (m, 1H), 1.54-1.37 (m, 1H), 1.14-1.02 (m, 6H), 0.79 (d, J=6.4 Hz, 3H).

### Example VII-22

### Synthesis of N5-(4-(chlorodifluoromethoxy)phenyl)-1-isopropyl-N2-(2-(methylsulfonyl)ethyl)-7-(pyrimidin-5-yl)indoline-2,5-dicarboxamide (Cpd. No. VII-22)

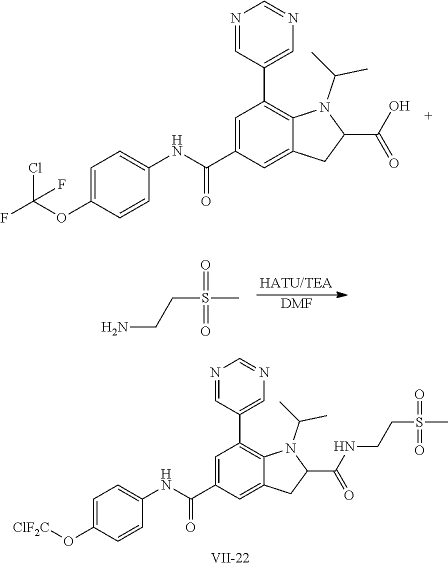

Essentially the same protocol of the preparation of Cpd. No. VII-14 in Example VII-14 was used to afford Cpd. No. VII-22 (9.3 mg) as a white solid. ¹H NMR (400 MHz, DMSO-d₆) δ 10.22 (s, 1H), 9.24 (s, 1H), 9.21 (s, 2H), 8.06 (t, J=6.0 Hz, 1H), 7.91-7.83 (m, 2H), 7.77 (d, J=2.2 Hz, 2H), 7.40-7.30 (d, J=9 Hz, 2H), 4.35 (dd, J=11.4, 5.0 Hz, 1H), 3.66-3.50 (m, 3H), 3.34-3.21 (m, 3H), 3.09 (dd, J=17.1, 5.0 Hz, 1H), 3.03 (s, 3H), 0.99 (d, J=6.8 Hz, 3H), 0.74 (d, J=6.3 Hz, 3H). MS: 608.4 (M+H)⁺.

### Example VII-23

### Synthesis of N-(4-(chlorodifluoromethoxy)phenyl)-1-isopropyl-2-(morpholine-4-carbonyl)-7-(pyrimidin-5-yl)indoline-5-carboxamide (Cpd. No. VII-23)

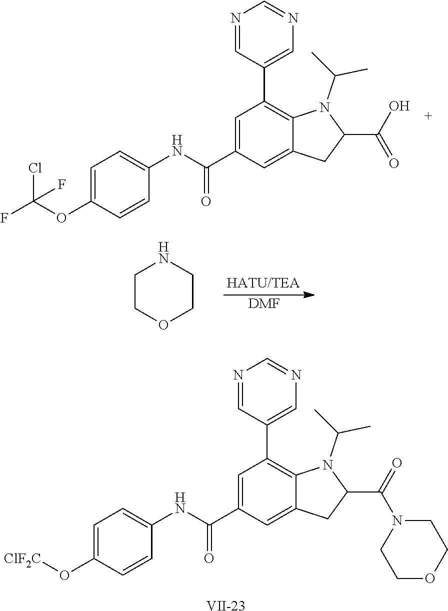

Essentially the same protocol of the preparation of Cpd. No. VII-14 in Example VII-14 was used to afford Cpd. No. VII-23 (5.0 mg) as a white solid. ¹H NMR (400 MHz, DMSO-d₆) δ 10.18 (s, 1H), 9.22 (s, 1H), 9.21 (s, 2H), 7.93-7.85 (m, 2H), 7.81 (d, J=1.8 Hz, 1H), 7.76-7.69 (m, 1H), 7.40-7.31 (m, 2H), 4.92 (dd, J=11.0, 3.8 Hz, 1H), 3.79-3.53 (m, 8H), 3.49-3.30 (m, 2H), 2.87 (dd, J=16.9, 3.7 Hz, 1H), 0.95 (d, J=6.8 Hz, 3H), 0.88 (d, J=6.4 Hz, 3H). MS: 572.4 (M+H)⁺.

### Example VII-24

### Synthesis of N-(4-(chlorodifluoromethoxy)phenyl)-3-ethyl-1-isopropyl-2-((methylamino)methyl)-7-(1H-pyrazol-5-yl)indoline-5-carboxamide (Cpd. No. VII-24)

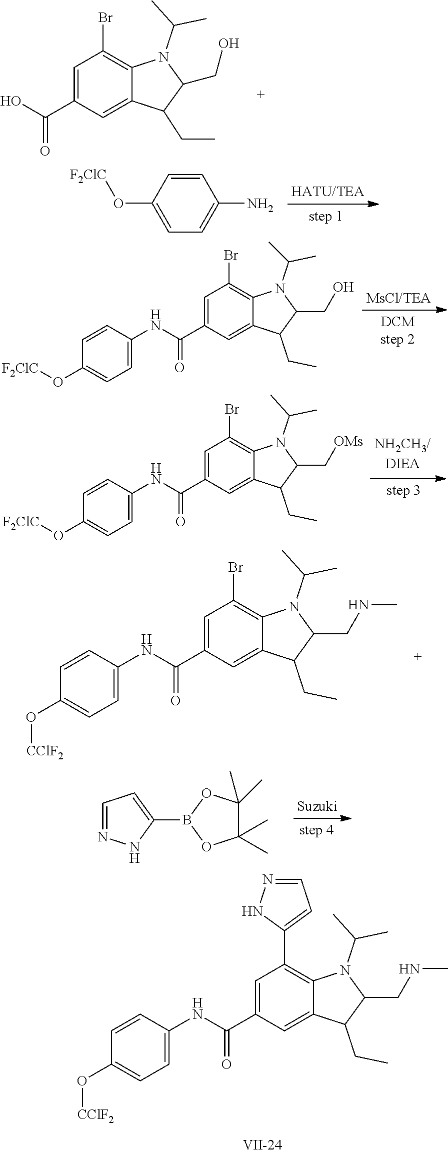

### Step 1: Synthesis of 7-bromo-N-(4-(chlorodifluoromethoxy)phenyl)-3-ethyl-2-(hydroxymethyl)-1-isopropylindoline-5-carboxamide

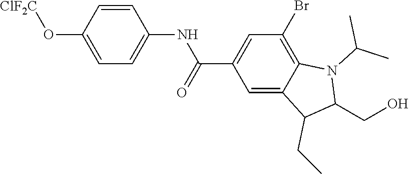

In a 50 mL round-bottomed flask, a solution of 7-bromo-3-ethyl-2-(hydroxymethyl)-1-isopropylindoline-5-carboxylic acid (102 mg, 0.526 mmol), DIEA (85.0 mg, 0.657 mmol) and 2-(7-azabenzotriazol-1-yl)-N,N,N′,N′-tetramethyluronium hexafluorophosphate (250.0 mg, 0.657 mmol), 4-(chlorodifluoromethoxy)aniline (370 mg, 1.910 mmol) in N,N-dimethylformamide (5 mL) was stirred at room temperature for overnight. The mixture was diluted with water (10 mL), then extracted with ethyl acetate (30 mL×2). The combined organic layers were washed with brine, dried over Na₂SO₄, filtered and concentrated to give a crude product that was eluted through a silica gel column (ethyl acetate/hexane from 10% to 50%) to give 7-bromo-N-(4-(chlorodifluoromethoxy)phenyl)-3-ethyl-2-(hydroxymethyl)-1-isopropylindoline-5-carboxamide (130.0 mg, 57.3%). MS: 518.06 (M+H)⁺.

### Step 2: Synthesis of (7-bromo-5-((4-(chlorodifluoromethoxy)phenyl)carbamoyl)-3-ethyl-1-isopropylindolin-2-yl)methyl methanesulfonate

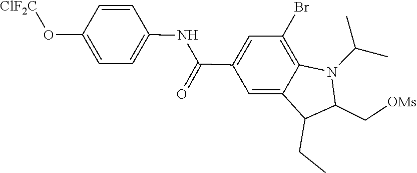

In a 50 mL two-necked round-bottomed flask, to a solution of 7-bromo-N-(4-(chlorodifluoromethoxy)phenyl)-3-ethyl-2-(hydroxymethyl)-1-isopropylindoline-5-carboxamide (130.0 mg, 0.251 mmol) and TEA (50.8 mg, 0.502 mmol) in dichloromethane (3 mL) under ice/water bath, was dropped MsCl (43.1 mg, 0.377 mmol). The mixture was stirred for 3 h under ice/water bath, then quenched with water (30 mL), followed by extraction with ethyl acetate (30 mL×3). The combined organic layers were dried over Na₂SO₄, filtered and concentrated to give a crude product that was eluted through a silica gel column (ethyl acetate/hexane from 10% to 70%) to give (7-bromo-5-((4-(chlorodifluoromethoxy)phenyl)carbamoyl)-3-ethyl-1-isopropylindolin-2-yl)methyl methanesulfonate (80.0 mg, 53.5%). MS: 596.04 (M+H)⁺.

### Step 3: Synthesis of 7-bromo-N-(4-(chlorodifluoromethoxy)phenyl)-3-ethyl-1-isopropyl-2-((methylamino) methyl)indoline-5-carboxamide

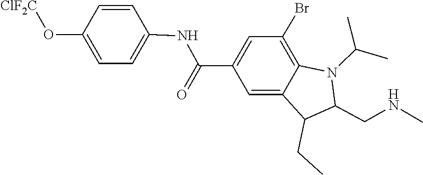

In a 50 mL round-bottomed flask, to a solution of (7-bromo-5-((4-(chlorodifluoromethoxy) phenyl)carbamoyl)-3-ethyl-1-isopropylindolin-2-yl)methyl methanesulfonate (80 mg, 0.134 mmol) and DIEA (34.7 mg, 0.269 mmol) in tetrahydrofuran (10 mL) was added methylamine (8.34 mg, 0.269 mmol) under nitrogen. The mixture was heated to reflux for 6 h, then concentrated to give a crude product that was eluted through a silica gel column (ethyl acetate/hexane from 10% to 100%) to give 7-bromo-N-(4-(chlorodifluoromethoxy)phenyl)-3-ethyl-1-isopropyl-2-((methylamino)methyl)indoline-5-carboxamide (40 mg, 56.1%). MS: 931.09 (M+H)⁺.

### Step 4: Synthesis of N-(4-(chlorodifluoromethoxy)phenyl)-3-ethyl-1-isopropyl-2-((methylamino) methyl)-7-(1H-pyrazol-5-yl)indoline-5-carboxamide (Cpd. No. VII-24)

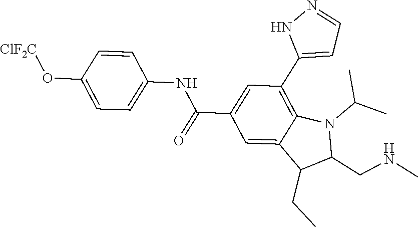

Essentially the same protocol of the preparation of Cpd. No. VII-34 in Example VII-34 was used to afford Cpd. No. VII-24 (26.0 mg) as a white solid. ¹H NMR (400 MHz, DMSO-d₆) δ 12.95 (s, 1H), 10.17 (s, 1H), 7.90 (d, J=9.1 Hz, 2H), 7.70 (s, 1H), 7.85-7.80 (m, 2H), 7.35 (d, J=8.7 Hz, 2H), 6.54 (s, 1H), 3.72-3.57 (m, 1H), 3.38 (s, 1H), 2.93-2.86 (m, 1H), 2.72-2.56 (m, 2H), 2.43 (s, 3H), 1.76-1.64 (m, 1H), 1.53-1.41 (m, 1H), 1.13 (d, J=6.8 Hz, 3H), 1.06 (t, J=7.3 Hz, 3H), 0.79 (d, J=6.5 Hz, 3H).

### Example VII-25

### Synthesis of N-(4-(chlorodifluoromethoxy)phenyl)-1-isopropyl-2-(morpholine-4-carbonyl)-7-(1H-pyrazol-5-yl)indoline-5-carboxamide (Cpd. No. VII-25)

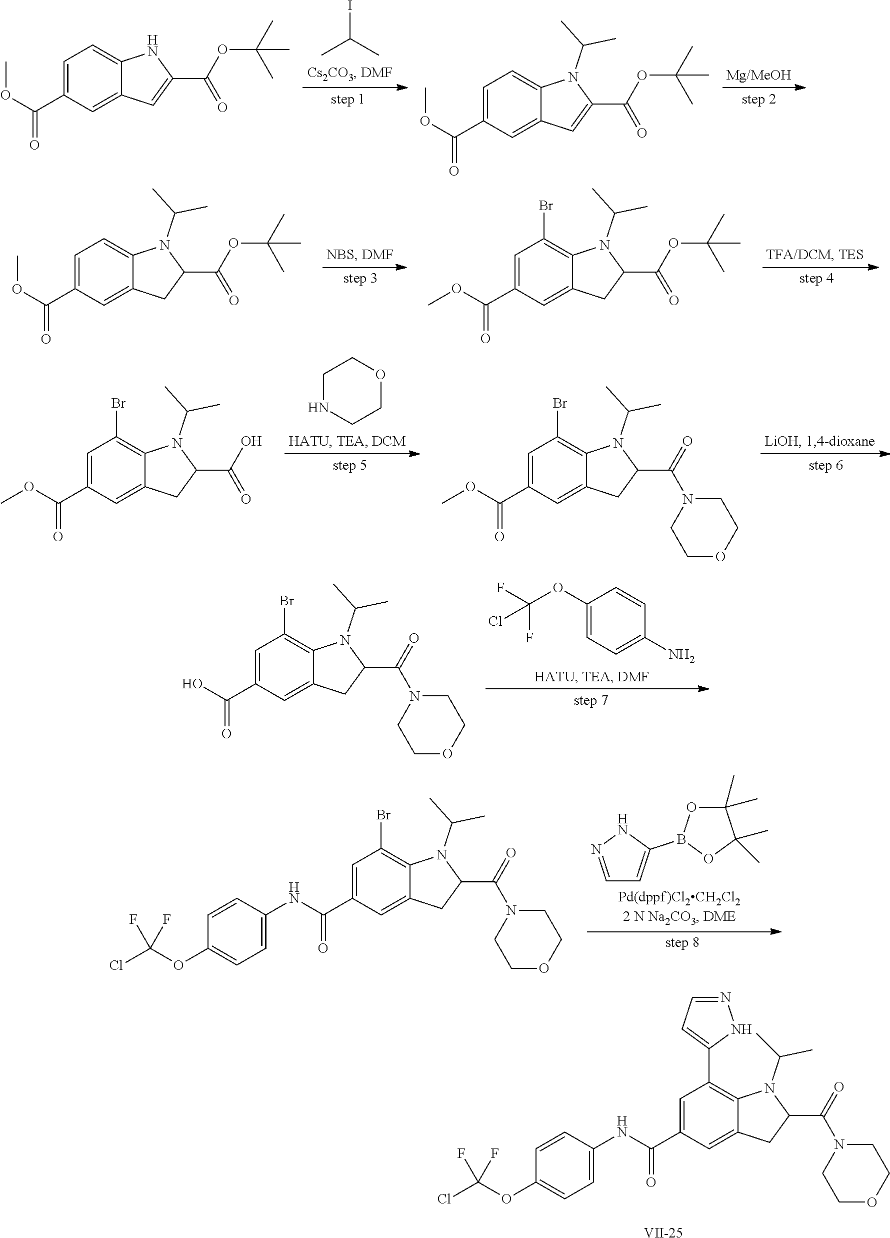

### Step 1: 2-tert-butyl 5-methyl 1-isopropyl-1H-indole-2,5-dicarboxylate

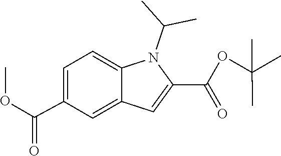 [SMILES: COC(=O)c1ccc2c(c1)cc(C(=O)OC(C)(C)C)n2C(C)C]

2-iodopropane (14.82 g, 87 mmol) and Cs₂CO₃ (28.4 g, 87 mmol) were added to a solution of 2-tert-butyl 5-methyl 1H-indole-2,5-dicarboxylate (6.0 g, 21.79 mmol) in N,N-dimethylformamide (80.0 mL). The mixture was stirred at 70° C. for 12 h, then diluted with ethyl acetate (200 mL), washed with water (100 mL) and brine (150 mL). The organic layer was dried over Na₂SO₄, filtered and concentrated to give a crude product that was eluted through a silica gel column (ethyl acetate/hexane from 0% to 15%) to give 2-(tert-butyl) 5-methyl 1-isopropyl-1H-indole-2,5-dicarboxylate (6.1 g, 88%) as a yellow solid. MS: 318.2 (M+H)⁺.

### Step 2: Synthesis of 2-tert-butyl 5-methyl 1-isopropylindoline-2,5-dicarboxylate

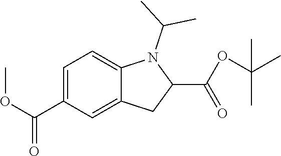 [SMILES: COC(=O)c1ccc2c(c1)CC(C(=O)OC(C)(C)C)N2C(C)C]

Magnesium (0.444 g, 18.27 mmol) was added to a solution of 2-(tert-butyl) 5-methyl 1-isopropyl-1H-indole-2,5-dicarboxylate (2.9 g, 9.15 mmol) in methanol (9.0 mL). The mixture was stirred at room temperature for 12 h, then quenched with NH₄HCl aqueous solution. The mixture was diluted with ethyl acetate (100 mL), and washed with water (100 mL) and brine (100 mL). The organic layer was dried over Na₂SO₄, filtered and concentrated to give a crude product that was eluted through a silica gel column (ethyl acetate/hexane from 0 to 30%) to give 2-(tert-butyl) 5-methyl 1-isopropylindoline-2,5-dicarboxylate (1.5 g, 51.4%) as a yellow oil. MS: 320.2 (M+H)⁺.

### Step 3: Synthesis of 2-tert-butyl 5-methyl 7-bromo-1-isopropylindoline-2,5-dicarboxylate

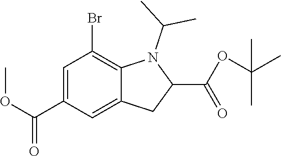 [SMILES: COC(=O)c1cc(Br)c2c(c1)CC(C(=O)OC(C)(C)C)N2C(C)C]

N-Bromosuccinimide (0.831 g, 4.70 mmol) was added to a solution of 2-(tert-butyl) 5-methyl 1-isopropylindoline-2,5-dicarboxylate (1.5 g, 4.7 mmol) in 1,4-dioxane (30.0 mL) at 0° C. The mixture was stirred at room temperature for 2 h. The mixture was quenched by NaHCO₃ aqueous solution, diluted with ethyl acetate (80 mL), then washed with water (80 mL) and brine (80 mL). The organic layer was dried over Na₂SO₄, filtered and concentrated to give a crude product that was eluted through a silica gel column (ethyl acetate/hexane from 0% to 30%) to give 2-(tert-butyl) 5-methyl 7-bromo-1-isopropylindoline-2,5-dicarboxylate (1.5 g, 80%) as a yellow oil. MS: 398.1 (M+H)⁺.

### Step 4: Synthesis of 7-bromo-1-isopropyl-5-(methoxycarbonyl)indoline-2-carboxylic acid

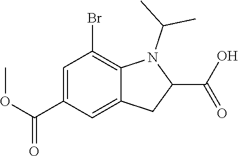 [SMILES: COC(=O)c1cc(Br)c2c(c1)CC(C(=O)O)N2C(C)C]

In a nitrogen flushed 25 mL round-bottomed flask 2-(tert-butyl) 5-methyl 7-bromo-1-isopropylindoline-2,5-dicarboxylate (0.5 g, 1.255 mmol) and triethylsilane (0.292 g, 2.51 mmol) were dissolved in dichloromethane (5 mL)/trifluoroacetic acid (5.00 mL) under nitrogen to give a yellow solution. The mixture was stirred at room temperature for 16 h, then concentrated to give crude product 0.3 g, which was used in the next step directly without purification. MS: 342.1 (M+H)⁺.

### Step 5: Synthesis of methyl 7-bromo-1-isopropyl-2-(morpholine-4-carbonyl)indoline-5-carboxylate

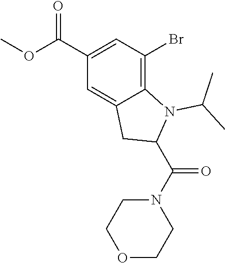 [SMILES: COC(=O)c1cc(Br)c2c(c1)CC(C(=O)N1CCOCC1)N2C(C)C]

Morpholine (0.102 g, 1.169 mmol), TEA (0.118 g, 1.169 mmol) and 2-(7-azabenzotriazol-1-yl)-N,N,N′,N′-tetramethyluronium hexafluorophosphate (0.333 g, 0.877 mmol) were added to a solution of 7-bromo-1-isopropyl-5-(methoxycarbonyl)indoline-2-carboxylic acid (0.2 g, 0.584 mmol) in N,N-dimethylformamide (5 mL). The mixture was stirred at 45° C. for 10 h. After being cooled to room temperature, the mixture was diluted with ethyl acetate (50 mL), washed with water (50 mL) and brine (50 mL). The organic layer was dried over Na₂SO₄, filtered and concentrate to give a crude product that was eluted through a silica gel column (ethyl acetate/hexane from 0% to 70%) to give methyl 7-bromo-1-isopropyl-2-(morpholine-4-carbonyl)indoline-5-carboxylate (0.19 g, 79%) as a yellow oil. MS: 411.1 (M+H)⁺.

### Step 6: Synthesis of 7-bromo-1-isopropyl-2-(morpholine-4-carbonyl)indoline-5-carboxylic acid

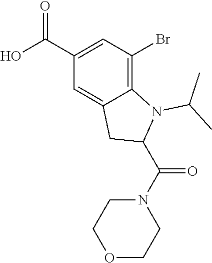 [SMILES: CC(C)N1c2c(Br)cc(C(=O)O)cc2CC1C(=O)N1CCOCC1]

Lithium hydroxide (0.033 g, 1.386 mmol) was added to a solution of methyl 7-bromo-1-isopropyl-2-(morpholine-4-carbonyl)indoline-5-carboxylate (0.19 g, 0.462 mmol) in 1,4-dioxane/water (4.0 mL/1.0 mL). The mixture was stirred 45° C. for 12 h. The mixture was concentrated, then acidified with 1N HCl aqueous solution. The collected precipitate was dried under vacuum to give product 7-bromo-1-isopropyl-2-(morpholine-4-carbonyl)indoline-5-carboxylic acid (150 mg, 82%) as a white solid.

### Step 7: Synthesis of 7-bromo-N-(4-(chlorodifluoromethoxy)phenyl)-1-isopropyl-2-(morpholine-4-carbonyl)indoline-5-carboxamide

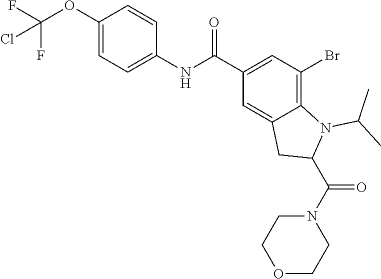 [SMILES: CC(C)N1c2c(Br)cc(C(=O)Nc3ccc(OC(F)(F)Cl)cc3)cc2CC1C(=O)N1CCOCC1]

4-(chlorodifluoromethoxy)aniline (146 mg, 0.755 mmol), TEA (76 mg, 0.755 mmol) and 2-(7-azabenzotriazol-1-yl)-N,N,N′,N′-tetramethyluronium hexafluorophosphate (215 mg, 0.566 mmol) were added to a solution of 0.7-bromo-1-isopropyl-2-(morpholine-4-carbonyl)indoline-5-carboxylic acid (150 mg, 0.378 mmol) in N,N-dimethylformamide (3.0 mL). The reaction mixture was stirred 45° C. for 12 h. The mixture was diluted with ethyl acetate (50 mL), washed with water (50 mL) and brine (50 mL). The organic layer was dried over Na₂SO₄, filtered and concentrated to give a crude product that was eluted through a silica gel column (methanol/dichloromethane from 0% to 10%) to give 7-bromo-N-(4 (chlorodifluoromethoxy)phenyl)-1-isopropyl-2-(morpholine-4-carbonyl)indoline-5-carboxamide (0.16 g, 74.0%) as a yellow MS: 574.2 (M+H)⁺.

### Step 8: Synthesis of N-(4-(chlorodifluoromethoxy)phenyl)-1-isopropyl-2-(morpholine-4-carbonyl)-7-(1H-pyrazol-5-yl)indoline-5-carboxamide (Cpd. No. VII-25)

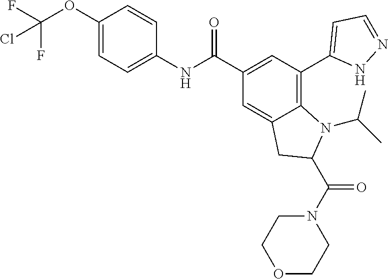 [SMILES: CC(C)N1c2c(cc(C(=O)Nc3ccc(OC(F)(F)Cl)cc3)cc2-c2ccn[nH]2)CC1C(=O)N1CCOCC1]

5-(4,4,5,5-tetramethyl-1,3,2-dioxaborolan-2-yl)-1H-pyrazole (54.2 mg, 0.279 mmol) and PdCl₂(dppf)-CH₂Cl₂ adduct (22.81 mg, 0.028 mmol) were added to a solution of 7-bromo-N-(4-(chlorodifluoromethoxy)phenyl)-1-isopropyl-2-(morpholine-4-carbonyl)indoline-5-carboxamide (80 mg, 0.140 mmol) in dimethoxyethane (0.9 mL)/2N Na₂CO₃ (0.3 mL). The mixture was purged with nitrogen for three times, then stirred at 100° C. under MW for 0.5 h. The mixture was diluted with ethyl acetate (30 mL), washed with water (30 mL) and brine (30 mL). The organic layer was dried over Na₂SO₄, filtered and concentrated to give a crude product that was purified using preparative HPLC to give N-(4-(chlorodifluoromethoxy)phenyl)-1-isopropyl-2-(morpholine-4-carbonyl)-7-(1H-pyrazol-5-yl)indoline-5-carboxamide (8 mg, 10.23%) as a white solid. ¹H NMR (400 MHz, DMSO-d₆) δ 13.26-12.81 (m, 1H), 10.33-10.02 (m, 1H), 7.87 (d, J=9.0 Hz, 2H), 7.85-7.73 (m, 2H), 7.69-7.48 (m, 1H), 7.31 (d, J=9.0 Hz, 2H), 6.62-6.35 (m, 1H), 4.82-4.77 (m, 1H), 3.79-3.41 (m, 10H), 2.85-2.79 (m, 1H), 0.94-0.92 (m, 3H), 0.86-0.72 (m, 3H). MS: 560.1 (M+H)⁺.

### Example VII-26

### Synthesis of N5-(4-(chlorodifluoromethoxy)phenyl)-1-isopropyl-N2,N2-dimethyl-7-(1H-pyrazol-5-yl)indoline-2,5-dicarboxamide (Cpd. No. VII-26)

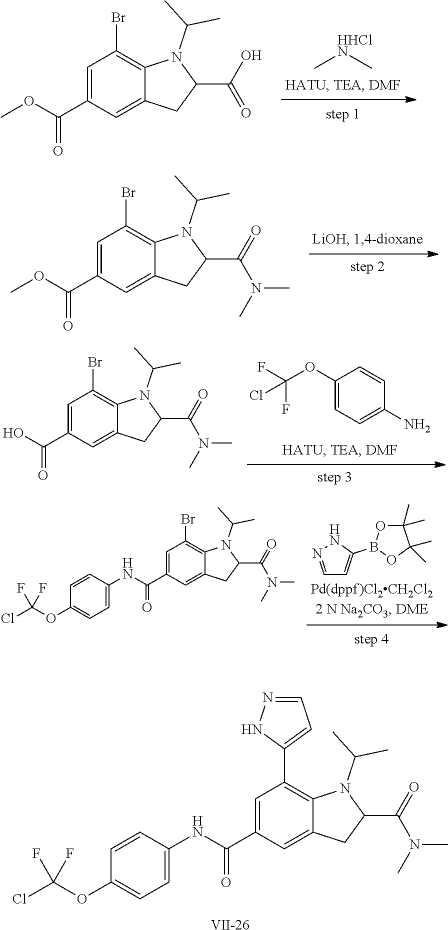

Essentially the same protocol of the preparation of Cpd. No. VII-25 in Example VII-25 was used to afford Cpd. No. VII-26 (9.0 mg) as a white solid. ¹H NMR (400 MHz, DMSO-d₆) δ 10.16 (s, 1H), 7.87 (d, J=9.0 Hz, 2H), 7.84-7.56 (m, 3H), 7.31 (d, J=9.0 Hz, 2H), 6.51 (s, 1H), 4.78-4.62 (m, 1H), 3.77-3.51 (m, 2H), 3.11 (s, 3H), 2.86 (s, 3H), 2.78 (dd, J=16.4, 5.2 Hz, 1H), 0.90 (d, J=6.4 Hz, 3H), 0.80 (d, J=6.4 Hz, 3H). MS: 518.2 (M+H)⁺.

### Example VII-27

### Synthesis of N-(4-(chlorodifluoromethoxy)phenyl)-3-((dimethylamino)methyl)-1-isopropyl-7-(1H-pyrazol-5-yl)indoline-5-carboxamide (Cpd. No. VII-27)

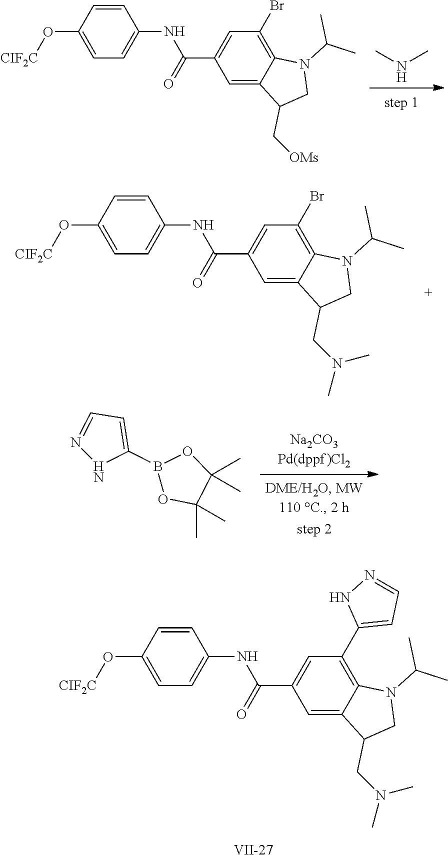

Essentially the same protocol of the preparation of Cpd. No. VII-28 in Example VII-28 was used to afford Cpd. No. VII-27 (33.0 mg) as a white solid. ¹H NMR (400 MHz, DMSO-d₆) δ 10.16 (s, 1H), 9.61 (s, 1H), 7.92-7.85 (m, 2H), 7.83-7.75 (m, 3H), 7.35 (d, J=8.7 Hz, 2H), 6.42 (d, J=2.1 Hz, 1H), 3.85-3.68 (m, 2H), 3.67-3.58 (m, 2H), 3.48-3.28 (m, 2H), 2.96 (d, J=4.6 Hz, 3H), 2.93 (d, J=4.6 Hz, 3H), 0.96 (d, J=6.7 Hz, 3H), 0.91 (d, J=6.6 Hz, 3H).

### Example VII-28

### Synthesis of N-(4-(chlorodifluoromethoxy)phenyl)-1-isopropyl-3-((methylamino)methyl)-7-(1H-pyrazol-5-yl)indoline-5-carboxamide (Cpd. No. VII-28)

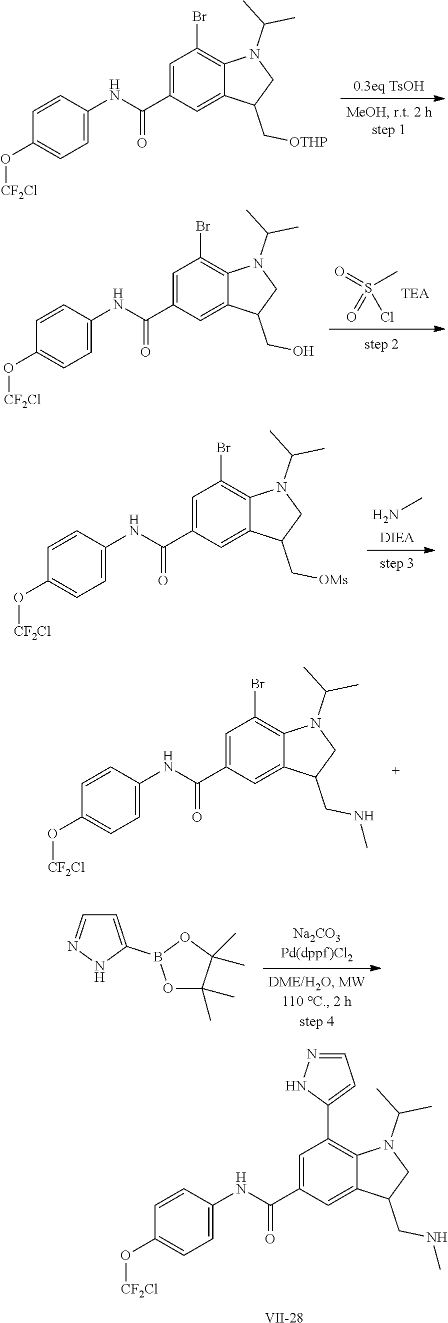

### Step 1: Synthesis of 7-bromo-N-(4-(chlorodifluoromethoxy)phenyl)-3-(hydroxymethyl)-1-isopropylindoline-5-carboxamide

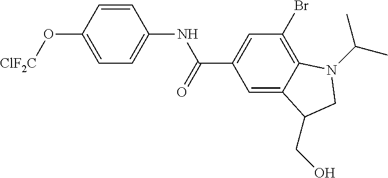

In a 25 mL round-bottomed flask, to a solution of 7-bromo-N-(4-(chlorodifluoromethoxy)phenyl)-1-isopropyl-3-(((tetrahydro-2H-pyran-2-yl)oxy)methyl)indoline-5-carboxamide (380.0 mg, 0.662 mmol) in 5 mL methanol was added p-toluenesulfonic acid (37.8 mg, 0.199 mmol). The mixture was stirred for overnight at room temperature, then concentrated to give a crude product that was eluted through a silica gel column (ethyl acetate/hexane from 0% to 20%) to give 7-bromo-N-(4-(chlorodifluoromethoxy)phenyl)-3-(hydroxymethyl)-1-isopropylindoline-5-carboxamide (80 mg, 24.7%). MS: 490.30 (M+H)⁺.

### Step 2: Synthesis of (7-bromo-5-((4-(chlorodifluoromethoxy)phenyl)carbamoyl)-1-isopropylindolin-3-yl)methyl methanesulfonate

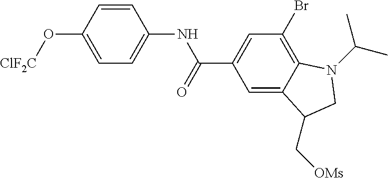

To a solution of 7-bromo-N-(4-(chlorodifluoromethoxy)phenyl)-3-(hydroxymethyl)-1-isopropylindoline-5-carboxamide (350 mg, 0.715 mmol) and TEA (145 mg, 1.429 mmol) in dichloromethane (3 mL) was added MsCl (123 mg, 1.072 mmol) dropwise at 0° C. Then the mixture was warmed up to room temperature and stirred for 3 h, quenched with water, followed by extraction with dichloromethane. The combined organic layers were concentrated to give a crude product that was eluted through a silica gel column (ethyl acetate/hexane from 10% to 80%) to give (7-bromo-5-((4-(chlorodifluoromethoxy)phenyl)carbamoyl)-1-isopropylindolin-3-yl)methyl methanesulfonate (320 mg, 79%). MS: 595.90, 597.95 (M+H)⁺.

### Step 3: Synthesis of 7-bromo-N-(4-(chlorodifluoromethoxy)phenyl)-1-isopropyl-3-((methylamino)methyl)indoline-5-carboxamide

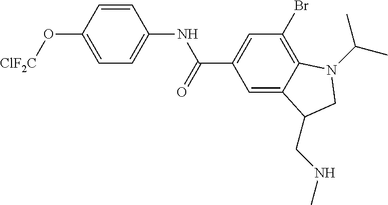

To a solution of (7-bromo-5-((4-(chlorodifluoromethoxy)phenyl)carbamoyl)-1-isopropylindolin-3-yl)methyl methanesulfonate (120 mg, 0.211 mmol) and DIEA (54.6 mg, 0.423 mmol) in 1.0 mL N,N-dimethylformamide was added methylamine (13.13 mg, 0.423 mmol). The mixture was stirred for 1 h at 150° C. under MW, then quenched with water, followed by extraction with ethyl acetate (30 mL×3). The combined organic layers were concentrated to give a crude product that was purified by silica gel column chromatography (ethyl acetate/hexane from 10% to 100%) to give 7-bromo-N-(4-(chlorodifluoromethoxy)phenyl)-1-isopropyl-3-((methylamino)methyl)indoline-5-carboxamide (70 mg, 65.9%). MS: 502.95, 504.95 (M+H)⁺.

### Step 4: Synthesis of N-(4-(chlorodifluoromethoxy)phenyl)-1-isopropyl-3-((methylamino)methyl)-7-(1H-pyrazol-5-yl)indoline-5-carboxamide (Cpd. No. VII-28)

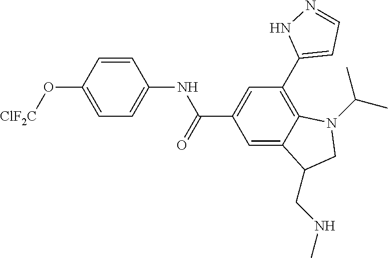

In a 5 mL microwave tube, to a solution of 7-bromo-N-(4-(chlorodifluoromethoxy)phenyl)-1-isopropyl-3-((methylamino)methyl)indoline-5-carboxamide (75.0 mg, 0.149 mmol) in dimethoxyethane (2.0 mL) and water (0.4 mL) was added 5-(4,4,5,5-tetramethyl-1,3,2-dioxaborolan-2-yl)-1H-pyrazole (43.4 mg, 0.224 mmol), PdCl₂(dppf)-CH₂Cl₂ adduct (12.18 mg, 0.015 mmol) and Na₂CO₃ (31.6 mg, 0.298 mmol). The mixture was stirred at 110° C. under MW for 2 h under nitrogen, then quenched with water, followed by extraction with ethyl acetate (30 mL×3). The combined organic layers were concentrated to give a crude product that was eluted through a silica gel column (ethyl acetate/hexane from 5% to 40%) to give N-(4-(chlorodifluoromethoxy)phenyl)-1-isopropyl-3-((methylamino)methyl)-7-(1H-pyrazol-5-yl)indoline-5-carboxamide (18.0 mg, 24.6%). ¹H NMR (400 MHz, DMSO-d₆) δ 10.15 (s, 1H), 8.61 (s, 1H), 8.53 (s, 1H), 7.88 (d, J=8.7 Hz, 2H), 7.82-7.75 (m, 3H), 7.35 (d, J=8.7 Hz, 2H), 6.42 (d, J=2.1 Hz, 1H), 3.69-3.57 (m, 3H), 3.46-3.36 (m, 2H), 3.18-3.11 (m, 1H), 2.70 (t, J=5.3 Hz, 3H), 0.95 (d, J=6.6 Hz, 3H), 0.93 (d, J=6.6 Hz, 3H). MS: 491.10 (M+H)⁺.

### Example VII-29

### Synthesis of N-(4-(chlorodifluoromethoxy)phenyl)-2-((R)-3-hydroxypyrrolidine-1-carbonyl)-1-isopropyl-7-(1H-pyrazol-5-yl)indoline-5-carboxamide (Cpd. No. VII-29)

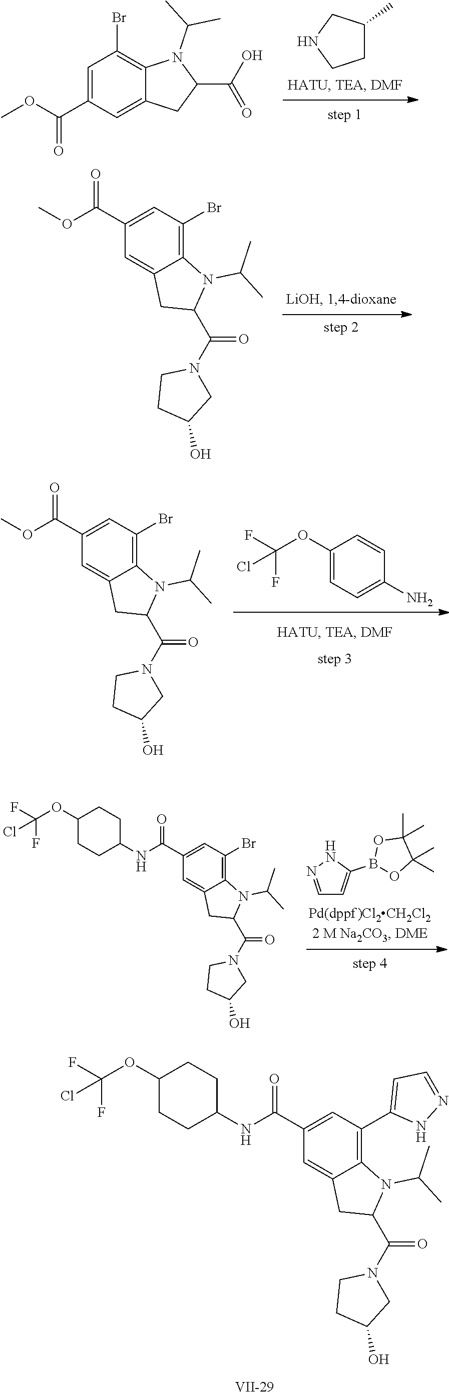

Essentially the same protocol of the preparation of Cpd. No. VII-25 in Example VII-25 was used to afford Cpd. No. VII-29 (12.0 mg) as a white solid. ¹H NMR (400 MHz, DMSO-d₆+D₂O) δ 7.80-7.69 (m, 4H), 7.65-7.57 (m, 1H), 7.35-7.25 (m, 2H), 6.58-6.49 (m, 1H), 4.80-4.54 (m, 1H), 4.43-4.20 (m, 1H), 3.76-3.24 (m, 6H), 2.96-2.72 (m, 1H), 2.09-1.71 (m, 2H), 0.94-0.92 (m, 3H), 0.81-0.65 (m, 3H). MS: 560.1 (M+H)⁺.

### Example VII-30

### Synthesis of N⁵-(4-(chlorodifluoromethoxy)phenyl)-1-isopropyl-N2,N2-bis(2-methoxyethyl)-7-(1H-pyrazol-5-yl)indoline-2,5-dicarboxamide (Cpd. No. VII-30)

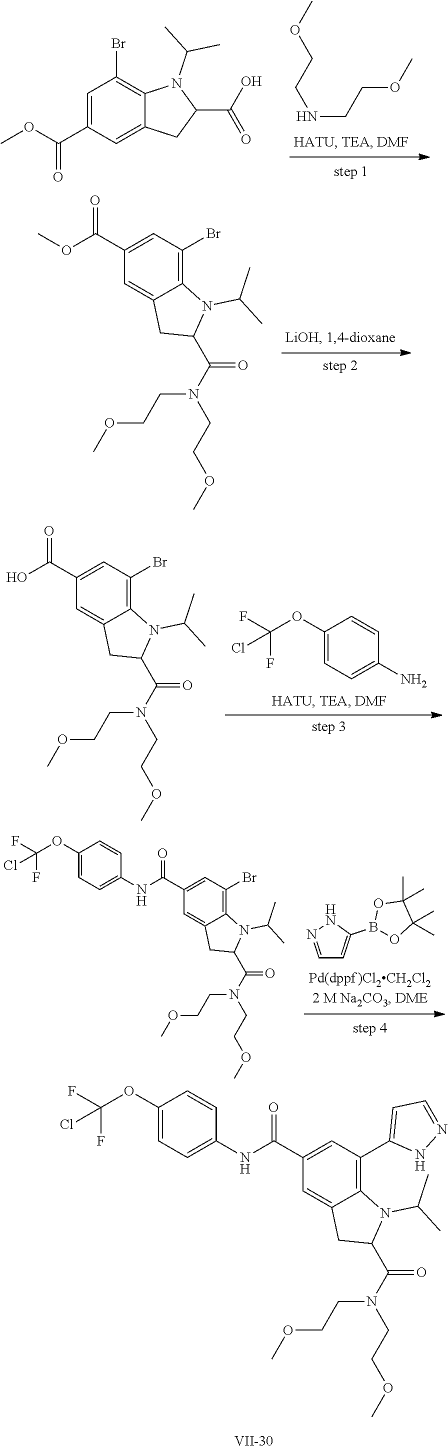

Essentially the same protocol of the preparation of Cpd. No. VII-25 in Example VII-25 was used to afford Cpd. No. VII-30 (15.0 mg) as a white solid. ¹H NMR (400 MHz, DMSO-d₆) δ 13.18-12.80 (m, 1H), 10.13 (s, 1H), 7.87 (d, J=9.0 Hz, 2H), 7.84-7.46 (m, 3H), 7.31 (d, J=9.0 Hz, 2H), 6.60-6.36 (m, 1H), 4.89-4.87 (m, 1H), 3.80-3.46 (m, 7H), 3.46-3.38 (m, 3H), 3.30 (s, 3H), 3.23 (s, 3H), 2.73-2.67 (m, 1H), 0.92-0.90 (m, 3H), 0.86-0.71 (m, 3H). MS: 606.2 (M+H)⁺.

### Example VII-31

### Synthesis of N-(4-(chlorodifluoromethoxy)phenyl)-2-(1,1-dioxidothiomorpholine-4-carbonyl)-1-isopropyl-7-(1H-pyrazol-5-yl)indoline-5-carboxamide (Cpd. No. VII-31)

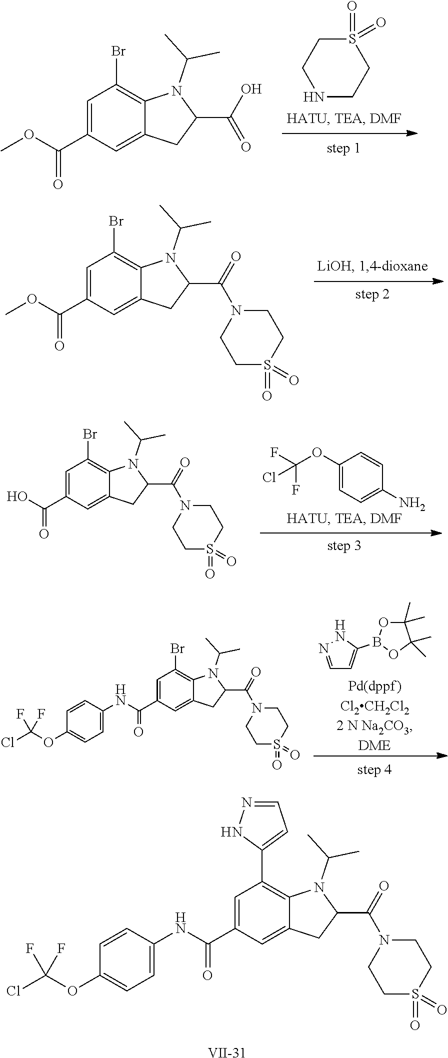

Essentially the same protocol of the preparation of Cpd. No. VII-25 in Example VII-25 was used to afford Cpd. No. VII-31 (12.0 mg) as a white solid. ¹H NMR (400 MHz, DMSO-d₆) δ 13.18-12.82 (m, 1H), 10.35-10.02 (m, 1H), 7.87 (d, J=8.8 Hz, 2H), 7.84-7.53 (m, 3H), 7.32 (d, J=8.8 Hz, 2H), 6.61-6.45 (m, 1H), 4.97-4.80 (m, 1H), 4.17-3.88 (m, 3H), 3.86-3.49 (m, 2H), 3.29-3.10 (m, 5H), 3.01-2.95 (m, 1H), 0.95-0.94 (m, 3H), 0.80-0.79 (m, 3H).

### Example VII-32

### Synthesis of N5-(4-(chlorodifluoromethoxy)phenyl)-N2-(2-(dimethylamino)ethyl)-1-isopropyl-7-(1H-pyrazol-5-yl)indoline-2,5-dicarboxamide (Cpd. No. VII-32)

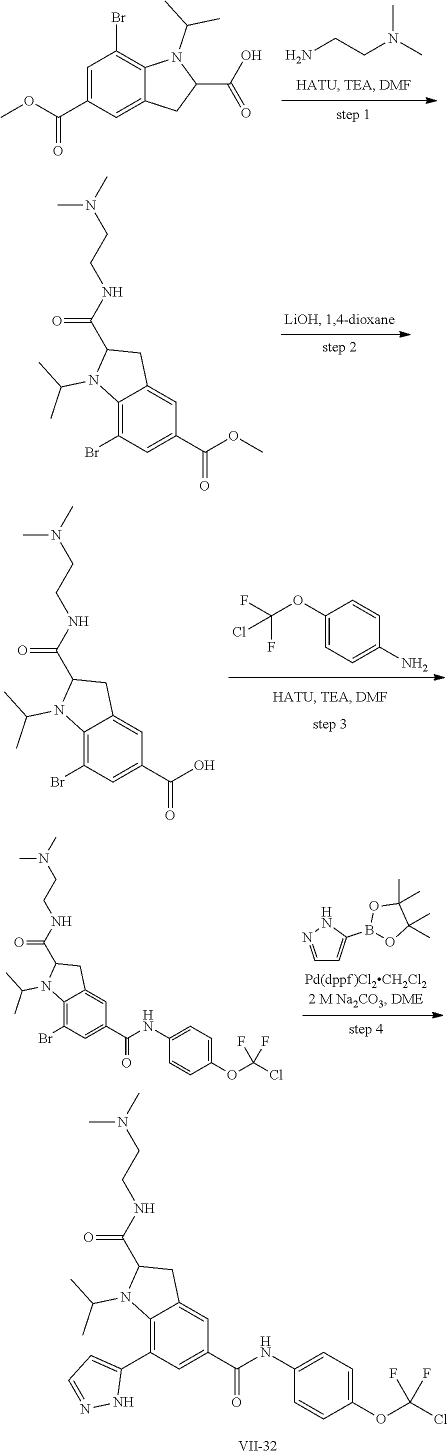

Essentially the same protocol of the preparation of Cpd. No. VII-25 in Example VII-25 was used to afford Cpd. No. VII-32 (6.0 mg) as a white solid. ¹H NMR (400 MHz, DMSO-d₆+D₂O) δ=7.90-7.79 (m, 3H), 7.78-7.73 (m, 1H), 7.68 (s, 1H), 7.32 (d, J=9.0 Hz, 2H), 6.73-6.71 (m, 1H), 4.32-4.28 (m, 1H), 3.57-3.40 (m, 4H), 3.22-3.00 (m, 3H), 2.79-2.78 (m, 6H), 1.03-1.01 (m, 3H), 0.72-0.71 (m, 3H). MS: 561.2 (M+H)⁺.

### Example VII-33

### Synthesis of N-(4-(chlorodifluoromethoxy)phenyl)-1-isopropyl-2-(4-methylpiperazine-1-carbonyl)-7-(1H-pyrazol-5-yl)indoline-5-carboxamide (Cpd. No. VII-33)

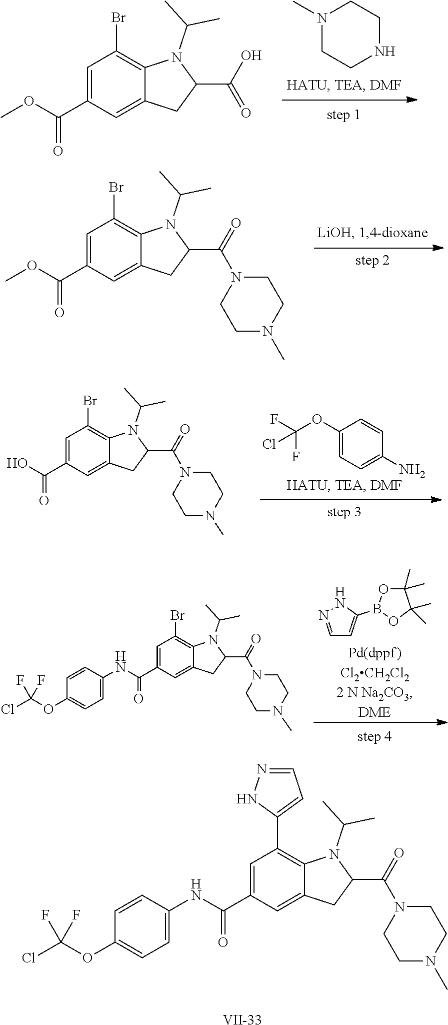

Essentially the same protocol of the preparation of Cpd. No. VII-25 in Example VII-25 was used to afford Cpd. No. VII-33 (16.0 mg) as a white solid. H NMR (400 MHI-z, DMSO-d₆) δ 10.18 (s, 1H), 10.00 (s, 1H), 7.92-7.82 (m, 3H), 7.75 (s, 1H), 7.62 (s, 1H), 7.32 (d, J=9.0 Hz, 2H), 6.55 (s., 1H), 5.00-4.75 (m, 1H), 4.54-4.23 (m, 2H), 3.88-3.29 (m, 7H), 2.99-2.97 (m, 2H), 2.86 (s, 3H), 0.95-0.94 (m, 3H), 0.82-0.79 (in, 3H). MS: 573.2 (M+H)⁺.

### Example VII-34

### Synthesis of N-(4-(chlorodifluoromethoxy)phenyl)-2-(hydroxymethyl)-1-isopropyl-7-(1H-pyrazol-5-yl)indoline-5-carboxamide (Cpd. No. VII-34)

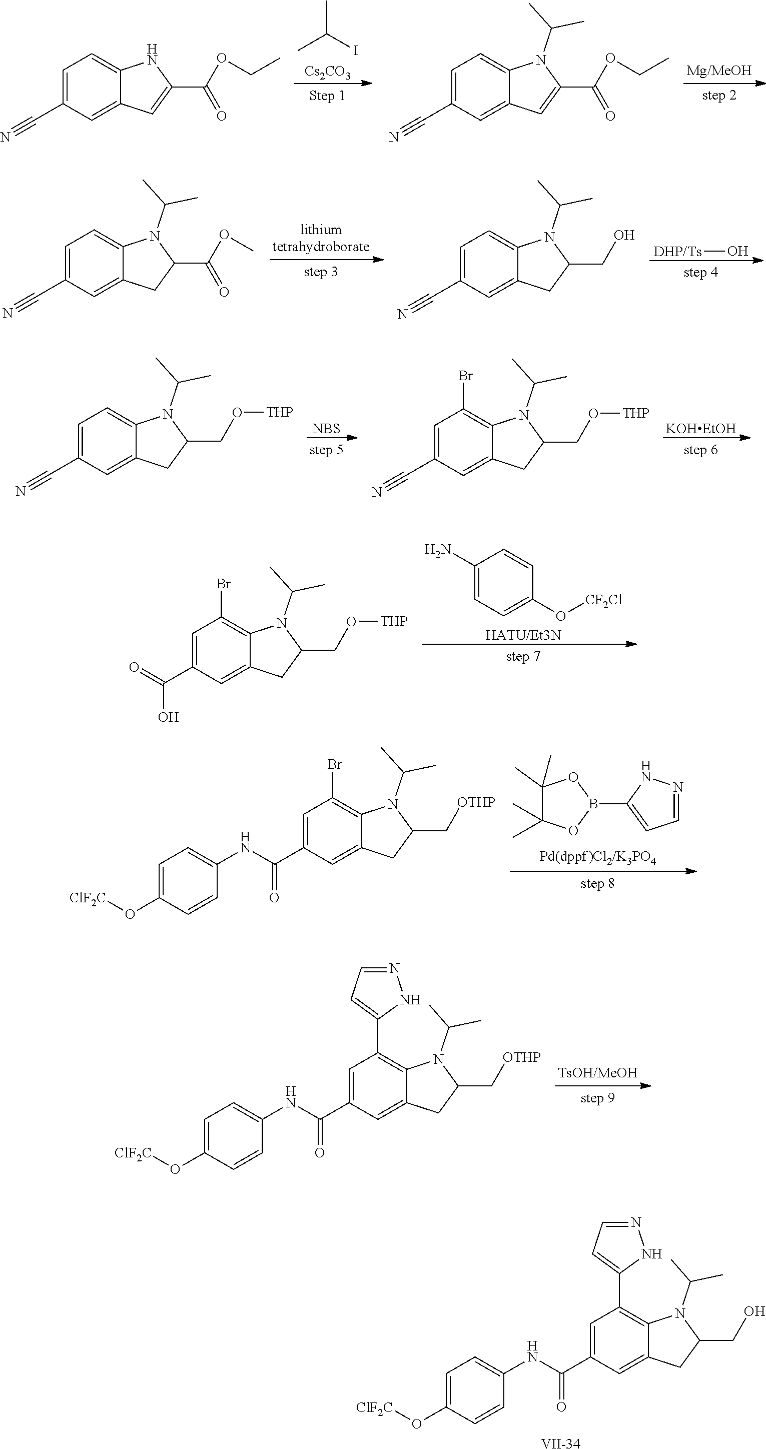

### Step 1: Synthesis of ethyl 5-cyano-1-isopropyl-1H-indole-2-carboxylate

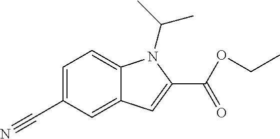 [SMILES: CCOC(=O)c1cc2cc(C#N)ccc2n1C(C)C]

Under Ar, a mixture of ethyl 5-cyano-1H-indole-2-carboxylate (4.5 g, 21.01 mmol), Cs₂CO₃ (13.69 g, 42.0 mmol) and 2-iodopropane (7.14 g, 42.0 mmol) in MeCN (50 mL) was stirred at 70° C. for 8 h. After cooling to room temperature, the filtrate was concentrated in vacuo to give a residue which was eluted through a silica gel column (hexane/ethyl acetate=5/1) to give ethyl 5-cyano-1-isopropyl-1H-indole-2-carboxylate (3.5 g, 65.0%) as a white solid. MS: 257.4 (M+H)⁺.

### Step 2: Synthesis of methyl 5-cyano-1-isopropylindoline-2-carboxylate

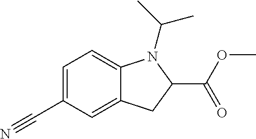 [SMILES: COC(=O)C1Cc2cc(C#N)ccc2N1C(C)C]

Under Ar, to a solution of ethyl 5-cyano-1-isopropyl-1H-indole-2-carboxylate (3.5 g, 13.66 mmol) in methanol (50 mL) was added magnesium (0.730 g, 30.0 mmol) under ice-water bath, then stirred at room temperature for 5 h. The mixture was quenched with HCl solution (1N), then concentrated to give a residue that was partitioned between ethyl acetate (40 mL)/water (15 mL), the organic layer was concentrated in vacuo to give a light yellow oil, which was eluted through a silica gel column (hexane/ethyl acetate=5/1) to give methyl 5-cyano-1-isopropylindoline-2-carboxylate (2 g, 60.0%) as a light yellow solid. ESI-MS: 245.4 (M+H)⁺.

### Step 3: Synthesis of 2-(hydroxymethyl)-1-isopropylindoline-5-carbonitrile

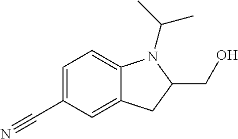 [SMILES: CC(C)N1c2ccc(C#N)cc2CC1CO]

Under Ar, a mixture of methyl 5-cyano-1-isopropylindoline-2-carboxylate (500 mg, 2.047 mmol) and lithium tetrahydroborate (66.9 mg, 3.07 mmol) in tetrahydrofuran (10 mL) was stirred at room temperature for over night. The reaction mixture was quenched with aqueous NH₄Cl, then partitioned between ethyl acetate (20 mL)/water (10 mL). The organic layer was washed with brine, dried over Na₂SO₄, filtered and concentrated in vacuo to give crude 2-(hydroxymethyl)-1-isopropylindoline-5-carbonitrile (480 mg, crude), which was used for the next without purification as a colourless oil. ESI-MS: 217.4 (M+H)⁺.

### Step 4: Synthesis of 1-isopropyl-2-(((tetrahydro-2H-pyran-2-yl)oxy)methyl)indoline-5-carbonitrile

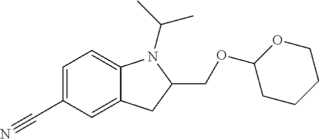 [SMILES: CC(C)N1c2ccc(C#N)cc2CC1COC1CCCCO1]

The mixture of 2-(hydroxymethyl)-1-isopropylindoline-5-carbonitrile (480 mg, 2.219 mmol), DHP (560 mg, 6.66 mmol) and p-toluenesulfonic acid (42.2 mg, 0.222 mmol) in tetrahydrofuran (10 mL) was stirred at room temperature for over night, then partitioned between ethyl acetate (20 mL)/water (10 mL). The organic layer was concentrated in vacuo to give the residual which was eluted through a silica gel column (hexane/ethyl acetate=10/1) to give 1-isopropyl-2-(((tetrahydro-2H-pyran-2-yl)oxy)methyl)indoline-5-carbonitrile (580 mg, 87%) as a colourless oil. ESI-MS: 301.4 (M+H)⁺.

### Step 5: Synthesis of 7-bromo-1-isopropyl-2-(((tetrahydro-2H-pyran-2-yl)oxy)methyl)indoline-5-carbonitrile

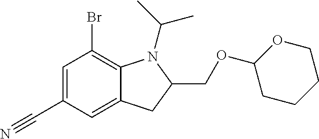 [SMILES: CC(C)N1c2c(Br)cc(C#N)cc2CC1COC1CCCCO1]

Under Ar, to a solution of 1-isopropyl-2-(((tetrahydro-2H-pyran-2-yl)oxy)methyl)indoline-5-carbonitrile (580 mg, 1.931 mmol) in 1,4-dioxane (10 mL) was added N-Bromosuccinimide (378 mg, 2.124 mmol) under ice-water bath, then stirred at room temperature for 2 h. The mixture was diluted with ethyl acetate (50 mL) and washed with water (100 mL). The organic layer was concentrated in vacuo to give a crude product that was eluted through a silica gel column (hexane/ethyl acetate=10/1) to give 7-bromo-1-isopropyl-2-(((tetrahydro-2H-pyran-2-yl)oxy)methyl)indoline-5-carbonitrile (460 mg, 62.8%) as light yellow solid. ESI-MS: 379.4 (M+H)⁺.

### Step 6: Synthesis of 7-bromo-1-isopropyl-2-(((tetrahydro-2H-pyran-2-yl)oxy)methyl)indoline-5-carboxylic acid

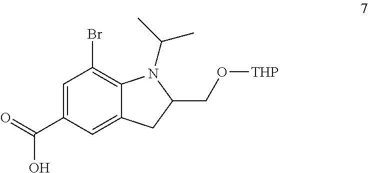

Under Ar, a mixture of 7-bromo-1-isopropyl-2-(((tetrahydro-2H-pyran-2-yl)oxy)methyl)indoline-5-carbonitrile (400 mg, 1.055 mmol) and KOH (592 mg, 10.55 mmol) in EtOH (10 mL)/water (2 mL) was stirred at refluxing for over night. The mixture was neutralized with HCl, evaporated under vacuum to remove ethanol, and partitioned between ethyl acetate/water. The organic layer was concentrated in vacuo to give 7-bromo-1-isopropyl-2-(((tetrahydro-2H-pyran-2-yl)oxy)methyl)indoline-5-carboxylic acid (400 mg, 95%) as a colourless oil which was used directly in the next step without purification. ESI-MS: 398.4 (M+H)⁺.

### Step 7: Synthesis of 7-bromo-N-(4-(chlorodifluoromethoxy)phenyl)-1-isopropyl-2-(((tetrahydro-2H-pyran-2-yl)oxy)methyl)indoline-5-carboxamide

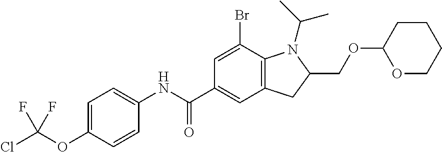 [SMILES: CC(C)N1c2c(Br)cc(C(=O)Nc3ccc(OC(F)(F)Cl)cc3)cc2CC1COC1CCCCO1]

A mixture of 7-bromo-1-isopropyl-2-(((tetrahydro-2H-pyran-2-yl)oxy)methyl)indoline-5-carboxylic acid (350 mg, 0.879 mmol), 4-(chlorodifluoromethoxy)aniline (340 mg, 1.758 mmol), Et₃N (266 mg, 2.637 mmol) and 2-(7-azabenzotriazol-1-yl)-N,N,N′,N′-tetramethyluronium hexafluorophosphate (668 mg, 1.758 mmol) in N,N-dimethylformamide (10 mL) was stirred at room temperature for over night. Then the mixture was partitioned between ethyl acetate/water. The organic layer was concentrated in vacuo to give a residue which was eluted through a silica gel column (hexane/ethyl acetate=2/1) to give 7-bromo-N-(4-(chlorodifluoromethoxy)phenyl)-1-isopropyl-2-(((tetrahydro-2H-pyran-2-yl)oxy)methyl)indoline-5-carboxamide (350 mg, 70%) as a white solid. ESI-MS: 495.5 (M+H)⁺.

### Step 8: Synthesis of N-(4-(chlorodifluoromethoxy)phenyl)-1-isopropyl-7-(1H-pyrazol-5-yl)-2-(((tetrahydro-2H-pyran-2-yl)oxy)methyl)indoline-5-carboxamide

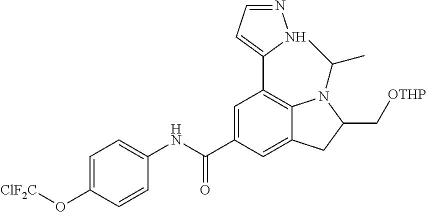

Under Ar, a mixture of 7-bromo-N-(4-(chlorodifluoromethoxy)phenyl)-1-isopropyl-2-(((tetrahydro-2H-pyran-2-yl)oxy)methyl)indoline-5-carboxamide (100 mg, 0.174 mmol), 5-(4,4,5,5-tetramethyl-1,3,2-dioxaborolan-2-yl)-1H-pyrazole (85 mg, 0.436 mmol) K₃PO₄ (148 mg, 0.697 mmol) and PdCl₂(dppf)-CH₂Cl₂ adduct (14.23 mg, 0.017 mmol) in 1,4-dioxane (5 mL)/water (1 mL) was stirred at 110° C. for 2 h under microwave. The mixture was partitioned between ethyl acetate/water. The organic layer was concentrated in vacuo to give crude N-(4-(chlorodifluoromethoxy)phenyl)-1-isopropyl-7-(1H-pyrazol-5-yl)-2-(((tetrahydro-2H-pyran-2-yl)oxy)methyl)indoline-5-carboxamide (100 mg, crude) as a black oil which was used directly in the next step. ESI-MS: 561.5 (M+H)⁺.

### Step 9: Synthesis of N-(4-(chlorodifluoromethoxy)phenyl)-2-(hydroxymethyl)-1-isopropyl-7-(1H-pyrazol-5-yl)indoline-5-carboxamide (Cpd. No. VII-34)

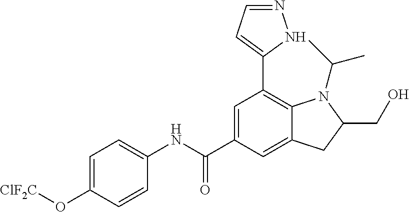

The mixture of N-(4-(chlorodifluoromethoxy)phenyl)-1-isopropyl-7-(1H-pyrazol-5-yl)-2-(((tetrahydro-2H-pyran-2-yl)oxy)methyl)indoline-5-carboxamide (100 mg, 0.178 mmol) and p-toluenesulfonic acid (10.17 mg, 0.053 mmol) in methanol (5 mL) was stirred at room temperature for over night. The solvent was removed in vacuo to give a crude product which was purified by preparative HPLC to give N-(4-(chlorodifluoromethoxy)phenyl)-2-(hydroxymethyl)-1-isopropyl-7-(1H-pyrazol-5-yl)indoline-5-carboxamide (12 mg, 14.12%) as a light grey solid. ¹H NMR (400 MHz, DMSO-d₆) δ 12.88 (s, 1H), 10.22 (s, 1H), 8.01-7.84 (m, 3H), 7.73-7.65 (m, 2H), 7.33 (d, J=8.7 Hz, 2H), 6.63 (s, 1H), 4.78 (t, J=6.0 Hz, 1H), 3.79 (t, J=7.0 Hz, 1H), 3.49-3.37 (m, 2H), 3.22-3.15 (m, 2H), 2.84 (d, J=16.5 Hz, 1H), 1.11 (d, J=6.8 Hz, 3H), 0.79 (d, J=6.4 Hz, 3H). MS: 477.5 (M+H)⁺.

### Example VII-35

### Synthesis of N-(4-(chlorodifluoromethoxy)phenyl)-1-isopropyl-2-(2-morpholino-2-oxoethyl)-7-(1H-pyrazol-5-yl)indoline-5-carboxamide (Cpd. No. VII-35)

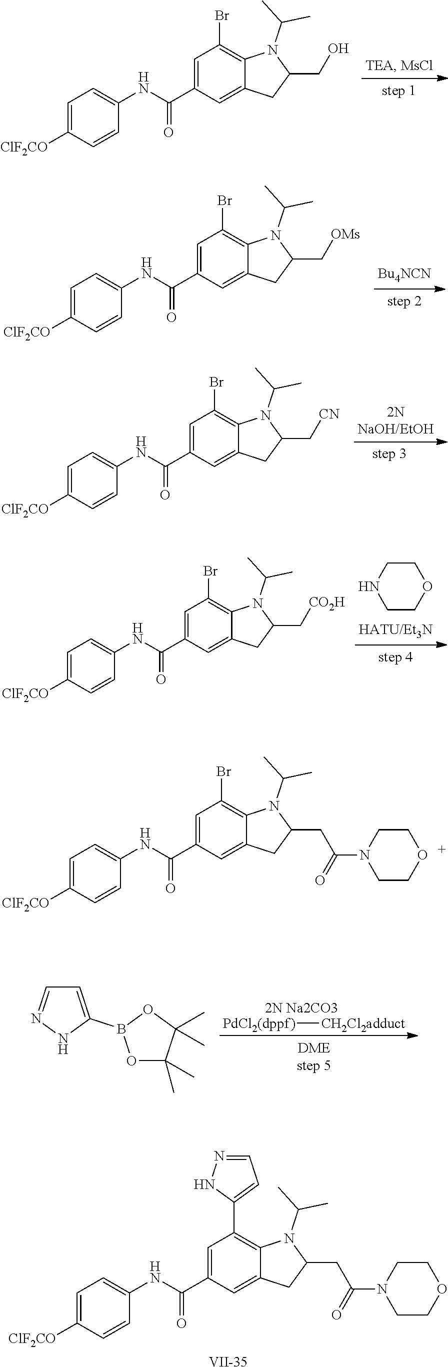

### Step 1: Synthesis of (7-bromo-5-((4-(chlorodifluoromethoxy)phenyl)carbamoyl)-1-isopropylindolin-2-yl)methyl methanesulfonate

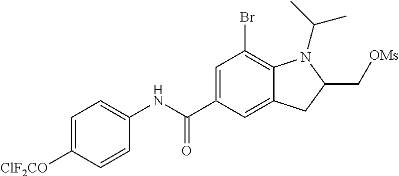

Under Ar, methanesulfonyl chloride (32.3 mg, 0.282 mmol) was added to a solution of 7-bromo-N-(4-(chlorodifluoromethoxy)phenyl)-2-(hydroxymethyl)-1-isopropylindoline-5-carboxamide (115 mg, 0.235 mmol) and TEA (47.5 mg, 0.470 mmol) in dichloromethane (10 mL) at 0° C. It was stirred at room temperature for 0.5 h. The reaction was quenched with NH₄Cl aqueous solution, the organic layer was separated, and the aqueous layer was extracted with ethyl acetate. The combined organic layers were washed with brine, dried over anhydrous Na₂SO₄, filtered and concentrated to give a residue which was eluted through a silica gel column (hexane:ethyl acetate=3:1) to afford the compound (7-bromo-5-((4-(chlorodifluoromethoxy)phenyl) carbamoyl)-1-isopropylindolin-2-yl)methyl methanesulfonate (140 mg, crude). MS: 567.1 (M+H)⁺.

### Step 2: Synthesis of 7-bromo-N-(4-(chlorodifluoromethoxy)phenyl)-2-(cyanomethyl)-1-isopropylindoline-5-carboxamide

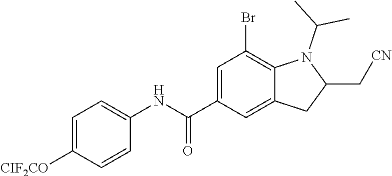

Under Ar, a mixture of tetrabutyl ammonium fluoride (414.43 mg, 1.585 mmol) and TMS-CN (175 mg, 1.761 mmol) in tetrahydrofuran (2.0 mL) was stirred at room temperature for 0.5 h. Then (7-bromo-5-((4-(chlorodifluoromethoxy)phenyl)carbamoyl)-1-isopropylindolin-2-yl)methyl methanesulfonate (200 mg, 0.352 mmol) in N,N-dimethylformamide (5.0 mL) was added. The resulting mixture was stirred at 80° C. for 18 h. The solution was partitioned between ethyl acetate/water, and the organic layers were washed with brine, dried over anhydrous Na₂SO₄, filtered and concentrated to give a residue which was eluted through a silica gel column (hexane:ethyl acetate=3:1) to afford the compound 7-bromo-N-(4-(chlorodifluoromethoxy)phenyl)-2-(cyanomethyl)-1-isopropylindoline-5-carboxamide (170 mg, 97%). MS: 498.1 (M+H)⁺.

### Step 3: Synthesis of 2-(7-bromo-5-((4-(chlorodifluoromethoxy)phenyl) carbamoyl)-1-isopropylindolin-2-yl)acetic acid

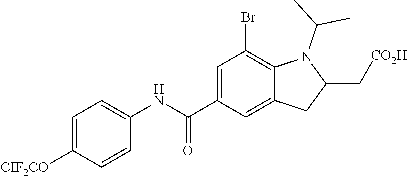

A solution of 7-bromo-N-(4-(chlorodifluoromethoxy)phenyl)-2-(cyanomethyl)-1-isopropylindoline-5-carboxamide (100 mg, 0.201 mmol) in aqueous 2N NaOH solution (4.0 mL) and EtOH (5.0 mL) was stirred at 70° C. for 18 h. The mixture was evaporated to remove ethanol, then neutralized with 2N HCl solution. The resulting precipitate was collected and dried to afford 2-(7-bromo-5-((4-(chlorodifluoromethoxy)phenyl)carbamoyl)-1-isopropylindolin-2-yl)acetic acid (50 mg, 48.2%) as a white powder which was used in the next step without purification. MS: 518.3 (M+H)⁺.

### Step 4: Synthesis of 7-bromo-N-(4-(chlorodifluoromethoxy)phenyl)-1-isopropyl-2-(2-morpholino-2-oxoethyl)indoline-5-carboxamide

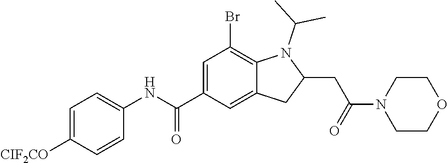

Morpholine (81.0 mg, 0.927 mmol), TEA (31.3 mg, 0.309 mmol) and 2-(7-azabenzotriazol-1-yl)-N,N,N′,N′-tetramethyluronium hexafluorophosphate (58.8 mg, 0.155 mmol) were added to a solution of 2-(7-bromo-5-((4-(chlorodifluoromethoxy)phenyl)carbamoyl)-1-isopropylindolin-2-yl)acetic acid (32.0 mg, 0.062 mmol) in N,N-dimethylformamide (3.0 mL). The reaction mixture was stirred at 50° C. for 5 h. The reaction mixture was diluted with ethyl acetate (40.0 mL), then washed with water (40.0 mL) and saturated aqueous NaCl (40.0 mL). The organic layer was dried over Na₂SO₄, filtered and concentrated to give a crude product that was eluted through a silica gel column (ethyl acetate/hexane from 0% to 30%) to give 7-bromo-N-(4-(chlorodifluoromethoxy)phenyl)-1-isopropyl-2-(2-morpholino-2-oxoethyl)indoline-5-carboxamide (21.0 mg, 57.9%).

### Step 5: Synthesis of N-(4-(chlorodifluoromethoxy)phenyl)-1-isopropyl-2-(2-morpholino-2-oxoethyl)-7-(1H-pyrazol-5-yl)indoline-5-carboxamide (Cpd. No. VII-35)

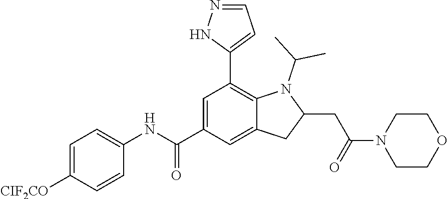

Essentially the same protocol of the preparation of Cpd. No. VII-34 in Example VII-34 was used to afford Cpd. No. VII-35 (6.4 mg) as a white solid. ¹H NMR (400 MHz, DMSO-d₆) δ 13.06-12.85 (m, 1H), 10.23 (s, 1H), 8.12-7.97 (m, 1H), 7.88 (d, J=8.9 Hz, 2H), 7.83-7.77 (m, 1H), 7.68 (s, 1H), 7.33 (d, J=8.6 Hz, 2H), 6.76-6.55 (m, 1H), 4.21-4.11 (m, 1H), 3.68-3.42 (m, 11H), 2.70-2.54 (m, 2H), 1.08 (d, J=6.6 Hz, 3H), 0.79 (d, J=6.0 Hz, 3H). MS: 575.8 (M+H)⁺.

### Example VII-36

### Synthesis of N-(4-(chlorodifluoromethoxy)phenyl)-2-(2-(dimethylamino)-2-oxoethyl)-1-isopropyl-7-(1H-pyrazol-5-yl)indoline-5-carboxamide (Cpd. No. VII-36)

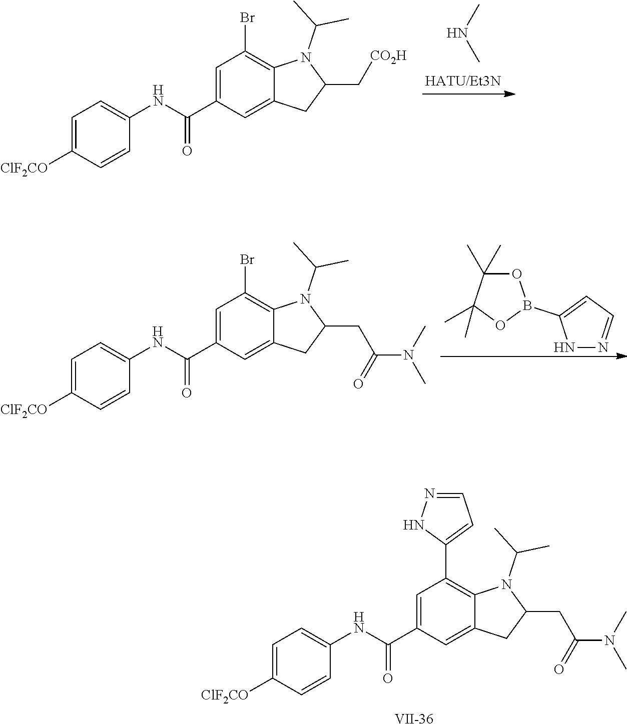

Essentially the same protocol of the preparation of Cpd. No. VII-35 in Example VII-35 was used to afford Cpd. No. VII-36 (21.0 mg) as a white solid. ¹H NMR (400 MHz, DMSO-d₆) δ 12.90 (br, 1H), 10.23 (s, 1H), 7.99 (s, 1H), 7.88 (d, J=9.1 Hz, 2H), 7.74 (s, 1H), 7.69 (s, 1H), 7.33 (d, J=8.9 Hz, 2H), 6.65 (s, 1H), 4.15 (dd, J=14.5, 7.1 Hz, 1H), 3.56-3.44 (m, 1H), 3.31-3.22 (m, 2H), 2.96 (s, 3H), 2.85 (s, 3H), 2.65-2.53 (m, 2H), 1.06 (d, J=6.8 Hz, 3H), 0.79 (d, J=6.5 Hz, 3H). MS: 533.7 (M+H)⁺.

### Example VII-37

### Synthesis of N-(4-(chlorodifluoromethoxy)phenyl)-1-isopropyl-3-methyl-7-(pyrimidin-5-yl)indoline-5-carboxamide (Cpd. No. VII-37)

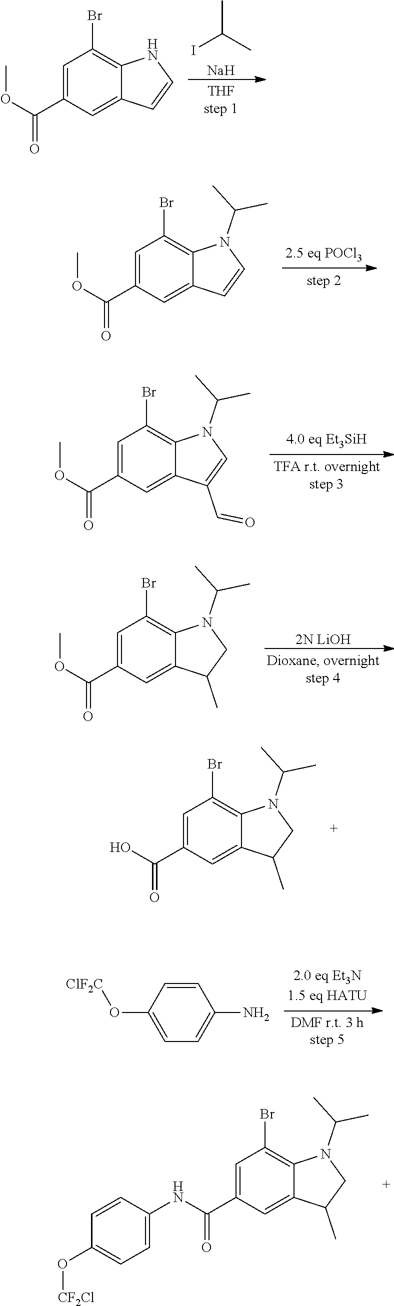

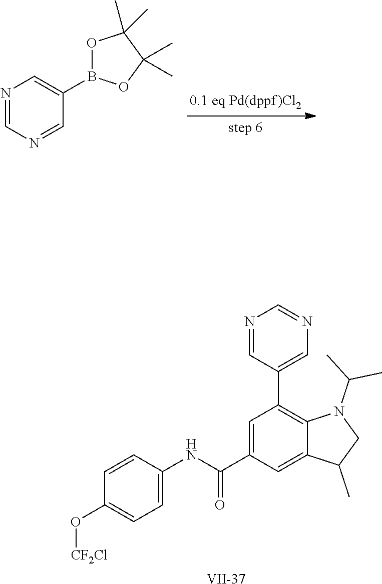

### Step 1: Synthesis of methyl 7-bromo-1-isopropyl-1H-indole-5-carboxylate

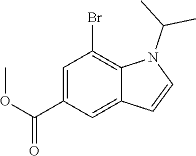 [SMILES: COC(=O)c1cc(Br)c2c(ccn2C(C)C)c1]

In a 25 mL round-bottomed flask, to a suspension of NaH (18.89 mg, 0.878 mmol) in tetrahydrofuran (10 mL) was added a solution of methyl 7-bromo-1H-indole-5-carboxylate (100 mg, 0.394 mmol) in tetrahydrofuran (20 mL) under ice/water bath. After being stirred at the same temperature, to the mixture was added 2-iodopropane (100 mg, 0.59 mmol), and the mixture was warmed to 30° C. and stirred for 4-5 h. The mixture was cooled to 0° C., quenched with NH₄Cl aqueous solution, and diluted with water (20 mL), followed by extraction with ethyl acetate (15 mL×3). The combined organic layers were dried over Na₂SO₄, filtered and concentrated. The crude product was eluted through a silica gel column (ethyl acetate/hexane from 10% to 40%) to give methyl 7-bromo-1-isopropyl-1H-indole-5-carboxylate (80 mg, 69.6%). MS: 297.02 (M+H)⁺.

### Step 2: Synthesis of methyl 7-bromo-3-formyl-1-isopropyl-1H-indole-5-carboxylate

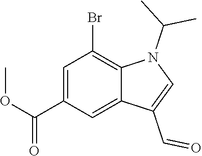 [SMILES: COC(=O)c1cc(Br)c2c(c1)c(C=O)cn2C(C)C]

To N,N-dimethylformamide (11 mL) was added phosphoryl trichloride (518 mg, 3.38 mmol) at 0° C., then was added a solution of methyl 7-bromo-1-isopropyl-1H-indole-5-carboxylate (400 mg, 1.351 mmol) in N,N-dimethylformamide (4 mL) at the same temperature. The mixture was stirred at 10° C. for 40 min, then warmed up to 35° C. and stirred for another 40 min. After the reaction was completed, the mixture was quenched with water (20 mL), followed by extraction with ethyl acetate (10 mL×3). The combined organic layers were washed with brine, dried over Na₂SO₄, filtered and concentrated to give a crude product that was eluted through a silica gel column (ethyl acetate/hexane from 10% to 40%) to give methyl 7-bromo-3-formyl-1-isopropyl-1H-indole-5-carboxylate (315 mg, 71.9%) as a white solid. ¹H NMR (400 MHz, chloroform-d) δ 10.08 (s, 1H), 9.01 (d, J=1.6 Hz, 1H), 8.23 (d, J=1.6 Hz, 1H), 8.02 (s, 1H), 5.99-5.84 (m, 1H), 3.97 (s, 3H), 1.64 (d, J=6.6 Hz, 6H). MS: 324.95 (M+H)⁺.

### Step 3: Synthesis of methyl 7-bromo-1-isopropyl-3-methylindoline-5-carboxylate

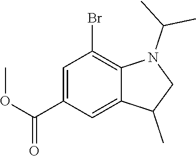 [SMILES: COC(=O)c1cc(Br)c2c(c1)C(C)CN2C(C)C]

To a solution of methyl 7-bromo-3-formyl-1-isopropyl-1H-indole-5-carboxylate (110 mg, 0.339 mmol) in trifluoroacetic acid (6 mL) was added triethylsilane (158 mg, 1.357 mmol) in a 25 mL round-bottomed flask. The mixture was stirred for overnight at room temperature, then evaporated to remove trifluoroacetic acid to give a crude product that was purified by silica gel column chromatography (ethyl acetate/hexane from 5% to 40%) to give methyl 7-bromo-1-isopropyl-3-methylindoline-5-carboxylate (100 mg, 94%) as a colorless oil. ¹H NMR (400 MHz, chloroform-d) δ 7.98-7.92 (m, 1H), 7.58-7.52 (m, 1H), 5.20-5.05 (m, 1H), 3.86 (s, 3H), 3.71 (t, J=9.5 Hz, 1H), 3.32-3.18 (m, 1H), 3.15-3.06 (m, 1H), 1.31 (d, J=6.8 Hz, 3H), 1.23 (d, J=6.7 Hz, 3H), 1.18 (d, J=6.6 Hz, 3H). MS: 312.10 (M+H)⁺.

### Step 4: Synthesis of 7-bromo-1-isopropyl-3-methylindoline-5-carboxylic acid

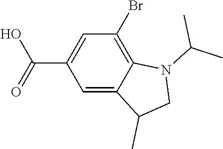 [SMILES: CC1CN(C(C)C)c2c(Br)cc(C(=O)O)cc21]

To a solution of methyl 7-bromo-1-isopropyl-3-methylindoline-5-carboxylate (110 mg, 0.352 mmol) in 1,4-dioxane (2 mL) was added lithium hydroxide (1N, 1 mL) in a 25 mL round-bottomed flask. After being stirred at 40° C. for overnight, the reaction mixture was acidified with 1N HCl, and extracted with ethyl acetate (30 mL*3). The organic layers were combined, washed with brine, dried over Na₂SO₄, filtered and concentrated to give a crude product that was eluted through a silica gel column (ethyl acetate/hexane from 5% to 40%) to give 7-bromo-1-isopropyl-3-methylindoline-5-carboxylic acid (100 mg, 95%) as a colorless oil. MS: 299.00, 300.95 (M+H)⁺.

### Step 5: Synthesis of 7-bromo-N-(4-(chlorodifluoromethoxy)phenyl)-1-isopropyl-3-methylindoline-5-carboxamide

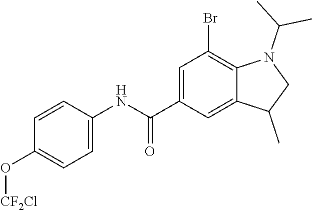

To a solution of methyl 7-bromo-1-isopropyl-3-methylindoline-5-carboxylate (80 mg, 0.256 mmol) in N,N-dimethylformamide (4 mL) were added Et₃N (51.9 mg, 0.512 mmol), 2-(7-azabenzotriazol-1-yl)-N,N,N′,N′-tetramethyluronium hexafluorophosphate (146 mg, 0.384 mmol), and 4-(chlorodifluoromethoxy)aniline (59.5 mg, 0.307 mmol) in a 25 mL round-bottomed flask. The reaction mixture was stirred for overnight, then quenched with water, followed by extraction with ethyl acetate (30 mL×3). The combined organic layers were concentrated to give a crude product that was eluted through a silica gel column (ethyl acetate/hexane from 5% to 40%) to give 7-bromo-N-(4-(chlorodifluoromethoxy)phenyl)-1-isopropyl-3-methylindoline-5-carboxamide (70 mg, 57.7%). ¹H NMR (400 MHz, chloroform-d) δ 7.74-7.71 (m, 1H), 7.70-7.65 (m, 3H), 7.49-7.43 (m, 1H), 7.27-7.23 (m, 2H), 5.16-5.03 (m, 1H), 3.73 (t, J=9.5 Hz, 1H), 3.37-3.22 (m, 1H), 3.18-3.09 (m, 1H), 1.34 (d, J=6.8 Hz, 3H), 1.24 (d, J=6.7 Hz, 3H), 1.19 (d, J=6.6 Hz, 3H). MS: 473.90, 475.90 (M+H)⁺.

### Step 6: Synthesis of N-(4-(chlorodifluoromethoxy)phenyl)-1-isopropyl-3-methyl-7-(pyrimidin-5-yl)indoline-5-carboxamide (Cpd. No. VII-37)

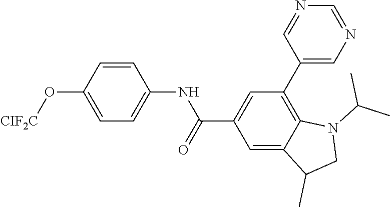

To a solution of methyl 7-bromo-1-isopropyl-3-methylindoline-5-carboxylate (48 mg, 0.154 mmol) in dimethoxyethane (1.5 mL) and water (0.5 mL) was added 5-(4,4,5,5-tetramethyl-1,3,2-dioxaborolan-2-yl)pyrimidine (47.5 mg, 0.231 mmol), PdCl₂(dppf)-CH₂Cl₂ adduct (12.56 mg, 0.015 mmol) and Na₂CO₃ (32.6 mg, 0.307 mmol) in a 5 mL microwave tube. The mixture was stirred at 110° C. under MW for 2 h under nitrogen. The mixture was quenched with water and extracted with ethyl acetate (30 mL×3). The combined organic layers were concentrated to give a crude product that was purified by silica gel column chromatography (ethyl acetate/hexane from 5% to 40%) to give N-(4-(chlorodifluoromethoxy)phenyl)-1-isopropyl-3-methyl-7-(pyrimidin-5-yl)indoline-5-carboxamide (3.5 mg, 4.81%). ¹H NMR (400 MHz, chloroform-d) δ 9.24 (s, 1H), 8.85 (s, 2H), 7.74-7.65 (m, 3H), 7.62-7.56 (m, 1H), 7.45-7.40 (m, 1H), 7.29-7.22 (m, 2H), 3.73 (t, J=9.7 Hz, 1H), 3.46-3.30 (m, 2H), 3.16-3.06 (m, 1H), 1.42 (d, J=6.8 Hz, 3H), 1.00 (d, J=6.6 Hz, 3H), 0.91 (d, J=6.4 Hz, 3H). MS: 474.00 (M+H)⁺.

### Example VII-38

### Synthesis of N-(4-(chlorodifluoromethoxy)phenyl)-1-isopropyl-3-methyl-7-(1H-pyrazol-5-yl)indoline-5-carboxamide (Cpd. No. VII-38)

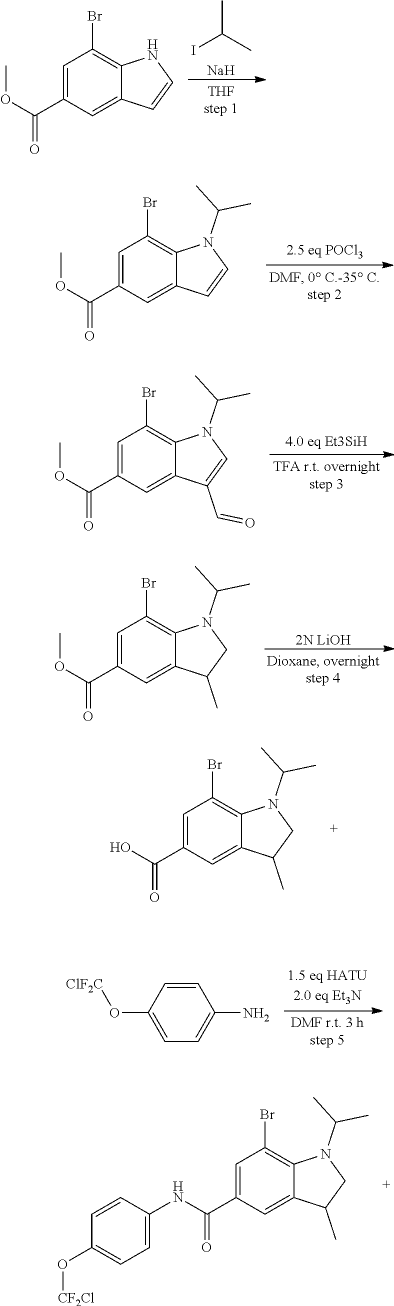

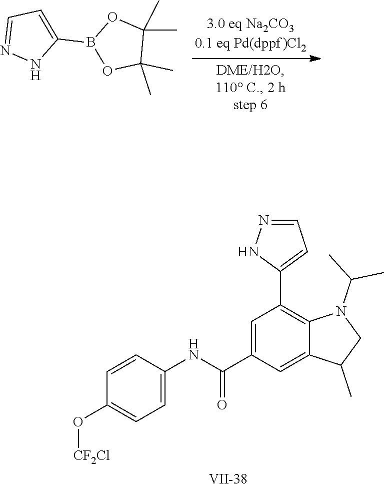

Essentially the same protocol of the preparation of Cpd. No. VII-37 in Example VII-37 was used to afford Cpd. No. VII-38 (5.0 mg, 4.52%) as a white solid. ¹H NMR (400 MHz, chloroform-d) δ 8.16 (s, 1H), 7.76-7.69 (m, 2H), 7.69-7.62 (m, 3H), 7.60 (s, 1H), 7.26-7.21 (m, 2H), 6.48 (s, 1H), 3.71 (t, J=9.7 Hz, 1H), 3.66-3.56 (m, 1H), 3.42-3.31 (m, 1H), 3.13-3.04 (m, 1H), 1.38 (d, J=6.8 Hz, 3H), 0.99 (d, J=6.6 Hz, 3H), 0.92 (d, J=6.5 Hz, 3H). MS: 462.00 (M+H)⁺.

### Example VII-39

### Synthesis of N-(4-(chlorodifluoromethoxy)phenyl)-1,3-dimethyl-7-(1H-pyrazol-5-yl)indoline-5-carboxamide (Cpd. No. VII-39)

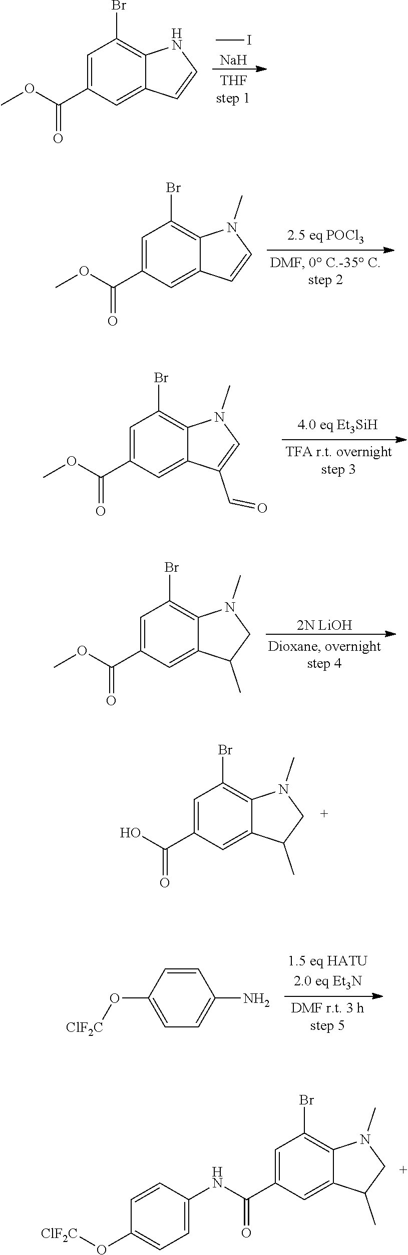

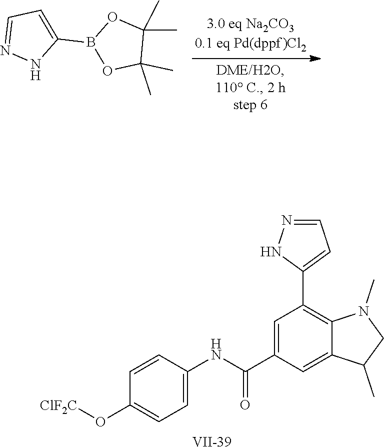

Essentially the same protocol of the preparation of Cpd. No. VII-37 in Example VII-37 was used to afford Cpd. No. VII-39 (30.0 mg, 28.1%) as a white solid. ¹H NMR (400 MHz, chloroform-d) δ 8.06 (s, 1H), 7.71 (d, J=8.9 Hz, 2H), 7.69-7.68 (m, 1H), 7.67-7.66 (m, 1H), 7.63-7.61 (m, 1H), 7.25 (d, J=8.7 Hz, 2H), 6.50-6.45 (m, 1H), 3.72 (t, J=9.4 Hz, 1H), 3.49-3.34 (m, 1H), 3.06 (t, J=8.8 Hz, 1H), 2.56 (d, J=0.8 Hz, 3H), 1.41 (d, J=6.9 Hz, 3H). MS: 434.00. (M+H)⁺.

### Example VII-40

### Synthesis of N-(4-(chlorodifluoromethoxy) phenyl)-1-isopropyl-3,3-dimethyl-7-(1H-pyrazol-5-yl) indoline-5-carboxamide (Cpd. No. VII-40)

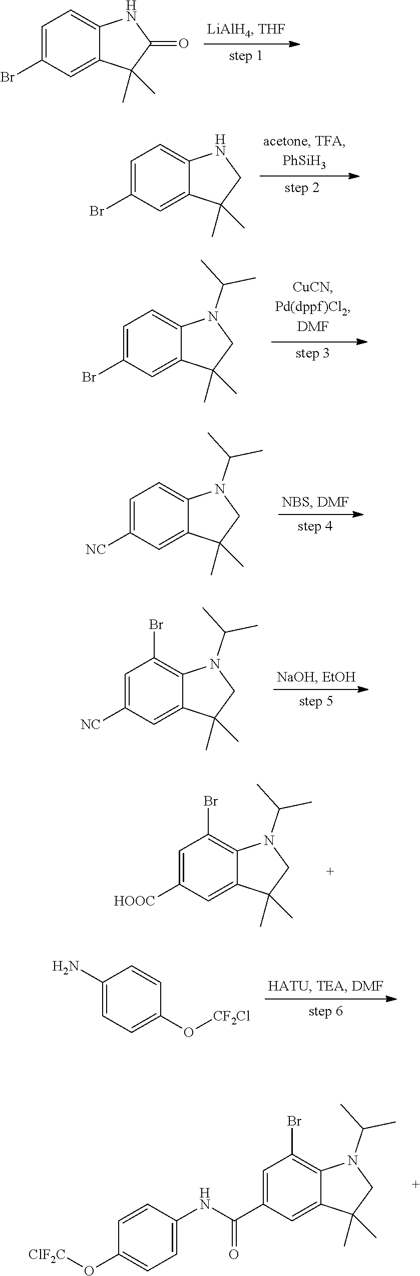

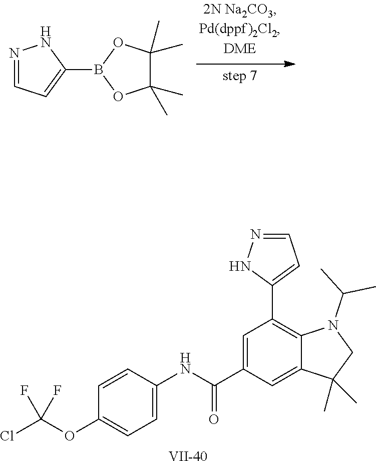

### Step 1: Synthesis of 5-bromo-3,3-dimethylindoline

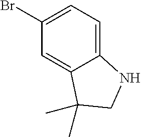

LiAlH₄ (1.18 g, 31.2 mmol) was added to a solution of 5-bromo-3,3-dimethylindolin-2-one (5.00 g, 20.8 mmol) in tetrahydrofuran (50.0 mL) dropwise at room temperature. The mixture was stirred at 70° C. for 2 h. After being cooled to room temperature, the mixture was quenched with Na₂SO₄ in water and filtered. The filtrate was dried over Na₂SO₄, filtered and concentrated to give a crude product (4.4 g, 93.4%) as a white solid which was used in the next step without purification. MS: 226.1 (M+H)⁺.

### Step 2: Synthesis of 5-bromo-1-isopropyl-3,3-dimethylindoline

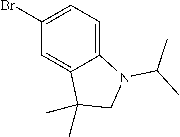

Phenylsilane (2.39 g, 22.1 mmol) was added to a solution of 5-bromo-3,3-dimethylindoline (1.00 g, 4.4 mmol) in dichloromethane/trifluoroacetic acid (2:1). The mixture was stirred at room temperature for 12 h. The mixture was concentrated to give a crude product that was purified by silica gel silica column chromatography (ethyl acetate/hexane from 0% to 30%) to give 5-bromo-1-isopropyl-3,3-dimethylindoline (0.90 g, 76.0%) as a yellow oil. MS: 268.2 (M+H)⁺.

### Step 3: Synthesis of 1-isopropyl-3,3-dimethylindoline-5-carbonitrile

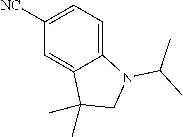 [SMILES: CC(C)N1CC(C)(C)c2cc(C#N)ccc21]

Cyanocopper (0.90 g, 10.1 mmol) and PdCl₂(dppf) (0.24 g, 0.3 mmol) were added to a solution of 5-bromo-1-isopropyl-3,3-dimethylindoline in DMA (20.0 mL). The mixture was purged with nitrogen, then stirred at 140° C. under MW for 1 h. The mixture was diluted with ethyl acetate (100.0 mL), washed with water (100.0 mL) and brine (100.0 mL). The organic layer was dried over Na₂SO₄, filtered and concentrated to give a crude product that was eluted through a silica gel column (ethyl acetate/hexane from 0% to 70%) to give 1-isopropyl-3,3-dimethylindoline-5-carbonitrile (0.50 g, 69.5%) as a yellow solid. MS: 215.2 (M+H)⁺.

### Step 4: Synthesis of 7-bromo-1-isopropyl-3,3-dimethylindoline-5-carbonitrile

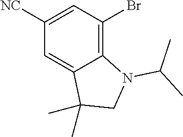 [SMILES: CC(C)N1CC(C)(C)c2cc(C#N)cc(Br)c21]

N-Bromosuccinimide (0.41 g, 2.3 mmol) was added to a solution of 1-isopropyl-3,3-dimethylindoline-5-carbonitrile (0.50 g, 2.3 mmol) in N,N-dimethylformamide (20.0 mL). The mixture was stirred at room temperature for 3 h and concentrated to give a crude product that was eluted through a silica gel column (ethyl acetate/hexane from 0% to 50%) to give 7-bromo-1-isopropyl-3,3-dimethylindoline-5-carbonitrile (0.50 g, 73.1%) as a yellow oil. MS: 293.1 (M+H)⁺.

### Step 5: Synthesis of 7-bromo-1-isopropyl-3,3-dimethylindoline-5-carboxylic acid

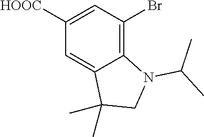 [SMILES: CC(C)N1CC(C)(C)c2cc(C(=O)O)cc(Br)c21]

Sodium hydroxide (7.9 mmol, 2.0 N) was added to a solution of 7-bromo-1-isopropyl-3,3-dimethylindoline-5-carbonitrile (0.50 g, 1.7 mmol) in EtOH (17.0 mL). The mixture was stirred at 95° C. for 50 h, concentrated, acidified with 1N HCl and filtered. The filtrate cake was washed with water and hexane, dried under vacuum to give product (0.4 g, 75.0%) as a white solid. MS: 312.1 (M+H)⁺.

### Step 6: Synthesis of 7-bromo-N-(4-(chlorodifluoromethoxy)phenyl)-1-isopropyl-3,3-dimethylindoline-5-carboxamide

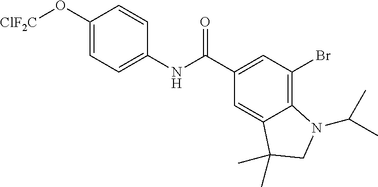

4-(Chlorodifluoromethoxy)aniline (0.25 g, 1.28 mmol), TEA (0.13 g, 1.28 mmol) and 2-(7-azabenzotriazol-1-yl)-N,N,N′,N′-tetramethyluronium hexafluorophosphate (0.49 g, 1.28 mmol) were added to a solution of 7-bromo-1-isopropyl-3,3-dimethylindoline-5-carboxylic acid (0.20 g, 0.64 mmol) in N,N-dimethylformamide (6.0 mL). The mixture was heated to 50° C. and stirred for 5 h. The mixture was diluted with ethyl acetate (40.0 mL), washed with water (40.0 mL) and washed with brine (40.0 mL). The organic layer was dried over Na₂SO₄, filtered and concentrated to give a crude product that was purified by silica gel column chromatography (ethyl acetate/hexane from 0% to 30%) to give 7-bromo-N-(4-(chlorodifluoromethoxy)phenyl)-1-isopropyl-3,3-dimethylindoline-5-carboxamide (0.20 g, 64.0%) as a white solid. MS: 489.1 (M+H)⁺.

### Step 7: Synthesis of N-(4-(chlorodifluoromethoxy)phenyl)-1-isopropyl-3,3-dimethyl-7-(1H-pyrazol-5-yl)indoline-5-carboxamide (Cpd. No. VII-40)

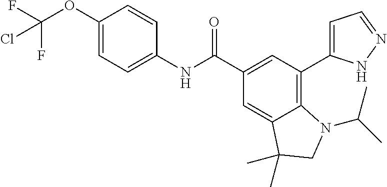 [SMILES: CC(C)N1CC(C)(C)c2cc(C(=O)Nc3ccc(OC(F)(F)Cl)cc3)cc(-c3ccn[nH]3)c21]

5-(4,4,5,5-Tetramethyl-1,3,2-dioxaborolan-2-yl)-1H-pyrazole (0.08 g, 0.4 mmol) and PdCl₂(dppf)-CH₂Cl₂ adduct (0.03 g, 0.04 mmol) were added to a solution of 7-bromo-N-(4-(chlorodifluoromethoxy)phenyl)-1-isopropyl-3,3-dimethylindoline-5-carboxamide (0.10 g, 0.2 mmol) in dimethoxyethane/2N Na₂CO₃ (3:1). The mixture was purged with nitrogen, stirred at 100° C. under MW for 0.5 h. The mixture was diluted with ethyl acetate (30.0 mL), washed with water (30.0 mL), and washed with brine (30.0 mL). The organic layer was dried over Na₂SO₄, filtered and concentrated to give a crude product that was purified using preparative HPLC to give N-(4-(chlorodifluoromethoxy)phenyl)-1-isopropyl-3,3-dimethyl-7-(1H-pyrazol-5-yl)indoline-5-carboxamide (26 mg, 26.7%) as a white solid. ¹H NMR (400 MHz, DMSO-d₆) δ 13.16-12.77 (m, 1H), 10.02 (s, 1H), 7.86 (d, J=9.0 Hz, 2H), 7.82-7.49 (m, 3H), 7.31 (d, J=9.0 Hz, 2H), 6.38-6.34 (m, 1H), 3.77-3.70 (m, 1H), 3.25 (s, 2H), 1.31 (s, 6H), 0.90-0.88 (m, 6H). MS: 475.2 (M+H)⁺.

### Example VII-41

### Synthesis of N-(4-(chlorodifluoromethoxy)phenyl)-3-(hydroxymethyl)-1-isopropyl-3-methyl-7-(1H-pyrazol-5-yl)indoline-5-carboxamide (Cpd. No. VII-41)

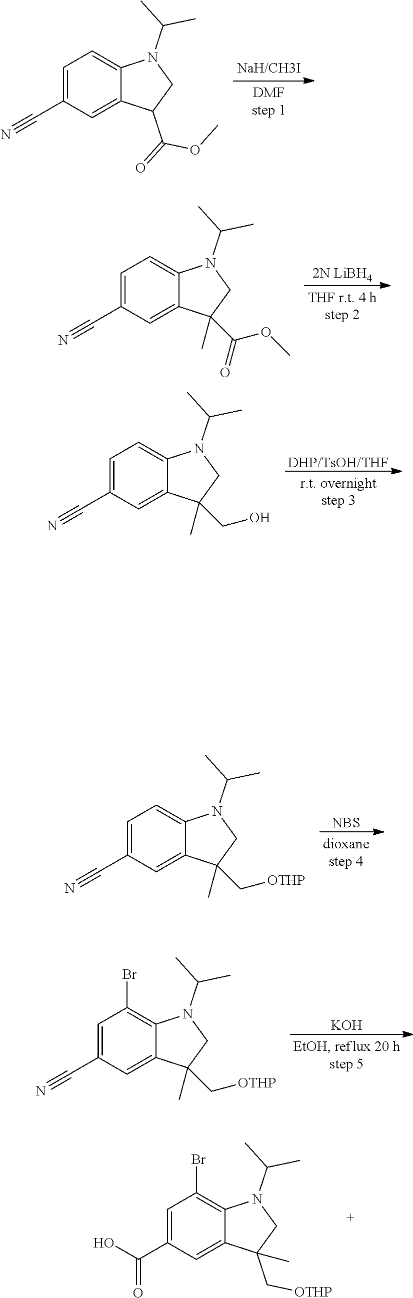

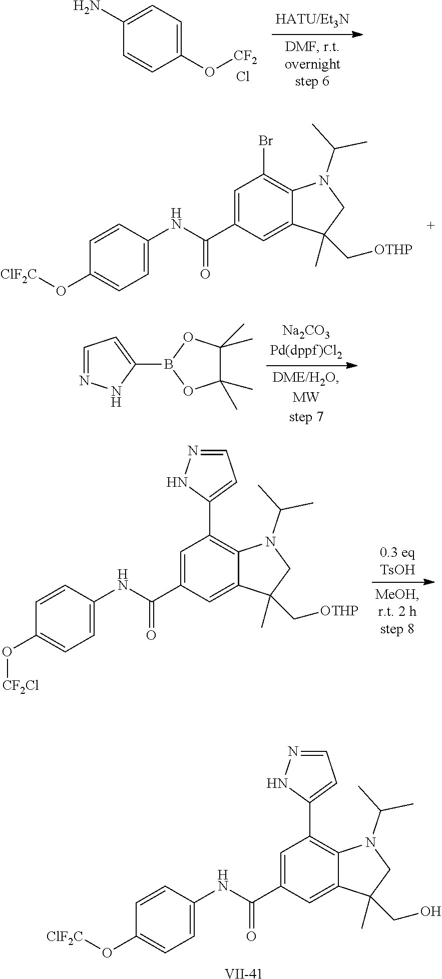

### Step 1: Synthesis of methyl 5-cyano-1-isopropyl-3-methylindoline-3-carboxylate

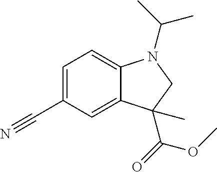 [SMILES: COC(=O)C1(C)CN(C(C)C)c2ccc(C#N)cc21]

In a 100 mL round-bottomed flask, to a suspension of sodium hydride (88 mg, 3.68 mmol) in N,N-dimethylformamide (20 mL) under nitrogen was added a solution of methyl 5-cyano-1-isopropylindoline-3-carboxylate (600 mg, 2.456 mmol) and iodomethane (697 mg, 4.91 mmol) in 10 mL N,N-dimethylformamide dropwise at 5° C. The mixture was stirred for 3 h at 25° C., then quenched with water (30 mL), followed by extraction with dichloromethane (30 mL×3). The combined organic layers were dried over Na₂SO₄, filtered and concentrated to give a crude product that was purified by silica gel column chromatography (ethyl acetate/hexane from 10% to 40%) to give methyl 5-cyano-1-isopropyl-3-methylindoline-3-carboxylate (350 mg, 55.2%) as a colorless oil. ¹H NMR (400 MHz, chloroform-d) δ 7.44 (d, J=1.7 Hz, 1H), 7.39 (dd, J=8.3, 1.7 Hz, 1H), 6.38 (d, J=8.3 Hz, 1H), 4.12 (d, J=9.7 Hz, 1H), 3.95-3.81 (m, 1H), 3.78 (s, 3H), 3.29 (d, J=9.7 Hz, 1H), 1.56 (s, 3H), 1.26-1.17 (m, 6H). MS: 259.10 (M+H)⁺.

### Step 2: Synthesis of 3-(hydroxymethyl)-1-isopropyl-3-methylindoline-5-carbonitrile

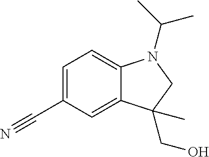 [SMILES: CC(C)N1CC(C)(CO)c2cc(C#N)ccc21]

In a 100 mL round-bottomed flask, to a solution of methyl 5-cyano-1-isopropyl-3-methylindoline-3-carboxylate (300 mg, 1.161 mmol) in tetrahydrofuran (10 mL) under nitrogen, was added LiBH₄ (40 mg, 1.836 mmol). The mixture was stirred at room temperature for 3 h, then quenched with water, followed by extraction with ethyl acetate (30 mL×3). The combined organic layers were washed with brine, dried over Na₂SO₄, filtered and concentrated to give a crude product that was eluted through a silica gel column (ethyl acetate/hexane from 10% to 40%) to give 3-(hydroxymethyl)-1-isopropyl-3-methylindoline-5-carbonitrile (250 mg, 93%) as a colorless oil. ¹H NMR (400 MHz, chloroform-d) δ 7.38 (dd, J=8.3, 1.7 Hz, 1H), 7.18 (d, J=1.7 Hz, 1H), 6.35 (d, J=8.3 Hz, 1H), 3.93-3.79 (m, 1H), 3.67-3.58 (m, 1H), 3.61-3.50 (m, 2H), 3.21 (d, J=9.5 Hz, 1H), 1.33 (s, 3H), 1.23 (d, J=6.7 Hz, 3H), 1.19 (d, J=6.6 Hz, 3H).

### Step 3: Synthesis of 1-isopropyl-3-methyl-3-(((tetrahydro-2H-pyran-2-yl)oxy)methyl)indoline-5-carbonitrile

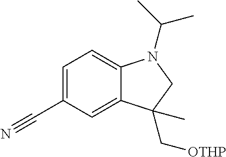

Essentially the same protocol of step 5 in EXAMPLE VII-43 was used to afford 1-isopropyl-3-methyl-3-(((tetrahydro-2H-pyran-2-yl)oxy)methyl)indoline-5-carbonitrile. MS: 314.20 (M+H)⁺.

### Step 4: Synthesis of 7-bromo-1-isopropyl-3-methyl-3-(((tetrahydro-2H-pyran-2-yl)oxy)methyl) indoline-5-carbonitrile

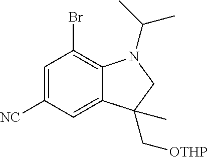

Essentially the same protocol of step 6 in EXAMPLE VII-43 to afford 7-bromo-1-isopropyl-3-methyl-3-(((tetrahydro-2H-pyran-2-yl)oxy)methyl) indoline-5-carbonitrile. MS: 394.11 (M+H)⁺.

### Step 5: Synthesis of 7-bromo-1-isopropyl-3-methyl-3-(((tetrahydro-2H-pyran-2-yl)oxy)methyl) indoline-5-carboxylic acid

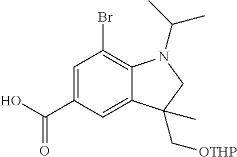

Essentially the same protocol of step 7 in EXAMPLE VII-43 was used to afford 7-bromo-1-isopropyl-3-methyl-3-(((tetrahydro-2H-pyran-2-yl)oxy)methyl) indoline-5-carboxylic acid. MS: 413.10 (M+H)⁺.

### Step 6: Synthesis of 7-bromo-N-(4-(chlorodifluoromethoxy)phenyl)-1-isopropyl-3-methyl-3-(((tetrahydro-2H-pyran-2-yl)oxy)methyl)indoline-5-carboxamide

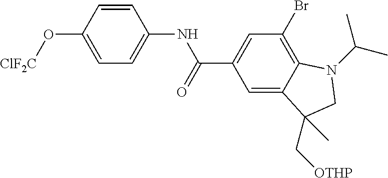

Essentially the same protocol of step 8 in EXAMPLE VII-43 to afford 7-bromo-N-(4-(chlorodifluoromethoxy)phenyl)-1-isopropyl-3-methyl-3-(((tetrahydro-2H-pyran-2-yl)oxy)methyl)indoline-5-carboxamide. MS: 588.10 (M+H)⁺.

### Step 7: Synthesis of N-(4-(chlorodifluoromethoxy)phenyl)-1-isopropyl-3-methyl-7-(1H-pyrazol-5-yl)-3-(((tetrahydro-2H-pyran-2-yl)oxy)methyl)indoline-5-carboxamide

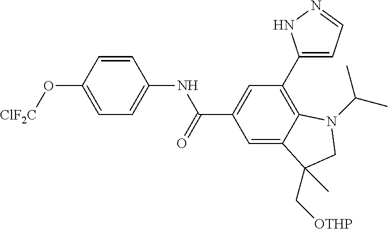

Essentially the same protocol of step 9 in EXAMPLE VII-43 to afford N-(4-(chlorodifluoromethoxy)phenyl)-1-isopropyl-3-methyl-7-(1H-pyrazol-5-yl)-3-(((tetrahydro-2H-pyran-2-yl)oxy)methyl)indoline-5-carboxamide. MS: 576.21 (M+H)⁺.

### Step 8: Synthesis of N-(4-(chlorodifluoromethoxy)phenyl)-3-(hydroxymethyl)-1-isopropyl-3-methyl-7-(1H-pyrazol-5-yl)indoline-5-carboxamide

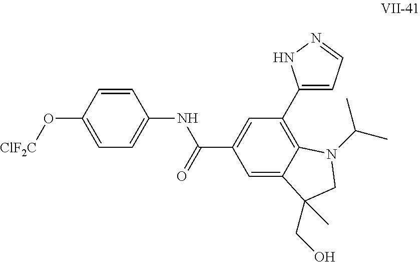

Essentially the same protocol of the preparation of Cpd. No. VII-43 in Example VII-43 was used to afford Cpd. No. VII-41 (4.6 mg) as a white solid. ¹H NMR (400 MHz, DMSO-d₆) δ 12.87 (s, 1H), 10.01 (s, 1H), 7.90-7.83 (m, 2H), 7.80 (s, 1H), 7.67 (d, J=2.0 Hz, 1H), 7.60 (s, 1H), 7.35-7.28 (m, 2H), 6.37 (s, 1H), 4.98 (s, 1H), 3.77-3.64 (m, 1H), 3.58-3.51 (m, 1H), 3.47-3.34 (m, 2H), 3.13-3.06 (m, 1H), 1.31 (s, 3H), 0.95-0.83 (m, 6H). MS: 492.00 (M+1)⁺.

### Example VII-42

### Synthesis of N-(4-(chlorodifluoromethoxy)phenyl)-1,3-diisopropyl-7-(1H-pyrazol-5-yl)indoline-5-carboxamide (Cpd. No. VII-42)

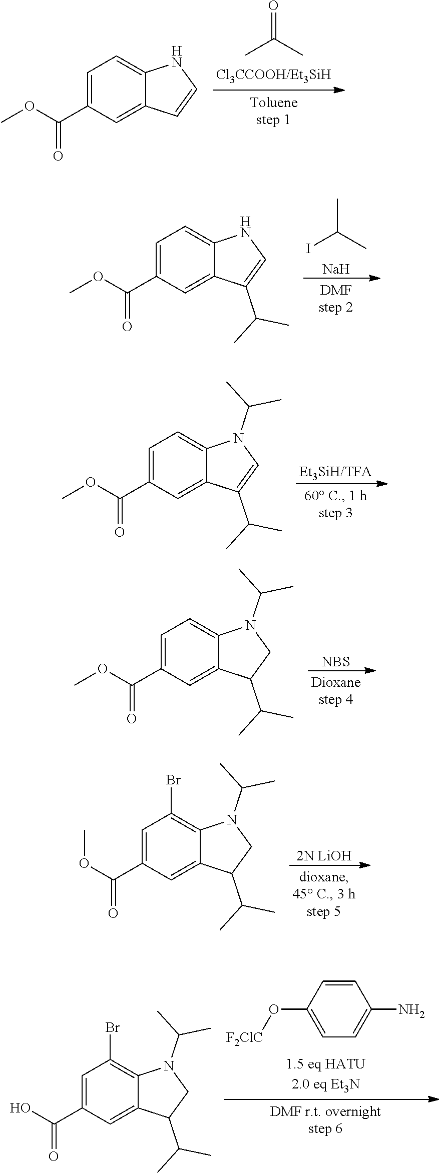

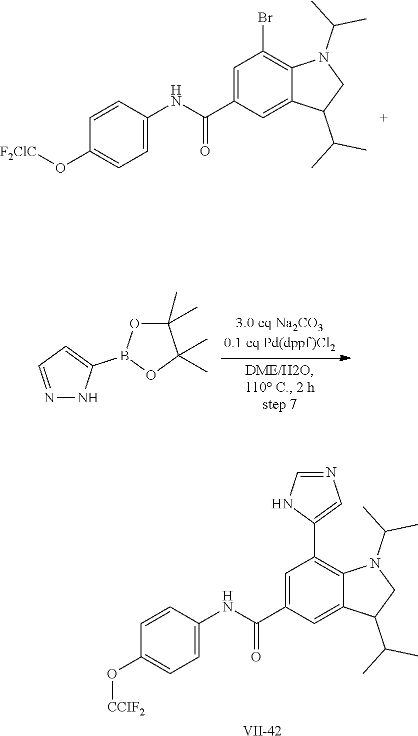

### Step 1: Synthesis of methyl 3-isopropyl-1H-indole-5-carboxylate

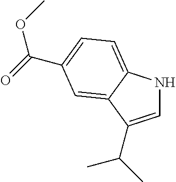 [SMILES: COC(=O)c1ccc2[nH]cc(C(C)C)c2c1]

In a 100 mL two-necked round-bottomed flask, to a solution of trichloroacetic acid (1.4 g, 8.56 mmol) and triethylsilane (2.58 g, 17.12 mmol) in toluene (15 mL) under nitrogen was added a solution of propan-2-one (0.398 g, 6.85 mmol) and methyl 1H-indole-5-carboxylate (1.0 g, 5.71 mmol) in toluene (6 mL) at 70° C. After addition, the mixture was stirred at 90° C. for overnight, then evaporated under vacuum to remove the solvent to give a crude product that was eluted through a silica gel column (ethyl acetate/hexane from 0% to 30%) to give methyl 3-isopropyl-1H-indole-5-carboxylate (0.8 g, 64.5%). MS: 218.0 (M+H)⁺.

### Step 2: Synthesis of methyl 1,3-diisopropyl-1H-indole-5-carboxylate

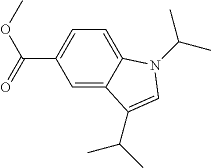 [SMILES: COC(=O)c1ccc2c(c1)c(C(C)C)cn2C(C)C]

In a 100 mL two-necked round-bottomed flask, to a suspension of sodium hydride (0.088 g, 3.68 mmol) in N,N-dimethylformamide (2 mL) under nitrogen was added a solution of methyl 3-isopropyl-1H-indole-5-carboxylate (0.4 g, 1.841 mmol) in N,N-dimethylformamide (3 mL) dropwise. The mixture was stirred for 15 min, then was added 2-iodopropane (0.469 g, 2.76 mmol), and stirred for another 3 h at room temperature, then quenched with aqueous NH₄Cl, followed by extraction with ethyl acetate (30 mL×3). The combined organic layers were concentrated to give a crude product that was eluted through a silica gel column (ethyl acetate/hexane from 0% to 30%) to give methyl 1,3-diisopropyl-1H-indole-5-carboxylate (0.3 g, 62.8%). MS: 259.0 (M+H)⁺.

### Step 3: Synthesis of methyl 1,3-diisopropylindoline-5-carboxylate

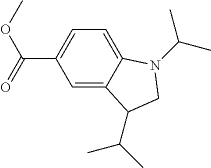 [SMILES: COC(=O)c1ccc2c(c1)C(C(C)C)CN2C(C)C]

In a 50 mL round-bottomed flask, to a solution of methyl 1,3-diisopropyl-1H-indole-5-carboxylate (0.4 g, 1.542 mmol) in trifluoroacetic acid (5 mL) was added triethylsilane (0.717 g, 6.17 mmol). The mixture was stirred for 1 h at 60° C., then concentrated to give a crude product that was purified by silica gel column chromatography (ethyl acetate/hexane from 0% to 30%) to give methyl 1,3-diisopropylindoline-5-carboxylate (0.3 g, 74.4%). MS: 262.10 (M+H)⁺.

### Step 4: Synthesis of methyl 7-bromo-1,3-diisopropylindoline-5-carboxylate

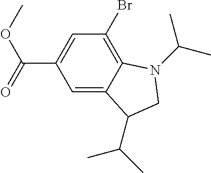 [SMILES: COC(=O)c1cc(Br)c2c(c1)C(C(C)C)CN2C(C)C]

In a 50 mL round-bottomed flask, to a solution of methyl 1,3-diisopropylindoline-5-carboxylate (210 mg, 0.803 mmol) in 1,4-dioxane (6 mL) was added N bromosuccinimide (157 mg, 0.884 mmol). The mixture was stirred for 1 h at room temperature, then quenched with water, followed by extraction with ethyl acetate (50 mL×2). The combined organic layers were washed with brine, dried over Na₂SO₄, filtered and concentrated to give a crude product that was eluted through a silica gel column (ethyl acetate/hexane from 0% to 20%) to give methyl 7-bromo-1,3-diisopropylindoline-5-carboxylate (170 mg, 62.2%). MS: 341.00, 343.00 (M+H)⁺.

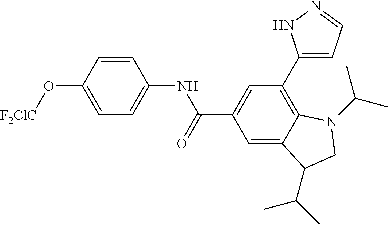

Step 5-7: Essentially the same protocol of the preparation of Cpd. No. VII-37 in Example VII-37 was used to afford Cpd. No. VII-42 (7.8 mg, 8%) as a white solid. ¹H NMR (400 MHz, DMSO-d₆) δ 12.87 (s, 1H), 10.03 (s, 1H), 7.89-7.83 (m, 2H), 7.80 (s, 1H), 7.69-7.64 (m, 1H), 7.62 (s, 1H), 7.31 (d, J=8.6 Hz, 2H), 6.37 (s, 1H), 3.79-3.62 (m, 1H), 3.58-3.43 (m, 1H), 3.30-3.19 (m, 2H), 2.17-2.05 (m, 1H), 1.00 (d, J=6.8 Hz, 3H), 0.96-0.85 (m, 6H), 0.83 (d, J=6.7 Hz, 3H). MS: 490.3 (M+H)⁺.

### Example VII-43

### Synthesis of N-(4-(chlorodifluoromethoxy)phenyl)-3-(hydroxymethyl)-1-isopropyl-7-(1H-pyrazol-5-yl)indoline-5-carboxamide (Cpd. No. VII-43)

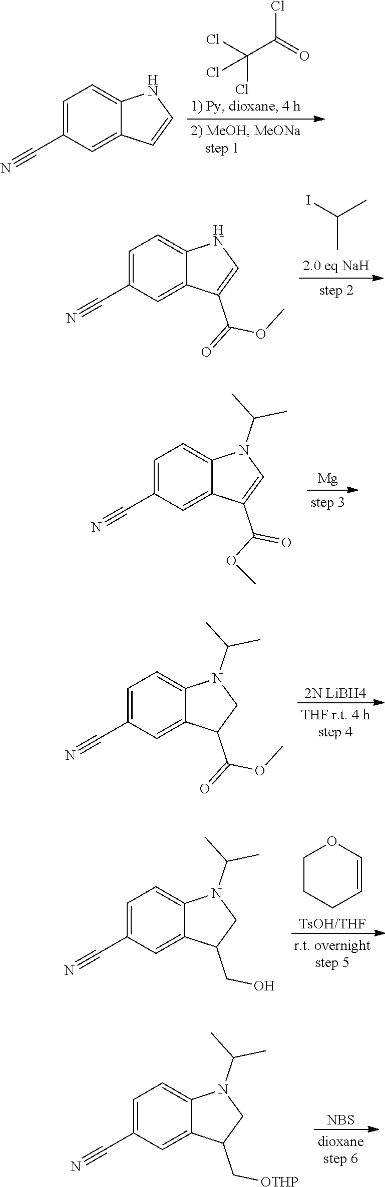

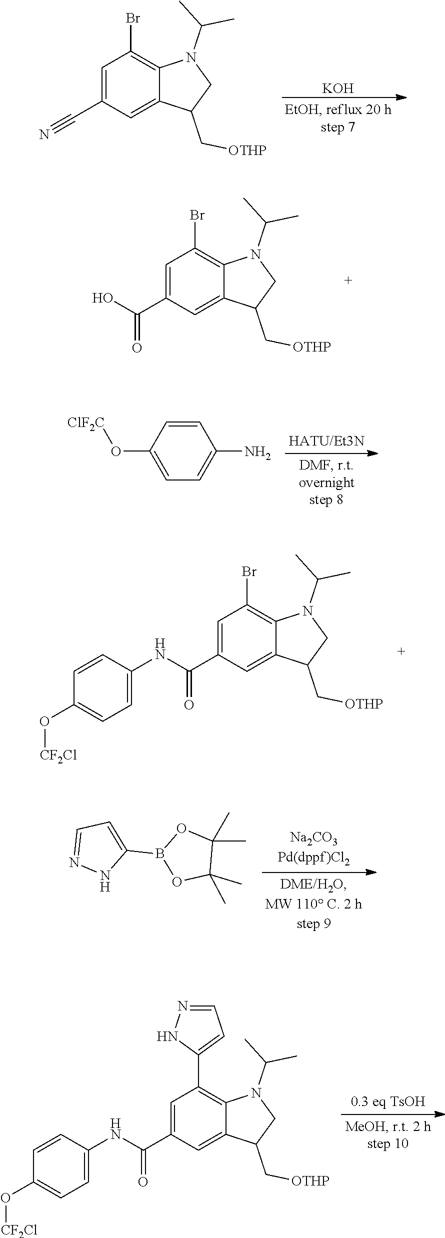

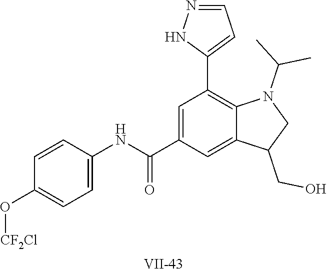

### Step 1: Synthesis of methyl 5-cyano-1H-indole-3-carboxylate

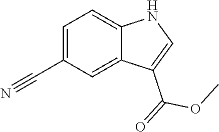

In a 100 mL round-bottomed flask, to a solution of 1H-indole-5-carbonitrile (1.5 g, 10.55 mmol) in 1,4-dioxane (10 mL) under nitrogen was added pyridine (16.69 g, 211 mmol) and 2,2,2-trichloroacetyl chloride (19.18 g, 106 mmol) dropwise at 5° C. The mixture was stirred at 80° C. for 3 h, then quenched with water (30 mL), followed by extraction with dichloromethane (10 mL×3). The combined organic layers were dried over Na₂SO₄, filtered and concentrated to give an oily residue. The residue was dissolved in methanol (20 mL) and added solution of NaOCH₃/methanol (1N, 2 mL) and heated to reflux for 1 h, then concentrated to give a crude product that was eluted through a silica gel column (ethyl acetate/hexane from 5% to 30%) to give methyl 5-cyano-1H-indole-3-carboxylate (1.9 g, 90%) as a white solid. MS: 201.00 (M+H)⁺.

### Step 2: Synthesis of methyl 5-cyano-1-isopropyl-1H-indole-3-carboxylate

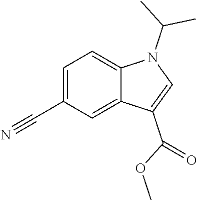 [SMILES: COC(=O)c1cn(C(C)C)c2ccc(C#N)cc12]

In a 100 mL round-bottomed flask, to a suspension of sodium hydride (0.799 g, 19.98 mmol) in N,N-dimethylformamide (10 mL) under nitrogen was added methyl 5-cyano-1H-indole-3-carboxylate (2.0 g, 9.99 mmol) in 10 mL N,N-dimethylformamide dropwise. After being stirred for 10 min, the mixture was added 2-iodopropane (2.55 g, 14.99 mmol) dropwise at 5° C., then stirred at 25° C. for 3 h. After the reaction was completed, the mixture was quenched with water (30 mL), followed by extraction with dichloromethane (30 mL×3). The combined organic layers were dried over Na₂SO₄, filtered and concentrated to give a crude product that was eluted through a silica gel column (ethyl acetate/hexane from 5% to 30%) to give methyl 5-cyano-1-isopropyl-1H-indole-3-carboxylate (2 g, 83%) as a white solid. ¹H NMR (400 MHz, chloroform-d) δ 8.56 (s, 1H), 8.07 (s, 1H), 7.54 (d, J=8.8 Hz, 1H), 7.48 (d, J=8.5 Hz, 1H), 4.79-4.68 (m, 1H), 3.96 (s, 3H), 1.61 (d, J=6.7 Hz, 6H).

### Step 3: Synthesis of methyl 5-cyano-1-isopropylindoline-3-carboxylate

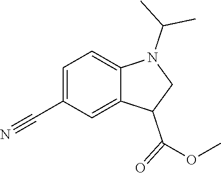 [SMILES: COC(=O)C1CN(C(C)C)c2ccc(C#N)cc21]

In a dried 100 mL round-bottomed flask, to a solution of methyl 5-cyano-1-isopropyl-1H-indole-3-carboxylate (2.0 g, 8.25 mmol) in methanol (30 mL) and dichloromethane (10 mL) under nitrogen was added magnesium (0.803 g, 33.0 mmol). The mixture was stirred overnight, acidified with 1N HCl, and extracted with ethyl acetate (50 mL×3). The resulting organic layers were combined and then concentrated to give a crude product that was purified by silica gel column chromatography (ethyl acetate/hexane from 10% to 40%) to give methyl 5-cyano-1-isopropylindoline-3-carboxylate (1.0 g, 49.6%) as a colorless oil. MS: 245.10 (M+H)⁺.

### Step 4: Synthesis of 3-(hydroxymethyl)-1-isopropylindoline-5-carbonitrile

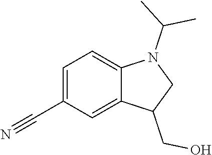 [SMILES: CC(C)N1CC(CO)c2cc(C#N)ccc21]

In a dried 100 mL round-bottomed flask, to a solution of methyl 5-cyano-1-iso-propylindoline-3-carboxylate (500 mg, 2.047 mmol) in tetrahydrofuran (10 mL) under nitrogen was added LiBH₄ (68 mg, 3.12 mmol) The mixture was stirred at room temperature for overnight, then concentrated to remove solvent, diluted with water (30 mL), followed by extraction with ethyl acetate (30 mL×3). The combined organic layers were concentrated to give a crude product that was purified by silica gel column chromatography (ethyl acetate/hexane from 5% to 60%) to give 3-(hydroxymethyl)-1-isopropylindoline-5-carbonitrile (300 mg, 67.8%). MS: 217.10 (M+H)⁺.

### Step 5: Synthesis of 1-isopropyl-3-(((tetrahydro-2H-pyran-2-yl)oxy)methyl)indoline-5-carbonitrile

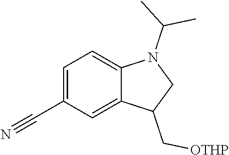

In a 100 mL round-bottomed flask, to a solution of 3-(hydroxymethyl)-1-isopropylindoline-5-carbonitrile (400 mg, 1.849 mmol) in tetrahydrofuran (30 mL) under nitrogen was added 3,4-dihydro-2H-pyran (389 mg, 4.62 mmol) and p-toluenesulfonic acid monohydrate (35.2 mg, 0.185 mmol). The mixture was stirred at room temperature for 2 h, then concentrated to give a crude product that was purified by silica gel column chromatography (ethyl acetate/hexane from 10% to 80%) to give 1-isopropyl-3-(((tetrahydro-2H-pyran-2-yl)oxy)methyl)indoline-5-carbonitrile (440 mg, 79%) as a colorless oil. MS: 301.00 (M+H)⁺.

### Step 6: Synthesis of 7-bromo-1-isopropyl-3-(((tetrahydro-2H-pyran-2-yl)oxy)methyl)indoline-5-carbonitrile

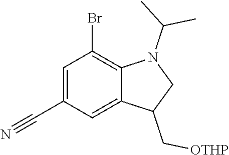

In a 50 mL round-bottomed flask, to a solution of 1-isopropyl-3-(((tetrahydro-2H-pyran-2-yl)oxy)methyl)indoline-5-carbonitrile (440.0 mg, 1.465 mmol) in 1,4-dioxane (30 mL) was added N-Bromosuccinimide (287 mg, 1.611 mmol). The mixture was stirred for 2 h at room temperature, then quenched with water, followed by extraction with ethyl acetate (30 mL×3). The combined organic layers were washed with brine, dried over Na₂SO₄, filtered and concentrated to give a crude product that was eluted through a silica gel column (ethyl acetate/hexane from 0% to 20%) to give 7-bromo-1-isopropyl-3-(((tetrahydro-2H-pyran-2-yl)oxy)methyl)indoline-5-carbonitrile (300 mg, 54%). ¹H NMR (400 MHz, chloroform-d) δ 7.51 (d, J=1.7 Hz, 1H), 7.26-7.19 (m, 1H), 5.18-5.06 (m, 1H), 4.66-4.56 (m, 1H), 3.89-3.75 (m, 2H), 3.75-3.64 (m, 1H), 3.57-3.37 (m, 2H), 1.91-1.68 (m, 3H), 1.68-1.49 (m, 5H), 1.25-1.17 (m, 6H). MS: 379.0, 381.0 (M+H)⁺.

### Step 7: Synthesis of 7-bromo-1-isopropyl-3-(((tetrahydro-2H-pyran-2-yl)oxy)methyl)indoline-5-carboxylic acid

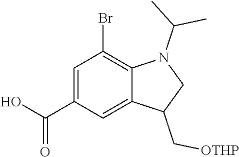

In a 100 mL round-bottomed flask, to a solution of 7-bromo-1-isopropyl-3-(((tetrahydro-2H-pyran-2-yl)oxy)methyl)indoline-5-carbonitrile (300 mg, 0.791 mmol) in EtOH (10 mL) was added 6 N KOH (3 mL, 19.96 mmol). The mixture was heated at reflux for overnight, then cooled to 0° C., acidified to pH 2-3 with 2N HCl, followed by extraction with ethyl acetate. The combined organic layers were concentrated to give a crude product that was purified by silica gel column chromatography (ethyl acetate/hexane from 10% to 80%) to give 7-bromo-1-isopropyl-3-(((tetrahydro-2H-pyran-2-yl) oxy)methyl) indoline-5-carboxylic acid (250 mg, 79%) as a colorless oil. ¹H NMR (400 MHz, chloroform-d) δ 8.03 (d, J=1.7 Hz, 1H), 7.74-7.65 (m, 1H), 5.25-5.10 (m, 1H), 4.68-4.58 (m, 1H), 3.95-3.68 (m, 3H), 3.61-3.38 (m, 3H), 2.09-1.42 (m, 7H), 1.23 (d, J=6.7 Hz, 6H). Ms: 398.00, 400.00 (M+H)⁺.

### Step 8: Synthesis of 7-bromo-N-(4-(chlorodifluoromethoxy)phenyl)-1-isopropyl-3-(((tetrahydro-2H-pyran-2-yl)oxy)methyl)indoline-5-carboxamide

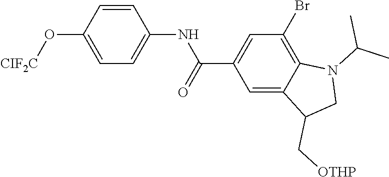

Essentially the same protocol of the step 5 in EXAMPLE III-1 was used to afford -bromo-N-(4-(chlorodifluoromethoxy)phenyl)-1-isopropyl-3-(((tetrahydro-2H-pyran-2-yl)oxy)methyl)indoline-5-carboxamide. MS: 572.9, 574.9 (M+H)⁺.

### Step 9: Synthesis of N-(4-(chlorodifluoromethoxy)phenyl)-1-isopropyl-7-(1H-pyrazol-5-yl)-3-(((tetrahydro-2H-pyran-2-yl)oxy)methyl)indoline-5-carboxamide

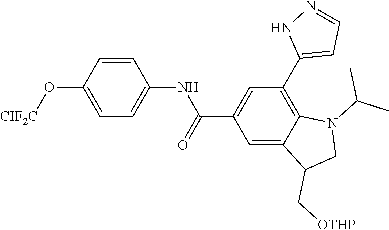

Essentially the same protocol of step 6 in EXAMPLE III-1 was used to afford N-(4-(chlorodifluoromethoxy)phenyl)-1-isopropyl-7-(1H-pyrazol-5-yl)-3-(((tetrahydro-2H-pyran-2-yl)oxy)methyl)indoline-5-carboxamide (100 mg, 59.1%) as a white solid. MS: 562.20 (M+H)⁺.

### Step 10: N-(4-(chlorodifluoromethoxy)phenyl)-3-(hydroxymethyl)-1-isopropyl-7-(1H-pyrazol-5-yl)indoline-5-carboxamide (Cpd. No. VII-43)

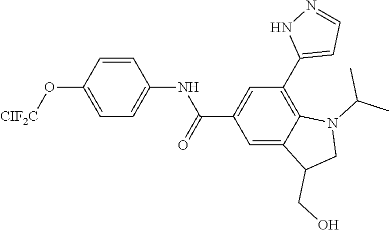

In a 25 mL round-bottomed flask, to a solution of N-(4-(chlorodifluoromethoxy)phenyl)-1-isopropyl-7-(1H-pyrazol-5-yl)-3-(((tetrahydro-2H-pyran-2-yl)oxy)methyl)indoline-5-carboxamide (70 mg, 0.125 mmol) in 5 mL methanol was added p-toluenesulfonic acid (7.12 mg, 0.037 mmol). The mixture was stirred for 3 h at room temperature, then concentrated to give crude product which was purified using preparative HPLC to give N-(4-(chlorodifluoromethoxy)phenyl)-3-(hydroxymethyl)-1-isopropyl-7-(1H-pyrazol-5-yl)indoline-5-carboxamide (8 mg, 13.44%). ¹H NMR (400 MHz, DMSO-d₆) δ 12.88 (s, 1H), 10.04 (s, 1H), 7.86 (d, J=8.6 Hz, 2H), 7.80 (s, 1H), 7.71-7.52 (m, 2H), 7.31 (d, J=8.6 Hz, 2H), 6.37 (s, 1H), 4.95 (s, 1H), 3.73-3.63 (m, 2H), 3.63-3.45 (m, 2H), 3.41-3.26 (m, 2H), 0.89 (d, J=6.6 Hz, 6H).

### Example VII-44

### Synthesis of 1-methyl-7-(1H-pyrazol-5-yl)-N-(4-((trifluoromethyl)thio)phenyl)indoline-5-carboxamide (Cpd. No. VII-44)

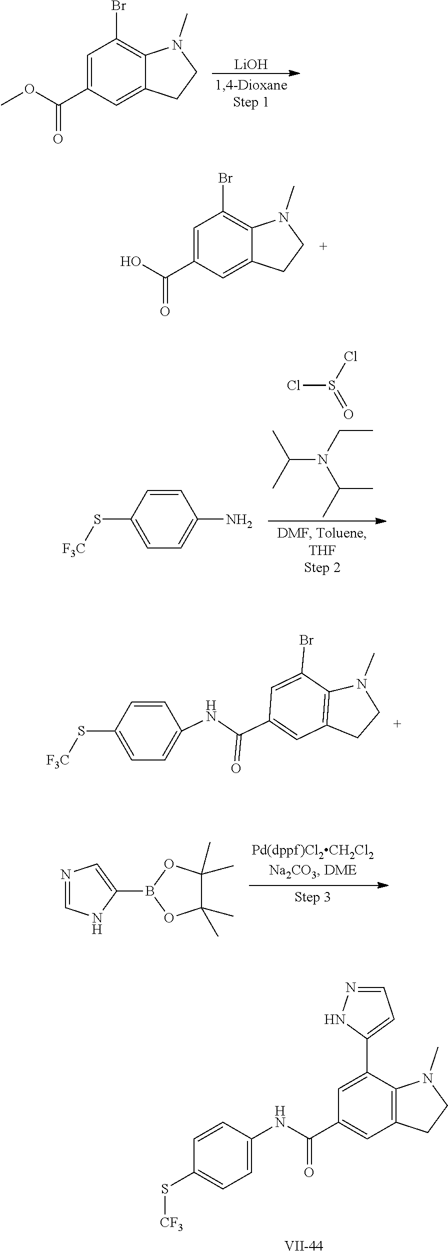

### Step 1: Synthesis of methyl 7-bromoindoline-5-carboxylate

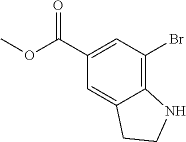

In an oven-dried 100 mL round-bottomed flask, methyl 7-bromo-1-methylindoline-5-carboxylate (1.1 g, 4.07 mmol) was dissolved in 1,4-dioxane (30 mL) under nitrogen to give a solution. Lithium hydroxide (0.488 g, 20.36 mmol) was added to the solution. The mixture was stirred at 45° C. for 1 h. 1N HCl (50 mL) was added to the mixture followed by extraction with ethyl acetate (20 mL×3). The combined organic layers were dried over Na₂SO₄, filtered and concentrated to give 7-bromo-1-methylindoline-5-carboxylic acid (1.13 g, quant), it was used in next step without purification. MS: 257.0 (M+H)⁺.

### Step 2: Synthesis of 7-bromo-1-methyl-N-(4-((trifluoromethyl)thio)phenyl)indoline-5-carboxamide

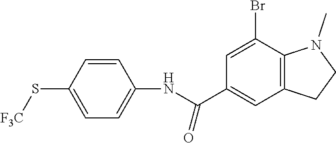 [SMILES: CN1CCc2cc(C(=O)Nc3ccc(SC(F)(F)F)cc3)cc(Br)c21]

In an oven-dried 50 mL round-bottomed flask, 7-bromo-1-methylindoline-5-carboxylic acid (508 mg, 1.984 mmol) and thionyl chloride (10 mL) were stirred at 80° C. for 1 h. The thionyl chloride was evaporated under reduced pressure and the residue was dissolved in tetrahydrofuran (0.4 mL) to which was added diisopropylethyl amine (513 mg, 3.97 mmol). The mixture was cooled to 0° C. under an ice/water bath, and it was added a solution of 4-((trifluoromethyl)thio)aniline (422 mg, 2.182 mmol) in tetrahydrofuran (1 mL) dropwise over 10 min. Then the mixture was stirred at 0° C. for another 2 h, and diluted with ethyl acetate (20 mL), washed with 1N HCl (15 mL) and 1N NaOH (15 mL). The organic layer was dried over Na₂SO₄, filtered and concentrated to give a crude product that was purified by silica gel column chromatography (ethyl acetate/hexane from 0% to 50%) to give 7-bromo-1-methyl-N-(4-((trifluoromethyl)thio)phenyl)indoline-5-carboxamide (600 mg, 70.1%) as a yellow solid. ¹H NMR (400 MHz, chloroform-d) δ 7.74-7.64 (m, 4H), 7.63 (d, J=8.7 Hz, 2H), 7.47 (m, 1H), 3.54 (t, J=8.8 Hz, 2H), 3.24 (s, 3H), 3.01 (t, J=8.8 Hz, 2H). MS: 431.0 (M+H)⁺.

### Step 3: Synthesis of 1-methyl-7-(1H-pyrazol-5-yl)-N-(4-((trifluoromethyl)thio)phenyl)indoline-5-carboxamide (Cpd. No. VII-44)

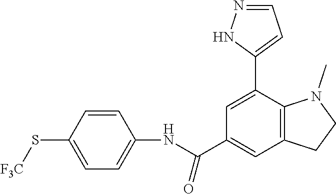 [SMILES: CN1CCc2cc(C(=O)Nc3ccc(SC(F)(F)F)cc3)cc(-c3ccn[nH]3)c21]

5-(4,4,5,5-tetramethyl-1,3,2-dioxaborolan-2-yl)-1H-pyrazole (135.0 mg, 0.696 mmol), Na₂CO₃ (147.0 mg, 1.391 mmol) and PdCl₂(dppf)-CH₂Cl₂ adduct (33.9 mg, 0.046 mmol) were added to a solution of 7-bromo-N-(4-(chlorodifluoromethoxy) phenyl)-1-isopropylindoline-5-carboxamide (200.0 mg, 0.464 mmol) in dimethoxyethane/water/EtOH (0.15 mL/0.3 mL/1.5 mL). The mixture was purged with nitrogen, then stirred at 110° C. under MW for 2 h. The reaction mixture was diluted with diethyl ether (20.0 mL), washed with water (20.0 mL) and saturated aqueous NaCl (20.0 mL). The organic layer was dried over Na₂SO₄, filtered and concentrated to give a crude product that was purified using preparative HPLC to give N-(4-(chlorodifluoromethoxy)phenyl)-1-isopropyl-7-(1H-pyrazol-5-yl)indoline-5-carboxamide (42.0 mg, 22%) as a white solid. ¹H NMR (400 MHz, chloroform-d) δ 8.13 (s, 1H), 7.75 (d, J=8.4 Hz, 2H), 7.72-7.60 (m, 5H), 6.47 (d, J=2.0 Hz, 1H), 3.53 (t, J=8.7 Hz, 2H), 3.10 (t, J=8.6 Hz, 2H), 2.57 (s, 3H). MS: 419.1 (M+H)⁺.

### Example VII-45

### Synthesis of 1-isopropyl-7-(1H-pyrazol-5-yl)-N-(4-((trifluoromethyl)thio)phenyl)indoline-5-carboxamide (Cpd. No. VII-45)

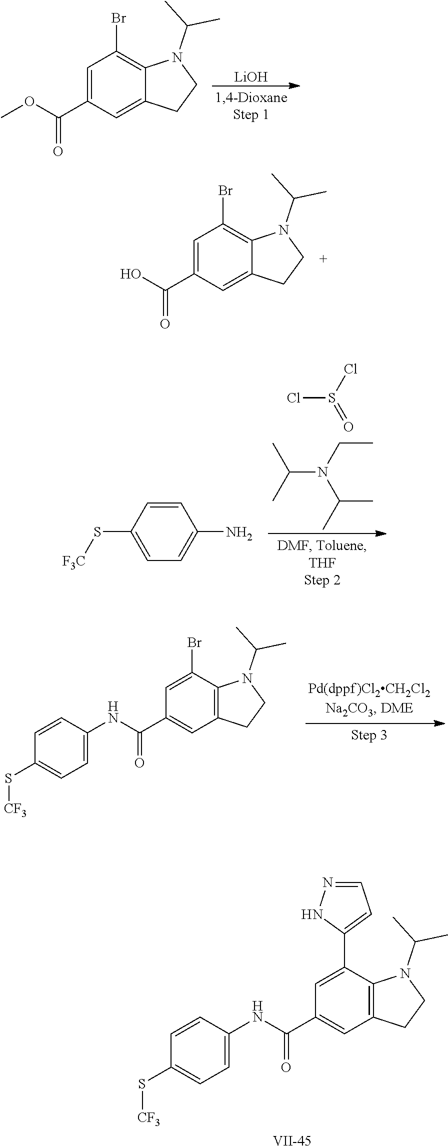

Essentially the same protocol of the preparation of Cpd. No. VII-44 in Example VII-44 was used to afford Cpd. No. VII-45 (130.0 mg) as a white solid. ¹H NMR (400 MHz, DMSO-d₆) δ 10.25 (s, 1H), 7.94 (d, J=8.7 Hz, 2H), 7.79-7.64 (m, 5H), 6.48 (d, J=2.0 Hz, 1H), 3.70-3.61 (m, 1H), 3.56 (t, J=8.6 Hz, 2H), 3.05 (t, J=8.6 Hz, 2H), 0.94 (d, J=6.6 Hz, 6H). MS: 447.1 (M+H)⁺.

### Example VII-46

### Synthesis of 1-isopropyl-7-(1H-pyrazol-5-yl)-N-(4-(trifluoromethoxy)phenyl)indoline-5-carboxamide (Cpd. No. VII-46)

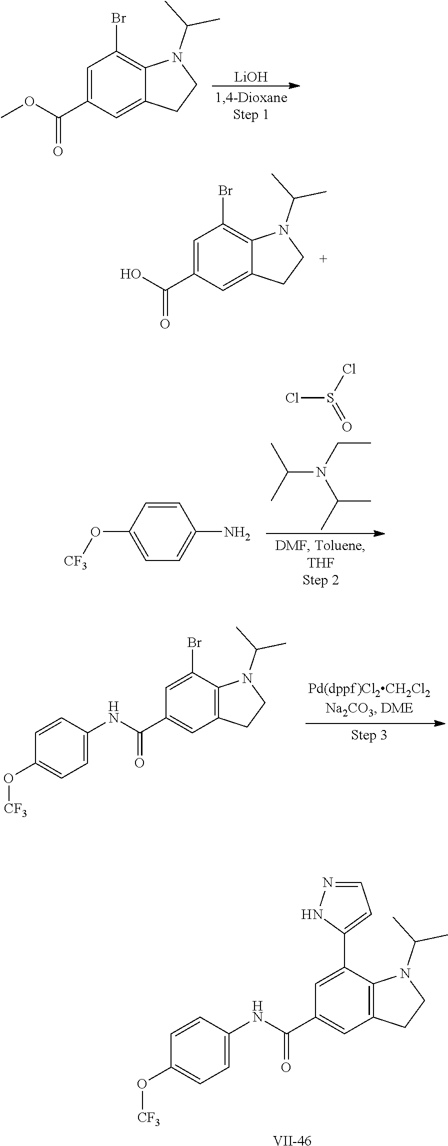

Essentially the same protocol of the preparation of Cpd. No. VII-44 in Example VII-44 was used to afford Cpd. No. VII-46 (130.0 mg) as a white solid. ¹H NMR (500 MHz, MeOD) δ 7.75 (d, J=9.0 Hz, 2H), 7.72-7.61 (m, 3H), 7.23 (d, J=8.7 Hz, 2H), 6.44 (s, 1H), 3.55 (t, J=8.8 Hz, 2H), 3.34-3.28 (m, 1H), 3.05 (t, J=8.7 Hz, 2H), 0.96 (d, J=6.6 Hz, 6H).

### Example VIII-1

### Synthesis of N-(4-(chlorodifluoromethoxy)phenyl)-1,3,3-trimethyl-2-oxo-7-(1H-pyrazol-5-yl)indoline-5-carboxamide (Cpd. No. VIII-1)

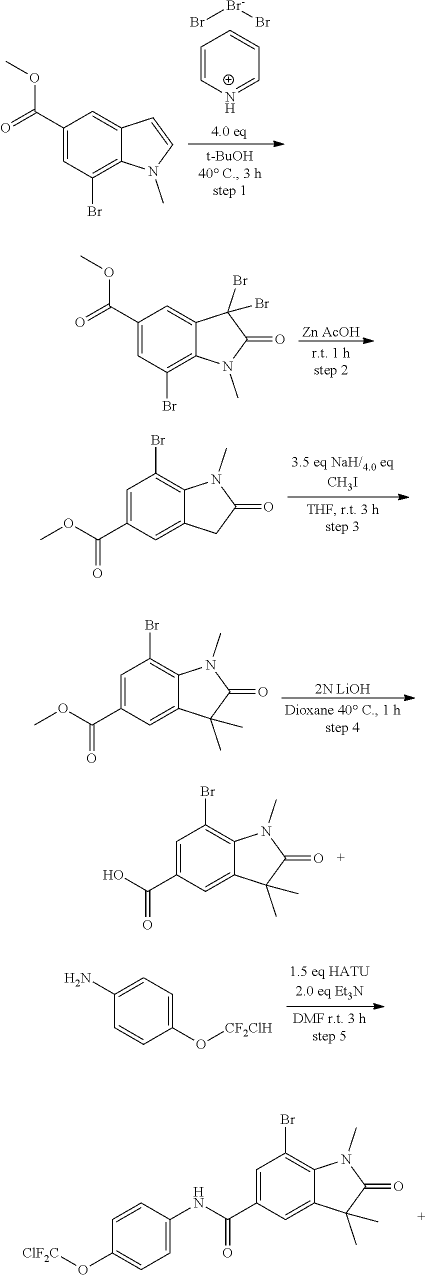

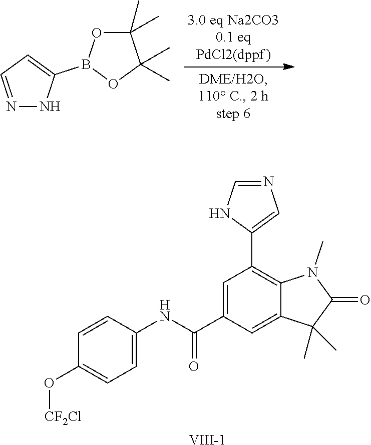

Essentially the same protocol of the preparation of Cpd. No. V-1 in EXAMPLE V-1 was used to afford Cpd. No. VIII-1 (30.0 mg) as a white solid. ¹H NMR (400 MHz, chloroform-d) δ 8.35 (s, 1H), 7.82 (d, J=1.9 Hz, 1H), 7.76-7.72 (m, 3H), 7.67 (d, J=2.3 Hz, 1H), 7.26 (d, J=8.7 Hz, 2H), 6.50 (d, J=2.3 Hz, 1H), 2.89 (s, 3H), 1.44 (s, 6H). MS: 462.00 (M+H)⁺.

### Example VIII-2

### Synthesis of N-(4-(chlorodifluoromethoxy)phenyl)-1-isopropyl-3,3-dimethyl-2-oxo-7-(pyrimidin-5-yl)indoline-5-carboxamide (Cpd. No. VIII-2)

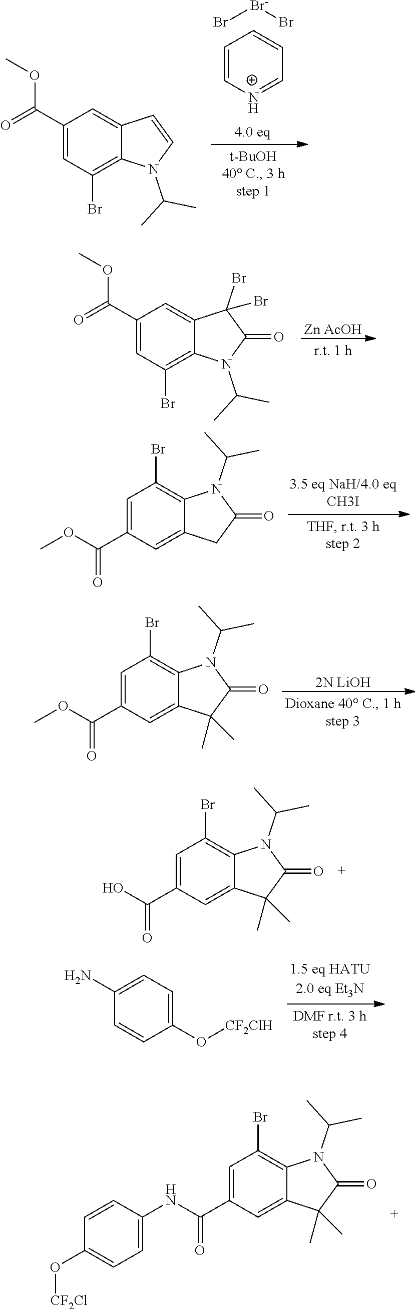

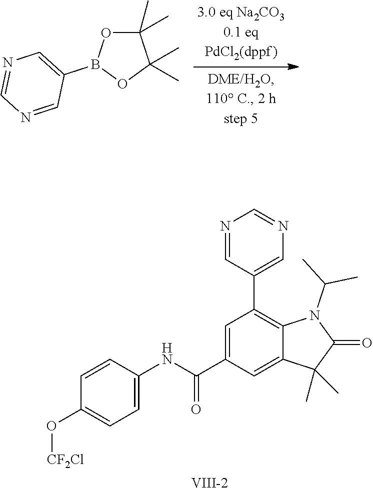

Essentially the same protocol of the preparation of Cpd. No. V-1 in EXAMPLE V-1 was used to afford Cpd. No. VIII-2 (45.0 mg) as a white solid. 1H NMR (400 MHz, methanol-d4) δ 9.29 (s, 1H), 9.00 (s, 2H), 8.05 (d, J=2.0 Hz, 1H), 7.85-7.81 (m, 2H), 7.79 (d, J=1.9 Hz, 1H), 7.35-7.26 (m, 2H), 3.67-3.55 (m, 1H), 1.45 (s, 6H), 1.34 (d, J=6.8 Hz, 6H). MS: 501.14 (M+H)⁺.

### Example VIII-3

### Synthesis of N-(4-(chlorodifluoromethoxy)phenyl)-1-isopropyl-3,3-dimethyl-2-oxo-7-(1H-pyrazol-5-yl)indoline-5-carboxamide (Cpd. No. VIII-3)

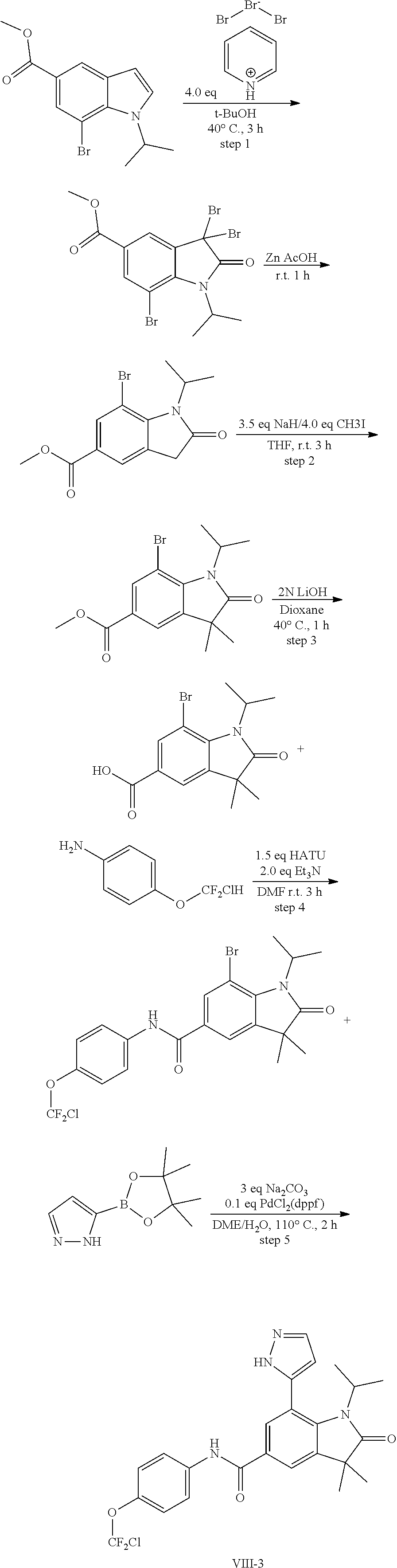

Essentially the same protocol of the preparation of Cpd. No. V-1 in Example V-1 was used to afford Cpd. No. VIII-3 as a white solid. 1H NMR (400 MHz, DMSO-d₆) δ 10.31 (s, 1H), 8.00 (d, J=2.0 Hz, 1H), 7.91-7.87 (m, 2H), 7.86-7.82 (m, 2H), 7.36 (d, J=8.7 Hz, 2H), 6.52 (d, J=2.1 Hz, 1H), 3.72-3.61 (m, 1H), 1.34 (s, 6H), 1.25 (d, J=6.7 Hz, 6H). MS: 489.00 (M+H)⁺.

### Example IX-1

### Synthesis of N-(4-(chlorodifluoromethoxy)phenyl)-1-isopropyl-8-(1H-pyrazol-5-yl)-1,2,3,4-tetrahydroquinoline-6-carboxamide (Cpd. No. IX-1)

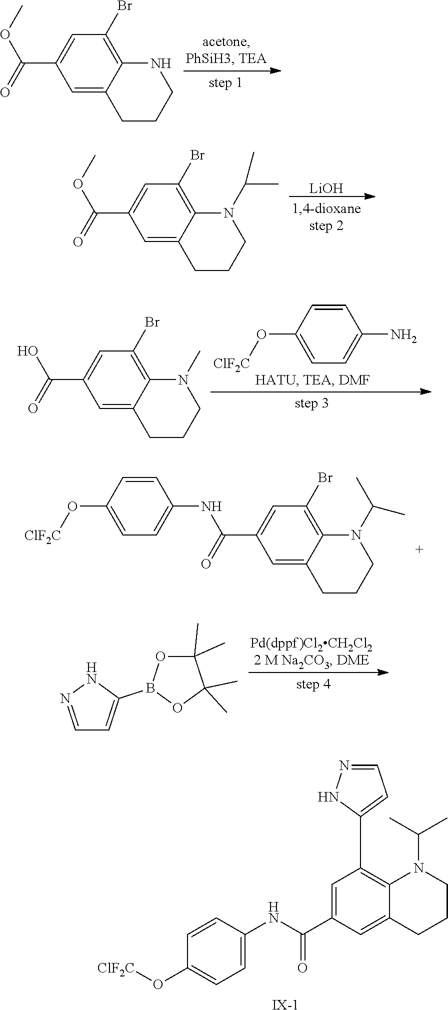

Essentially the same protocol of the preparation of Cpd. No. VII-2 in Example VII-2 was used to afford Cpd. No. IX-1 (3.3 mg) as a white solid. MS: 461.00 (M+H)⁺.

### Example IX-2 N-(4-(chlorodifluoromethoxy)phenyl)-1-methyl-8-(1H-pyrazol-5-yl)-1,2,3,4-tetrahydroquinoline-6-carboxamide (Cpd. No. IX-2)

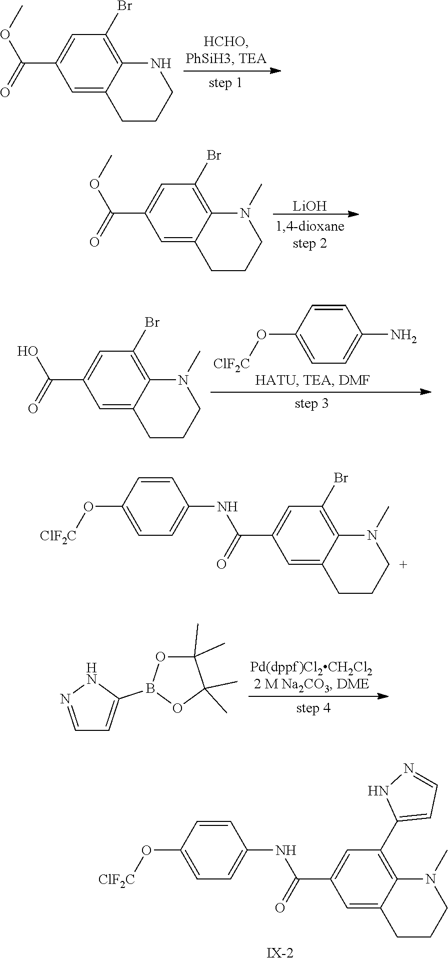

Essentially the same protocol of the preparation of Cpd. No. VII-2 in Example VII-2 was used to afford Cpd. No. IX-1 (50.0 mg) as a white solid. ¹H NMR (400 MHz, DMSO-d₆) δ 10.17 (s, 1H), 7.89 (d, J=8.5 Hz, 2H), 7.79 (s, 1H), 7.58 (d, J=29.5 Hz, 2H), 7.33 (d, J=8.5 Hz, 2H), 6.50 (s, 1H), 3.18 (t, J=5.1 Hz, 2H), 2.82 (d, J=6.3 Hz, 2H), 2.46 (s, 3H), 1.88 (d, J=7.1 Hz, 2H). MS: 433.00 (M+H)⁺.

### Example IX-3

### Synthesis of N-(4-(chlorodifluoromethoxy) phenyl)-4-isopropyl-5-(1H-pyrazol-5-yl)-3,4-dihydro-2H-benzo[b][1,4] oxazine-7-carboxamide (Cpd. No. IX-3)

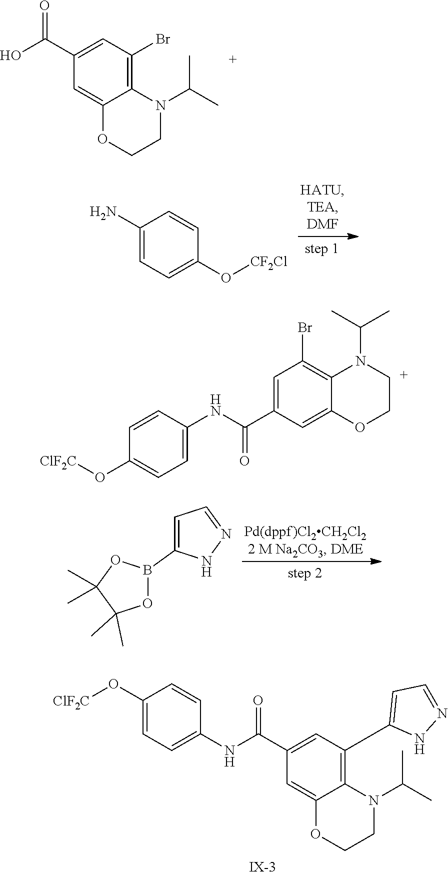

Essentially the same protocol of the preparation of Cpd. No. VII-2 in Example VII-2 was used to afford Cpd. No. IX-3 (9.0 mg) as a white solid. ¹H NMR (400 MHz, DMSO-d₆) δ 13.15-12.77 (m, 1H), 10.22 (s, 1H), 7.88 (d, J=9.0 Hz, 2H), 7.81-7.36 (m, 3H), 7.32 (d, J=9.0 Hz, 2H), 6.61-6.42 (m, 1H), 4.14-4.08 (m, 2H), 3.30-3.28 (m, 2H), 3.17-3.16 (m, 1H), 0.91-0.89 (m, 6H). MS: 463.1 (M+H)⁺.

### Example IX-4

### Synthesis of N-(4-(chlorodifluoromethoxy)phenyl)-4-isopropyl-2-methyl-5-(1H-pyrazol-5-yl)-3,4-dihydro-2H-benzo[b][1,4]oxazine-7-carboxamide (Cpd. No. IX-4)

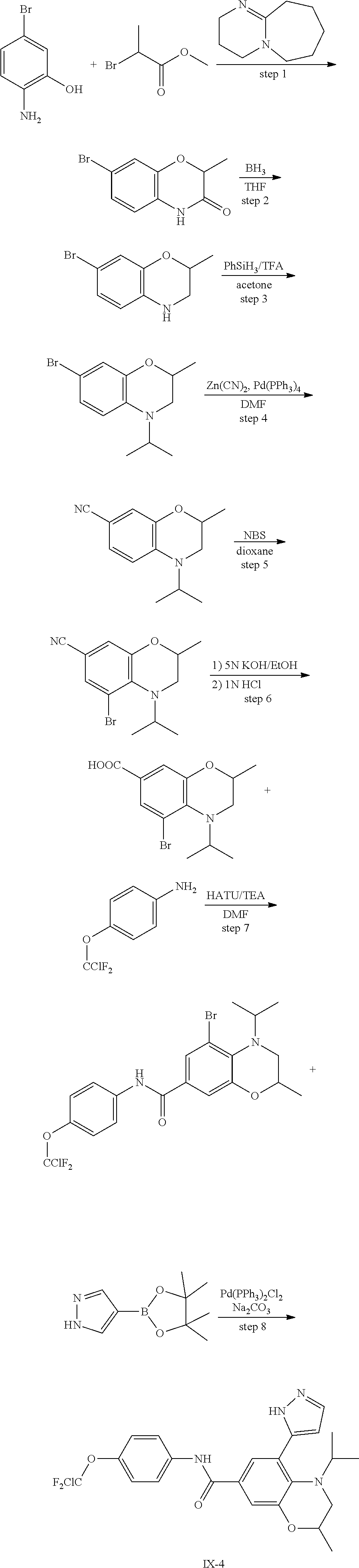

### Step 1: Synthesis of 7-bromo-2-methyl-2H-benzo[b][1,4]oxazin-3(4H)-one

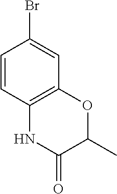

In an oven-dried 25 mL microwave reaction tube, 2-amino-5-bromophenol (2.8 g, 14.89 mmol), methyl 2-bromopropanoate (2.487 g, 14.89 mmol) and NMP (10.30 g, 104 mmol) were dissolved in DBU (2.121 g, 13.93 mmol) under nitrogen to give a solution. The mixture was stirred at 180° C. under microwave for 5 min. The reaction mixture was quenched with water (20 mL), followed by extraction with ethyl acetate (20 mL×3). The combined organic layers were dried over Na₂SO₄, filtered and concentrated to give a crude product that was eluted through a silica gel column (ethyl acetate/hexane from 30% to 50%) to give 7-bromo-2-methyl-2H-benzo[b][1,4]oxazin-3(4H)-one (2.82 g, 78%) as a solid. ¹H NMR (400 MHz, chloroform-d) δ 8.90-8.54 (m, 1H), 7.14 (d, J=1.6 Hz, 1H), 7.08 (dt, J=8.4, 1.6 Hz, 1H), 6.70 (dd, J=8.3 Hz, 1H), 4.77-4.57 (m, 1H), 1.57 (d, J=6.8 Hz, 3H). MS: 241.9 (M+H)⁺.

### Step 2: Synthesis of 7-bromo-2-methyl-3,4-dihydro-2H-benzo[b][1,4]oxazine

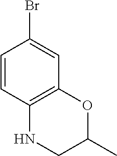

In an oven-dried 250 mL two-necked round-bottomed flask, 7-bromo-2-methyl-2H-benzo[b][1,4]oxazin-3(4H)-one (3.72 g, 15.37 mmol) was dissolved in tetrahydrofuran (60 mL) under nitrogen to give a solution. The mixture was cooled in an ice/water bath to 0° C. BH₃ in tetrahydrofuran (1N, 61.5 mL, 61.5 mmol) was added to the mixture dropwise over 10 min using an addition funnel. The mixture was stirred at room temperature for overnight. Water (10 mL) was added to the mixture followed by extraction with ethyl acetate (20 mL×3). The combined organic layers were dried over Na₂SO₄, filtered and concentrated to give a crude product that was eluted through a silica gel column (ethyl acetate/hexane from 30%-40%) to give 7-bromo-2-methyl-3,4-dihydro-2H-benzo[b][1,4]oxazine (2.65 g, 76%) as a solid. ¹H NMR (400 MHz, DMSO-d₆) δ 6.84-6.75 (m, 2H), 6.51 (d, J=8.8 Hz, 1H), 5.95 (s, 1H), 4.08-4.01 (m, 1H), 3.01-2.75 (m, 2H), 1.26 (d, J=6.2 Hz, 3H). MS: 228.0 (M+H)⁺.

### Step 3: Synthesis of 7-bromo-4-isopropyl-2-methyl-3,4-dihydro-2H-benzo[b][1,4]oxazine

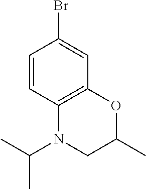

In a nitrogen flushed 50 mL two-necked round-bottomed flask 7-bromo-2-methyl-3,4-dihydro-2H-benzo[b][1,4]oxazine (2.6 g, 11.40 mmol) was dissolved in acetone (10 mL) and trifluoroacetic acid (5 mL) under nitrogen to give a yellow solution. Phenylsilane (6.17 g, 57.0 mmol) was added to the mixture. The mixture was stirred at room temperature for 1 day, concentrated to give a crude product that was eluted through a silica gel column (ethyl acetate/hexane from 0% to 40%) to give 7-bromo-4-isopropyl-2-methyl-3,4-dihydro-2H-benzo[b][1,4]oxazine (2.2 g, 71.4%) as a yellow oil. ¹H NMR (400 MHz, chloroform-d) δ 6.96-6.89 (m, 2H), 6.61 (d, J=9.3 Hz, 1H), 4.26-4.14 (m, 1H), 4.05-4.01 (m, 1H), 3.24 (dd, J=11.7, 2.5 Hz, 1H), 2.81 (dd, J=11.7, 8.0 Hz, 1H), 1.37 (d, J=6.3 Hz, 3H), 1.21 (d, J=6.6 Hz, 3H), 1.14 (d, J=6.6 Hz, 3H). MS: 270.0 (M+H)⁺.

### Step 4: Synthesis of 4-isopropyl-2-methyl-3,4-dihydro-2H-benzo[b][1,4]oxazine-7-carbonitrile

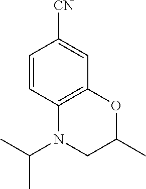 [SMILES: CC1CN(C(C)C)c2ccc(C#N)cc2O1]

In an oven-dried 25 mL round-bottomed flask 7-bromo-4-isopropyl-2-methyl-3,4-dihydro-2H-benzo[b][1,4]oxazine (100 mg, 0.370 mmol) was dissolved in N,N-dimethylformamide (5 mL) under nitrogen to give a solution. Dicyanozinc (43.5 mg, 0.370 mmol) and Pd(PPh₃)₄ (42.8 mg, 0.037 mmol) were added to the mixture. The mixture was stirred at 110° C. for 16 h. Water (10 mL) was added to the mixture, followed by extraction with ethyl acetate (15 mL×3). The combined organic layers were dried over Na₂SO₄, filtered and concentrated to give a crude product that was eluted through a silica gel column (ethyl acetate/hexane from 30% to 55%) to give 4-isopropyl-2-methyl-3,4-dihydro-2H-benzo[b][1,4]oxazine-7-carbonitrile (67 mg, 84%) as an oil. MS: 217.1 (M+H)⁺.

### Step 5: Synthesis of 5-bromo-4-isopropyl-2-methyl-3,4-dihydro-2H-benzo[b][1,4]oxazine-7-carbonitrile

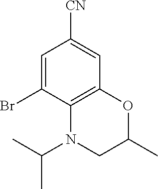 [SMILES: CC1CN(C(C)C)c2c(Br)cc(C#N)cc2O1]

In a nitrogen flushed 25 mL two-necked round-bottomed flask, 4-isopropyl-2-methyl-3,4-dihydro-2H-benzo[b][1,4]oxazine-7-carbonitrile (820 mg, 3.79 mmol) was dissolved in dioxane (10 mL) under nitrogen to give a colorless solution. 1-bromopyrrolidine-2,5-dione (742 mg, 4.17 mmol) was added to the mixture at 0° C. The mixture was stirred at room temperature for 1 h. Water (10 mL) was added to the mixture, followed by extraction with ethyl acetate (15 mL×3). The combined organic layers were dried over Na₂SO₄, filtered and concentrated to give a crude product that was eluted through a silica gel column (ethyl acetate/hexane from 0% to 40%) to give 5-bromo-4-isopropyl-2-methyl-3,4-dihydro-2H-benzo[b][1,4]oxazine-7-carbonitrile (1.1 g, 98%) as a yellow oil. MS: 294.9 (M+H)⁺.

### Step 6: Synthesis of 5-bromo-4-isopropyl-2-methyl-3,4-dihydro-2H-benzo[b][1,4]oxazine-7-carboxylic acid

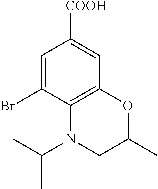 [SMILES: CC1CN(C(C)C)c2c(Br)cc(C(=O)O)cc2O1]

In an oven-dried 100 mL round-bottomed flask, 5-bromo-4-isopropyl-2-methyl-3,4-dihydro-2H-benzo[b][1,4]oxazine-7-carbonitrile (1.1 g, 3.73 mmol) was dissolved in AcOH (8 mL) to give a solution. KOH (3 g, 53.5 mmol) was added to the mixture. The mixture was refluxed for overnight. The mixture was acidified with 1N HCl (16 mL), extracted with ethyl acetate (15 mL×3), dried over Na₂SO₄, filtered and concentrated to give title compound 1.20 g, which was used in next step without purification. MS: 314.0 (M+H)⁺.

### Step 7: Synthesis of N-(4-(chlorodifluoromethoxy)phenyl)-4-isopropyl-2-methyl-5-(1H-pyrazol-5-yl)-3,4-dihydro-2H-benzo[b][1,4]oxazine-7-carboxamide

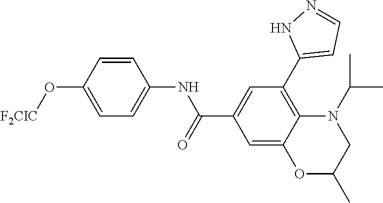

Essentially the same protocol of the preparation of Cpd. No. III-1 in EXAMPLE III-1 was used to afford Cpd. No. IX-4 (100.0 mg) as a white solid. ¹H NMR (400 MHz, DMSO-d₆) δ 12.94 (s, 1H), 10.20 (s, 1H), 7.94-7.86 (m, 2H), 7.84-7.39 (m, 3H), 7.33 (d, J=8.6 Hz, 2H), 6.52 (s, 1H), 3.98 (m, 1H), 3.49 (dd, J=13.9, 2.5 Hz, 2H), 2.72 (dd, J=13.9, 9.9 Hz, 1H), 1.34 (d, J=6.0 Hz, 3H), 0.92 (d, J=6.6 Hz, 3H), 0.90 (d, J=6.7 Hz, 3H). MS: 477.1 (M+H)⁺.

### Example IX-5 N-(4-(chlorodifluoromethoxy)phenyl)-4-isopropyl-2,2-dimethyl-5-(1H-pyrazol-5-yl)-3,4-dihydro-2H-benzo[b][1,4]oxazine-7-carboxamide (Cpd. No. IX-5)

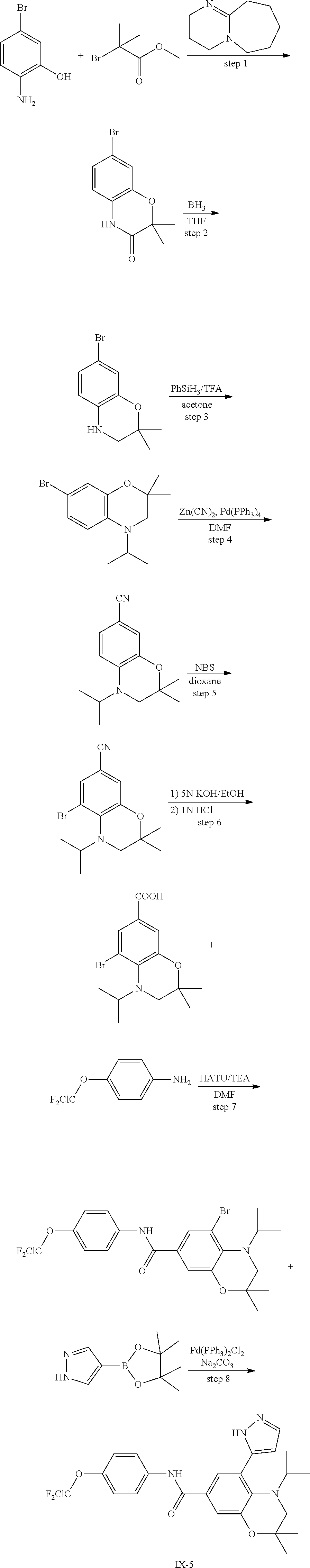

Essentially the same protocol of the preparation of Cpd. No. IX-4 in Example IX-4 was used to afford Cpd. No. IX-5 (280.0 mg) as a white solid. ¹H NMR (400 MHz, DMSO-d₆) δ 12.94 (s, 1H), 10.20 (s, 1H), 7.94-7.86 (m, 2H), 7.84-7.39 (m, 3H), 7.33 (d, J=8.6 Hz, 2H), 6.52 (s, 1H), 3.98 (m, 1H), 3.49 (dd, J=13.9, 2.5 Hz, 2H), 2.72 (dd, J=13.9, 9.9 Hz, 1H), 1.35 (s, 3H), 1.33 (s, 3H), 0.93 (d, J=6.6 Hz, 3H), 0.91 (d, J=6.6 Hz, 3H). MS: 477.1 (M+H)⁺.

### Example IX-6

### Synthesis of 4-isopropyl-5-(1H-pyrazol-5-yl)-N-(4-(trifluoromethoxy)phenyl)-3,4-dihydro-2H-benzo[b][1,4]oxazine-7-carboxamide (Cpd. No. IX-6)

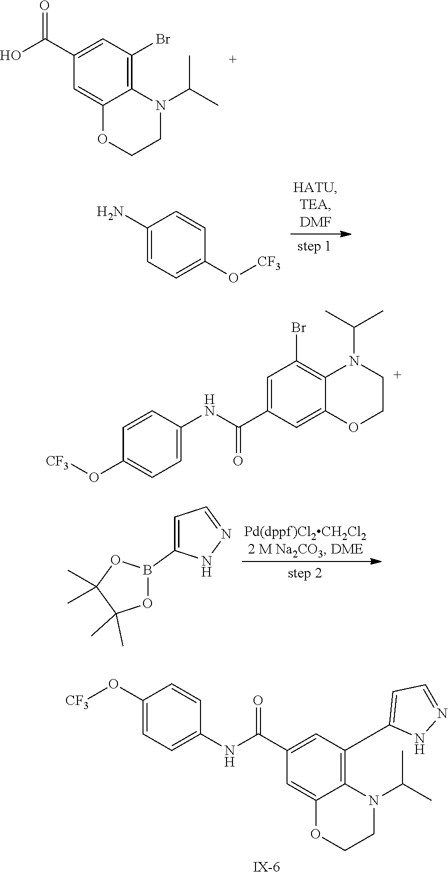

Essentially the same protocol of the preparation of Cpd. No. VII-2 in Example VII-2 was used to afford Cpd. No. IX-6 (9.0 mg) as a white solid. ¹H NMR (400 MHz, DMSO-d₆) δ 12.82 (s, 1H), 10.20 (s, 1H), 7.87 (d, J=8.7 Hz, 2H), 7.68 (s, 1H), 7.58 (s, 1H), 7.41 (s, 1H), 7.33 (d, J=8.7 Hz, 2H), 6.51 (s, 1H), 4.11 (t, J=4.3 Hz, 2H), 3.31-3.23 (m, 3H), 0.90 (d, J=6.6 Hz, 6H).

### Example X-1

### Synthesis of N-(4-(chlorodifluoromethoxy)phenyl)-1-isopropyl-9-(1H-pyrazol-5-yl)-2,3,4,5-tetrahydro-1H-benzo[b]azepine-7-carboxamide (Cpd. No. X-1)

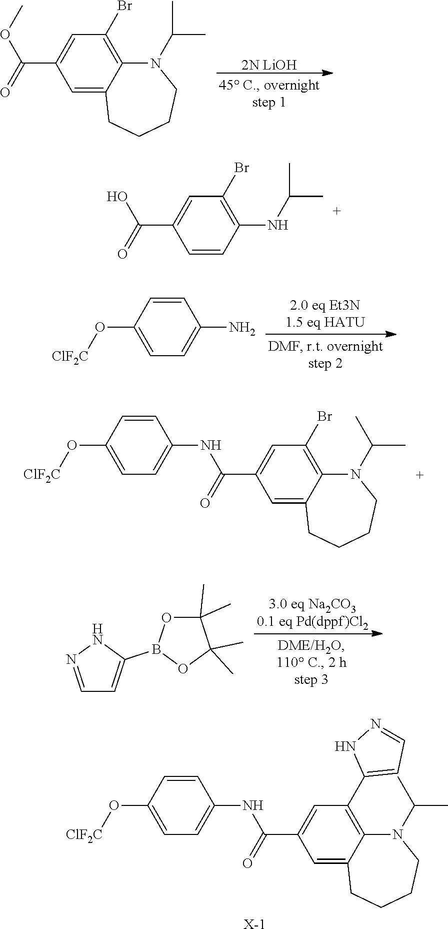

Essentially the same protocol of the preparation of Cpd. No. VII-2 in EXAMPLE VII-2 was used to afford Cpd. No. X-1 as a white solid. ¹H NMR (400 MHz, DMSO-d₆) δ 10.51 (s, 1H), 8.17 (s, 1H), 7.92-7.90 (m, 3H), 7.81 (d, J=2.1 Hz, 1H), 7.40 (d, J=8.6 Hz, 2H), 6.88 (s, 1H), 3.52-3.48 (m, 2H), 3.03-3.25 (m, 2H), 1.98-1.90 (m, 2H), 1.77-1.68 (m, 2H), 1.26-1.21 (m, 1H), 1.04 (d, J=6.6 Hz, 6H). MS: 475.1 (M+H)⁺.

### Example XII-1

### Synthesis of N-(4-(chlorodifluoromethoxy)phenyl)-1-isopropyl-7-(1H-pyrazol-5-yl)-1H-indole-5-carboxamide (Cpd. No. XII-1)

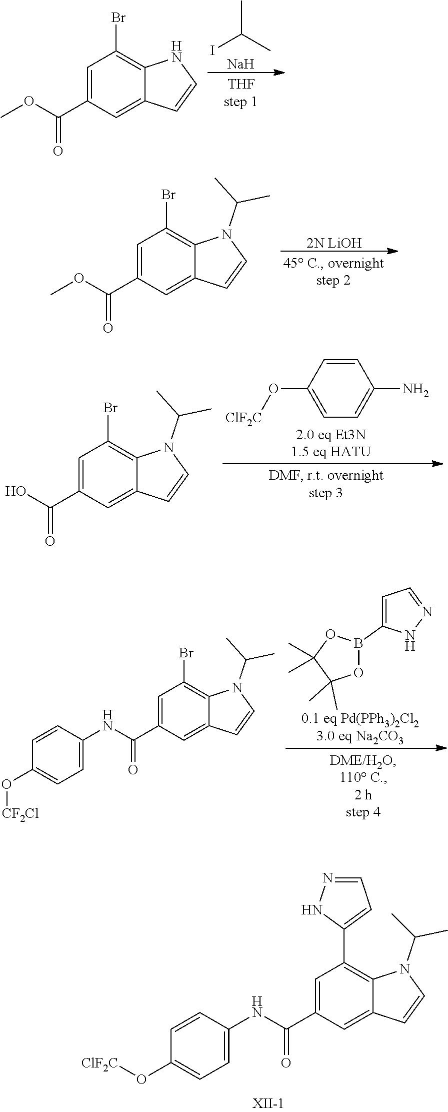

Essentially the same protocol of the preparation of Cpd. No. III-1 in Example III-1 was used to afford Cpd. No. XII-1 (30.0 mg) as a white solid. ¹H NMR (400 MHz, methanol-d₄) δ 8.33 (d, J=1.9 Hz, 1H), 7.89-7.79 (m, 3H), 7.71 (d, J=1.9 Hz, 1H), 7.55 (d, J=3.4 Hz, 1H), 7.34-7.26 (m, 2H), 6.77 (d, J=3.4 Hz, 1H), 6.58 (d, J=2.1 Hz, 1H), 4.19-4.07 (m, 1H), 1.31 (d, J=6.7 Hz, 6H). MS: 446.00 (M+H)⁺.

### Example XII-2

### Synthesis of N-(4-(chlorodifluoromethoxy)phenyl)-1-isopropyl-7-(pyrimidin-5-yl)-1H-indole-5-carboxamide (Cpd. No. XII-2)

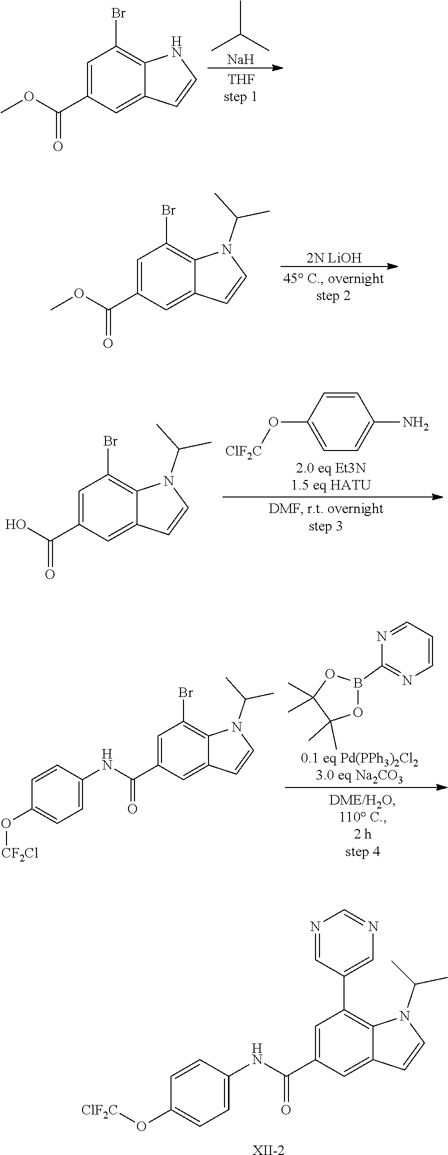

Essentially the same protocol of the preparation of Cpd. No. XII-1 in Example XII-1 was used to afford Cpd. No. XII-2 (10.0 mg) as a white solid. ¹H NMR (400 MHz, chloroform-d) δ 9.35 (s, 1H), 8.92 (s, 2H), 8.25 (d, J=1.8 Hz, 1H), 7.96 (s, 1H), 7.78-7.69 (m, 2H), 7.53 (d, J=1.9 Hz, 1H), 7.41 (d, J=3.4 Hz, 1H), 7.32-7.24 (m, 2H), 6.81 (d, J=3.4 Hz, 1H), 4.19-4.07 (m, 1H), 1.30 (d, J=6.6 Hz, 6H). MS: 458.00 (M+H)⁺.

### Example XII-3

### Synthesis of 3-chloro-N-(4-(chlorodifluoromethoxy)phenyl)-1-isopropyl-7-(1H-pyrazol-5-yl)-1H-indole-5-carboxamide (Cpd. No. XII-3)

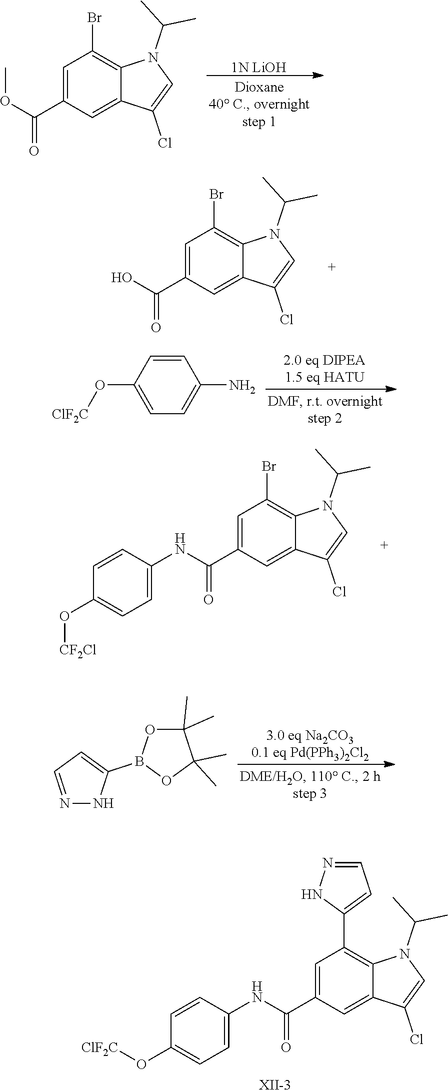

Essentially the same protocol of the preparation of Cpd. No. XII-1 in Example XII-1 was used to afford Cpd. No. XII-3 (45.0 mg) as a white solid. ¹H NMR (400 MHz, methanol-d₄) δ 8.33 (d, J=1.9 Hz, 1H), 7.89-7.82 (m, 3H), 7.79 (d, J=1.9 Hz, 1H), 7.63 (s, 1H), 7.34-7.26 (m, 2H), 6.60 (d, J=2.2 Hz, 1H), 4.19-4.07 (m, 1H), 1.30 (d, J=6.7 Hz, 6H). MS: 479.80 (M+H)⁺.

### Example XII-4

### 3-chloro-N-(4-(chlorodifluoromethoxy)phenyl)-1-isopropyl-7-(pyrimidin-5-yl)-1H-indole-5-carboxamide (Cpd. No. XII-4)

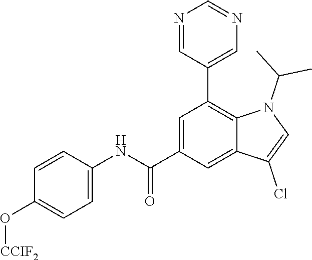

Essentially the same protocol of the preparation of Cpd. No. XII-1 in Example XII-1 was used to afford Cpd. No. XII-4 (80.0 mg) as a white solid. ¹H NMR (400 MHz, methanol-d₄) δ 9.31 (s, 1H), 9.04 (s, 2H), 8.40 (d, J=1.8 Hz, 1H), 7.91-7.82 (m, 2H), 7.74-7.68 (m, 2H), 7.35-7.27 (m, 2H), 4.15-4.10 (m, 1H), 1.32 (d, J=6.6 Hz, 6H).

### Example XII-5

### Synthesis of N-(4-(chlorodifluoromethoxy)phenyl)-1-isopropyl-3-methyl-7-(pyrimidin-5-yl)-1H-indole-5-carboxamide (Cpd. No. XII-5)

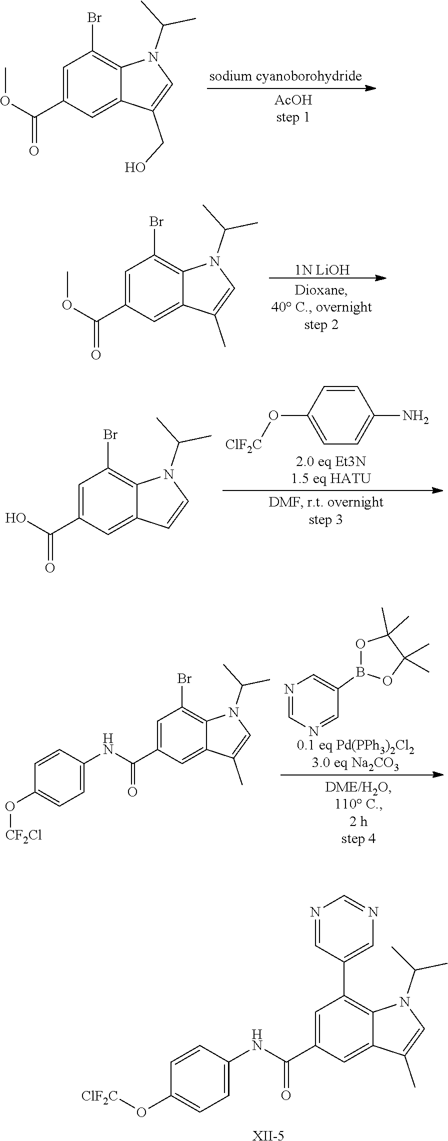

### Step 1: Synthesis of methyl 7-bromo-1-isopropyl-3-methyl-1H-indole-5-carboxylate

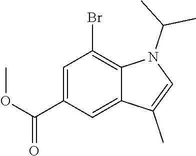 [SMILES: COC(=O)c1cc(Br)c2c(c1)c(C)cn2C(C)C]

In an oven-dried 50 mL round-bottomed flask methyl methyl 7-bromo-3-(hydroxymethyl)-1-isopropyl-1H-indole-5-carboxylate was dissolved in AcOH (5 mL), sodium cyanoborohydride was added to the mixture. The mixture was stirred for 3 h, then quenched by water, followed by extraction with ethyl acetate (50 mL×2). The combined organic layers were washed with brine, dried over Na₂SO₄, filtered and concentrated to give a crude product that was eluted through a silica gel column (ethyl acetate/hexane from 0 to 50%) to give methyl 7-bromo-1-isopropyl-3-methyl-1H-indole-5-carboxylate as a solid. MS: 309.90, 311.90.

### Step 2: Synthesis of 7-bromo-1-isopropyl-3-methyl-1H-indole-5-carboxylic acid

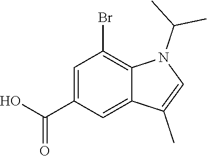 [SMILES: Cc1cn(C(C)C)c2c(Br)cc(C(=O)O)cc12]

Essentially the same protocol of step 2 in EXAMPLE XII-1 was used to afford 7-bromo-1-isopropyl-3-methyl-1H-indole-5-carboxylic acid. MS: 297.02 (M+H)⁺

### Step 3: Synthesis of 7-bromo-N-(4-(chlorodifluoromethoxy)phenyl)-1-isopropyl-3-methyl-1H-indole-5-carboxamide

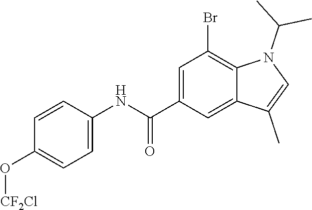

Essentially the same protocol of step 3 in EXAMPLE XII-1 was used to afford 7-bromo-N-(4-(chlorodifluoromethoxy)phenyl)-1-isopropyl-3-methyl-1H-indole-5-carboxamide. MS: 472.02 (M+H)⁺

### Step 4: Synthesis of N-(4-(chlorodifluoromethoxy)phenyl)-1-isopropyl-3-methyl-7-(pyrimidin-5-yl)-1H-indole-5-carboxamide

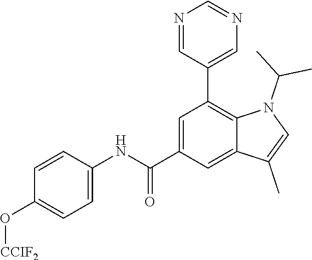

Essentially the same protocol of the preparation of Cpd. No. XII-1 in Example XII-1 was used to afford Cpd. No. XII-5 (35.0 mg) as a white solid. ¹H NMR (400 MHz, chloroform-d) δ 9.34 (s, 1H), 8.91 (s, 2H), 8.19 (d, J=1.8 Hz, 1H), 7.96 (s, 1H), 7.79-7.72 (m, 2H), 7.49 (d, J=1.8 Hz, 1H), 7.17 (s, 1H), 4.15-4.10 (m, 1H), 2.45 (s, 3H), 1.27 (d, J=6.8 Hz, 6H). MS: 472.00 (M+H)⁺.

### Example XII-6

### Synthesis of N-(4-(chlorodifluoromethoxy)phenyl)-1-isopropyl-3-methyl-7-(1H-pyrazol-5-yl)-1H-indole-5-carboxamide (Cpd. No. XII-6)

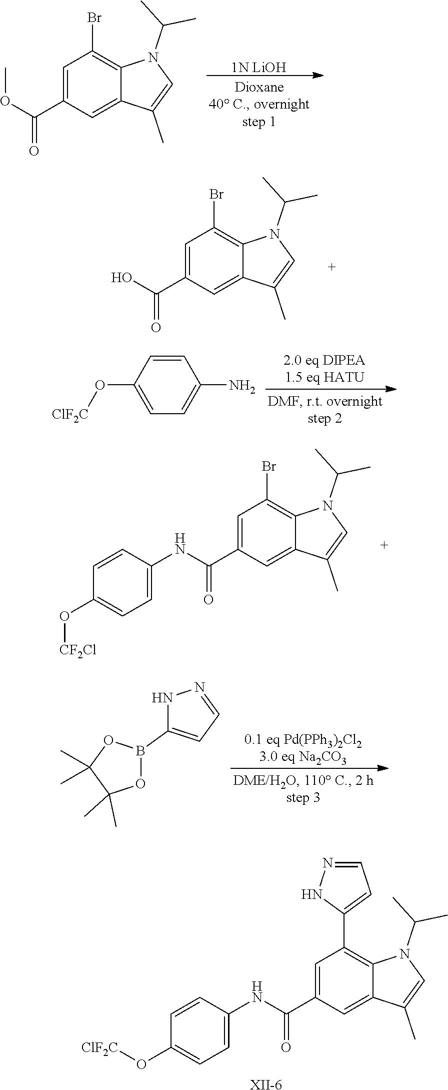

Essentially the same protocol of the preparation of Cpd. No. XII-1 in Example XII-1 was used to afford Cpd. No. XII-6 (60.0 mg) as a white solid. ¹H NMR (400 MHz, chloroform-d) δ 8.25 (s, 1H), 8.21 (d, J=1.9 Hz, 1H), 7.79-7.74 (m, 2H), 7.72 (d, J=2.1 Hz, 1H), 7.67 (d, J=1.9 Hz, 1H), 7.26 (s, 1H), 7.14 (s, 1H), 6.54 (d, J=2.1 Hz, 1H), 4.39 (p, J=6.4 Hz, 1H), 2.42 (d, J=1.1 Hz, 3H), 2.07-1.99 (m, 1H), 1.94 (s, 1H), 1.26 (s, 10H), 0.90 (t, J=6.6 Hz, 1H). MS: 459.95 (M+H)⁺.

### Example XII-7

### Synthesis of N-(4-(chlorodifluoromethoxy)phenyl)-3-(hydroxymethyl)-1-isopropyl-7-(pyrimidin-5-yl)-1H-indole-5-carboxamide (Cpd. No. XII-7)

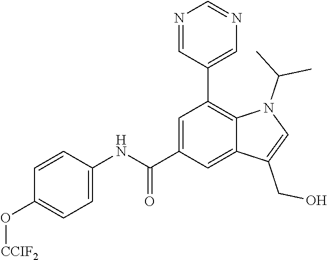

Essentially the same protocol of the preparation of Cpd. No. VII-41 in Example VII-41 was used to afford Cpd. No. XII-7 as a white solid. MS: 488.12 (M+H)⁺

### Example XII-8

### Synthesis of N-(4-(chlorodifluoromethoxy)phenyl)-3-(hydroxymethyl)-1-isopropyl-7-(1H-pyrazol-5-yl)-1H-indole-5-carboxamide (Cpd. No. XII-8)

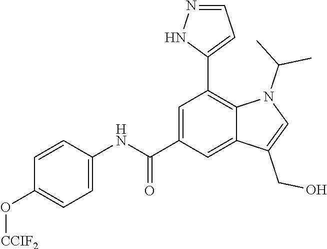

Essentially the same protocol of the preparation of Cpd. No. VII-41 in Example VII-41 was used to afford Cpd. No. XII-8 (5.0 mg) as a white solid. ¹H NMR (400 MHz, DMSO-d₆) δ 10.39 (s, 1H), 8.43-8.35 (m, 1H), 7.94 (d, J=8.6 Hz, 2H), 7.84 (s, 1H), 7.71 (d, J=1.8 Hz, 1H), 7.57 (s, 1H), 7.35 (d, J=8.6 Hz, 2H), 6.51 (s, 1H), 4.75 (s, 2H), 4.39-4.34 (m, 1H), 1.22 (d, J=6.6 Hz, 6H).

### Example XIII-1

### Synthesis of N-(4-(chlorodifluoromethoxy)phenyl)-1-isopropyl-7-(1H-pyrazol-5-yl)-1H-indazole-5-carboxamide (Cpd. No. XIII-1)

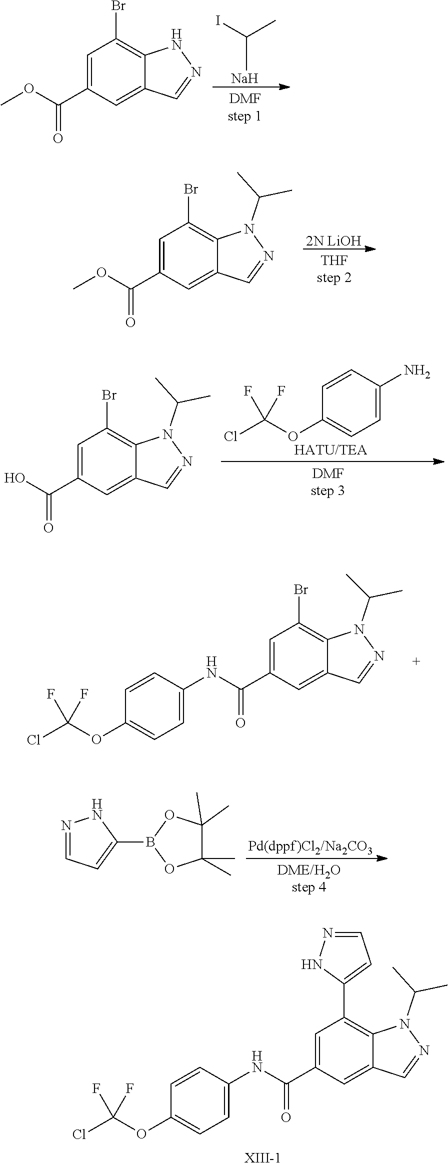

Essentially the same protocol of the preparation of Cpd. No. III-1 in EXAMPLE III-1 was used to afford Cpd. No. XIII-1 (11.0 mg) as a white solid. ¹H NMR (400 MHz, CD₃OD) δ 8.49 (s, 1H), 8.38-8.20 (m, 1H), 7.98-7.91 (m, 1H), 7.84-7.64 (m, 3H), 7.30 (d, J=6.1 Hz, 2H), 6.63 (dd, J=4.7, 2.5 Hz, 1H), 4.63-4.58 (m, 1H), 1.36 (d, J=6.6 Hz, 6H). MS: 446.3 (M+H)⁺.

### Example XIV

### Synthesis of N-(4-(chlorodifluoromethoxy)phenyl)-1-isopropyl-2-methyl-2-(morpholine-4-carbonyl)-7-(1H-pyrazol-5-yl)indoline-5-carboxamide (Cpd. No. XIV)

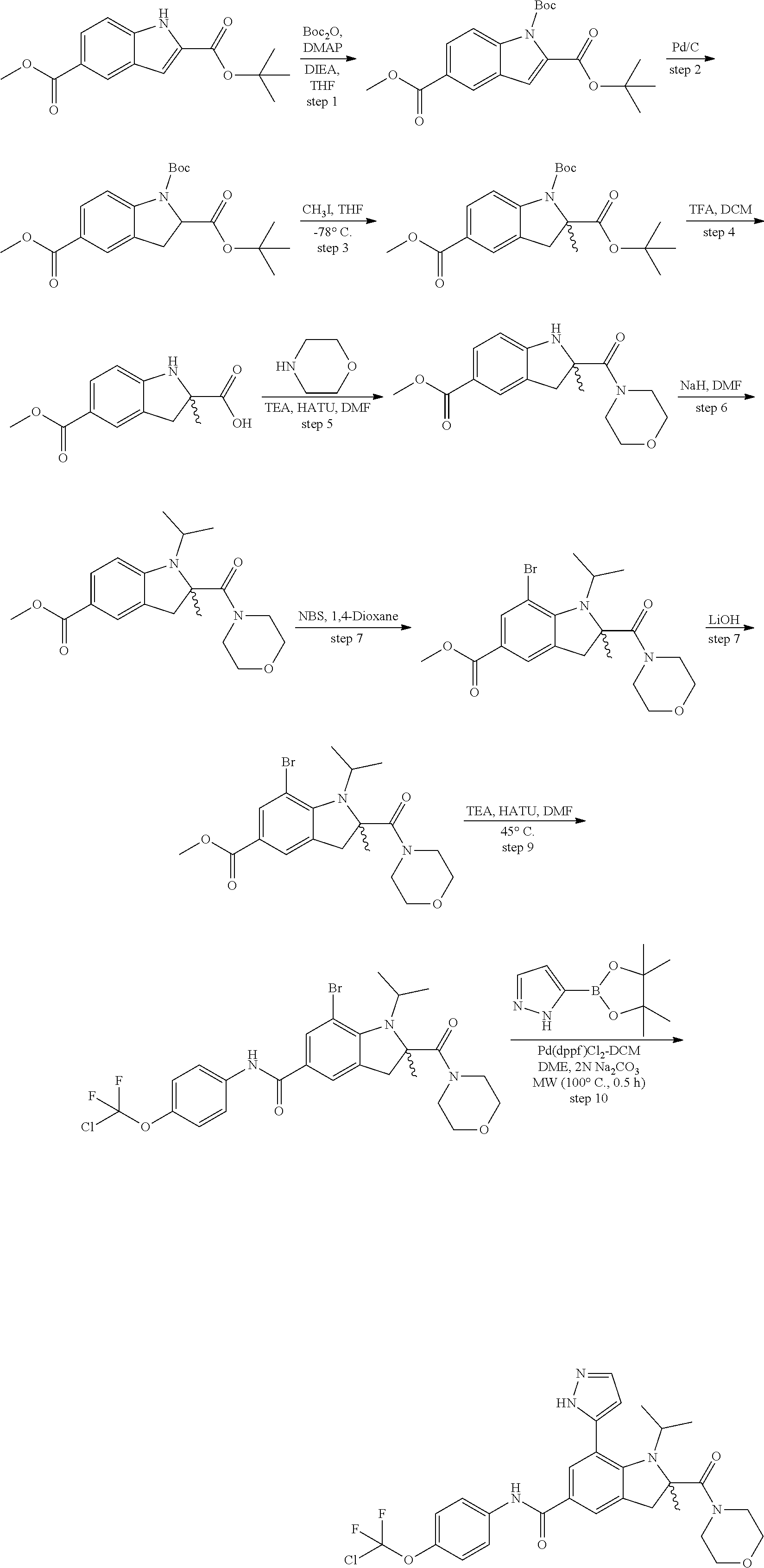

### Step 1: Synthesis of 1,2-di-tert-butyl 5-methyl 1H-indole-1,2,5-tricarboxylate

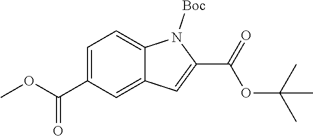 [SMILES: COC(=O)c1ccc2c(c1)cc(C(=O)OC(C)(C)C)n2C(=O)OC(C)(C)C]

Under Ar, a solution of 2-(tert-butyl) 5-methyl 1H-indole-2,5-dicarboxylate (5.1 g, 18.52 mmol), Boc₂O (6.7 g, 30.7 mmol) and DMAP (0.453 g, 3.70 mmol) in THF (100 mL) was stirred at 70° C. for 24 h. The mixture was portioned between ethyl acetate/water. The organic layer was washed with brine, dried over anhydrous Na₂SO₄, filtered and concentrated to give crude product, which was purified by column chromatography (hexane/ethyl acetate=5:1) to afford the compound 1,2-di-tert-butyl 5-methyl 1H-indole-1,2,5-tricarboxylate (2.9 g, 41.7%) as a yellow oil. ¹H NMR (400 MHz, DMSO-d₆) δ 8.37 (d, J=1.0 Hz, 1H), 8.03 (m, 2H), 7.39 (s, 1H), 3.88 (s, 3H), 1.60 (s, 9H), 1.55 (s, 9H). MS: 398.24 (M+Na)⁺.

### Step 2: Synthesis of 1,2-di-tert-butyl 5-methyl indoline-1,2,5-tricarboxylate

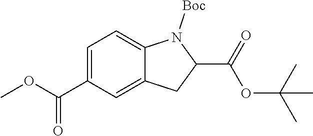 [SMILES: COC(=O)c1ccc2c(c1)CC(C(=O)OC(C)(C)C)N2C(=O)OC(C)(C)C]

A mixture of 1,2-di-tert-butyl 5-methyl 1H-indole-1,2,5-tricarboxylate (1.7 g, 4.53 mmol) and palladium on carbon (920 mg) in MeOH (60 mL) was hydrogenated at room temperature for 18 h. The reaction solution was filtered, and the filtrate was concentrated to give a residue which was purified by column chromatography (hexane/ethyl acetate=4:1) to afford the compound 1,2-di-tert-butyl 5-methyl indoline-1,2,5-tricarboxylate (1.43 g, 83.7%) as a white solid. ¹H NMR (400 MHz, DMSO-d₆) δ 7.87-7.78 (m, 2H), 7.76 (s, 1H), 4.82 (dd, J=11.6, 3.6 Hz, 1H), 3.81 (s, 3H), 3.67-3.49 (m, 1H), 3.05 (dd, J=16.8, 3.2 Hz, 1H), 1.45 (s, 6H), 1.42 (s, 12H). MS: 400.33 (M+Na)⁺.

### Step 3: Synthesis of 1,2-di-tert-butyl 5-methyl 2-methylindoline-1,2,5-tricarboxylate

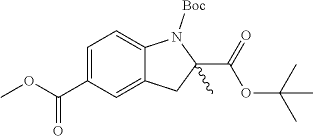 [SMILES: COC(=O)c1ccc2c(c1)CC(C)(C(=O)OC(C)(C)C)N2C(=O)OC(C)(C)C]

Under Ar, a solution of LiHMDS (17.93 mL, 17.93 mmol, 1M in THF) was dropped into a solution of 1,2-di-tert-butyl 5-methyl indoline-1,2,5-tricarboxylate (1.1 g, 2.91 mmol, 1.0 eq.) in anhydrous THF (23 mL) at −78° C. After being stirred for 0.5 h at 0° C., into the mixture was dropped iodomethane (9.08 g, 64.0 mmol), and the mixture was allowed to warm to room temperature. The mixture was stirred at room temperature for 1 h, then quenched with saturated NH₄Cl solution, and extracted with ethyl acetate (30 mL×3). The combined organic layers were dried over anhydrous Na₂SO₄, filtered and concentrated to give a crude product that was purified by column chromatography (hexane/ethyl acetate=4:1) to afford the compound 1,2-di-tert-butyl 5-methyl 2-methylindoline-1,2,5-tricarboxylate (1.14 g, 100%, crude). MS: 392.27 (M+H)⁺.

### Step 4: Synthesis of 5-(methoxycarbonyl)-2-methylindoline-2-carboxylic acid

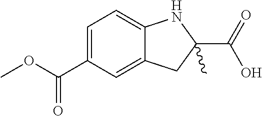 [SMILES: COC(=O)c1ccc2c(c1)CC(C)(C(=O)O)N2]

Under Ar, to a solution of 1,2-di-tert-butyl 5-methyl 2-methylindoline-1,2,5-tricarboxylate (1.2 g, 3.07 mmol) in anhydrous dichloromethane (17 mL) were added trifluoroacetic acid (10 mL) and triethylsilane (1.07 g, 9.20 mmol) at 0° C. The solution was stirred at room temperature for 18 h, then evaporated in vacuo to afford crude product 5-(methoxycarbonyl)-2-methylindoline-2-carboxylic acid (750 mg) which was used directly for the next step. MS: 236.52 (M+H)⁺.

### Step 5: Synthesis of methyl 2-methyl-2-(morpholine-4-carbonyl)indoline-5-carboxylate

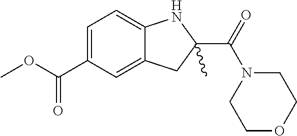 [SMILES: COC(=O)c1ccc2c(c1)CC(C)(C(=O)N1CCOCC1)N2]

Under Ar, 2-(7-azabenzotriazol-1-yl)-N,N,N′,N′-tetramethyluronium hexafluorophosphate (2.1 g, 5.52 mmol) was dropped to a solution of 5-(methoxycarbonyl)-2-methylindoline-2-carboxylic acid (721 mg, 3.06 mmol), morpholine (801 mg, 9.19 mmol) and triethylamine (1.55 g, 15.32 mmol) in anhydrous DMF (8 mL) at 0° C. The mixture was stirred at this temperature for 0.5 h, then portioned between ethyl acetate and water. The organic layer was washed with brine, dried over anhydrous Na₂SO₄, filtered and concentrated to give crude product, which was purified by column chromatography (hexane/ethyl acetate=1:2) to afford the compound methyl 2-methyl-2-(morpholine-4-carbonyl)indoline-5-carboxylate (828 mg, 89%). MS: 305.19 (M+H)⁺.

### Step 6: Synthesis of methyl 1-isopropyl-2-methyl-2-(morpholine-4-carbonyl)indoline-5-carboxylate

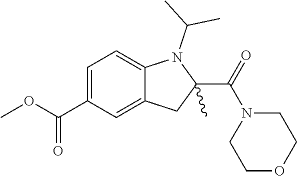 [SMILES: COC(=O)c1ccc2c(c1)CC(C)(C(=O)N1CCOCC1)N2C(C)C]

Under Ar, NaH (1.0 g, 41.7 mmol) was dropped to a solution of methyl 2-methyl-2-(morpholine-4-carbonyl)indoline-5-carboxylate (700 mg, 2.30 mmol) in anhydrous DMF (18 mL) at 0° C. The mixture was stirred for 30 minutes at the same temperature, then was added 2-iodopropane (4.57 g, 26.9 mmol). The mixture was stirred at room temperature for 18 h, then poured into ice water and partitioned between ethyl acetate/water. The organic layer was neutralized to pH=6, then extracted with ethyl acetate (50 mL×3). The organic layers were dried over anhydrous Na₂SO₄, filtered and concentrated to afford crude product 1-isopropyl-2-methyl-2-(morpholine-4-carbonyl)indoline-5-carboxylic acid (700 mg, 92%). Under Ar, iodomethane (3.422 g, 24.11 mmol) was added to a mixture of 2-methyl-2-(morpholine-4-carbonyl)indoline-5-carboxylic acid (700 mg, 2.411 mmol) and K₂CO₃ (3.332 g, 24.11 mmol) in anhydrous acetonitrile (30 mL) at room temperature. The reaction was stirred for 30 minutes then filtered. The filtrate was concentrated to give the crude product, which was purified by column chromatography (hexane/ethyl acetate=1:1) to afford methyl 1-isopropyl-2-methyl-2-(morpholine-4-carbonyl)indoline-5-carboxylate (270 mg, 32.3%). MS: 347.25 (M+H)⁺.

### Step 7: Synthesis of methyl 7-bromo-1-isopropyl-2-methyl-2-(morpholine-4-carbonyl)indoline-5-carboxylate

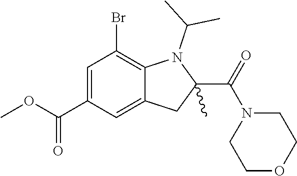 [SMILES: COC(=O)c1cc(Br)c2c(c1)CC(C)(C(=O)N1CCOCC1)N2C(C)C]

Under Ar, N-Bromosuccinimide (150 mg, 0.843 mmol) was dropped to a solution of methyl 1-isopropyl-2-methyl-2-(morpholine-4-carbonyl)indoline-5-carboxylate (230 mg, 0.664 mmol) in 1,4-dioxane (10 mL) under 5° C. The mixture was stirred at room temperature for 3 h. The solution was portioned between ethyl acetate/water. The organic layer was dried over anhydrous Na₂SO₄, filtered and concentrated to give a crude product that was purified by column chromatography (hexane/ethyl acetate=2:1) to afford methyl 7-bromo-1-isopropyl-2-methyl-2-(morpholine-4-carbonyl)indoline-5-carboxylate (200 mg, 70.8%). MS: 425.35 (M+H)⁺.

### Step 8: Synthesis of 7-bromo-1-isopropyl-2-methyl-2-(morpholine-4-carbonyl)indoline-5-carboxylic acid

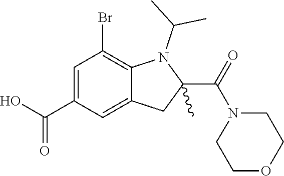 [SMILES: CC(C)N1c2c(Br)cc(C(=O)O)cc2CC1(C)C(=O)N1CCOCC1]

To a solution of methyl 7-bromo-1-isopropyl-2-methyl-2-(morpholine-4-carbonyl)indoline-5-carboxylate (200 mg, 0.47 mmol) in 1,4-dioxane (15 mL) was added a LiOH (5.68 mL, 5.68 mmol, 1 N in water) solution. The mixture was stirred at 45° C. for 18 h, then evaporated in vacuo. The resulting residue was diluted with water then acidified to pH=6. The resulting precipitate was collected and dried in vacuo to afford 7-bromo-1-isopropyl-2-methyl-2-(morpholine-4-carbonyl)indoline-5-carboxylic acid (180 mg, 93%), which was used directly for the next step. MS: 411.28 (M+H)⁺.

### Step 9: Synthesis of 7-bromo-N-(4-(chlorodifluoromethoxy)phenyl)-1-isopropyl-2-methyl-2-(morpholine-4-carbonyl)indoline-5-carboxamide

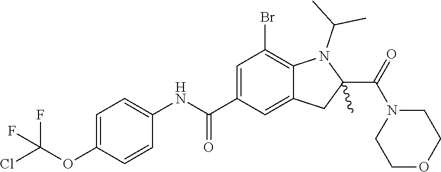 [SMILES: CC(C)N1c2c(Br)cc(C(=O)Nc3ccc(OC(F)(F)Cl)cc3)cc2CC1(C)C(=O)N1CCOCC1]

Under Ar, to a solution of 7-bromo-1-isopropyl-2-methyl-2-(morpholine-4-carbonyl)indoline-5-carboxylic acid (200 mg, 0.486 mmol), triethylamine (1.135 g, 11.22 mmol) and 4-(chlorodifluoromethoxy)aniline (527 mg, 2.72 mmol) in anhydrous N,N-dimethylformamide (6 mL) was added 2-(7-azabenzotriazol-1-yl)-N,N,N′,N′-tetramethyluronium hexafluorophosphate (570 mg, 1.50 mmol) at 0° C. The mixture was stirred at 45° C. for 4 h, then partitioned between ethyl acetate/water. The organic layer was dried over Na₂SO₄, filtered and concentrated to give a crude product that was purified by column chromatography (eluting with hexane/ethyl acetate=1:1) to afford 7-bromo-N-(4-(chlorodifluoromethoxy)phenyl)-1-isopropyl-2-methyl-2-(morpholine-4-carbonyl)indoline-5-carboxamide (205 mg, 71.8%) as a yellow oil. MS: 586.21 (M+H)⁺.

### Step 10: Synthesis of N-(4-(chlorodifluoromethoxy)phenyl)-1-isopropyl-2-methyl-2-(morpholine-4-carbonyl)-7-(1H-pyrazol-5-yl)indoline-5-carboxamide (Cpd. No. XIV)

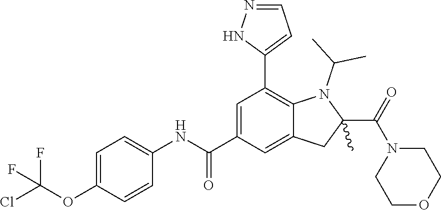 [SMILES: CC(C)N1c2c(cc(C(=O)Nc3ccc(OC(F)(F)Cl)cc3)cc2-c2ccn[nH]2)CC1(C)C(=O)N1CCOCC1]

Under Ar, a mixture of 7-bromo-N-(4-(chlorodifluoromethoxy)phenyl)-1-isopropyl-2-methyl-2-(morpholine-4-carbonyl)indoline-5-carboxamide (88 mg, 0.15 mmol), 5-(4,4,5,5-tetramethyl-1,3,2-dioxaborolan-2-yl)-1H-pyrazole (184 mg, 0.948 mmol) and PdCl₂(dppf)-CH₂Cl₂ adduct (25 mg, 0.03 mmol) in dimethoxyethane (7.5 mL) and 2N Na₂CO₃ (2.5 mL) solution was stirred at 100° C. under microwave for 0.5 h. The mixture was diluted with ethyl acetate, then washed with water and brine. The organic layer was dried over anhydrous Na₂SO₄, filtered and concentrated to give the crude product, which was purified by prep-HPLC to afford N-(4-(chlorodifluoromethoxy)phenyl)-1-isopropyl-2-methyl-2-(morpholine-4-carbonyl)-7-(1H-pyrazol-5-yl)indoline-5-carboxamide (26.2 mg, 30.4%) as a white solid. ¹H NMR (400 MHz, DMSO-d₆) δ 13.11-12.75 (m, 1H), 10.10 (s, 1H), 7.85 (d, J=8.7 Hz, 2H), 7.82-7.69 (m, 2H), 7.66 (s, 1H), 7.31 (d, J=8.7 Hz, 2H), 6.43-6.27 (m, 1H), 4.17-3.85 (m, 1H), 3.81-3.43 (m, 9H), 3.05 (d, J=17.0 Hz, 1H), 1.44 (s, 3H), 1.08 (d, J=6.5 Hz, 3H), 0.75 (d, J=6.6 Hz, 3H). MS: 575.40 (M+H)⁺.

### Example 2—Biological Activity

WST Assay

The anti-proliferative effect of representative Compounds of the Disclosure was determined by a water-soluble tetrazolium (WST)-based assay using Cell Counting Kit-8 (CCK-8). Cells were seeded in 96-well plates and treated with different concentrations of test articles for 72 h. Each treatment was tested in triplicates. Briefly, a series of 9 concentrations were chosen for each test article and added at 100 μl/well into a 96-well plate. Each concentration was tested in triplicates. 100 μl of diluent was added into 3-6 wells in the same plate for cell control group, and another set of 3-6 wells was used as a blank control.

In each well, 100 μl of cell suspension (containing optimal cell numbers, which creates approximately 100% confluence of cells in control wells based on by the absorbance (OD) value) was dispensed into the same 96-well plate except for the blank well. The plate was then cultured at 37° C. in an incubator in an atmosphere with 5% CO₂ for 72 h. At the end of treatments, 20 μl/well of CCK-8 reagent was directly added to each wells. The plate was then incubated at 37° C. in an incubator in an atmosphere with 5% CO₂ for 2-4 hour. The OD value was then detected at 450 nm on a microplate reader (SpectraMax Plus 384, Molecular Devices, LLC. US).

The cells viability was calculated using the mean OD value of triplicated wells following the equation below: (OD sample−OD blank)/(OD cell control−OD blank)×100 IC₅₀ values were calculated with Graphpad Prism 6.0 software using nonlinear regression (curve fitting) type data analysis.

In this assay, ABL001 ((R)—N-(4-(chlorodifluoromethoxy)phenyl)-6-(3-hydroxy-pyrrolidin-1-yl)-5-(1H-pyrazol-5-yl)nicotinamide) was used as control reference. ABL001 is the first allosteric BCR-ABL inhibitor in clinical trial that selectively inhibits growth of BCR-ABL1-driven cells, and is commercially available.

The results of cell-based assays are listed provided in Table 2.

TABLE 2

| Cpd. No. | BaF3(WT, nM) | BaF3(T315I, nM) |
|---|---|---|
| ABL001 (control) | 42 | 294 |
| III-1 | 119 | 3248 |
| III-2 | 78 | 2057 |
| III-3 | 94 | 790 |
| III-4 | 22 | 176 |
| III-5 | 61 | 418 |
| IV-1 | 24 | 244 |
| IV-2 | 399 | 3340 |
| IV-3 | 132 | 2379 |
| IV-4 | 129 | 581 |
| IV-5 | 121 | 1062 |
| IV-6 | 501 | 1572 |
| IV-7 | 364 | 1750 |
| IV-8 | 100 | 6018 |
| IV-9 | 77 | 107 |
| V-1 | 45 | 71 |
| V-2 | 55 | 354 |
| V-3 | 22 | 136 |
| V-4 | 43 | 292 |
| V-5 | 144 | 335 |
| V-6 | 364 | 2856 |
| V-7 | 324 | 1535 |
| VII-1 | 26 | 91 |
| VII-2 | 29 | 272 |
| VII-3 | 123 | 574 |
| VII-4 | 42 | 136 |
| VII-5 | 34 | 295 |
| VII-6 | 112 | 250 |
| VII-7 | 113 | 144 |
| VII-8 | 23 | 468 |
| VII-9 | 403 | 4965 |
| VII-10 | 1158 | 3841 |
| VII-11 | 29 | 946 |
| VII-12 | 112 | 123 |
| VII-13 | 266 | 1411 |
| VII-14 | 56 | 660 |
| VII-15 | 19 | 131 |
| VII-16 | 28 | 278 |
| VII-17 | 584 | 3229 |
| VII-18 | 124 | 484 |
| VII-19 | 133 | 1087 |
| VII-20 | 165 | 2177 |
| VII-21 | 158 | 1610 |
| VII-22 | 145 | 1351 |
| VII-23 | 17 | 146 |
| VII-24 | 1686 | 3858 |
| VII-25 | 58 | 243 |
| VII-26 | 39 | 199 |
| VII-27 | 85 | 366 |
| VII-28 | 132 | 575 |
| VII-29 | 113 | 940 |
| VII-30 | 16 | 94 |
| VII-31 | 78 | 423 |
| VII-32 | 308 | 2156 |
| VII-33 | 49 | 163 |
| VII-34 | 45 | 648 |
| VII-35 | 173 | 1186 |
| VII-36 | 231 | 1738 |
| VII-37 | 41 | 395 |
| VII-38 | 20 | 223 |
| VII-39 | 62 | 1051 |
| VII-40 | 20 | 256 |
| VII-41 | 46 | 168 |
| VII-42 | 16 | 1179 |
| VII-43 | 16 | 332 |
| VII-44 | 66 | 666 |
| VII-45 | 18 | 474 |
| VIII-1 | 458 | 3680 |
| VIII-2 | 359 | 1987 |
| VIII-3 | 313 | 1014 |
| IX-1 | 235 | 1054 |
| IX-2 | 162 | 332 |
| IX-3 | 80 | 535 |
| IX-4 | 33 | 3109 |
| IX-5 | 17 | 333 |
| X-1 | 67 | 1288 |
| XII-1 | 115 | 654 |
| XII-2 | 571 | 716 |
| XII-3 | 505 | 3274 |
| XII-4 | 128 | 726 |
| XII-5 | 103 | 3179 |
| XII-6 | 22 | 511 |
| XII-7 | 82 | 552 |
| XII-8 | 57 | 344 |
| V-11 | 8 | 40 |
| V-8 | 1546 | 10,000 |
| V-9 | 108 | 910 |
| V-10 | 110 | 921 |

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof.

All patents, patent applications, and publications cited herein are fully incorporated by reference herein in their entirety.

We claim:

1. A compound of Formula I: 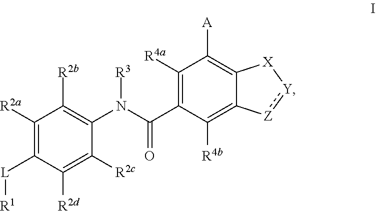
wherein:
R¹ is C₁-C₃ haloalkyl;
L is selected from the group consisting of —S— and —O—;
R^{2a}, R^{2b}, R^{2c}, and R^{2d} are independently selected from the group consisting of hydrogen, halo, C₁-C₃ alkyl, and C₁-C₃ alkoxy;
R³ is selected from the group consisting of hydrogen and C₁-C₃ alkyl;
R^{4a} and R^{4b} are independently selected from the group consisting of hydrogen, halo, C₁-C₃ alkyl, and C₁-C₃ alkoxy;
A is selected from the group consisting of optionally substituted 5-membered heteroaryl and optionally substituted 6-membered heteroaryl;
X is —C(R^{5a})(R^{5b})—; Y is —C(R^{5c})(R^{5d}); Z is —N(R^{5c})—; and [symbol] is a single bond;
R^{5a} and R^{5b} are independently selected from the group consisting of hydrogen and C₁-C₄ alkyl; or
R^{5a} and R^{5b} taken together with the carbon atom to which they are attached form an optionally substituted C₃-C₈ cycloalkyl or optionally substituted 4- to 8-membered heterocyclo;
R^{5c} and R^{5d} are independently selected from the group consisting of hydrogen and C₁-C₄ alkyl;
R^{5c} is selected from the group consisting of hydrogen, C₁-C₆ alkyl, hydroxyalkyl, (amino)alkyl, (heterocyclo)alkyl, optionally substituted C₃-C₆ cycloalkyl, optionally substituted 4- to 8-membered heterocyclo, —C(═O)R⁶, and —S(═O)₂R⁷;
R⁶ is selected from the group consisting of C₁-C₆ alkyl, optionally substituted C₃-C₆ cycloalkyl, and optionally substituted 4- to 8-membered heterocyclo;
R⁷ is selected from the group consisting of C₁-C₆ alkyl optionally substituted C₃-C₆ cycloalkyl, and optionally substituted 4- to 8-membered heterocyclo; or
X is —C(R^{8a})(R^{8b})—; Y is —N(R^{8c})—; Z is —C(═O)—; and [symbol] is a single bond;
R^{8a} and R^{8b} are independently selected from the group consisting of hydrogen and C₁-C₄ alkyl; or
R^{8a} and R^{8b} taken together with the carbon atom to which they are attached form an optionally substituted C₃-C₈ cycloalkyl or optionally substituted 4- to 8-membered heterocyclo;
R^{8c} is selected from the group consisting of hydrogen, optionally substituted C₁-C₆ alkyl, hydroxyalkyl, (amino)alkyl, optionally substituted C₃-C₆ cycloalkyl, optionally substituted 4- to 8-membered heterocyclo, and (heterocyclo)alkyl; or
X is —C(R^{9a})(R^{9b})—; Y is —C(═O)—; Z is —N(R^{9c})—; and [symbol] is a single bond;
R^{9a} and R^{9b} are independently selected from the group consisting of hydrogen and C₁-C₄ alkyl; or
R^{9a} and R^{9b} taken together with the carbon atom to which they are attached form an optionally substituted C₃-C₈ cycloalkyl or optionally substituted 4- to 8-membered heterocyclo;
R^{9c} is selected from the group consisting of hydrogen, optionally substituted C₁-C₆ alkyl, hydroxyalkyl, (amino)alkyl, optionally substituted C₃-C₆ cycloalkyl, optionally substituted 4- to 8-membered heterocyclo, and (heterocyclo)alkyl; or
X is —N(R^{10a})—; Y is —C(R^{10b})(R^{10c})—; Z is —C(R^{10d})(R^{10e})—; and [symbol] is a single bond;
R^{10a} is selected from the group consisting of hydrogen, C₁-C₆ alkyl, hydroxyalkyl, (amino)alkyl, (heterocyclo)alkyl, optionally substituted C₃-C₆ cycloalkyl, optionally substituted 4- to 8-membered heterocyclo, —C(═O)R^{11a}, and —S(═O)₂R^{12a};
R^{10b} is selected from the group consisting of hydrogen, —CO₂H, C₁-C₄ alkyl, hydroxyalkyl, (amino)alkyl, (heterocyclo)alkyl, optionally substituted C₃-C₆ cycloalkyl, optionally substituted 4- to 8-membered heterocyclo, —C(═O)R^{11b}—S(═O)₂R^{12b}, —(CH₂)ₘ—C(═O)N(R^{13a})(R^{13b}), and —(CH₂)ₙ—N(R¹⁴)C(═O)R¹⁵;
R^{11c} is selected from the group consisting of hydrogen and C₁-C₄ alkyl;
R^{10d} and R^{10e} are independently selected from the group consisting of hydrogen, C₁-C₄ alkyl, (amino)alkyl, and hydroxyalkyl;
R^{11a} is selected from the group consisting of C₁-C₆ alkyl, optionally substituted C₃-C₆ cycloalkyl, and optionally substituted 4- to 8-membered heterocyclo;
R^{11b} is selected from the group consisting of hydroxy, C₁-C₆ alkyl and optionally substituted C₃-C₆ cycloalkyl;
R^{12a} is selected from the group consisting of C₁-C₆ alkyl, optionally substituted C₃-C₆ cycloalkyl, and optionally substituted 4- to 8-membered heterocyclo;
R^{12b} is selected from the group consisting of C₁-C₆ alkyl, optionally substituted C₃-C₆ cycloalkyl, and optionally substituted 4- to 8-membered heterocyclo;
R^{13a} is selected from the group consisting of hydrogen, optionally substituted C₁-C₆ alkyl, alkoxyalkyl, (amino)alkyl, (heterocyclo)alkyl, substituted C₃-C₆ cycloalkyl, and optionally substituted 4- to 8-membered heterocyclo;
R^{13b} is selected from the group consisting of hydrogen, C₁-C₆ alkyl, and alkoxyalkyl; or
R^{13a} and R^{13b} taken together form an optionally substituted 4- to 8-membered heterocyclo;
R¹⁴ is selected from the group consisting of hydrogen and C₁-C₄ alkyl;
R¹⁵ is selected from the group consisting of C₁-C₆ alkyl, substituted C₃-C₆ cycloalkyl, optionally substituted 4- to 8-membered heterocyclo;
m is 0, 1, or 2; and
n is 0, 1, or 2; or
X is —N(R^{16a})—; Y is —C(═O)—; Z is —C(R^{16b})(R^{16c})—; and [symbol] is a single bond;
R^{16a} is selected from the group consisting of hydrogen, C₁-C₆ alkyl, and optionally substituted C₃-C₆ cycloalkyl;
R^{16b} is selected from the group consisting of hydrogen and C₁-C₄ alkyl;
R^{16c} is selected from the group consisting of hydrogen and C₁-C₄ alkyl; or
R^{16b} and R^{16c} taken together with the carbon atom to which they are attached form an optionally substituted C₃-C₈ cycloalkyl or optionally substituted 4- to 8-membered heterocyclo; or
X is —N(R^{17a})—; Y is —C(R^{17b})(R^{17c})ₒ—; Z is selected from the group consisting of —O—, —S—, —N(R^{17d})— and —C(R^{17e})(R^{17f})—; and [symbol] is a single bond;
R^{17a} is selected from the group consisting of hydrogen, C₁-C₆ alkyl, and optionally substituted C₃-C₆ cycloalkyl;
each R^{17b} is independently selected from the group consisting of hydrogen and C₁-C₄ alkyl;
each R^{17c} is independently selected from the group consisting of hydrogen and C₁-C₄ alkyl;
R^{17d} is selected from the group consisting of hydrogen, C₁-C₆ alkyl, and optionally substituted C₃-C₆ cycloalkyl;
R¹⁷, is selected from the group consisting of hydrogen and C₁-C₄ alkyl;
R^{17f} is selected from the group consisting of hydrogen and C₁-C₄ alkyl; and
is 1 or 2; or
X is —N(R^{18a})—; Y is —C(R^{18b})═; Z is —C(R^{18c})═; and [symbol] is a double bond;
R^{18a} is selected from the group consisting of hydrogen, C₁-C₆ alkyl, and optionally substituted C₃-C₆ cycloalkyl;
R^{18b} is selected from the group consisting of hydrogen and C₁-C₄ alkyl; and
R^{18c} is selected from the group consisting of hydrogen, halo, C₁-C₄ alkyl, and hydroxyalkyl; or
X is —N(R^{19a})—; Y is —N═; Z is —C(R^{19b})═; and [symbol] is a double bond;
R^{19a} is selected from the group consisting of hydrogen, C₁-C₆ alkyl, and optionally substituted C₃-C₆ cycloalkyl; and
R^{19b} is selected from the group consisting of hydrogen and C₁-C₄ alkyl, or a pharmaceutically acceptable salt or solvate thereof.

2. (canceled)

3. (canceled)

4. The compound of claim 1 of Formula III: 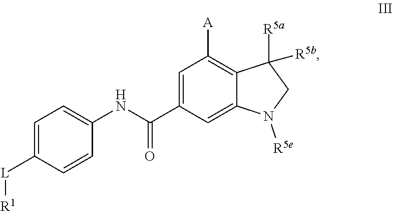
or a pharmaceutically acceptable salt or solvate thereof, wherein
R¹ is —CF₃ or —CF₂Cl; and
A is

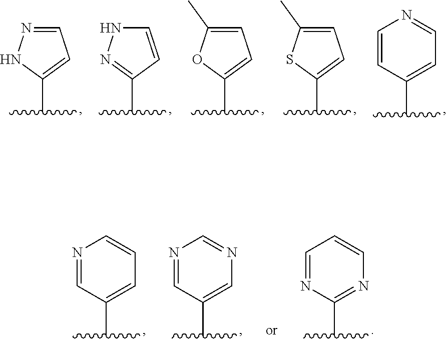

5. The compound of claim 4, wherein R^{5a} and R^{5b} are independently selected from the group consisting of hydrogen and C₁-C₄ alkyl; and R^{Se} is selected from the group consisting of hydrogen, C₁-C₆ alkyl, —C(═O)R⁶, and —S(═O)₂R⁷, or a pharmaceutically acceptable salt or solvate thereof.

6. (canceled)

7. The compound of claim 1 of Formula IV: 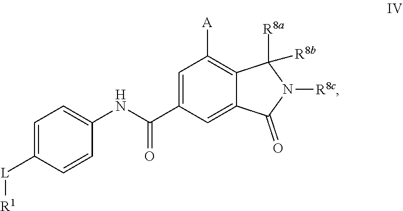
or a pharmaceutically acceptable salt or solvate thereof wherein
R¹ is —CF₃ or —CF₂Cl; and
A is

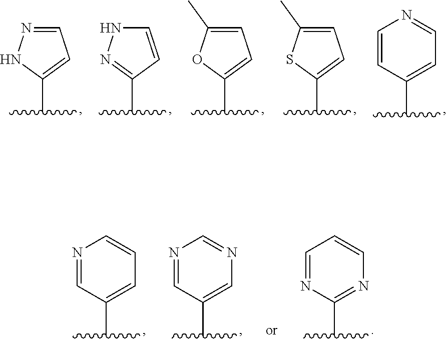

8. The compound of claim 7, wherein R^{8a} and R^{8b} are independently selected from the group consisting of hydrogen and C₁-C₄ alkyl; and R^{8c} is selected from the group consisting of hydrogen, C₁-C₆ alkyl, hydroxyalkyl, and (heterocyclo)alkyl, or a pharmaceutically acceptable salt or solvate thereof.

9. (canceled)

10. The compound of claim 1 of Formula V: 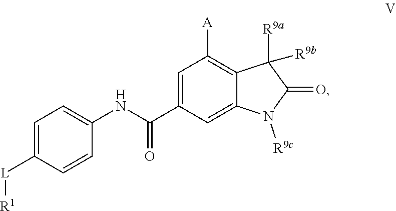
or a pharmaceutically acceptable salt or solvate thereof, wherein
R¹ is —CF₃ or —CF₂Cl; and
A is and

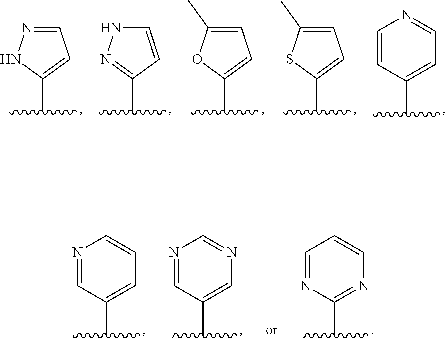

11. The compound of claim 10, wherein R^{9a} and R^{9b} are independently C₁-C₄ alkyl, or R^{9a} and R^{9b} taken together with the carbon atom to which they are attached form an optionally substituted C₃-C₆ cycloalkyl; and R^{9c} is selected from the group consisting of hydrogen, C₁-C₆ alkyl, hydroxyalkyl, and (amino)alkyl, or a pharmaceutically acceptable salt or solvate thereof.

12. (canceled)

13. (canceled)

14. The compound of claim 1 of Formula VI: 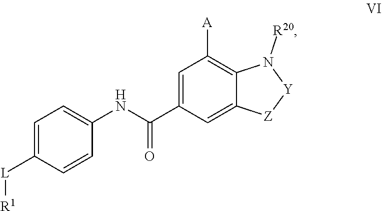
wherein
R¹ is —CF₃ or —CF₂Cl; and
A is 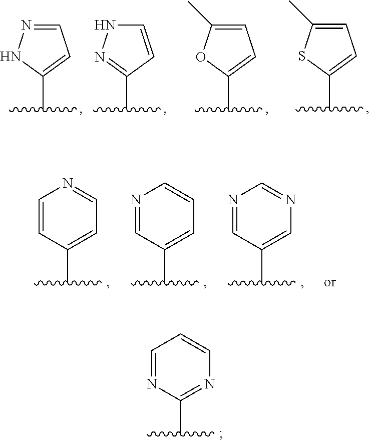
and
R²⁰ is selected from the group consisting of hydrogen, C₁-C₆ alkyl, hydroxyalkyl, (amino)alkyl, (heterocyclo)alkyl, optionally substituted C₃-C₆ cycloalkyl, optionally substituted 4- to 8-membered heterocyclo, or a pharmaceutically acceptable salt or solvate thereof.

15. The compound of claim 1 of Formula VII: 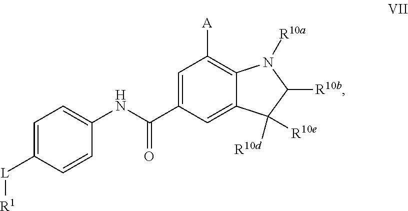
or a pharmaceutically acceptable salt or solvate thereof, wherein
R¹ is —CF₃ or —CF₂Cl; and
A is

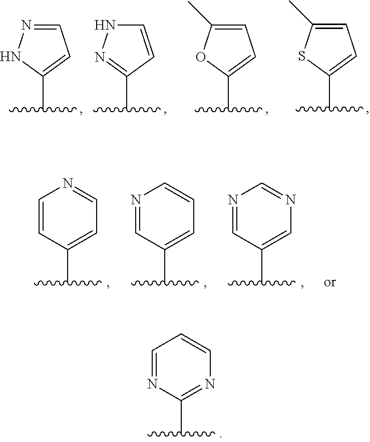

16. The compound of claim 15, wherein:
R^{10a} is selected from the group consisting of C₁-C₆ alkyl, hydroxyalkyl, (amino)alkyl, (heterocyclo)alkyl, optionally substituted C₃-C₆ cycloalkyl, —C(═O)R^{11a} or —S(═O)₂R^{12a}; and R^{10b} is selected from the group consisting of hydrogen, C₁-C₄ alkyl, hydroxyalkyl, (amino)alkyl, (heterocyclo)alkyl, optionally substituted C₃-C₆ cycloalkyl, optionally substituted 4- to 8-membered heterocyclo, —(CH₂)ₘ—C(═O)N(R^{13a})(R^{13b}), and —(CH₂)ₙ—N(H)C(═O)R¹⁵;
m is 0 or 1; and
n is 0 or 1,
or a pharmaceutically acceptable salt or solvate thereof.

17. The compound of claim 16, wherein R^{10d} and R^{10e} are independently selected from the group consisting of hydrogen, C₁-C₃ alkyl, (amino)alkyl, and hydroxyalkyl, or a pharmaceutically acceptable salt or solvate thereof.

18. (canceled)

19. The compound of claim 1 of Formula VIII: 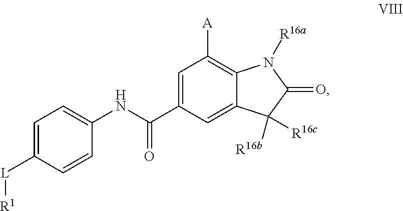
or a pharmaceutically acceptable salt or solvate thereof, wherein
R¹ is —CF₃ or —CF₂Cl; and
A is

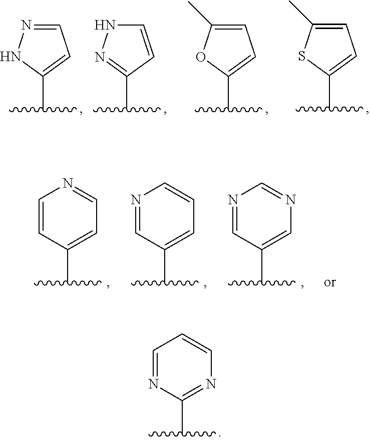

20. The compound of claim 19, wherein R^{16b} and R^{16c} are independently C₁-C₄ alkyl; and R^{16a} is selected from the group consisting of C₁-C₆ alkyl and C₃-C₆ cycloalkyl, or a pharmaceutically acceptable salt or solvate thereof.

21. (canceled)

22. The compound of claim 1 of Formula IX: 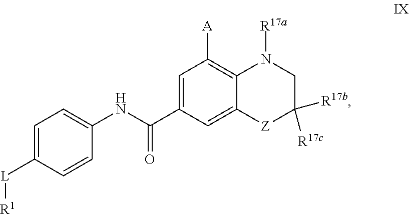
or a pharmaceutically acceptable salt or solvate thereof, wherein
R¹ is —CF₃ or —CF₂Cl; and
A is

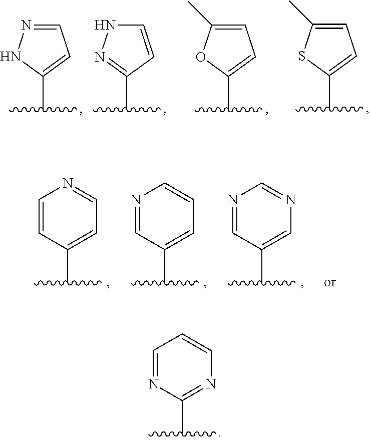

23. The compound of claim 22, wherein R^{17b} and R^{17c} are independently selected from the group consisting of hydrogen and C₁-C₄ alkyl; R^{17a} is C₁-C₆ alkyl; and Z is —O— or —CH₂—, or a pharmaceutically acceptable salt or solvate thereof.

24. (canceled)

25. (canceled)

26. The compound of claim 1 of Formula X: 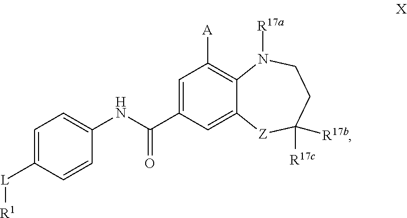
or a pharmaceutically acceptable salt or solvate thereof, wherein
R¹ is —CF₃ or —CF₂Cl; and
A is

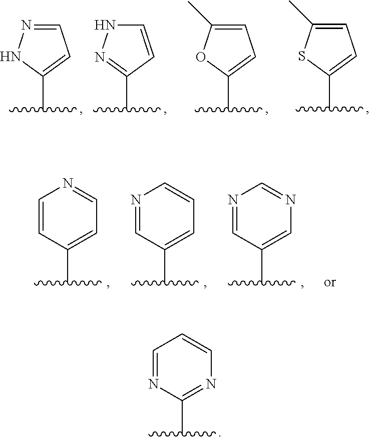

27. The compound of claim 26, wherein R^{17a} is C₁-C₆ alkyl; R^{17b} and R^{17c} are independently selected from the group consisting of hydrogen and C₁-C₄ alkyl; and Z is —C(R^{17e})(R^{17f})—, or a pharmaceutically acceptable salt or solvate thereof.

28. (canceled)

29. (canceled)

30. The compound of claim 1 of Formula XI: 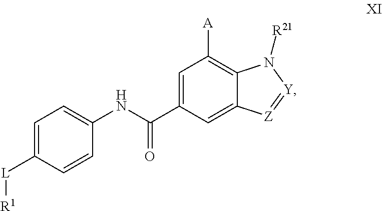
or a pharmaceutically acceptable salt or solvate thereof, wherein
R¹ is —CF₃ or —CF₂Cl; and
A is 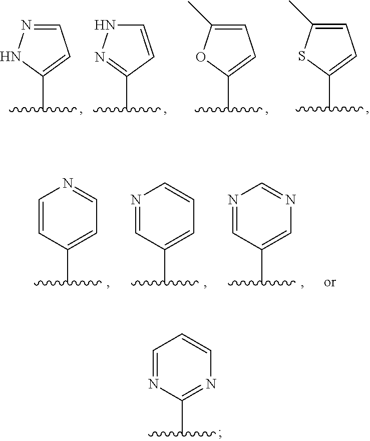
R²¹ is selected from the group consisting of hydrogen, C₁-C₆ alkyl, and optionally substituted C₃-C₆ cycloalkyl, or a pharmaceutically acceptable salt or solvate thereof.

31. The compound of claim 1 of Formula XII: 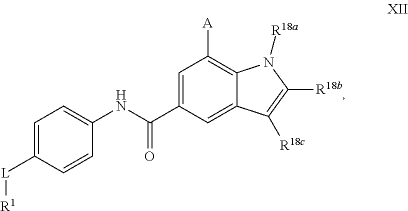
or a pharmaceutically acceptable salt or solvate thereof, wherein
R¹ is —CF₃ or —CF₂Cl; and
A is

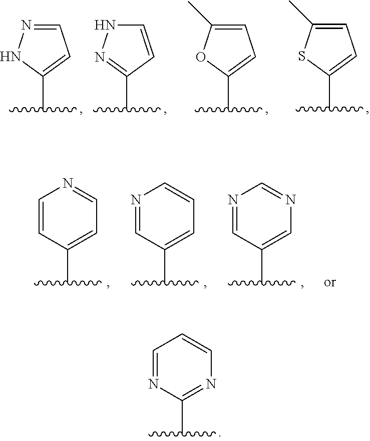

32. The compound of claim 31, wherein R^{18a} and R^{18b} are independently selected from the group consisting of hydrogen and C₁₋₄ alkyl, or a pharmaceutically acceptable salt or solvate thereof.

33. The compound of claim 1 of Formula XIII: 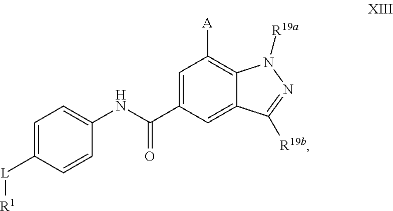
or a pharmaceutically acceptable salt or solvate thereof, wherein
R¹ is —CF₃ or —CF₂Cl; and
A is

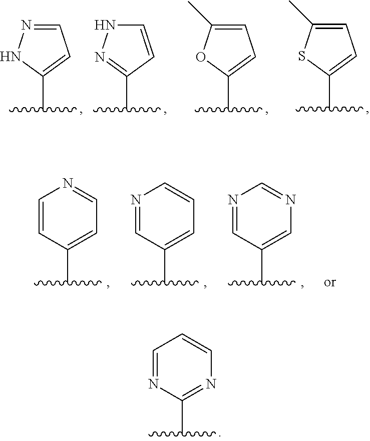

34. The compound of claim 33, wherein R^{19b} is selected from the group consisting of hydrogen and C₁₋₄ alkyl, or a pharmaceutically acceptable salt or solvate thereof.

35.-43. (canceled)

44. The compound of claim 1 selected from the group consisting of
N-(4-(chlorodifluoromethoxy)phenyl)-1-isopropyl-3-methyl-4-(1H-pyrazol-5-yl)indoline-6-carboxamide;
N-(4-(chlorodifluoromethoxy)phenyl)-1,3,3-trimethyl-4-(1H-pyrazol-5-yl)indoline-6-carboxamide;
N-(4-(chlorodifluoromethoxy)phenyl)-3,3-dimethyl-4-(1H-pyrazol-5-yl)indoline-6-carboxamide;
1-acetyl-N-(4-(chlorodifluoromethoxy)phenyl)-3,3-dimethyl-4-(1H-pyrazol-5-yl)indoline-6-carboxamide;
N-(4-(chlorodifluoromethoxy)phenyl)-3,3-dimethyl-1-(methylsulfonyl)-4-(1H-pyrazol-5-yl)indoline-6-carboxamide;
N-(4-(chlorodifluoromethoxy)phenyl)-1,1,2-trimethyl-3-oxo-7-(1H-pyrazol-5-yl)isoindoline-5-carboxamide;
N-(4-(chlorodifluoromethoxy)phenyl)-1,1,2-trimethyl-7-(5-methylfuran-2-yl)-3-oxoisoindoline-5-carboxamide;
N-(4-(chlorodifluoromethoxy)phenyl)-1,1,2-trimethyl-7-(5-methylthiophen-2-yl)-3-oxoisoindoline-5-carboxamide;
N-(4-(chlorodifluoromethoxy)phenyl)-1,1,2-trimethyl-3-oxo-7-(pyridin-4-yl)isoindoline-5-carboxamide;
N-(4-(chlorodifluoromethoxy)phenyl)-2-(2-hydroxyethyl)-1,1-dimethyl-3-oxo-7-(1H-pyrazol-5-yl)isoindoline-5-carboxamide;
N-(4-(chlorodifluoromethoxy)phenyl)-1,1-dimethyl-2-(2-(4-methylpiperazin-1-yl)ethyl)-3-oxo-7-(1H-pyrazol-5-yl)isoindoline-5-carboxamide;
N-(4-(chlorodifluoromethoxy)phenyl)-2-(2-(1,1-dioxidothiomorpholino)ethyl)-1,1-dimethyl-3-oxo-7-(1H-pyrazol-5-yl)isoindoline-5-carboxamide;
(R)—N-(4-(chlorodifluoromethoxy)phenyl)-2-(2-(3-hydroxypyrrolidin-1-yl)ethyl)-1,1-dimethyl-3-oxo-7-(1H-pyrazol-5-yl)isoindoline-5-carboxamide;
N-(4-(chlorodifluoromethoxy)phenyl)-1,1,2-trimethyl-3-oxo-7-(pyridin-3-yl)isoindoline-5-carboxamide;
N-(4-(chlorodifluoromethoxy)phenyl)-3,3-dimethyl-2-oxo-4-(1H-pyrazol-5-yl)indoline-6-carboxamide;
N-(4-(chlorodifluoromethoxy)phenyl)-1,3,3-trimethyl-2-oxo-4-(1H-pyrazol-5-yl)indoline-6-carboxamide;
N-(4-(chlorodifluoromethoxy)phenyl)-2′-oxo-4′-(1H-pyrazol-5-yl)spiro[cyclopentane-1,3′-indoline]-6′-carboxamide;
N-(4-(chlorodifluoromethoxy)phenyl)-2′-oxo-4′-(1H-pyrazol-5-yl)spiro[cyclopentane-1,3′-indolin]-3-ene-6′-carboxamide;
N-(4-(chlorodifluoromethoxy)phenyl)-3-hydroxy-2′-oxo-4′-(1H-pyrazol-3-yl)spiro[cyclopentane-1,3′-indoline]-6′-carboxamide;
N-(4-(chlorodifluoromethoxy)phenyl)-1-(2-hydroxyethyl)-3,3-dimethyl-2-oxo-4-(1H-pyrazol-5-yl)indoline-6-carboxamide;
N-(4-(chlorodifluoromethoxy)phenyl)-1-(2-(dimethylamino)ethyl)-3,3-dimethyl-2-oxo-4-(1H-pyrazol-5-yl)indoline-6-carboxamide;
N-(4-(chlorodifluoromethoxy)phenyl)-1′-methyl-2-oxo-4-(1H-pyrazol-5-yl)spiro[indoline-3,3′-pyrrolidine]-6-carboxamide;
(3R,4S)—N-(4-(chlorodifluoromethoxy)phenyl)-3,4-dihydroxy-2′-oxo-4′-(1H-pyrazol-5-yl)spiro[cyclopentane-1,3′-indoline]-6′-carboxamide;
N-(4-(chlorodifluoromethoxy)phenyl)-1′-methyl-2-oxo-4-(1H-pyrazol-5-yl)spiro[indoline-3,4′-piperidine]-6-carboxamide;
N-(4-(chlorodifluoromethoxy)phenyl)-2′-oxo-4′-(1H-pyrazol-5-yl)spiro[cyclohexane-1,3′-indoline]-6′-carboxamide;
N-(4-(chlorodifluoromethoxy)phenyl)-1-isopropyl-7-(1H-pyrazol-5-yl)indoline-5-carboxamide;
N-(4-(chlorodifluoromethoxy)phenyl)-1-cyclopentyl-7-(1H-pyrazol-5-yl)indoline-5-carboxamide;
N-(4-(chlorodifluoromethoxy)phenyl)-1-cyclohexyl-7-(1H-pyrazol-5-yl)indoline-5-carboxamide;
N-(4-(chlorodifluoromethoxy)phenyl)-1-isopropyl-7-(pyrimidin-5-yl)indoline-5-carboxamide;
N-(4-(chlorodifluoromethoxy)phenyl)-1-cyclopropyl-7-(1H-pyrazol-5-yl)indoline-5-carboxamide;
N-(4-(chlorodifluoromethoxy)phenyl)-1-(2-hydroxyethyl)-7-(1H-pyrazol-5-yl)indoline-5-carboxamide;
N-(4-(chlorodifluoromethoxy)phenyl)-1-methyl-7-(1H-pyrazol-5-yl)indoline-5-carboxamide;
N-(4-(chlorodifluoromethoxy)phenyl)-1-cyclobutyl-7-(1H-pyrazol-5-yl)indoline-5-carboxamide;
N-(4-(chlorodifluoromethoxy)phenyl)-1-(methylsulfonyl)-7-(1H-pyrazol-5-yl)indoline-5-carboxamide;
N-(4-(chlorodifluoromethoxy)phenyl)-1-isobutyryl-7-(1H-pyrazol-5-yl)indoline-5-carboxamide;
N-(4-(chlorodifluoromethoxy)phenyl)-1-(3-(chloromethyl)cyclobutyl)-7-(1H-pyrazol-5-yl)indoline-5-carboxamide;
N-(4-(chlorodifluoromethoxy)phenyl)-1-(3-(hydroxymethyl)cyclobutyl)-7-(1H-pyrazol-5-yl)indoline-5-carboxamide;
N-(4-(chlorodifluoromethoxy)phenyl)-2-((dimethylamino)methyl)-1-isopropyl-7-(1H-pyrazol-5-yl)indoline-5-carboxamide;
N-(4-(chlorodifluoromethoxy)phenyl)-2-(3-hydroxyazetidine-1-carbonyl)-1-isopropyl-7-(pyrimidin-5-yl)indoline-5-carboxamide;
N5-(4-(chlorodifluoromethoxy)phenyl)-1-isopropyl-N2,N2-dimethyl-7-(pyrimidin-5-yl)indoline-2,5-dicarboxamide;
N5-(4-(chlorodifluoromethoxy)phenyl)-1-isopropyl-N2-methyl-7-(pyrimidin-5-yl)indoline-2,5-dicarboxamide;
N-(4-(chlorodifluoromethoxy)phenyl)-1-isopropyl-2-((methylamino)methyl)-7-(1H-pyrazol-5-yl)indoline-5-carboxamide;
N-(4-(chlorodifluoromethoxy)phenyl)-1-isopropyl-2-((N-methylacetamido)methyl)-7-(1H-pyrazol-5-yl)indoline-5-carboxamide;
N-(4-(chlorodifluoromethoxy)phenyl)-2-(hydroxymethyl)-1-isopropyl-3-methyl-7-(1H-pyrazol-5-yl)indoline-5-carboxamide;
N-(4-(chlorodifluoromethoxy)phenyl)-3-ethyl-2-(hydroxymethyl)-1-isopropyl-7-(1H-pyrazol-5-yl)indoline-5-carboxamide;
5-((4-(chlorodifluoromethoxy)phenyl)carbamoyl)-1-isopropyl-7-(pyrimidin-5-yl)indoline-2-carboxylic acid;
N5-(4-(chlorodifluoromethoxy)phenyl)-1-isopropyl-N2-(2-(methylsulfonyl)ethyl)-7-(pyrimidin-5-yl)indoline-2,5-dicarboxamide;
N-(4-(chlorodifluoromethoxy)phenyl)-1-isopropyl-2-(morpholine-4-carbonyl)-7-(pyrimidin-5-yl)indoline-5-carboxamide;
N-(4-(chlorodifluoromethoxy)phenyl)-3-ethyl-1-isopropyl-2-((methylamino)methyl)-7-(1H-pyrazol-5-yl)indoline-5-carboxamide;
N-(4-(chlorodifluoromethoxy)phenyl)-1-isopropyl-2-(morpholine-4-carbonyl)-7-(1H-pyrazol-5-yl)indoline-5-carboxamide;
N5-(4-(chlorodifluoromethoxy)phenyl)-1-isopropyl-N2,N2-dimethyl-7-(1H-pyrazol-5-yl)indoline-2,5-dicarboxamide;
N-(4-(chlorodifluoromethoxy)phenyl)-3-((dimethylamino)methyl)-1-isopropyl-7-(1H-pyrazol-5-yl)indoline-5-carboxamide;
N-(4-(chlorodifluoromethoxy)phenyl)-1-isopropyl-3-((methylamino)methyl)-7-(1H-pyrazol-5-yl)indoline-5-carboxamide;
N-(4-(chlorodifluoromethoxy)phenyl)-2-((R)-3-hydroxypyrrolidine-1-carbonyl)-1-isopropyl-7-(1H-pyrazol-5-yl)indoline-5-carboxamide;
N5-(4-(chlorodifluoromethoxy)phenyl)-1-isopropyl-N2,N2-bis(2-methoxyethyl)-7-(1H-pyrazol-5-yl)indoline-2,5-dicarboxamide;
N-(4-(chlorodifluoromethoxy)phenyl)-2-(1,1-dioxidothiomorpholine-4-carbonyl)-1-isopropyl-7-(1H-pyrazol-5-yl)indoline-5-carboxamide;
N5-(4-(chlorodifluoromethoxy)phenyl)-N2-(2-(dimethylamino)ethyl)-1-isopropyl-7-(1H-pyrazol-5-yl)indoline-2,5-dicarboxamide;
N-(4-(chlorodifluoromethoxy)phenyl)-1-isopropyl-2-(4-methylpiperazine-1-carbonyl)-7-(1H-pyrazol-5-yl)indoline-5-carboxamide;
N-(4-(chlorodifluoromethoxy)phenyl)-2-(hydroxymethyl)-1-isopropyl-7-(1H-pyrazol-5-yl)indoline-5-carboxamide;
N-(4-(chlorodifluoromethoxy)phenyl)-1-isopropyl-2-(2-morpholino-2-oxoethyl)-7-(1H-pyrazol-5-yl)indoline-5-carboxamide;
N-(4-(chlorodifluoromethoxy)phenyl)-2-(2-(dimethylamino)-2-oxoethyl)-1-isopropyl-7-(1H-pyrazol-5-yl)indoline-5-carboxamide;
N-(4-(chlorodifluoromethoxy)phenyl)-1-isopropyl-3-methyl-7-(pyrimidin-5-yl)indoline-5-carboxamide;
N-(4-(chlorodifluoromethoxy)phenyl)-1-isopropyl-3-methyl-7-(1H-pyrazol-5-yl)indoline-5-carboxamide;
N-(4-(chlorodifluoromethoxy)phenyl)-1,3-dimethyl-7-(1H-pyrazol-5-yl)indoline-5-carboxamide;
N-(4-(chlorodifluoromethoxy)phenyl)-1-isopropyl-3,3-dimethyl-7-(1H-pyrazol-5-yl)indoline-5-carboxamide;
N-(4-(chlorodifluoromethoxy)phenyl)-3-(hydroxymethyl)-1-isopropyl-3-methyl-7-(1H-pyrazol-5-yl)indoline-5-carboxamide;
N-(4-(chlorodifluoromethoxy)phenyl)-1,3-diisopropyl-7-(1H-pyrazol-5-yl)indoline-5-carboxamide;
N-(4-(chlorodifluoromethoxy)phenyl)-3-(hydroxymethyl)-1-isopropyl-7-(1H-pyrazol-5-yl)indoline-5-carboxamide;
1-methyl-7-(1H-pyrazol-5-yl)-N-(4-((trifluoromethyl)thio)phenyl)indoline-5-carboxamide;
1-isopropyl-7-(1H-pyrazol-5-yl)-N-(4-((trifluoromethyl)thio)phenyl)indoline-5-carboxamide;
1-isopropyl-7-(1H-pyrazol-5-yl)-N-(4-(trifluoromethoxy)phenyl)indoline-5-carboxamide;
N-(4-(chlorodifluoromethoxy)phenyl)-1,3,3-trimethyl-2-oxo-7-(1H-pyrazol-5-yl)indoline-5-carboxamide;
N-(4-(chlorodifluoromethoxy)phenyl)-1-isopropyl-3,3-dimethyl-2-oxo-7-(pyrimidin-5-yl)indoline-5-carboxamide;
N-(4-(chlorodifluoromethoxy)phenyl)-1-isopropyl-3,3-dimethyl-2-oxo-7-(1H-pyrazol-5-yl)indoline-5-carboxamide;
N-(4-(chlorodifluoromethoxy)phenyl)-1-isopropyl-8-(1H-pyrazol-5-yl)-1,2,3,4-tetrahydroquinoline-6-carboxamide;
N-(4-(chlorodifluoromethoxy)phenyl)-1-methyl-8-(1H-pyrazol-5-yl)-1,2,3,4-tetrahydroquinoline-6-carboxamide;
N-(4-(chlorodifluoromethoxy)phenyl)-4-isopropyl-5-(1H-pyrazol-5-yl)-3,4-dihydro-2H-benzo[b][1,4]oxazine-7-carboxamide;
N-(4-(chlorodifluoromethoxy)phenyl)-4-isopropyl-2-methyl-5-(1H-pyrazol-5-yl)-3,4-dihydro-2H-benzo[b][1,4]oxazine-7-carboxamide;
N-(4-(chlorodifluoromethoxy)phenyl)-4-isopropyl-2,2-dimethyl-5-(1H-pyrazol-5-yl)-3,4-dihydro-2H-benzo[b][1,4]oxazine-7-carboxamide;
4-isopropyl-5-(1H-pyrazol-5-yl)-N-(4-(trifluoromethoxy)phenyl)-3,4-dihydro-2H-benzo[b][1,4]oxazine-7-carboxamide;
N-(4-(chlorodifluoromethoxy)phenyl)-1-isopropyl-9-(1H-pyrazol-5-yl)-2,3,4,5-tetrahydro-1H-benzo[b]azepine-7-carboxamide;
N-(4-(chlorodifluoromethoxy)phenyl)-1-isopropyl-7-(1H-pyrazol-5-yl)-1H-indole-5-carboxamide;
N-(4-(chlorodifluoromethoxy)phenyl)-1-isopropyl-7-(pyrimidin-5-yl)-1H-indole-5-carboxamide;
3-chloro-N-(4-(chlorodifluoromethoxy)phenyl)-1-isopropyl-7-(1H-pyrazol-5-yl)-1H-indole-5-carboxamide;
3-chloro-N-(4-(chlorodifluoromethoxy)phenyl)-1-isopropyl-7-(pyrimidin-5-yl)-1H-indole-5-carboxamide;
N-(4-(chlorodifluoromethoxy)phenyl)-1-isopropyl-3-methyl-7-(pyrimidin-5-yl)-1H-indole-5-carboxamide;
N-(4-(chlorodifluoromethoxy)phenyl)-1-isopropyl-3-methyl-7-(1H-pyrazol-5-yl)-1H-indole-5-carboxamide;
N-(4-(chlorodifluoromethoxy)phenyl)-3-(hydroxymethyl)-1-isopropyl-7-(pyrimidin-5-yl)-1H-indole-5-carboxamide;
N-(4-(chlorodifluoromethoxy)phenyl)-3-(hydroxymethyl)-1-isopropyl-7-(1H-pyrazol-5-yl)-1H-indole-5-carboxamide;
N-(4-(chlorodifluoromethoxy)phenyl)-1-isopropyl-7-(1H-pyrazol-5-yl)-1H-indazole-5-carboxamide;
N-(4-(chlorodifluoromethoxy)phenyl)-1-isopropyl-2-methyl-2-(morpholine-4-carbonyl)-7-(1H-pyrazol-5-yl)indoline-5-carboxamide;
N-(4-(chlorodifluoromethoxy)phenyl)-3,3-dimethyl-1-(2-(methylsulfonyl)ethyl)-2-oxo-4-(1H-pyrazol-5-yl)indoline-6-carboxamide;
N-(4-(chlorodifluoromethoxy)phenyl)-2′-oxo-4′-(1H-pyrazol-5-yl)spiro[cyclobutane-1,3′-indoline]-6′-carboxamide;
N-(4-(chlorodifluoromethoxy)phenyl)-2-oxo-4-(1H-pyrazol-5-yl)-2′,3′,5′,6′-tetrahydrospiro[indoline-3,4′-pyran]-6-carboxamide;
1′-acetyl-N-(4-(chlorodifluoromethoxy)phenyl)-2-oxo-4-(1H-pyrazol-5-yl)spiro[indoline-3,3′-pyrrolidine]-6-carboxamide;
1′-acetyl-N-(4-(chlorodifluoromethoxy)phenyl)-2-oxo-4-(pyridin-3-yl)spiro[indoline-3,3′-pyrrolidine]-6-carboxamide;
1′-acetyl-N-(4-(chlorodifluoromethoxy)phenyl)-2-oxo-4-(pyrimidin-5-yl)spiro[indoline-3,3′-pyrrolidine]-6-carboxamide;
N-(4-(chlorodifluoromethoxy)phenyl)-2-oxo-4-(1H-pyrazol-5-yl)-2′,3′,5′,6′-tetrahydrospiro[indoline-3,4′-thiopyran]-6-carboxamide 1′,1′-dioxide;
N-(4-(chlorodifluoromethoxy)phenyl)-2-oxo-4-(pyridin-3-yl)-2′,3′,5′,6′-tetrahydrospiro[indoline-3,4′-thiopyran]-6-carboxamide 1′,1′-dioxide;
N-(4-(chlorodifluoromethoxy)phenyl)-2-oxo-4-(pyrimidin-5-yl)-2′,3′,5′,6′-tetrahydrospiro[indoline-3,4′-thiopyran]-6-carboxamide 1′,1′-dioxide;
N-(4-(chlorodifluoromethoxy)phenyl)-2′-oxo-4′-(pyridin-3-yl)spiro[cyclohexane-1,3′-indoline]-6′-carboxamide;
N-(4-(chlorodifluoromethoxy)phenyl)-2′-oxo-4′-(pyrimidin-5-yl)spiro[cyclohexane-1,3′-indoline]-6′-carboxamide;
N-(4-(chlorodifluoromethoxy)phenyl)-2′-oxo-4′-(1H-pyrazol-5-yl)-4,5-dihydro-2H-spiro[furan-3,3′-indoline]-6′-carboxamide;
N-(4-(chlorodifluoromethoxy)phenyl)-1′-methyl-2-oxo-4-(1H-pyrazol-5-yl)spiro[indoline-3,4′-piperidine]-6-carboxamide;
1′-acetyl-N-(4-(chlorodifluoromethoxy)phenyl)-2-oxo-4-(1H-pyrazol-5-yl)spiro[indoline-3,4′-piperidine]-6-carboxamide; and
N-(4-(chlorodifluoromethoxy)phenyl)-1′-(methylsulfonyl)-2-oxo-4-(1H-pyrazol-5-yl)spiro[indoline-3,4′-piperidine]-6-carboxamide,
or a pharmaceutically acceptable salt or solvate thereof.

45. (canceled)

46. A method of treating cancer in a subject in need thereof, the method comprising administering to the subject a therapeutically effective amount of the compound of claim 1, or a pharmaceutically acceptable salt or solvate thereof.

47.-57. (canceled)
